(19) United States
(12) Patent Application Publication
(10) Pub. No.: US 2022/0211743 A1
(43) Pub. Date: Jul. 7, 2022

(54) ORAL DELIVERY OF OLIGONUCLEOTIDES

(71) Applicant: ALNYLAM PHARMACEUTICALS, INC., Cambridge, MA (US)

(72) Inventors: Jayaprakash K. NAIR, Cambridge, MA (US); Xiaojun QIN, Cambridge, MA (US); Mikyung YU, Cambridge, MA (US); Vasant JADHAV, Cambridge, MA (US); Martin MAIER, Cambridge, MA (US); Diane RAMSDEN, Cambridge, MA (US)

(21) Appl. No.: 17/611,761

(22) PCT Filed: May 15, 2020
(86) PCT No.: PCT/US2020/033156

§ 371 Date: Nov. 16, 2021

Related U.S. Application Data
(60) Provisional application No. 62/911,512, filed on Oct. 7, 2019.
(60) Provisional application No. 62/849,605, filed on May 17, 2019.

(51) Int. Cl. A61K 31/713; C12N 15/113; A61K 9/00

(52) U.S. Cl. CPC A61K 31/713; C12N 15/113; A61K 9/0053; C12N 2310/14; C12N 2320/35; C12N 2310/315; C12N 2310/344; C12N 2310/346; C12N 2320/32; C12N 2310/312

(57) ABSTRACT

One aspect of the present invention relates to an oral formulation for reducing or inhibiting the expression of a target gene in a subject, comprising a) double stranded iRNA agent comprising an antisense strand which is complementary to a target gene; a sense strand which is complementary to said antisense strand; 2′-OMe modifications to more than fifteen, more than twenty, more than twenty-five, or more than thirty nucleotides; and a carbohydrate-based ligand conjugated to at least one strand, optionally via a linker or carrier; and b) a penetration enhancer. Another aspect of the invention relates to a method of gene silencing, comprising orally administering to a subject in need thereof the oral formulation.

This application claims benefit of priority to U.S. Provisional Application No. 62/849,605 filed May 17, 2019; and U.S. Provisional Application No. 62/911,512 filed Oct. 7, 2019, which are herein incorporated by reference in their entirety.

### FIELD OF INVENTION

This invention relates to the field of oral delivery using ligand-conjugated oligonucleotides.

### BACKGROUND

To date, an efficient and effective oral delivery of an iRNA agent has remained a challenge. Despite continuous efforts in this area, sufficient oral bioavailability of intact and functional oligonucleotide (the fraction of an administered dose of unchanged drug that reaches the synstemic circulation) and the resulting therapeutic benefit have not yet been validated, limiting the use of siRNA-based therapies.

Oral administration of hydrophilic macromolecules with a molecular weight (MW) above 1000 Da, such as an iRNA agent, are particularly problematic due to combined challenges such as degradation at low gastric pH or by gastrointestinal enzymes, poor permeability across the gastrointestinal membranes, and the first-pass metabolism (the process of absorption in liver and gut wall).

One possible solution to the permeability problem may be to use intestinal permeation enhancers for oral delivery of drugs. For instance, medium chain fatty acid, such as sodium caprate (C10) has been reported to increase permeability of the cells in delivery of FITC-dextrans (MW 4K), polysucrose (MW 15K), and insulin. See Cano-Cebrian et al., Current Drug Delivery 2(1): 9-22 (2005). However, in that report, the effect was significant only for subtances of molecular weight of less than 1.2K; permeability enhancement would not result in a significant increase in larger molecules in dose fraction absorbed. Salcaprozate sodium (SNAC) and sodium caprate (C10) have been used as permeation enhancers for oral delivery of peptides and proteins. See Twarog et al., Pharmaceutics 11(2): E78 (2019). However, to this date, the formulation using permeation enhancers for oral delivery of macromolecules and appropriate dose regimen are still not clear. An efficient and effective oral delivery of an iRNA agent at clinically relevant dose regimen has yet to be established.

Thus, there is a continuing need for new and improved methods for oral delivery of siRNA molecules in vivo, to achieve and enhance the therapeutic potential of iRNA agents.

### SUMMARY

One aspect of the invention relates to an oral formulation for reducing or inhibiting the expression of a target gene in a subject. The oral formulation comprises a) a double-stranded iRNA agent and b) a penetration enhancer. The double-stranded iRNA agent comprises an antisense strand which is complementary to the target gene; a sense strand which is complementary to said antisense strand; a carbohydrate-based ligand conjugated to at least one of the strands, optionally via a linker or carrier. The double-stranded iRNA agent also comprises 2′-OMe modifications to more than fifteen, more than twenty, more than twenty-five, or more than thirty nucleotides.

In some embodiments, the concentration of the penetration enhancer in the oral formulation is no more than about 200 mM, for instance, no more than about 150 mM, no more than about 100 mM, no more than about 80 mM, no more than about 60 mM, no more than about 50 mM, no more than about 45 mM, no more than about 40 mM, no more than about 35 mM, or no more than about 30 mM.

The carbohydrate-based ligand may be conjugated to the iRNA agent via a direct attachment to the ribosugar of the iRNA agent. Alternatively, the carbohydrate-based ligand may be conjugated to the iRNA agent via a linker or a carrier.

In certain embodiments, the carbohydrate-based ligand is conjugated to the iRNA agent via one or more linkers (tethers).

In some embodiments, the carbohydrate-based ligand is conjugated to the double-stranded iRNA agent via a linker a linker containing an ether, thioether, urea, carbonate, amine, amide, maleimide-thioether, disulfide, phosphodiester, sulfonamide linkage, a product of a click reaction (e.g., a triazole from the azide-alkyne cycloaddition), or carbamate.

In some embodiments, at least one of the linkers (tethers) is a redox cleavable linker (such as a reductively cleavable linker; e.g., a disulfide group), an acid cleavable linker (e.g., a hydrazone group, an ester group, an acetal group, or a ketal group), an esterase cleavable linker (e.g., an ester group), a phosphatase cleavable linker (e.g., a phosphate group), or a peptidase cleavable linker (e.g., a peptide bond).

In other embodiments, at least one of the linkers (tethers) is a bio-clevable linker selected from the group consisting of DNA, RNA, disulfide, amide, functionalized monosaccharides or oligosaccharides of galactosamine, glucosamine, glucose, galactose, mannose, and combinations thereof.

In certain embodiments, the carbohydrate-based ligand is conjugated to the double-stranded iRNA agent via a carrier that replaces one or more nucleotide(s). The carrier can be a cyclic group or an acyclic group. In one embodiment, the cyclic group is selected from the group consisting of cyclohexyl, pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl, and decalin. In one embodiment, the acyclic group is a moiety based on a serinol backbone or a diethanolamine backbone.

In some embodiments, the carrier replaces one or more nucleotide(s) in the internal position(s) of the double-stranded iRNA agent.

In other embodiments, the carrier replaces the nucleotides at the terminal end of the sense strand or antisense strand. In one embodiment, the carrier replaces the terminal nucleotide on the 3′ end of the sense strand, thereby functioning as an end cap protecting the 3′ end of the sense strand. In one embodiment, the carrier is a cyclic group having an amine, for instance, the carrier may be cyclohexyl, pyrrolidinyl, pyrazolinyl, pyrazolidinyl, imidazolinyl, imidazolidinyl, piperidinyl, piperazinyl, [1,3]dioxolanyl, oxazolidinyl, isoxazolidinyl, morpholinyl, thiazolidinyl, isothiazolidinyl, quinoxalinyl, pyridazinonyl, tetrahydrofuranyl, or decalinyl.

In some embodiments, the carbohydrate-based ligand is attached to the terminal end of a sense or antisense strand. In one embodiment, the carbohydrate-based ligand is attached to 3′ end of the antisense strand. In one embodiment, the carbohydrate-based ligand is attached to 5′ end of the antisense strand. In one embodiment, the carbohydrate-based ligand is attached to 5′ end of the sense strand. In one embodiment, the carbohydrate-based ligand is attached to 3′ end of the sense strand.

In some embodiments, the carbohydrate-based ligand is D-galactose, multivalent galactose, N-acetyl-D-galactosamine (GalNAc), multivalent GalNAc, D-mannose, multivalent mannose, multivalent lactose, N-acetyl-glucosamine, glucose, multivalent glucose, multivalent fucose, glycosylated polyaminoacids, or lectins.

In some embodiments, the carbohydrate-based ligand is an ASGPR ligand. For example, the ASGPR ligand is one or more GalNAc derivatives attached through a bivalent or trivalent branched linker, such as:

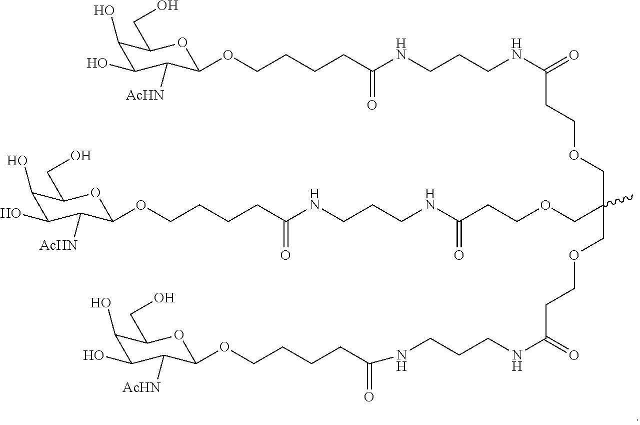 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In one embodiment, the ASGPR ligand is attached to the 3′ end or the 5′ end of the sense strand.

In some embodiments, the sense and antisense strands of the double-stranded iRNA agent are each 15 to 30 nucleotides in length. In one embodiment, the sense and antisense strands of a double-stranded iRNA agent are each 19 to 25 nucleotides in length. In one embodiment, the sense and antisense strands of the double-stranded iRNA agent are each 21 to 23 nucleotides in length.

In some embodiments, the double-stranded iRNA agent comprises a single-stranded overhang on at least one of the termini, e.g., 3′ and/or 5′ overhang(s) of 1-10 nucleotides in length, for instance, an overhang of 1, 2, 3, 4, 5, or 6 nucleotides. In some embodiments, both strands have at least one stretch of 1-5 (e.g., 1, 2, 3, 4, or 5) single-stranded nucleotides in the double stranded region. In one embodiment, the single-stranded overhang is 1, 2, or 3 nucleotides in length on at least one of the termini.

In some embodiments, the double-stranded iRNA agent may also have a blunt end, located at the 5′-end of the antisense strand (or the 3′-end of the sense strand), or vice versa. In one embodiment, the double-stranded iRNA agent comprises a 3′ overhang at the 3′-end of the antisense strand, and optionally a blunt end at the 5′-end of the antisense strand. In one embodiment, the double-stranded iRNA agent has a 5′ overhang at the 5′-end of the sense strand, and optionally a blunt end at the 5′-end of the antisense strand. In one embodiment, the double-stranded iRNA agent has two blunt ends at both ends of the iRNA duplex.

In one embodiment, the sense strand of the double-stranded iRNA agent is 21-nucleotides in length, and the antisense strand is 23-nucleotides in length, wherein the strands form a double-stranded region of 21 consecutive base pairs having a 2-nucleotide long single-stranded overhangs at the 3′-end.

In some embodiments, the carbohydrate-based ligand is conjugated to a nucleobase, sugar moiety, or internucleosidic linkage of the double-stranded iRNA agent.

In some embodiments, the double-stranded iRNA agent further comprises a phosphate mimic at the 5′-end of a strand, either at the sense strand or antisense strand or both. In one embodiment, the phosphate mimic is at the 5′ end of the antisense strand.

The phosphate mimic can be 5′-end phosphorodithioate (5′-PS₂), 5′-end vinylphosphonate (5′-VP), 5′-end methylphosphonate (MePhos), or 5′-deoxy-5′-C-malonyl

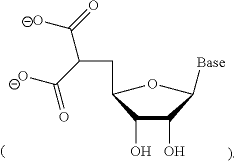

In one embodiment, the phosphate mimic is a 5′-vinylphosphonate (VP). The 5′-VP can be either 5′-E-VP isomer (i.e., trans-vinylphosphate,

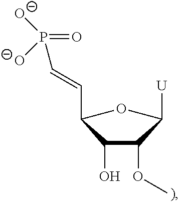

5′-Z-VP isomer (i.e., cis-vinylphosphate,

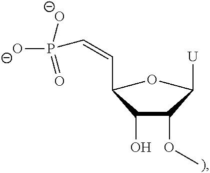

or mixtures thereof.

In some embodiments, the 5′-end of a strand, either the sense strand or the antisense strand or both strands, of the double-stranded iRNA agent does not contain a 5′-vinyl phosphonate (VP).

In some embodiments, the double-stranded iRNA agent further comprises at least one terminal, chiral phosphorus atom.

A site specific, chiral modification to the internucleotide linkage may occur at the 5′ end, 3′ end, or both the 5′ end and 3′ end of a strand. This is being referred to herein as a “terminal” chiral modification. The terminal modification may occur at a 3′ or 5′ terminal position in a terminal region, e.g., at a position on a terminal nucleotide or within the last 2, 3, 4, 5, 6, 7, 8, 9 or 10 nucleotides of a strand. A chiral modification may occur on the sense strand, antisense strand, or both the sense strand and antisense strand. Each of the chiral pure phosphorus atoms may be in either Rp configuration or Sp configuration, and combination thereof. More details regarding chiral modifications and chirally-modified dsRNA agents can be found in PCT/US18/67103, entitled “Chirally-Modified Double-Stranded RNA Agents,” filed Dec. 21, 2018, which is incorporated herein by reference in its entirety.

In some embodiments, the double-stranded iRNA agent further comprises a terminal, chiral modification occurring at the first internucleotide linkage at the 3′ end of the antisense strand, having the linkage phosphorus atom in Sp configuration; a terminal, chiral modification occurring at the first internucleotide linkage at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp configuration or Sp configuration.

In one embodiment, the double-stranded iRNA agent further comprises a terminal, chiral modification occurring at the first and second internucleotide linkages at the 3′ end of the antisense strand, having the linkage phosphorus atom in Sp configuration; a terminal, chiral modification occurring at the first internucleotide linkage at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp or Sp configuration.

In one embodiment, the double-stranded iRNA agent further comprises a terminal, chiral modification occurring at the first, second, and third internucleotide linkages at the 3′ end of the antisense strand, having the linkage phosphorus atom in Sp configuration; a terminal, chiral modification occurring at the first internucleotide linkage at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp or Sp configuration.

In one embodiment, the double-stranded iRNA agent further comprises a terminal, chiral modification occurring at the first and second internucleotide linkages at the 3′ end of the antisense strand, having the linkage phosphorus atom in Sp configuration; a terminal, chiral modification occurring at the third internucleotide linkages at the 3′ end of the antisense strand, having the linkage phosphorus atom in Rp configuration; a terminal, chiral modification occurring at the first internucleotide linkage at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp or Sp configuration.

In one embodiment, the double-stranded iRNA agent further comprises a terminal, chiral modification occurring at the first and second internucleotide linkages at the 3′ end of the antisense strand, having the linkage phosphorus atom in Sp configuration; a terminal, chiral modification occurring at the first, and second internucleotide linkages at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp or Sp configuration.

In some embodiments, the double-stranded iRNA agent has at least two blocks of two consecutive phosphorothioate or methylphosphonate internucleotide linkage modifications.

In some embodiments, the antisense strand comprises two blocks of one, two, or three phosphorothioate internucleotide linkages separated by 1, 2, 3, 4, 5, 6, 7, 8, 9, 10, 11, 12, 13, 14, 15, 16, 17, or 18 phosphate internucleotide linkages.

In some embodiments, the antisense strand comprises at least two consecutive phosphorothioate internucleotide linkage modifications within positions 18-23 of the antisense strand, counting from the 5′-end of the antisense strand. In some embodiments, the sense strand comprises at least two consecutive phosphorothioate internucleotide linkage modifications within position 1-5 of the sense strand, counting from the 5′-end of the sense strand. In one embodiment, the antisense strand comprises at least two consecutive phosphorothioate internucleotide linkage modifications within positions 18-23 of the antisense strand, counting from the 5′-end of the antisense strand; and the sense strand comprises at least two consecutive phosphorothioate internucleotide linkage modifications within position 1-5 of the sense strand, counting from the 5′-end of the sense strand.

In some embodiments, the double-stranded iRNA agent has at least 15 nucleotides with a 2′-modification, selected from the group consisting of a 2′-O-alkyl, 2′-substituted alkoxy, 2′-substituted alkyl, 2′-O-allyl, ENA, and BNA/LNA modification. In one embodiment, the double-stranded iRNA agent has at least 15 nucleotides with a 2′-O-alkyl modification, such as 2′-O-methyl modification. The double-stranded iRNA agent can have 2′-OMe modifications to more than fifteen, more than twenty, more than twenty-five, or more than thirty nucleotides.

In some embodiments, the double-stranded iRNA agent comprises at least two blocks of two consecutive phosphorothioate or methylphosphonate internucleotide linkage modifications.

In some embodiments, 100%, 95%, 90%, 85%, 80%, 75%, 70%, 65%, 60%, 55%, 50%, 45%, 40%, 35% or 30% of all nucleotides of the double-stranded iRNA agent is modified. For example, when 50% of all nucleotides of the double-stranded iRNA agent is modified, 50% of all nucleotides of the double-stranded iRNA agent contain a modification as described herein.

In one embodiment, at least 50% of the nucleotides of the double-stranded iRNA agent is independently modified with 2′-O-methyl, 2′-O-allyl, 2′-deoxy, or 2′-fluoro.

In one embodiment, at least 50% of the nucleotides of the antisense is independently modified with LNA, CeNA, 2′-methoxyethyl, or 2′-deoxy.

In some embodiments, the double-stranded iRNA agent comprises less than 12, less than 11, less than 10, less than 9, less than 8, less than 7, less than 6, less than 5, less than 4, or less than 3 of 2′-F modifications. In some embodiments, the double-stranded iRNA agent has less than 12, less than 11, less than 10, less than 9, less than 8, less than 7, less than 6, less than 5, less than 4, less than 3, less than 2, or no 2′-F modifications on the sense strand. In some embodiments, the double-stranded iRNA agent has less than 12, less than 11, less than 10, less than 9, less than 8, less than 7, less than 6, less than 5, less than 4, less than 3, less than 2, or no 2′-F modifications on the antisense strand.

In some embodiments, the double-stranded iRNA agent has one or more 2′-F modifications on any position of the sense strand or antisense strand.

In some embodiments, the sense strand comprises at least four 2′-F modifications, for instance, at positions 7 and 9-11, counting from the 5′-end of the sense strand.

In some embodiments, the antisense strand comprises at least four 2′-F modifications, for instance, at positions 2, 6, 14, and 16, counting from the 5′-end of the antisense strand. In some embodiments, the antisense strand comprises at least six 2′-F modifications, for instance, at positions 2, 6, 8-9, 14, and 16, counting from the 5′-end of the antisense strand.

In some embodiments, the double-stranded iRNA agent has less than 20%, less than 15%, less than 10%, less than 5% non-natural nucleotide, or substantially no non-natural nucleotide. Examples of non-natural nucleotide include acyclic nucleotides, LNA, HNA, CeNA, 2′-O-methoxyalkyl (e.g., 2′-O-methoxymethyl, 2′-O-methoxyethyl, or 2′-O-2-methoxypropanyl), 2′-O-allyl, 2′-C-allyl, 2′-fluoro, 2′-O—N-methylacetamido (2′-O-NMA), a 2′-O-dimethylaminoethoxyethyl (2′-O-DMAEOE), 2′-O-aminopropyl (2′-O-AP), 2′-ara-F, L-nucleoside modification (such as 2′-modified L-nucleoside, e.g., 2′-deoxy-L-nucleoside), BNA abasic sugar, abasic cyclic and open-chain alkyl.

In some embodiments, the double-stranded iRNA agent has greater than 80%, greater than 85%, greater than 90%, greater than 95%, or virtually 100% natural nucleotides. For the purpose of these embodiments, natural nucleotides can include those having 2′-OH, 2′-deoxy, and 2′-OMe.

In one embodiment, the double-stranded iRNA agent comprises a sense strand and antisense strand each having a length of 15-30 nucleotides; at least two consecutive phosphorothioate internucleotide linkages within positions 18-23 on the antisense strand (counting from the 5′ end); wherein the duplex region is between 19 to 25 base pairs (preferably 19, 20, 21 or 22); wherein the double-stranded iRNA agent has less than 20%, less than 15%, less than 10%, less than 5% non-natural nucleotide, or substantially no non-natural nucleotide.

In one embodiment, the double-stranded iRNA agent comprises a sense strand and antisense strand each having a length of 15-30 nucleotides; at least two consecutive phosphorothioate internucleotide linkages within positions 18-23 on the antisense strand (counting from the 5′ end); wherein the duplex region is between 19 to 25 base pairs (preferably 19, 20, 21 or 22); wherein the double-stranded iRNA agent has greater than 80%, greater than 85%, greater than 95%, or virtually 100% natural nucleotides, such as those having 2′-OH, 2′-deoxy, or 2′-OMe.

In some embodiments, the penetration enhancer is selected from the group consisting of a fatty acid or pharmaceutically acceptable salt thereof, a fatty acid derivative or pharmaceutically acceptable salt thereof, a bile acid or pharmaceutically acceptable salt thereof, a chelating agent, a surfactant, a non-chelating non-surfactant agent, and a chitosan or derivative thereof.

In some embodiments, the penetration enhancer is a fatty acid or pharmaceutically acceptable salt thereof, selected from the group consisting of arachidonic acid, oleic acid, lauric acid, capric acid, caprylic acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, a C₁₋₁₀ alkyl ester, monoglyceride, diglyceride, and a pharmaceutically acceptable salt thereof.

In some embodiments, the penetration enhancer is caprylic acid (C8), capric acid (C10), lauric acid (C12), oleic acid (C18), or pharmaceutically acceptable salt thereof, such as a sodium salt of the aforementioned fatty acid.

In one embodiment, the penetration enhancer is salcaprozate sodium.

In one embodiment, the penetration enhancer is chitosan or trimethyl chitosan chloride.

In some embodiments, the oral formulation is adapted for delivery as a capsule, soft elastic gelatin capsule, hard gelatin capsule, caplet, aerosol, spray, solution, suspension, or an emulsion.

Another aspect of the invention relates to a method of reducing the expression of a target gene in a subject, comprising orally administering to the subject in need thereof a formulation comprising: a) a double-stranded iRNA agent and b) a penetration enhancer. The double-stranded iRNA agent comprises an antisense strand which is complementary to a target gene; a sense strand which is complementary to said antisense strand; and a carbohydrate-based ligand conjugated to at least one strand, optionally via a linker or carrier. The double-stranded iRNA agent also comprises 2′-OMe modifications to more than fifteen, more than twenty, more than twenty-five, or more than thirty nucleotides.

In some embodiments, the unit dose of the double-stranded iRNA agent is administered at no more than about 50 mg per kg body weight, for instance, no more than about 40 mg per kg body weight, no more than about 30 mg per kg body weight, no more than about 25 mg per kg body weight, no more than about 20 mg per kg body weight, no more than about 15 mg per kg body weight, no more than about 10 mg per kg body weight, no more than about 5 mg per kg body weight, no more than about 3 mg per kg body weight, no more than about 2 mg per kg body weight, no more than about 1 mg per kg body weight, no more than about 0.5 mg per kg body weight, or no more than about 0.1 mg per kg body weight. In some embodiments, the unit dose of the double-stranded iRNA agent is administered at about 1 to about 30 mg per kg body weight, for instance, about 3 to about 25 mg per kg body weight. In one embodiment, the dosage is calculated according to the oral bioavailability of the individual oligomer, to obtain a dosage that will allow maintenance of an effective concentration of the oligomer in the target tissue.

In some embodiments, the concentration of the penetration enhancer in the formulation is no more than about 200 mM, for instance, no more than about 150 mM, no more than about 100 mM, no more than about 80 mM, no more than about 60 mM, no more than about 50 mM, no more than about 45 mM, or no more than about 40 mM, no more than about 35 mM, or no more than about 30 mM.

In some embodiments, the formulation is adapted for delivery as a capsule, soft elastic gelatin capsule, hard gelatin capsule, caplet, aerosol, spray, solution, suspension, or an emulsion.

All the above embodiments relating to the double-stranded iRNA agent and their chemical modifications, including the carbohydrate-based ligand and their conjugation to the double-stranded iRNA agent, and the penetration enhancer in the first aspect of the invention relating to the oral formulation for reducing or inhibiting the expression of a target gene in a subject are suitable in this aspect of the invention relating to a method of reducing the expression of a target gene in a subject.

### BRIEF DESCRIPTION OF THE DRAWINGS

FIG. 1 is a graph showing the relative F12 levels following oral delivery to fasting mice of a dose of a formulation containing a GalNAc-siRNA of 3 mg/kg (with 45.4 mM C10 in solution), 10 mg/kg (with 150 mM C10), and 25 mg/kg (with 37.5 mM C10), respectively, at days 0, 2, and 5. A comparison was made employing the same siRNA administered subcutaneously at a single dose of 0.75 mg/kg.

FIG. 2 is a graph showing the relative F12 levels following oral delivery to fasting mice of a dose of a formulation containing a GalNAc-siRNA of 25 mg/kg (with 37.5 mM C10) at days 0, 2, and 5, as compared against the relative F12 levels following oral delivery to fasting mice of a dose of the same siRNA of 25 mg/kg (without C10) at days 0, 2, and 5. A comparison was also made employing the same siRNA administered subcutaneously at a single dose of 0.75 mg/kg.

FIG. 3 is a graph showing the relative F12 levels following oral delivery to fasting mice of a dose of a formulation containing a GalNAc-siRNA of 25 mg/kg (with 37.5 mM C10) at days 0, 2, and 5, as compared against the relative F12 levels following oral delivery to non-fasting mice of a dose of the same siRNA of 25 mg/kg (with 37.5 mM C10) at days 0, 2, and 5. A comparison was also made employing the same siRNA administered subcutaneously at a single dose of 0.75 mg/kg.

FIG. 4 is a graph showing the relative F12 levels following oral delivery to fasting mice of a dose of a formulation containing a GalNAc-conjugated siRNA of 25 mg/kg (with 37.5 mM C10) at days 0, 2, and 5, as compared against the relative F12 levels following oral delivery to fasting mice of a dose of a formulation containing an unconjugated siRNA of 25 mg/kg (with 37.5 mM C10) at days 0, 2, and 5. A comparison was also made employing the same siRNA administered subcutaneously at a single dose of 0.75 mg/kg.

FIG. 5 is a graph showing the relative F12 levels following oral delivery to fasting mice of a dose of a formulation containing a GalNAc-siRNA of 25 mg/kg (with 37.5 mM C10) at days 0, 2, and 5, as compared against the relative F12 levels following oral delivery to fasting mice of a dose of a formulation containing the same siRNA of 25 mg/kg (with 37.5 mM C10) at days 0, 8, and 14. A comparison was also made employing the same siRNA administered subcutaneously at a single dose of 0.75 mg/kg.

FIG. 6 is a graph showing the relative plasma F12 levels following oral delivery to fasting mice of a dose of formulation containing a GalNAc-siRNA of 10 mg/kg (with 150 mM C10) on days 0, 2, and 5, as compared against the relative plasma F12 levels following oral delivery to fasting mice of a dose of formulation containing the same siRNA without GalNAc conjugation of 10 mg/kg (with 150 mM C10, sodium caproate) on days 0, 2, and 5. Oral administration of PBS and subcutaneous administration of the same GalNAc-siRNA at a single dose of 0.75 mg/kg were employed as comparisons.

FIG. 7 is a graph showing the relative plasma F12 levels following oral delivery to fasting or fed wild type C57/BL6 mice of a dose of formulation containing GalNAc-siRNA at a single dose of 30 mg/kg (with 150 mM C10, sodium caproate), as compared against the relative plasma F12 levels following oral delivery to fasting or fed asialoglycoprotein receptor (ASGR) knockout (KO) mice of a dose of formulation containing the same GalNAc-siRNA at a single dose of 30 mg/kg (with 150 mM C10).

FIG. 8 is a graph showing the relative plasma F12 levels following oral delivery to fasting mice of a dose of formulation containing GalNAc-siRNA at a single dose of 1, 3, and 10 mg/kg (with 150 mM C10, sodium caproate), respectively, as compared against the relative plasma F12 levels following oral delivery to fasting mice of a dose of formulation containing the same GalNAc-siRNA at 1, 3, 10 mg/kg (with 150 mM C10), respectively, on days 0, 2, and 5. An oral formulation containing the same GalNAc-siRNA, but without vinylphosphonate (VP) at the 5′-end of the antisense sequence, was also employed and administered to fasting mice at 3 mg/kg (with 150 mM C10) on days 0, 2, and 5.

FIG. 9 is a graph showing the relative plasma F12 levels following oral delivery to fasting mice of a dose of formulation containing GalNAc-siRNA at 3 mg/kg (with 150 mM C10) on days 0, 2, and 5; the relative plasma F12 levels following oral delivery to fed mice of a dose of formulation containing the same GalNAc-siRNA at 3 mg/kg (with 150 mM C10) for every 4 hours on day 0; and the relative plasma F12 levels following oral delivery to fasting mice of a dose of formulation containing the same GalNAc-siRNA at 3 mg/kg (with 150 mM C10, sodium caproate) on days 0, 1, 2, respectively. In all groups, the formulations containing GalNAc-siRNA were orally administered at the same dosing level with three times dosing.

FIG. 10 is a graph showing the relative F12 levels following oral delivery to a NHP of a formulation containing a GalNAc-siRNA (with 150 mM C10 in solution).

FIG. 11 is a graph showing the relative plasma F12 levels following oral delivery to cynomolgus monkey (N=4) of a dose of formulation containing GalNAc-siRNA at 10 mg/kg (with 150 mM C10) on days 0, 2, and 5.

FIG. 12 is a graph showing the relative plasma TTR levels following oral delivery to cynomolgus monkey of a dose of formulation containing GalNAc-siRNA (AD-157687 with 150 mM C10) at 3 mg/kg and 10 mg/kg, respectively, on days 0, 2, and 5. A comparison was made employing the same GalNAc-siRNA administered subcutaneously at a single dose of 1 mg/kg.

FIG. 13 is a graph showing the relative plasma TTR levels following oral delivery to cynomolgus monkey of a dose of formulation containing GalNAc-siRNA (AD-87404 with 150 mM C10) at 3, 10, and 30 mg/kg, respectively, on days 0, 2, and 5. A comparison was made employing the same GalNAc-siRNA administered subcutaneously at a single dose of 3 mg/kg.

FIG. 14 is a graph showing the amounts of siRNA in plasma following oral delivery to cynomolgus monkey of a dose of formulation containing GalNAc-siRNA (AD-157687, with 150 mM C10) at a single dose of 3 and 10 mg/kg, respectively. A comparison was made employing the same GalNAc-siRNA administered subcutaneously at a single dose of 1 mg/kg.

FIG. 15 is a graph showing the amounts of siRNA in plasma following oral delivery to cynomolgus monkey of a dose of formulation containing GalNAc-siRNA (AD-87404, with 150 mM C10) at a single dose of 3, 10, and 30 mg/kg, respectively. A comparison was made employing the same GalNAc-siRNA administered subcutaneously at a single dose of 3 mg/kg.

FIG. 16 is a graph showing the amount of siRNA in liver following oral delivery to cynomolgus monkey of a dose of formulation containing GalNAc-siRNA (AD-157687, with 150 mM C10) at 3 and 10 mg/kg, respectively. A comparison was made employing the same GalNAc-siRNA administered subcutaneously at a single dose of 1 mg/kg.

FIG. 17 is a graph showing the amount of siRNA in liver following oral delivery to cynomolgus monkey of a dose of formulation containing GalNAc-siRNA (AD-87404, with 150 mM C10) at 3, 10, and 30 mg/kg, respectively, on days 0, 2, and 5. A comparison was made employing the same GalNAc-siRNA administered subcutaneously at a single dose of 3 mg/kg.

FIG. 18 is a graph showing the relative F12 levels following oral delivery to fasting mice of a dose of a formulation containing a GalNAc-siRNA of 3 mg/kg (with C10 at 150 mM or 75 mM) at days 0, 2, and 5; the relative F12 levels following oral delivery to fasting mice of a dose of a formulation containing the same siRNA of 3 mg/kg (with C8 at 150 mM or 75 mM) at days 0, 2, and 5; the relative F12 levels following oral delivery to fasting mice of a dose of a formulation containing the same siRNA of 3 mg/kg (with C12 at 150 mM or 75 mM) at days 0, 2, and 5; and the relative F12 levels following oral delivery to fasting mice of a dose of a formulation containing the same siRNA of 3 mg/kg (with C18:1 at 150 mM or 75 mM) at days 0, 2, and 5; and the relative F12 levels following oral delivery to fasting mice of a dose of a formulation containing the same siRNA of 3 mg/kg (with 75 mM C10 in combination with 75 mM C8) at days 0, 2, and 5. A comparison was made employing the same siRNA administered subcutaneously at a single dose of 0.15 mg/kg.

FIG. 19 is a graph showing the relative F12 levels following oral delivery to fasting mice of a dose of a formulation containing a GalNAc-siRNA of 3 mg/kg (with C10 at 150 mM or 75 mM) at days 0, 2, and 5, as compared against the relative F12 levels following oral delivery to fasting mice of a dose of the same siRNA of 3 mg/kg (with C8 at 150 mM or 75 mM) at days 0, 2, and 5. A comparison was also made employing the same siRNA administered subcutaneously at a single dose of 0.15 mg/kg.

FIG. 20 is a graph showing the relative F12 levels following oral delivery to fasting mice of a dose of a formulation containing a GalNAc-siRNA of 3 mg/kg (with C10 at 150 mM or 75 mM) at days 0, 2, and 5, as compared against the relative F12 levels following oral delivery to fasting mice of a dose of the same siRNA of 3 mg/kg (with C12 at 150 mM or 75 mM) at days 0, 2, and 5. A comparison was also made employing the same siRNA administered subcutaneously at a single dose of 0.15 mg/kg.

FIG. 21 is a graph showing the relative F12 levels following oral delivery to fasting mice of a dose of a formulation containing a GalNAc-siRNA of 3 mg/kg (with C10 at 150 mM or 75 mM) at days 0, 2, and 5, as compared against the relative F12 levels following oral delivery to fasting mice of a dose of the same siRNA of 3 mg/kg (with C18:1 at 150 mM or 75 mM) at days 0, 2, and 5. A comparison was also made employing the same siRNA administered subcutaneously at a single dose of 0.15 mg/kg.

FIG. 22 is a graph showing the relative F12 levels following oral delivery to fasting mice of a dose of a formulation containing a GalNAc-siRNA of 3 mg/kg (with C10 at 150 mM or 75 mM) at days 0, 2, and 5, as compared against the relative F12 levels following oral delivery to fasting mice of a dose of the same siRNA of 3 mg/kg (with 75 mM C10 in combination with 75 mM C8) at days 0, 2, and 5. A comparison was also made employing the same siRNA administered subcutaneously at a single dose of 0.15 mg/kg.

FIG. 23 is a graph showing the relative plasma F12 levels following oral delivery to fasting mice of a dose of formulation containing GalNAc-siRNA with 75 mM of various permeation enhancers, including sodium caproate (C10), salcaprozate sodium (SNAC), ethylenediaminetetraacetic acid (EDTA), sodium oleate (C18:1), sodium laurate (C12), and sodium caprylate (C8), respectively, at 3 mg/kg on days 0, 2, and 5. A comparison was made employing the same GalNAc-siRNA administered subcutaneously at a single dose of 0.15 mg/kg.

### DETAILED DESCRIPTION

The inventors have found, inter alia, that the formulation described herein provides surprisingly good and robust results for in vivo oral delivery of a double-stranded iRNA agent, achieving effective and efficient oral delivery of the double-stranded iRNA agent at clinically relevant dose regimen.

One aspect of the invention provides an oral formulation for reducing or inhibiting the expression of a target gene in a subject. The oral formulation comprises a) a double-stranded iRNA agent and b) a penetration enhancer. The double-stranded iRNA agent comprises an antisense strand which is complementary to a target gene; a sense strand which is complementary to said antisense strand; and a carbohydrate-based ligand conjugated to at least one of the strands, optionally via a linker or carrier. The double-stranded iRNA agent also comprises 2′-OMe modifications to more than fifteen, more than twenty, more than twenty-five, or more than thirty nucleotides.

### Ligands

The double-stranded iRNA agent of the invention is further modified by covalent attachment of one or more targeting ligands, such as carbohydrate-based ligands.

As used herein the term “targeting ligand” refers to any molecule that provides an enhanced affinity for a selected target, e.g., a cell, cell type, tissue, organ, region of the body, or a compartment, e.g., a cellular, tissue or organ compartment. Some exemplary targeting ligands include, but are not limited to, antibodies, antigens, folates, receptor ligands, carbohydrates, aptamers, integrin receptor ligands, chemokine receptor ligands, transferrin, biotin, serotonin receptor ligands, PSMA, endothelin, GCPII, somatostatin, LDL and HDL ligands.

Carbohydrate based targeting ligands include, but are not limited to, D-galactose, multivalent galactose, N-acetyl-D-galactosamine (GalNAc), multivalent GalNAc, e.g. GalNAc₂ and GalNAc₃ (GalNAc and multivalent GalNAc are collectively referred to herein as GalNAc conjugates); D-mannose, multivalent mannose, multivalent lactose, N-acetyl-glucosamine, glucose, multivalent Glucose, multivalent fucose, glycosylated polyaminoacids and lectins. The term multivalent indicates that more than one monosaccharide unit is present. Such monosaccharide subunits can be linked to each other through glycosidic linkages or linked to a scaffold molecule.

A number of folate and folate analogs amenable to the present invention as ligands are described in U.S. Pat. Nos. 2,816,110; 5,552,545; 6,335,434 and 7,128,893, contents of which are herein incorporated in their entireties by reference.

When two or more ligands are present, the ligands can all have same properties, all have different properties or some ligands have the same properties while others have different properties. For example, a ligand can have targeting properties, have endosomolytic activity or have PK modulating properties. In a preferred embodiment, all the ligands have different properties.

As used herein, the terms “PK modulating ligand” and “PK modulator” refers to molecules which can modulate the pharmacokinetics of the composition of the invention. Some exemplary PK modulator include, but are not limited to, lipophilic molecules, bile acids, sterols, phospholipid analogues, peptides, protein binding agents, vitamins, fatty acids, phenoxazine, aspirin, naproxen, ibuprofen, suprofen, ketoprofen, (S)-(+)-pranoprofen, carprofen, PEGs, biotin, and transthyretia-binding ligands (e.g., tetraiidothyroacetic acid, 2, 4, 6-triiodophenol and flufenamic acid). Oligomeric compounds that comprise a number of phosphorothioate intersugar linkages are also known to bind to serum protein, thus short oligomeric compounds, e.g. oligonucleotides of comprising from about 5 to 30 nucleotides (e.g., 5 to 25 nucleotides, preferably 5 to 20 nucleotides, e.g., 5, 6, 7, 8, 9, 10, 11, 12, 13, 14, 15, 16, 17, 18, 19, or 20 nucleotides), and that comprise a plurality of phosphorothioate linkages in the backbone are also amenable to the present invention as ligands (e.g. as PK modulating ligands). The PK modulating oligonucleotide can comprise at least 3, 4, 5, 6, 7, 8, 9, 10, 11, 12, 13, 14, 15 or more phosphorothioate and/or phosphorodithioate linkages. In some embodiments, all internucleotide linkages in PK modulating oligonucleotide are phosphorothioate and/or phosphorodithioates linkages. In addition, aptamers that bind serum components (e.g. serum proteins) are also amenable to the present invention as PK modulating ligands. Binding to serum components (e.g. serum proteins) can be predicted from albumin binding assays, such as those described in Oravcova, et al., Journal of Chromatography B (1996), 677: 1-27.

In general, conjugate groups modify one or more properties of the attached double-stranded iRNA agent including but not limited to pharmacodynamic, pharmacokinetic, binding, absorption, cellular distribution, cellular uptake, charge and clearance. Conjugate groups are routinely used in the chemical arts and are linked directly or via an optional linking moiety or linking group to a parent compound such as an oligomeric compound.

A preferred list of conjugate groups includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes.

Preferred conjugate groups amenable to the present invention include lipid moieties such as a cholesterol moiety (Letsinger et al., Proc. Natl. Acad. Sci. USA, 1989, 86, 6553); cholic acid (Manoharan et al., Bioorg. Med. Chem. Lett., 1994, 4, 1053); a thioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765); a thiocholesterol (Oberhauser et al., Nucl. Acids Res., 1992, 20, 533); an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49); a phospholipid, e.g., di-hexadecyl-rac-glycerol or triethylammonium-1,2-di-O-hexadecyl-rac-glycero-3-H-phosphonate (Manoharan et al., Tetrahedron Lett., 1995, 36, 3651; Shea et al., Nucl. Acids Res., 1990, 18, 3777); a polyamine or a polyethylene glycol chain (Manoharan et al., Nucleosides & Nucleotides, 1995, 14, 969); adamantane acetic acid (Manoharan et al., Tetrahedron Lett., 1995, 36, 3651); a palmityl moiety (Mishra et al., Biochim. Biophys. Acta, 1995, 1264, 229); or an octadecylamine or hexylamino-carbonyl-oxycholesterol moiety (Crooke et al., J. Pharmacol. Exp. Ther., 1996, 277, 923).

Generally, a wide variety of entities, e.g., ligands, can be coupled to the oligomeric compounds described herein. Ligands can include naturally occurring molecules, or recombinant or synthetic molecules. Exemplary ligands include, but are not limited to, polylysine (PLL), poly L-aspartic acid, poly L-glutamic acid, styrene-maleic acid anhydride copolymer, poly(L-lactide-co-glycolide) copolymer, divinyl ether-maleic anhydride copolymer, N-(2-hydroxypropyl)methacrylamide copolymer (HMPA), polyethylene glycol (PEG, e.g., PEG-2K, PEG-5K, PEG-10K, PEG-12K, PEG-15K, PEG-20K, PEG-40K), MPEG, [MPEG]₂, polyvinyl alcohol (PVA), polyurethane, poly(2-ethylacrylic acid), N-isopropylacrylamide polymers, polyphosphazine, polyethylenimine, cationic groups, spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, thyrotropin, melanotropin, lectin, glycoprotein, surfactant protein A, mucin, glycosylated polyaminoacids, transferrin, bisphosphonate, polyglutamate, polyaspartate, aptamer, asialofetuin, hyaluronan, procollagen, immunoglobulins (e.g., antibodies), insulin, transferrin, albumin, sugar-albumin conjugates, intercalating agents (e.g., acridines), cross-linkers (e.g. psoralen, mitomycin C), porphyrins (e.g., TPPC4, texaphyrin, Sapphyrin), polycyclic aromatic hydrocarbons (e.g., phenazine, dihydrophenazine), artificial endonucleases (e.g., EDTA), lipophilic molecules (e.g, steroids, bile acids, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine), peptides (e.g., an alpha helical peptide, amphipathic peptide, RGD peptide, cell permeation peptide, endosomolytic/fusogenic peptide), alkylating agents, phosphate, amino, mercapto, polyamino, alkyl, substituted alkyl, radiolabeled markers, enzymes, haptens (e.g. biotin), transport/absorption facilitators (e.g., naproxen, aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, AP, antibodies, hormones and hormone receptors, lectins, carbohydrates, multivalent carbohydrates, vitamins (e.g., vitamin A, vitamin E, vitamin K, vitamin B, e.g., folic acid, B12, riboflavin, biotin and pyridoxal), vitamin cofactors, lipopolysaccharide, an activator of p38 MAP kinase, an activator of NF-κB, taxon, vincristine, vinblastine, cytochalasin, nocodazole, japlakinolide, latrunculin A, phalloidin, swinholide A, indanocine, myoservin, tumor necrosis factor alpha (TNFalpha), interleukin-1 beta, gamma interferon, natural or recombinant low density lipoprotein (LDL), natural or recombinant high-density lipoprotein (HDL), and a cell-permeation agent (e.g., a.helical cell-permeation agent).

Peptide and peptidomimetic ligands include those having naturally occurring or modified peptides, e.g., D or L peptides; α, β, or γ peptides; N-methyl peptides; azapeptides; peptides having one or more amide, i.e., peptide, linkages replaced with one or more urea, thiourea, carbamate, or sulfonyl urea linkages; or cyclic peptides. A peptidomimetic (also referred to herein as an oligopeptidomimetic) is a molecule capable of folding into a defined three-dimensional structure similar to a natural peptide. The peptide or peptidomimetic ligand can be about 5-50 amino acids long, e.g., about 5, 10, 15, 20, 25, 30, 35, 40, 45, or 50 amino acids long.

Exemplary amphipathic peptides include, but are not limited to, cecropins, lycotoxins, paradaxins, buforin, CPF, bombinin-like peptide (BLP), cathelicidins, ceratotoxins, S. clava peptides, hagfish intestinal antimicrobial peptides (HFIAPs), magainines, brevinins-2, dermaseptins, melittins, pleurocidin, H₂A peptides, Xenopus peptides, esculentinis-1, and caerins.

As used herein, the term “endosomolytic ligand” refers to molecules having endosomolytic properties. Endosomolytic ligands promote the lysis of and/or transport of the composition of the invention, or its components, from the cellular compartments such as the endosome, lysosome, endoplasmic reticulum (ER), Golgi apparatus, microtubule, peroxisome, or other vesicular bodies within the cell, to the cytoplasm of the cell. Some exemplary endosomolytic ligands include, but are not limited to, imidazoles, poly or oligoimidazoles, linear or branched polyethyleneimines (PEIs), linear and branched polyamines, e.g. spermine, cationic linear and branched polyamines, polycarboxylates, polycations, masked oligo or poly cations or anions, acetals, polyacetals, ketals/polyketals, orthoesters, linear or branched polymers with masked or unmasked cationic or anionic charges, dendrimers with masked or unmasked cationic or anionic charges, polyanionic peptides, polyanionic peptidomimetics, pH-sensitive peptides, natural and synthetic fusogenic lipids, natural and synthetic cationic lipids.

Exemplary endosomolytic/fusogenic peptides include, but are not limited to, AALEALAEALEALAEALEALAEAAAAGGC (GALA); AALAEALAEALAEALAEALAEALAAAAGGC (EALA); ALEALAEALEALAEA; GLFEAIEGFIENGWEGMIWDYG (INF-7); GLFGAIAGFIENGWEGMIDGWYG (Inf HA-2); GLFEAIEGFIENGWEGMIDGWYGCGLFEAIEGFIENGWEGMID GWYGC (diINF-7); GLFEAIEGFIENGWEGMIDGGCGLFEAIEGFIENGWEGMIDGGC (diINF-3); GLFGALAEALAEALAEHLAEALAEALEALAAGGSC (GLF); GLFEAIEGFIENGWEGLAEALAEALEALAAGGSC (GALA-INF3); GLF EAI EGFI ENGW EGnI DG K GLF EAI EGFI ENGW EGnI DG (INF-5, n is norleucine); LFEALLELLESLWELLLEA (JTS-1); GLFKALLKLLKSLWKLLLKA (ppTG1); GLFRALLRLLRSLWRLLLRA (ppTG20); WEAKLAKALAKALAKHLAKALAKALKACEA (KALA); GLFFEAIAEFIEGGWEGLIEGC (HA); GIGAVLKVLTTGLPALISWIKRKRQQ (Melittin); H₅WYG; and CHK₆HC.

Without wishing to be bound by theory, fusogenic lipids fuse with and consequently destabilize a membrane. Fusogenic lipids usually have small head groups and unsaturated acyl chains. Exemplary fusogenic lipids include, but are not limited to, 1,2-dileoyl-sn-3-phosphoethanolamine (DOPE), phosphatidylethanolamine (POPE), palmitoyloleoylphosphatidylcholine (POPC), (6Z,9Z,28Z,31Z)-heptatriaconta-6,9,28,31-tetraen-19-ol (Di-Lin), N-methyl(2,2-di((9Z,12Z)-octadeca-9,12-dienyl)-1,3-dioxolan-4-yl)methanamine (DLin-k-DMA) and N-methyl-2-(2,2-di((9Z,12Z)-octadeca-9,12-dienyl)-1,3-dioxolan-4-yl)ethanamine (also referred to as XTC herein).

Synthetic polymers with endosomolytic activity amenable to the present invention are described in U.S. Pat. App. Pub. Nos. 2009/0048410; 2009/0023890; 2008/0287630; 2008/0287628; 2008/0281044; 2008/0281041; 2008/0269450; 2007/0105804; 20070036865; and 2004/0198687, contents of which are hereby incorporated by reference in their entirety.

Exemplary cell permeation peptides include, but are not limited to, RQIKIWFQNRRMKWKK (penetratin); GRKKRRQRRRPPQC (Tat fragment 48-60); GALFLGWLGAAGSTMGAWSQPKKKRKV (signal sequence based peptide); LLIILRRRIRKQAHAHSK (PVEC); GWTLNSAGYLLKINLKALAALAKKIL (transportan); KLALKLALKALKAALKLA (amphiphilic model peptide); RRRRRRRRR (Arg9); KFFKFFKFFK (Bacterial cell wall permeating peptide); LLGDFFRKSKEKIGKEFKRIVQRIKDFLRNLVPRTES (LL-37); SWLSKTAKKLENSAKKRISEGIAIAIQGGPR (cecropin P1); ACYCRIPACIAGERRYGTCIYQGRLWAFCC (α-defensin); DHYNCVSSGGQCLYSACPIFTKIQGTCYRGKAKCCK (β-defensin); RRRPRPPYLPRPRPPPFFPPRLPPRIPPGFPPRFPPRFPGKR-NH2 (PR-39); ILPWKWPWWPWRR-NH2 (indolicidin); AAVALLPAVLLALLAP (RFGF); AALLPVLLAAP (RFGF analogue); and RKCRIVVIRVCR (bactenecin).

Exemplary cationic groups include, but are not limited to, protonated amino groups, derived from e.g., O-AMINE (AMINE=NH₂; alkylamino, dialkylamino, heterocyclyl, arylamino, diaryl amino, heteroaryl amino, or diheteroaryl amino, ethylene diamine, polyamino); aminoalkoxy, e.g., O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino); amino (e.g. NH₂; alkylamino, dialkylamino, heterocyclyl, arylamino, diaryl amino, heteroaryl amino, diheteroaryl amino, or amino acid); and NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino).

The ligand or tethered ligand can be present on a monomer when said monomer is incorporated into a component of the double-stranded iRNA agent of the invention (e.g., a double-stranded iRNA agent of the invention or linker). In some embodiments, the ligand can be incorporated via coupling to a “precursor” monomer after said “precursor” monomer has been incorporated into a component of the double-stranded iRNA agent of the invention (e.g., a double-stranded iRNA agent of the invention or linker). For example, a monomer having, e.g., an amino-terminated tether (i.e., having no associated ligand), e.g., monomer-linker-NH₂ can be incorporated into a component of the compounds of the invention (e.g., a double-stranded iRNA agent of the invention or linker). In a subsequent operation, i.e., after incorporation of the precursor monomer into a component of the compounds of the invention (e.g., a double-stranded iRNA agent of the invention or linker), a ligand having an electrophilic group, e.g., a pentafluorophenyl ester or aldehyde group, can subsequently be attached to the precursor monomer by coupling the electrophilic group of the ligand with the terminal nucleophilic group of the precursor monomer's tether.

In another example, a monomer having a chemical group suitable for taking part in Click Chemistry reaction can be incorporated e.g., an azide or alkyne terminated tether/linker. In a subsequent operation, i.e., after incorporation of the precursor monomer into the strand, a ligand having complementary chemical group, e.g. an alkyne or azide can be attached to the precursor monomer by coupling the alkyne and the azide together.

In some embodiments, ligand can be conjugated to nucleobases, sugar moieties, or internucleosidic linkages of the double-stranded iRNA agent of the invention. Conjugation to purine nucleobases or derivatives thereof can occur at any position including, endocyclic and exocyclic atoms. In some embodiments, the 2-, 6-, 7-, or 8-positions of a purine nucleobase are attached to a conjugate moiety. Conjugation to pyrimidine nucleobases or derivatives thereof can also occur at any position. In some embodiments, the 2-, 5-, and 6-positions of a pyrimidine nucleobase can be substituted with a conjugate moiety. When a ligand is conjugated to a nucleobase, the preferred position is one that does not interfere with hybridization, i.e., does not interfere with the hydrogen bonding interactions needed for base pairing.

Conjugation to sugar moieties of nucleosides can occur at any carbon atom. Example carbon atoms of a sugar moiety that can be attached to a conjugate moiety include the 2′, 3′, and 5′ carbon atoms. The 1′ position can also be attached to a conjugate moiety, such as in an abasic residue. Internucleosidic linkages can also bear conjugate moieties. For phosphorus-containing linkages (e.g., phosphodiester, phosphorothioate, phosphorodithioate, phosphoroamidate, and the like), the conjugate moiety can be attached directly to the phosphorus atom or to an O, N, or S atom bound to the phosphorus atom. For amine- or amide-containing internucleosidic linkages (e.g., PNA), the conjugate moiety can be attached to the nitrogen atom of the amine or amide or to an adjacent carbon atom.

There are numerous methods for preparing conjugates of oligonuclotides. Generally, an oligonucleotide is attached to a conjugate moiety by contacting a reactive group (e.g., OH, SH, amine, carboxyl, aldehyde, and the like) on the oligonucleotide with a reactive group on the conjugate moiety. In some embodiments, one reactive group is electrophilic and the other is nucleophilic.

For example, an electrophilic group can be a carbonyl-containing functionality and a nucleophilic group can be an amine or thiol. Methods for conjugation of nucleic acids and related oligomeric compounds with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

The ligand can be attached to the double-stranded iRNA agent of the inventions via a linker or a carrier monomer, e.g., a ligand carrier. The carriers include (i) at least one “backbone attachment point,” preferably two “backbone attachment points” and (ii) at least one “tethering attachment point.” A “backbone attachment point” as used herein refers to a functional group, e.g. a hydroxyl group, or generally, a bond available for, and that is suitable for incorporation of the carrier monomer into the backbone, e.g., the phosphate, or modified phosphate, e.g., sulfur containing, backbone, of an oligonucleotide. A “tethering attachment point” (TAP) in refers to an atom of the carrier monomer, e.g., a carbon atom or a heteroatom (distinct from an atom which provides a backbone attachment point), that connects a selected moiety. The selected moiety can be, e.g., a carbohydrate, e.g. monosaccharide, disaccharide, trisaccharide, tetrasaccharide, oligosaccharide and polysaccharide. Optionally, the selected moiety is connected by an intervening tether to the carrier monomer. Thus, the carrier will often include a functional group, e.g., an amino group, or generally, provide a bond, that is suitable for incorporation or tethering of another chemical entity, e.g., a ligand to the constituent atom.

Representative U.S. patents that teach the preparation of conjugates of nucleic acids include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,149,782; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; 5,672,662; 5,688,941; 5,714,166; 6,153,737; 6,172,208; 6,300,319; 6,335,434; 6,335,437; 6,395,437; 6,444,806; 6,486,308; 6,525,031; 6,528,631; 6,559,279; contents of which are herein incorporated in their entireties by reference.

In some embodiments, the double-stranded iRNA agent may further comprise one or more other ligands, such as lipophilic moieties, conjugated to one or more internal positions on at least one of the strands, optionally via a linker or carrier. The term “lipophile” or “lipophilic moiety” broadly refers to any compound or chemical moiety having an affinity for lipids. One way to characterize the lipophilicity of the lipophilic moiety is by the octanol-water partition coefficient, log K_{ow}, where K_{ow} is the ratio of a chemical's concentration in the octanol-phase to its concentration in the aqueous phase of a two-phase system at equilibrium. The octanol-water partition coefficient is a laboratory-measured property of a substance. However, it may also be predicted by using coefficients attributed to the structural components of a chemical which are calculated using first-principle or empirical methods (see, for example, Tetko et al., J. Chem. Inf Comput. Sci. 41:1407-21 (2001), which is incorporated herein by reference in its entirety). It provides a thermodynamic measure of the tendency of the substance to prefer a non-aqueous or oily milieu rather than water (i.e. its hydrophilic/lipophilic balance). In principle, a chemical substance is lipophilic in character when its log K_{ow} exceeds 0. Typically, the lipophilic moiety possesses a log K_{ow} exceeding 1, exceeding 1.5, exceeding 2, exceeding 3, exceeding 4, exceeding 5, or exceeding 10. For instance, the log K_{ow} of 6-amino hexanol, for instance, is predicted to be approximately 0.7. Using the same method, the log K_{ow} of cholesteryl N-(hexan-6-ol) carbamate is predicted to be 10.7.

The lipophilicity of a molecule can change with respect to the functional group it carries. For instance, adding a hydroxyl group or amine group to the end of a lipophilic moiety can increase or decrease the partition coefficient (e.g., log K_{ow}) value of the lipophilic moiety.

Alternatively, the hydrophobicity of the double-stranded iRNA agent, conjugated to one or more lipophilic moieties, can be measured by its protein binding characteristics. For instance, the unbound fraction in the plasma protein binding assay of the double-stranded iRNA agent can be determined to positively correlate to the relative hydrophobicity of the double-stranded iRNA agent, which can positively correlate to the silencing activity of the double-stranded iRNA agent.

In one embodiment, the plasma protein binding assay determined is an electrophoretic mobility shift assay (EMSA) using human serum albumin protein. The hydrophobicity of the double-stranded iRNA agent, measured by fraction of unbound siRNA in the binding assay, exceeds 0.15, exceeds 0.2, exceeds 0.25, exceeds 0.3, exceeds 0.35, exceeds 0.4, exceeds 0.45, or exceeds 0.5 for an enhanced in vivo delivery of siRNA.

Accordingly, conjugating the lipophilic moieties to the internal position(s) of the double-stranded iRNA agent provides optimal hydrophobicity for the enhanced in vivo delivery of siRNA.

In certain embodiments, the lipophilic moiety is an aliphatic, cyclic such as alicyclic, or polycyclic such as polyalicyclic compound, such as a steroid (e.g., sterol) or a linear or branched aliphatic hydrocarbon. The lipophilic moiety may generally comprises a hydrocarbon chain, which may be cyclic or acyclic. The hydrocarbon chain may comprise various substituents and/or one or more heteroatoms, such as an oxygen or nitrogen atom. Such lipophilic aliphatic moieties include, without limitation, saturated or unsaturated C₄-C₃₀ hydrocarbon (e.g., C₆-C₁₈ hydrocarbon), saturated or unsaturated fatty acids, waxes (e.g., monohydric alcohol esters of fatty acids and fatty diamides), terpenes (e.g., C₁₀ terpenes, C₁₅ sesquiterpenes, C₂₀ diterpenes, C₃₀ triterpenes, and C₄₀ tetraterpenes), and other polyalicyclic hydrocarbons. For instance, the lipophilic moiety may contain a C₄-C₃₀ hydrocarbon chain (e.g., C₄-C₃₀ alkyl or alkenyl). In some embodiment the lipophilic moiety contains a saturated or unsaturated C₆-C₁₈ hydrocarbon chain (e.g., a linear C₆-C₁₈ alkyl or alkenyl). In one embodiment, the lipophilic moiety contains a saturated or unsaturated C₁₆ hydrocarbon chain (e.g., a linear C₁₆ alkyl or alkenyl).

The lipophilic moiety may be attached to the iRNA agent by any method known in the art, including via a functional grouping already present in the lipophilic moiety or introduced into the iRNA agent, such as a hydroxy group (e.g., —CO—CH₂—OH). The functional groups already present in the lipophilic moiety or introduced into the iRNA agent include, but are not limited to, hydroxyl, amine, carboxylic acid, sulfonate, phosphate, thiol, azide, and alkyne.

Conjugation of the iRNA agent and the lipophilic moiety may occur, for example, through formation of an ether or a carboxylic or carbamoyl ester linkage between the hydroxy and an alkyl group R—, an alkanoyl group RCO— or a substituted carbamoyl group RNHCO—. The alkyl group R may be cyclic (e.g., cyclohexyl) or acyclic (e.g., straight-chained or branched; and saturated or unsaturated). Alkyl group R may be a butyl, pentyl, hexyl, heptyl, octyl, nonyl, decyl, undecyl, dodecyl, tridecyl, tetradecyl, pentadecyl, hexadecyl, heptadecyl or octadecyl group, or the like.

In some embodiments, the lipophilic moiety is conjugated to the double-stranded iRNA agent via a linker a linker containing an ether, thioether, urea, carbonate, amine, amide, maleimide-thioether, disulfide, phosphodiester, sulfonamide linkage, a product of a click reaction (e.g., a triazole from the azide-alkyne cycloaddition), or carbamate.

In another embodiment, the lipophilic moiety is a steroid, such as sterol. Steroids are polycyclic compounds containing a perhydro-1,2-cyclopentanophenanthrene ring system. Steroids include, without limitation, bile acids (e.g., cholic acid, deoxycholic acid and dehydrocholic acid), cortisone, digoxigenin, testosterone, cholesterol, and cationic steroids, such as cortisone. A “cholesterol derivative” refers to a compound derived from cholesterol, for example by substitution, addition or removal of substituents.

In another embodiment, the lipophilic moiety is an aromatic moiety. In this context, the term “aromatic” refers broadly to mono- and polyaromatic hydrocarbons. Aromatic groups include, without limitation, C₆-C₁₄ aryl moieties comprising one to three aromatic rings, which may be optionally substituted; “aralkyl” or “arylalkyl” groups comprising an aryl group covalently linked to an alkyl group, either of which may independently be optionally substituted or unsubstituted; and “heteroaryl” groups. As used herein, the term “heteroaryl” refers to groups having 5 to 14 ring atoms, preferably 5, 6, 9, or 10 ring atoms; having 6, 10, or 14π electrons shared in a cyclic array, and having, in addition to carbon atoms, between one and about three heteroatoms selected from the group consisting of nitrogen (N), oxygen (O), and sulfur (S).

As employed herein, a “substituted” alkyl, cycloalkyl, aryl, heteroaryl, or heterocyclic group is one having between one and about four, preferably between one and about three, more preferably one or two, non-hydrogen substituents. Suitable substituents include, without limitation, halo, hydroxy, nitro, haloalkyl, alkyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups.

In some embodiments, the lipophilic moiety is an aralkyl group, e.g., a 2-arylpropanoyl moiety. The structural features of the aralkyl group are selected so that the lipophilic moiety will bind to at least one protein in vivo. In certain embodiments, the structural features of the aralkyl group are selected so that the lipophilic moiety binds to serum, vascular, or cellular proteins. In certain embodiments, the structural features of the aralkyl group promote binding to albumin, an immunoglobulin, a lipoprotein, α-2-macroglubulin, or α-1-glycoprotein.

In certain embodiments, the ligand is naproxen or a structural derivative of naproxen. Procedures for the synthesis of naproxen can be found in U.S. Pat. Nos. 3,904,682 and 4,009,197, which are hereby incorporated by reference in their entirety. Naproxen has the chemical name (S)-6-Methoxy-α-methyl-2-naphthaleneacetic acid and the structure is

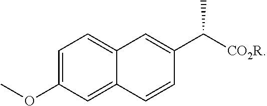

In certain embodiments, the ligand is ibuprofen or a structural derivative of ibuprofen. Procedures for the synthesis of ibuprofen can be found in U.S. Pat. No. 3,228,831, which are hereby incorporated by reference in their entirety. The structure of ibuprofen is

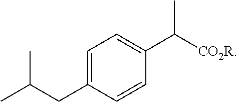

Additional exemplary aralkyl groups are illustrated in U.S. Pat. No. 7,626,014, which is incorporated herein by reference in its entirety.

In another embodiment, suitable lipophilic moieties include lipid, cholesterol, retinoic acid, cholic acid, adamantane acetic acid, 1-pyrene butyric acid, dihydrotestosterone, 1,3-bis-O(hexadecyl)glycerol, geranyloxyhexyanol, hexadecylglycerol, borneol, menthol, 1,3-propanediol, heptadecyl group, palmitic acid, myristic acid, O3-(oleoyl)lithocholic acid, O3-(oleoyl)cholenic acid, ibuprofen, naproxen, dimethoxytrityl, or phenoxazine.

In some embodiments, the lipophilic moiety is a C₆-C₃₀ acid (e.g., hexanoic acid, heptanoic acid, octanoic acid, nonanoic acid, decanoic acid, undecanoic acid, dodcanoic acid, tridecanoic acid, tetradecanoic acid, pentadecanoic acid, hexadecanoic acid, heptadecanoic acid, octadecanoic acid, oleic acid, linoleic acid, arachidonic acid, cis-4,7,10,13,16,19-docosahexanoic acid, vitamin A, vitamin E, cholesterol etc.) or a C₆-C₃₀ alcohol (e.g., hexanol, heptanol, octanol, nonanol, decanol, undecanol, dodcanol, tridecanol, tetradecanol, pentadecanol, hexadecanol, heptadecanol, octadecanol, oleyl alcohol, linoleyl alcohol, arachidonic alcohol, cis-4,7,10,13,16,19-docosahexanol, retinol, vitamin E, cholesterol etc.).

In certain embodiments, more than one lipophilic moieties can be incorporated into the double-strand iRNA agent, particularly when the lipophilic moiety has a low lipophilicity or hydrophobicity. In one embodiment, two or more lipophilic moieties are incorporated into the same strand of the double-strand iRNA agent. In one embodiment, each strand of the double-strand iRNA agent has one or more lipophilic moieties incorporated. In one embodiment, two or more lipophilic moieties are incorporated into the same position (i.e., the same nucleobase, same sugar moiety, or same internucleosidic linkage) of the double-strand iRNA agent. This can be achieved by, e.g., conjugating the two or more lipophilic moieties via a carrier, and/or conjugating the two or more lipophilic moieties via a branched linker, and/or conjugating the two or more lipophilic moieties via one or more linkers, with one or more linkers linking the lipophilic moieties consecutively.

The ligand may be conjugated to the iRNA agent via a direct attachment to the ribosugar of the iRNA agent. Alternatively, the ligand may be conjugated to the double-strand iRNA agent via a linker or a carrier.

In certain embodiments, the ligand may be conjugated to the iRNA agent via one or more linkers (tethers).

In one embodiment, the ligand is conjugated to the double-stranded iRNA agent via a linker containing an ether, thioether, urea, carbonate, amine, amide, maleimide-thioether, disulfide, phosphodiester, sulfonamide linkage, a product of a click reaction (e.g., a triazole from the azide-alkyne cycloaddition), or carbamate.

### Linkers Tethers

Linkers/Tethers are connected to the ligand at a “tethering attachment point (TAP).” Linkers/Tethers may include any C₁-C₁₀₀ carbon-containing moiety, (e.g. C₁-C₇₅, C₁-C₅₀, C₁-C₂₀, C₁-C₁₀; C₁, C₂, C₃, C₄, C₅, C₆, C₇, C₈, C₉, or C₁₀), and may have at least one nitrogen atom. In certain embodiments, the nitrogen atom forms part of a terminal amino or amido (NHC(O)—) group on the linker/tether, which may serve as a connection point for the ligand. Non-limited examples of linkers/tethers (underlined) include TAP-(CH₂)ₙNH—; TAP-C(O)(CH₂)ₙNH—; TAP-NR″″(CH₂)ₙNH—, TAP-C(O)-(CH₂)ₙ—C(O)—; TAP-C(O)—(CH₂)ₙ—C(O)O—; TAP-C(O)—O—; TAP-C(O)—(CH₂)ₙ—NH—C(O)—; TAP-C(O)—(CH₂)ₙ—; TAP-C(O)—NH—; TAP-C(O)—; TAP-(CH₂)ₙ—C(O)—; TAP-(CH₂)ₙ—C(O)O—; TAP-(CH₂)ₙ—; or TAP-(CH₂)ₙ—NH—C(O)—; in which n is 1-20 (e.g., 1, 2, 3, 4, 5, 6, 7, 8, 9, 10, 11, 12, 13, 14, 15, 16, 17, 18, 19, or 20) and R″″ is C₁-C₆ alkyl. Preferably, n is 5, 6, or 11. In other embodiments, the nitrogen may form part of a terminal oxyamino group, e.g., —ONH₂, or hydrazino group, —NHNH₂. The linker/tether may optionally be substituted, e.g., with hydroxy, alkoxy, perhaloalkyl, and/or optionally inserted with one or more additional heteroatoms, e.g., N, O, or S. Preferred tethered ligands may include, e.g., TAP-(CH₂)ₙNH(LIGAND); TAP-C(O)(CH₂)ₙNH(LIGAND); TAP-NR″″(CH₂)ₙNH(LIGAND); TAP-(CH₂)ₙONH(LIGAND); TAP-C(O)(CH₂)ₙONH(LIGAND); TAP-NR″″(CH₂)ₙONH(LIGAND); TAP-(CH₂)ₙNHNH₂(LIGAND), TAP-C(O)(CH₂)ₙNHNH₂(LIGAND); TAP-NR″″(CH₂)ₙNHNH₂(LIGAND); TAP-C(O)—(CH₂)ₙ—C(O)(LIGAND); TAP-C(O)—(CH₂)ₙ—C(O)O(LIGAND); TAP-C(O)—O(LIGAND); TAP-C(O)—(CH₂)ₙ—NH—C(O)(LIGAND); TAP-C(O)—(CH₂)ₙ(LIGAND); TAP-C(O)—NH(LIGAND); TAP-C(O)(LIGAND); TAP-(CH₂)ₙ—C(O) (LIGAND); TAP-(CH₂)ₙ—C(O)O(LIGAND); TAP-(CH₂)ₙ(LIGAND); or TAP-(CH₂)ₙ—NH—C(O)(LIGAND). In some embodiments, amino terminated linkers/tethers (e.g., NH₂, ONH₂, NH₂NH₂) can form an imino bond (i.e., C═N) with the ligand. In some embodiments, amino terminated linkers/tethers (e.g., NH₂, ONH₂, NH₂NH₂) can acylated, e.g., with C(O)CF₃.

In some embodiments, the linker/tether can terminate with a mercapto group (i.e., SH) or an olefin (e.g., CH═CH₂). For example, the tether can be TAP-(CH₂)ₙ—SH, TAP-C(O)(CH₂)ₙSH, TAP-(CH₂)ₙ—(CH═CH₂), or TAP-C(O)(CH₂)ₙ(CH═CH₂), in which n can be as described elsewhere. The tether may optionally be substituted, e.g., with hydroxy, alkoxy, perhaloalkyl, and/or optionally inserted with one or more additional heteroatoms, e.g., N, O, or S. The double bond can be cis or trans or E or Z.

In other embodiments, the linker/tether may include an electrophilic moiety, preferably at the terminal position of the linker/tether. Exemplary electrophilic moieties include, e.g., an aldehyde, alkyl halide, mesylate, tosylate, nosylate, or brosylate, or an activated carboxylic acid ester, e.g. an NHS ester, or a pentafluorophenyl ester. Preferred linkers/tethers (underlined) include TAP-(CH₂)ₙCHO; TAP-C(O)(CH₂)ₙCHO; or TAP-NR″″(CH₂)ₙCHO, in which n is 1-6 and R″″ is C₁-C₆ alkyl; or TAP-(CH₂)ₙC(O)ONHS; TAP-C(O)(CH₂)ₙC(O)ONHS; or TAP-NR″″(CH₂)ₙC(O)ONHS, in which n is 1-6 and R″″ is C₁-C₆ alkyl; TAP-(CH₂)ₙC(O)OC₆F₅; TAP-C(O)(CH₂)ₙC(O)OC₆F₅; or TAP-NR″″(CH₂)ₙC(O)OC₆F₅, in which n is 1-11 and R″″ is C₁-C₆ alkyl; or —(CH₂)ₙCH₂LG; TAP-C(O)(CH₂)ₙCH₂LG; or TAP-NR″″(CH₂)ₙCH₂LG, in which n can be as described elsewhere and R″″ is C₁-C₆ alkyl (LG can be a leaving group, e.g., halide, mesylate, tosylate, nosylate, brosylate). Tethering can be carried out by coupling a nucleophilic group of a ligand, e.g., a thiol or amino group with an electrophilic group on the tether.

In other embodiments, it can be desirable for the monomer to include a phthalimido group (K) at the terminal position of the linker/tether.

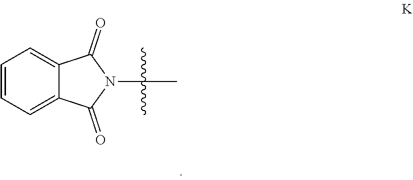 [SMILES: *N1C(=O)c2ccccc2C1=O]

In other embodiments, other protected amino groups can be at the terminal position of the linker/tether, e.g., alloc, monomethoxy trityl (MMT), trifluoroacetyl, Fmoc, or aryl sulfonyl (e.g., the aryl portion can be ortho-nitrophenyl or ortho, para-dinitrophenyl).

Any of the linkers/tethers described herein may further include one or more additional linking groups, e.g., —O—(CH₂)ₙ—, —(CH₂)ₙ—SS—, —(CH₂)ₙ—, or —(CH═CH)—.

### Cleavable Linkers Tethers

In some embodiments, at least one of the linkers/tethers can be a redox cleavable linker, an acid cleavable linker, an esterase cleavable linker, a phosphatase cleavable linker, or a peptidase cleavable linker.

In one embodiment, at least one of the linkers/tethers can be a reductively cleavable linker (e.g., a disulfide group).

In one embodiment, at least one of the linkers/tethers can be an acid cleavable linker (e.g., a hydrazone group, an ester group, an acetal group, or a ketal group).

In one embodiment, at least one of the linkers/tethers can be an esterase cleavable linker (e.g., an ester group).

In one embodiment, at least one of the linkers/tethers can be a phosphatase cleavable linker (e.g., a phosphate group).

In one embodiment, at least one of the linkers/tethers can be a peptidase cleavable linker (e.g., a peptide bond).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some tethers will have a linkage group that is cleaved at a preferred pH, thereby releasing the iRNA agent from a ligand (e.g., a targeting or cell-permeable ligand, such as cholesterol) inside the cell, or into the desired compartment of the cell.

A chemical junction (e.g., a linking group) that links a ligand to an iRNA agent can include a disulfide bond. When the iRNA agent/ligand complex is taken up into the cell by endocytosis, the acidic environment of the endosome will cause the disulfide bond to be cleaved, thereby releasing the iRNA agent from the ligand (Quintana et al., Pharm Res. 19:1310-1316, 2002; Patri et al., Curr. Opin. Curr. Biol. 6:466-471, 2002). The ligand can be a targeting ligand or a second therapeutic agent that may complement the therapeutic effects of the iRNA agent.

A tether can include a linking group that is cleavable by a particular enzyme. The type of linking group incorporated into a tether can depend on the cell to be targeted by the iRNA agent. For example, an iRNA agent that targets an mRNA in liver cells can be conjugated to a tether that includes an ester group. Liver cells are rich in esterases, and therefore the tether will be cleaved more efficiently in liver cells than in cell types that are not esterase-rich. Cleavage of the tether releases the iRNA agent from a ligand that is attached to the distal end of the tether, thereby potentially enhancing silencing activity of the iRNA agent. Other cell-types rich in esterases include cells of the lung, renal cortex, and testis.

Tethers that contain peptide bonds can be conjugated to iRNA agents target to cell types rich in peptidases, such as liver cells and synoviocytes. For example, an iRNA agent targeted to synoviocytes, such as for the treatment of an inflammatory disease (e.g., rheumatoid arthritis), can be conjugated to a tether containing a peptide bond.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue, e.g., tissue the iRNA agent would be exposed to when administered to a subject. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In preferred embodiments, useful candidate compounds are cleaved at least 2, 4, 10 or 100 times faster in the cell (or under in vitro conditions selected to mimic intracellular conditions) as compared to blood or serum (or under in vitro conditions selected to mimic extracellular conditions).

### Redox Cleavable Linking Groups

One class of cleavable linking groups are redox cleavable linking groups that are cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In a preferred embodiment, candidate compounds are cleaved by at most 10% in the blood. In preferred embodiments, useful candidate compounds are degraded at least 2, 4, 10 or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

### Phosphate-Based Cleavable Linking Groups

Phosphate-based linking groups are cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—. Preferred embodiments are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, —O—P(S)(H)—S—. A preferred embodiment is —O—P(O)(OH)—O—. These candidates can be evaluated using methods analogous to those described above.

### Acid Cleavable Linking Groups

Acid cleavable linking groups are linking groups that are cleaved under acidic conditions. In preferred embodiments acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, ketals, acetals, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

### Ester-Based Linking Groups

Ester-based linking groups are cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

### Peptide-Based Cleaving Groups

Peptide-based linking groups are cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynylene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide cleavable linking groups have the general formula —NHCHR¹C(O)NHCHR²C(O)—, where R¹ and R² are the R groups of the two adjacent amino acids. These candidates can be evaluated using methods analogous to those described above.

### Biocleavable Linkers/Tethers

The linkers can also includes biocleavable linkers that are nucleotide and non-nucleotide linkers or combinations thereof that connect two parts of a molecule, for example, one or both strands of two individual siRNA molecule to generate a bis(siRNA). In some embodiments, mere electrostatic or stacking interaction between two individual siRNAs can represent a linker. The non-nucleotide linkers include tethers or linkers derived from monosaccharides, disaccharides, oligosaccharides, and derivatives thereof, aliphatic, alicyclic, heterocyclic, and combinations thereof.

In some embodiments, at least one of the linkers (tethers) is a bio-clevable linker selected from the group consisting of DNA, RNA, disulfide, amide, functionalized monosaccharides or oligosaccharides of galactosamine, glucosamine, glucose, galactose, and mannose, and combinations thereof.

In one embodiment, the bio-cleavable carbohydrate linker may have 1 to 10 saccharide units, which have at least one anomeric linkage capable of connecting two siRNA units. When two or more saccharides are present, these units can be linked via 1-3, 1-4, or 1-6 sugar linkages, or via alkyl chains.

Exemplary Bio-Cleavable Linkers Include:

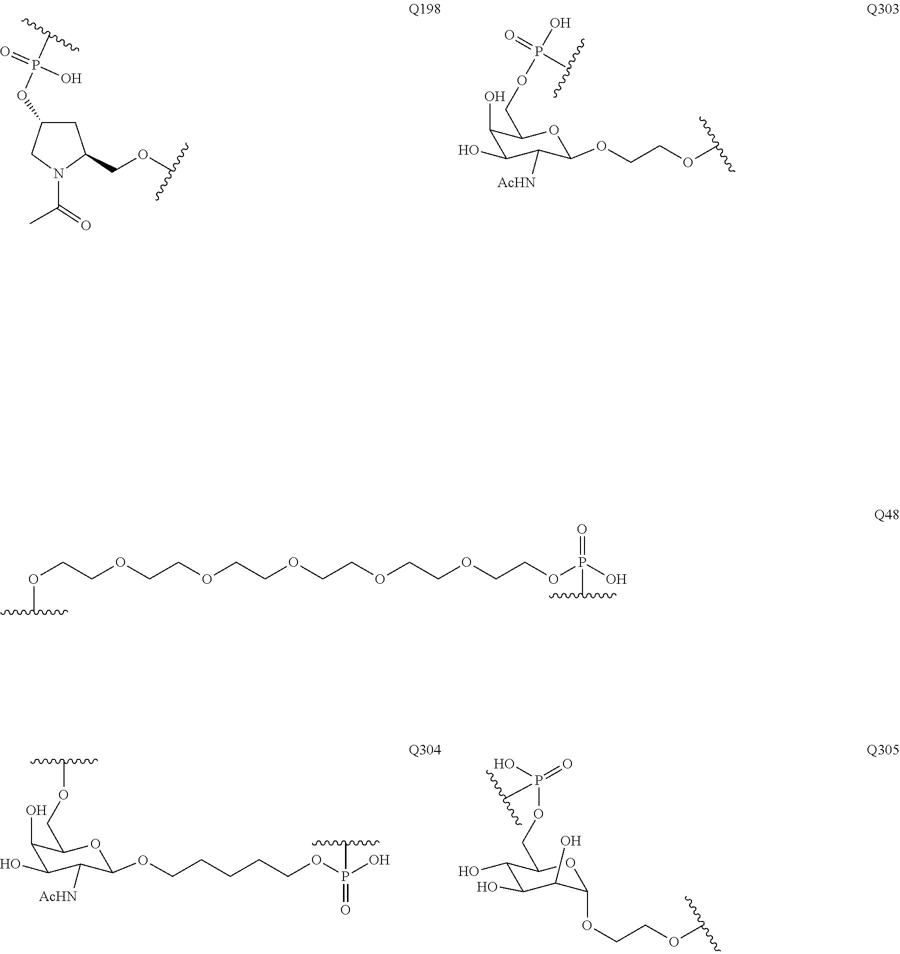

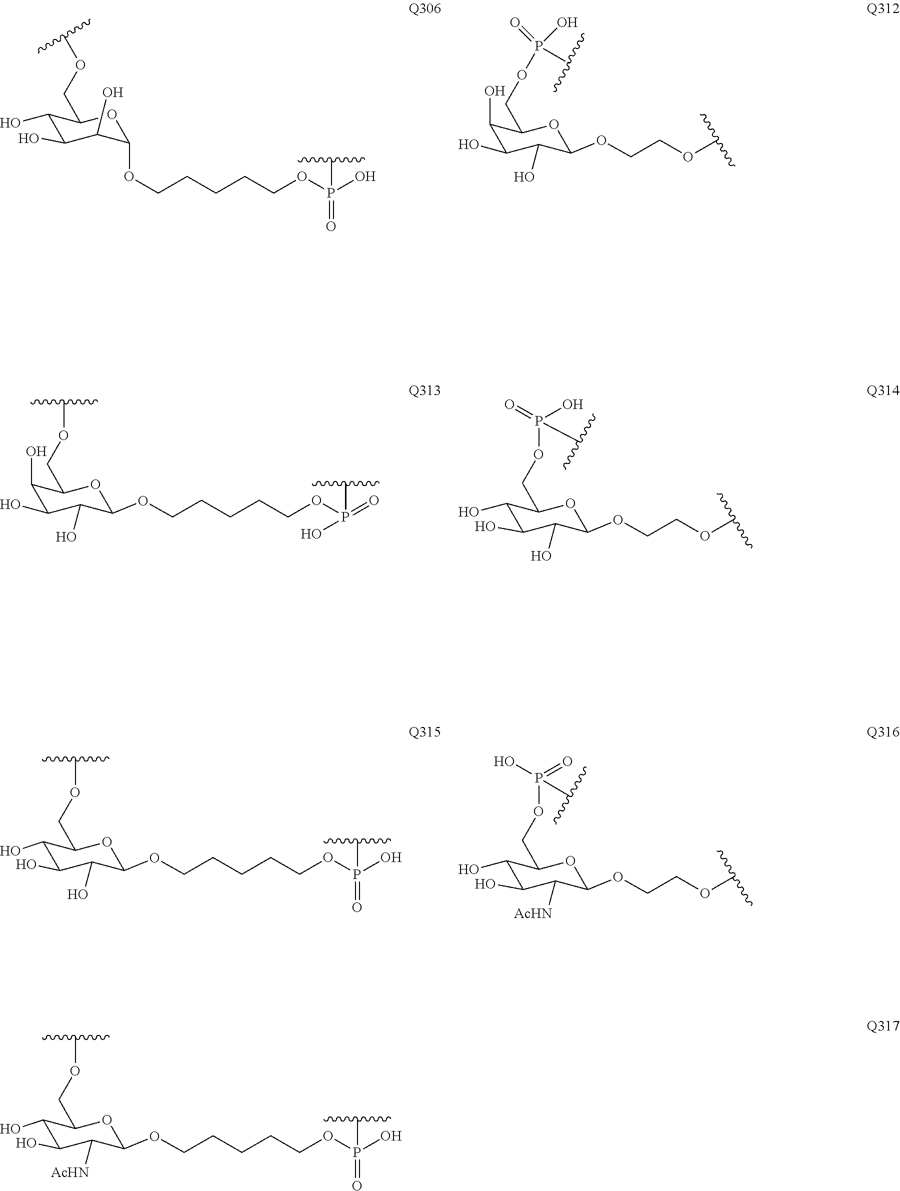

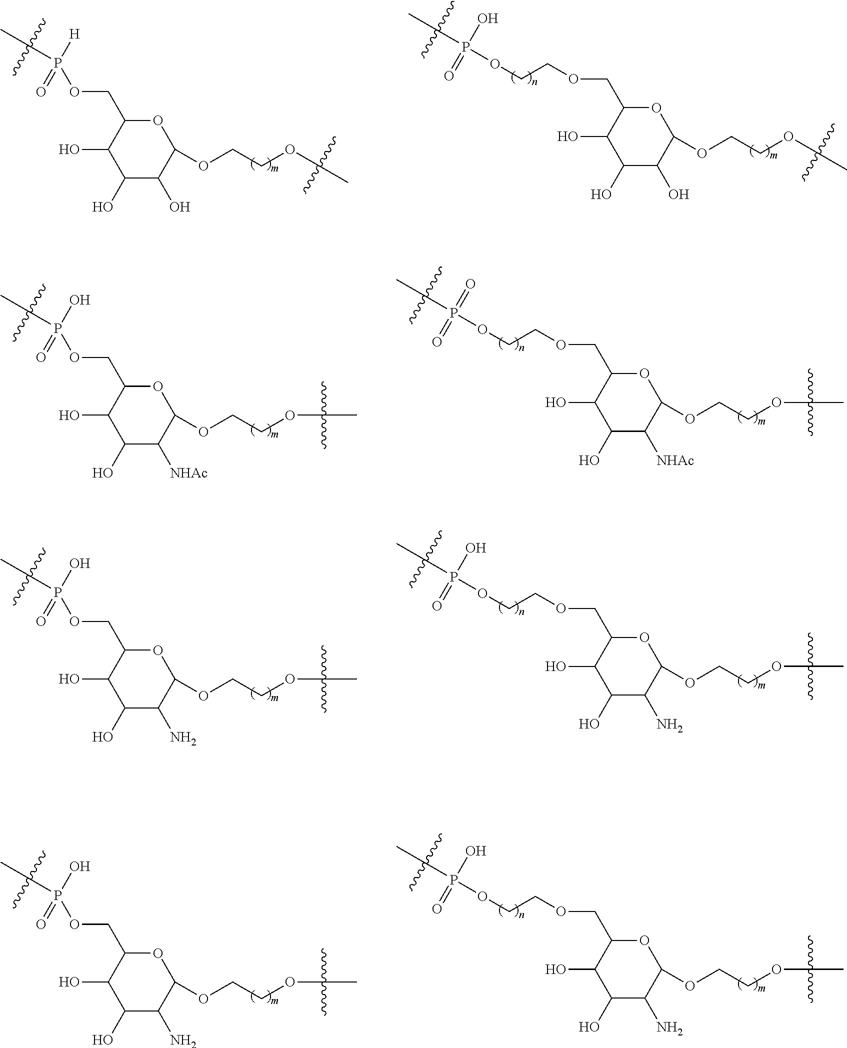

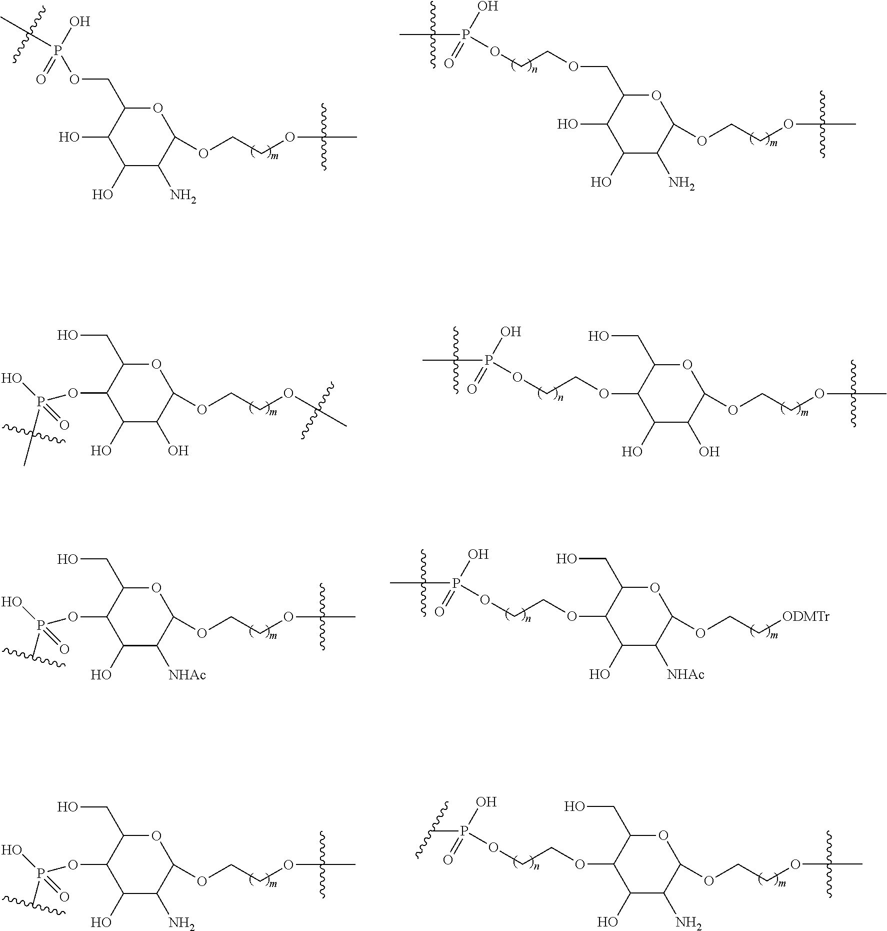

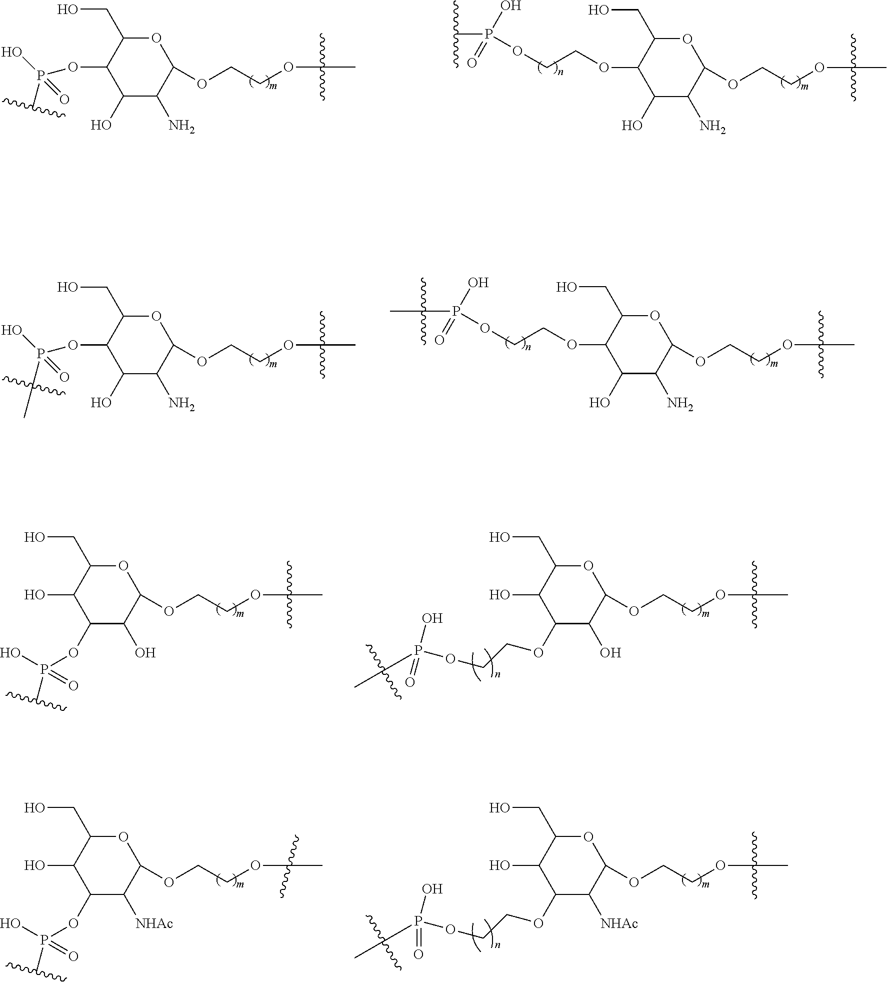

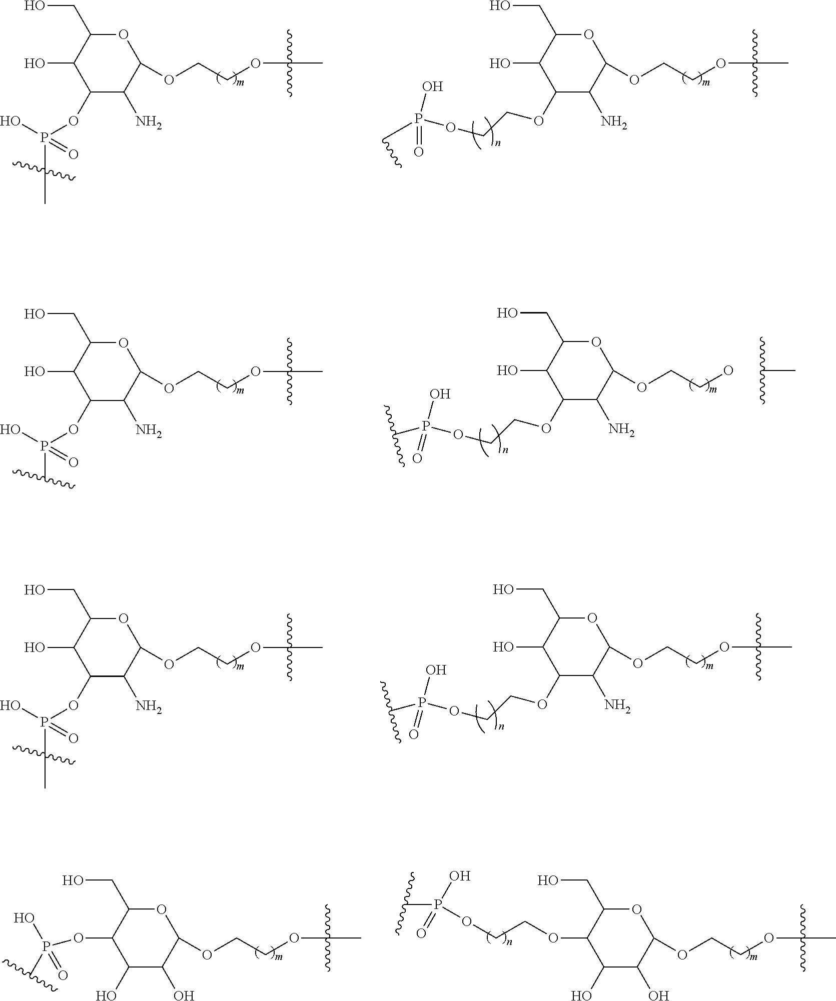

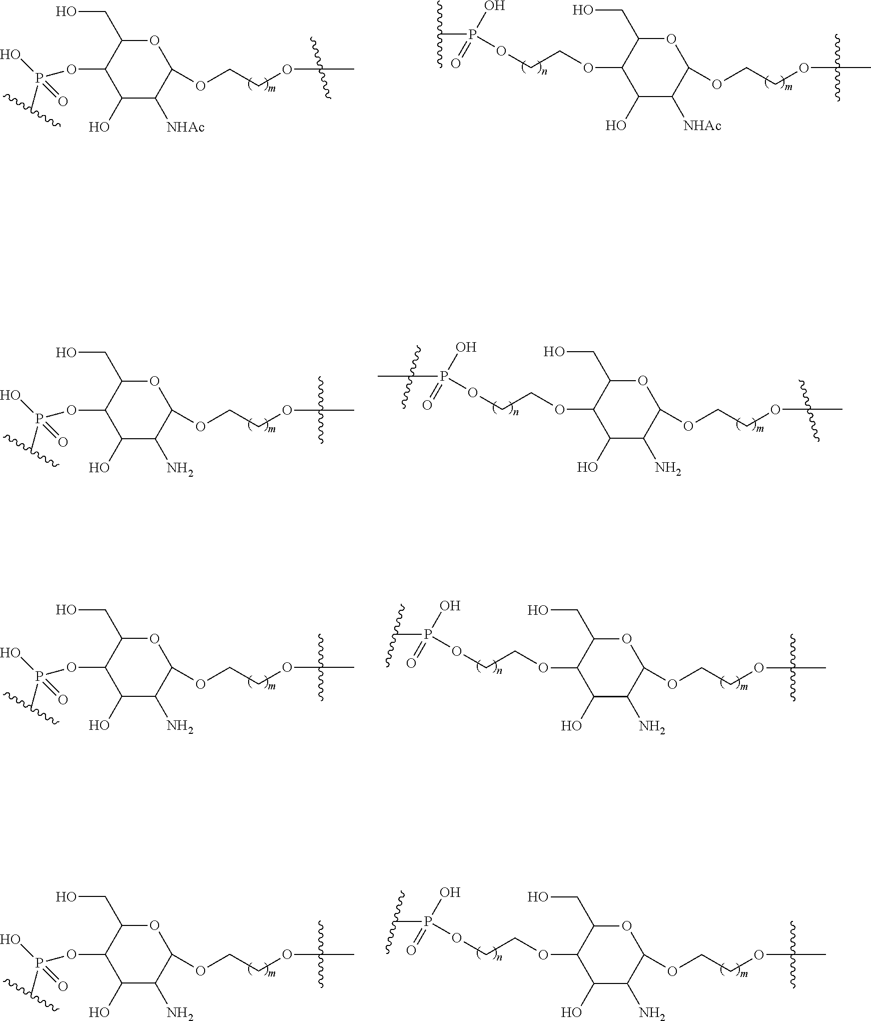

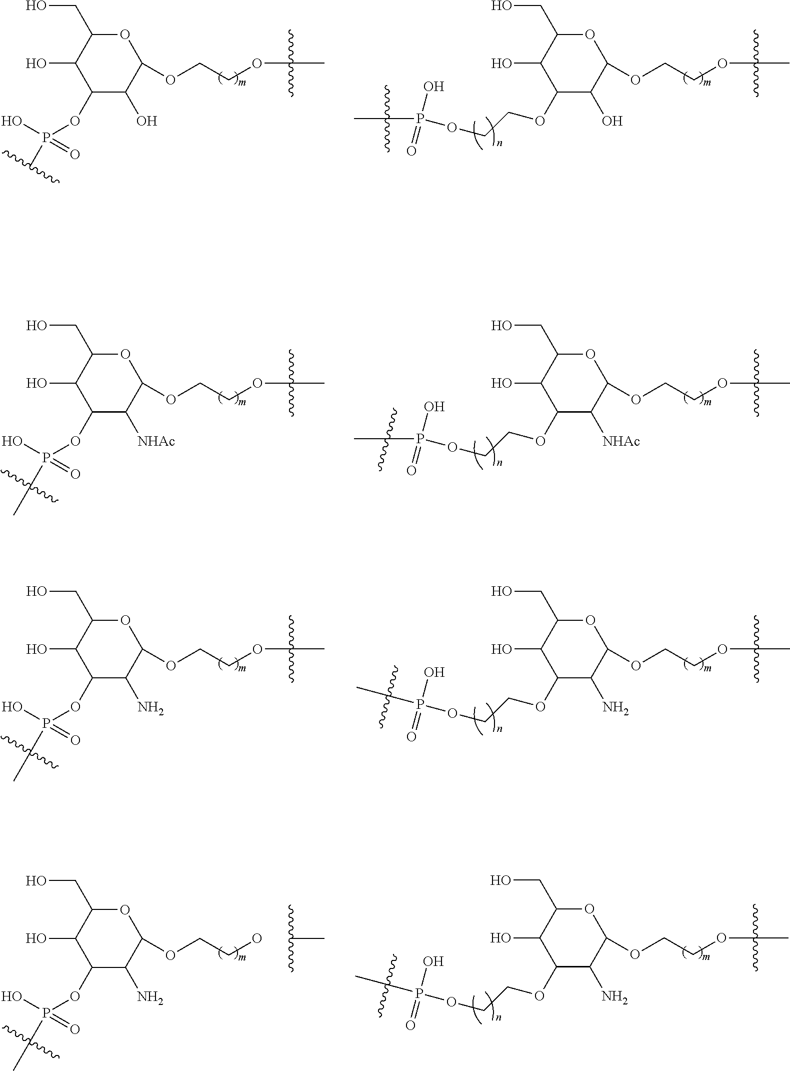

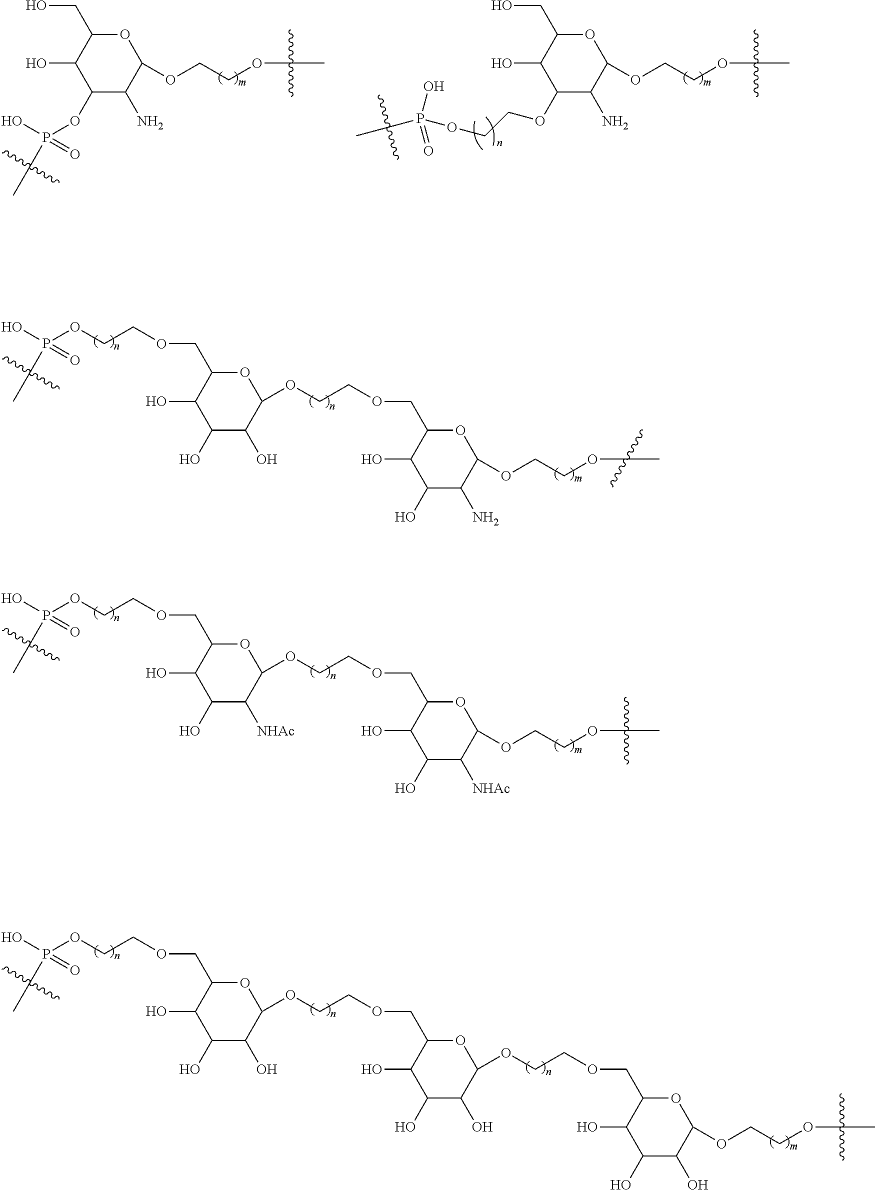

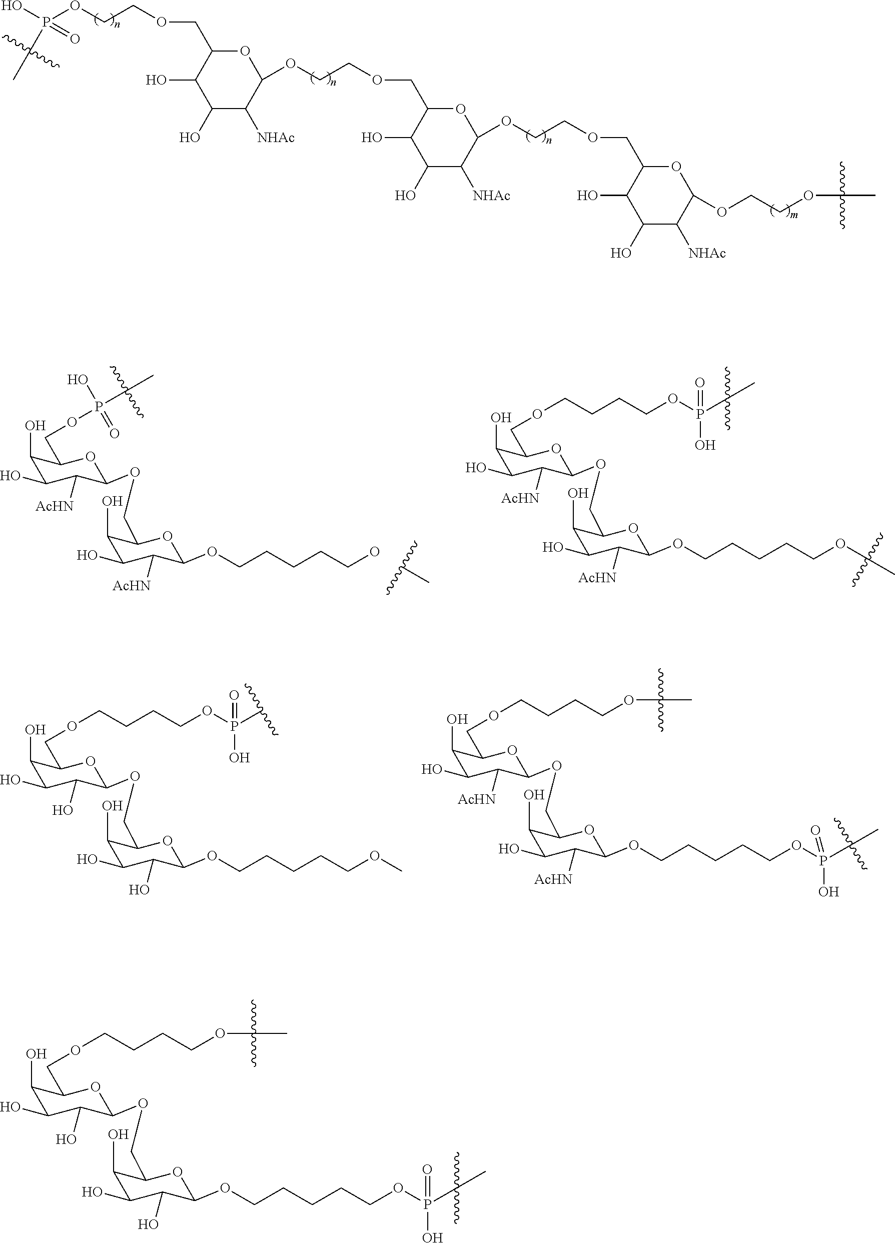

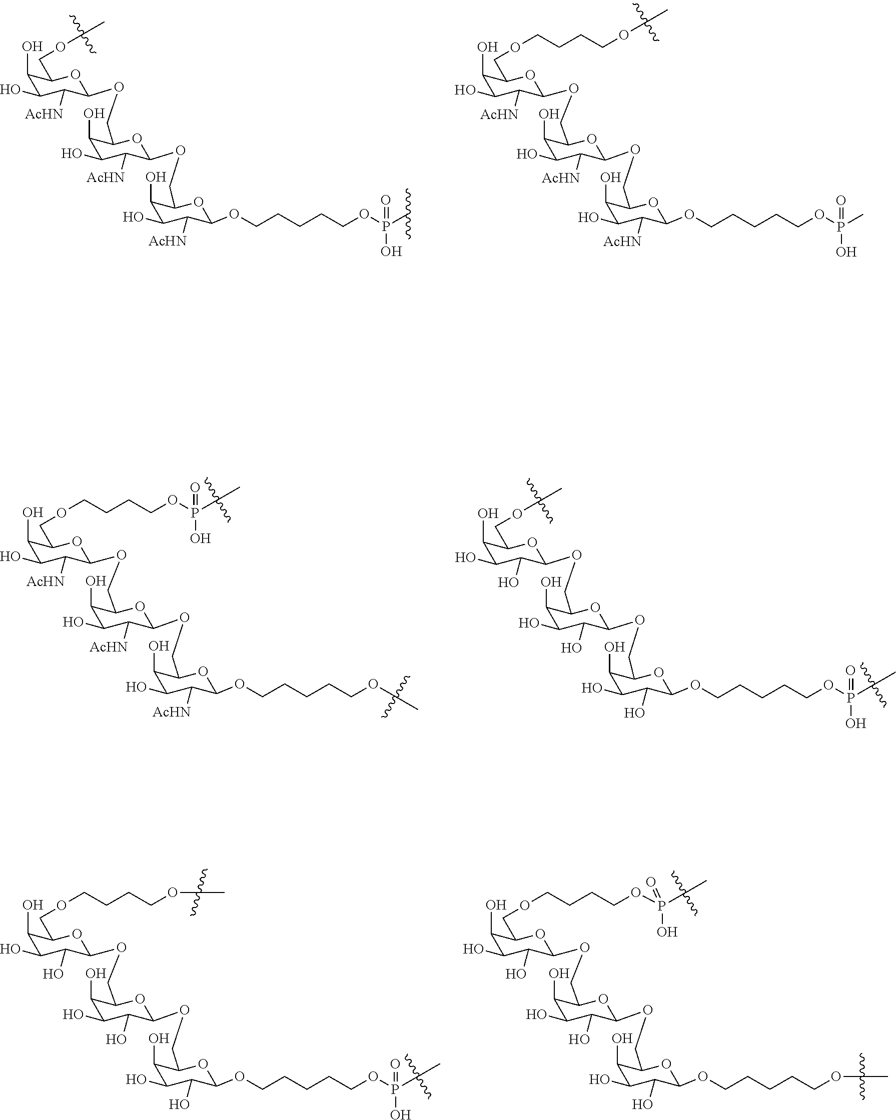

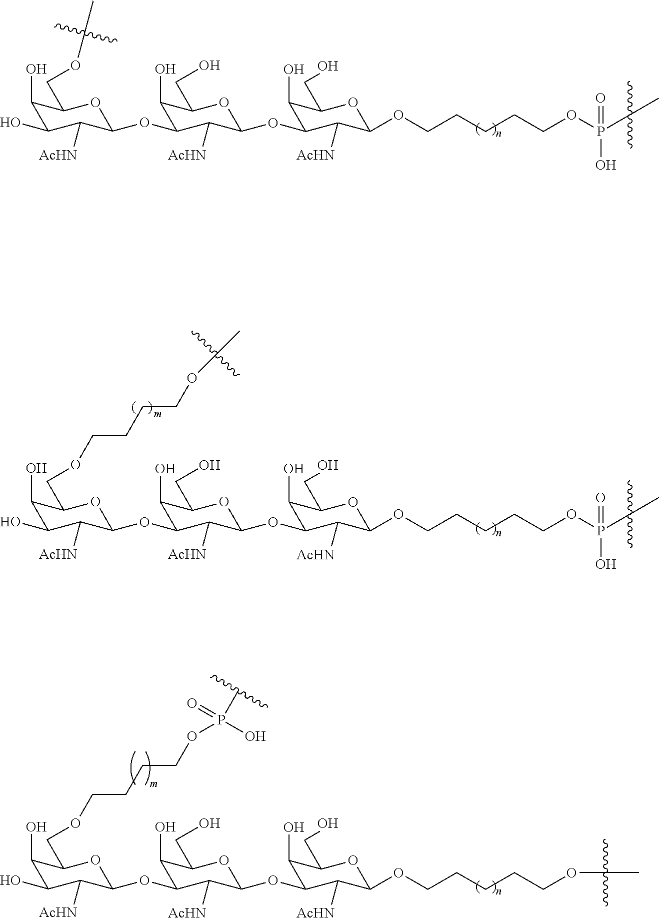

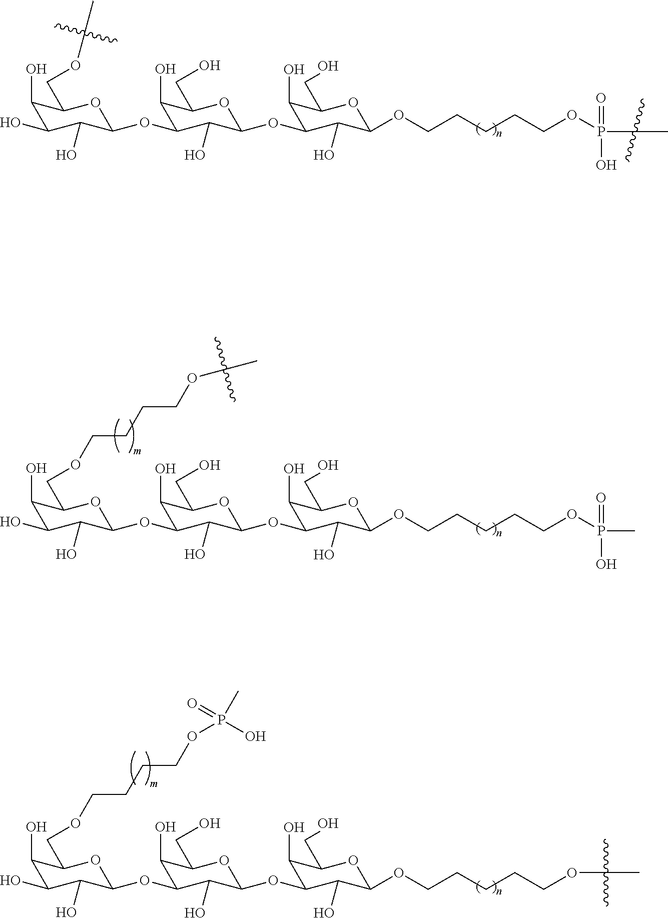

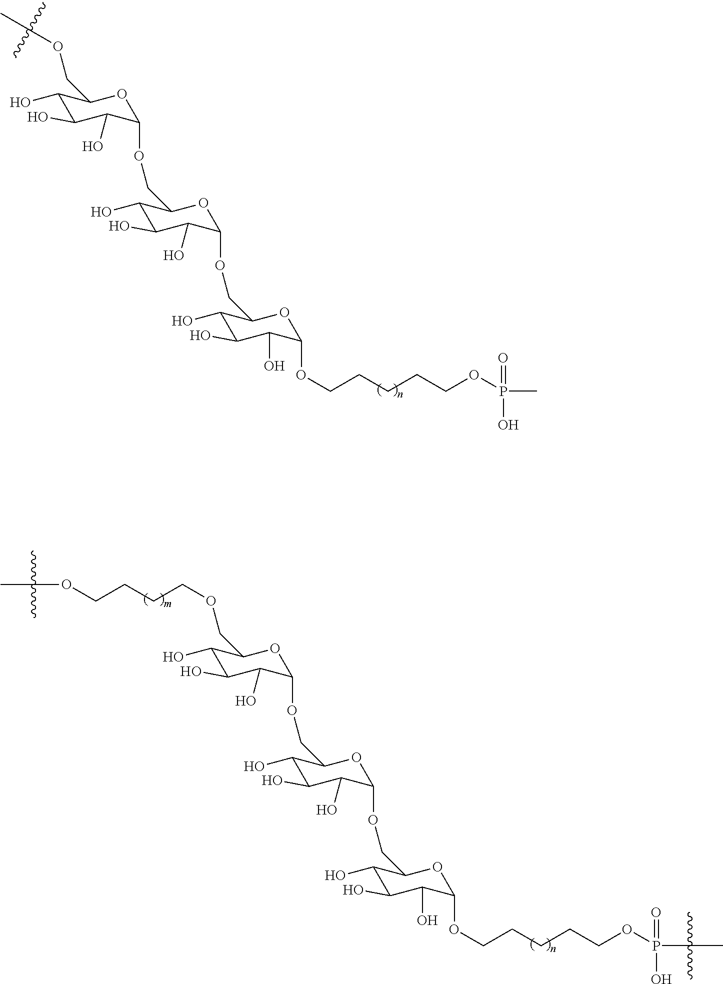

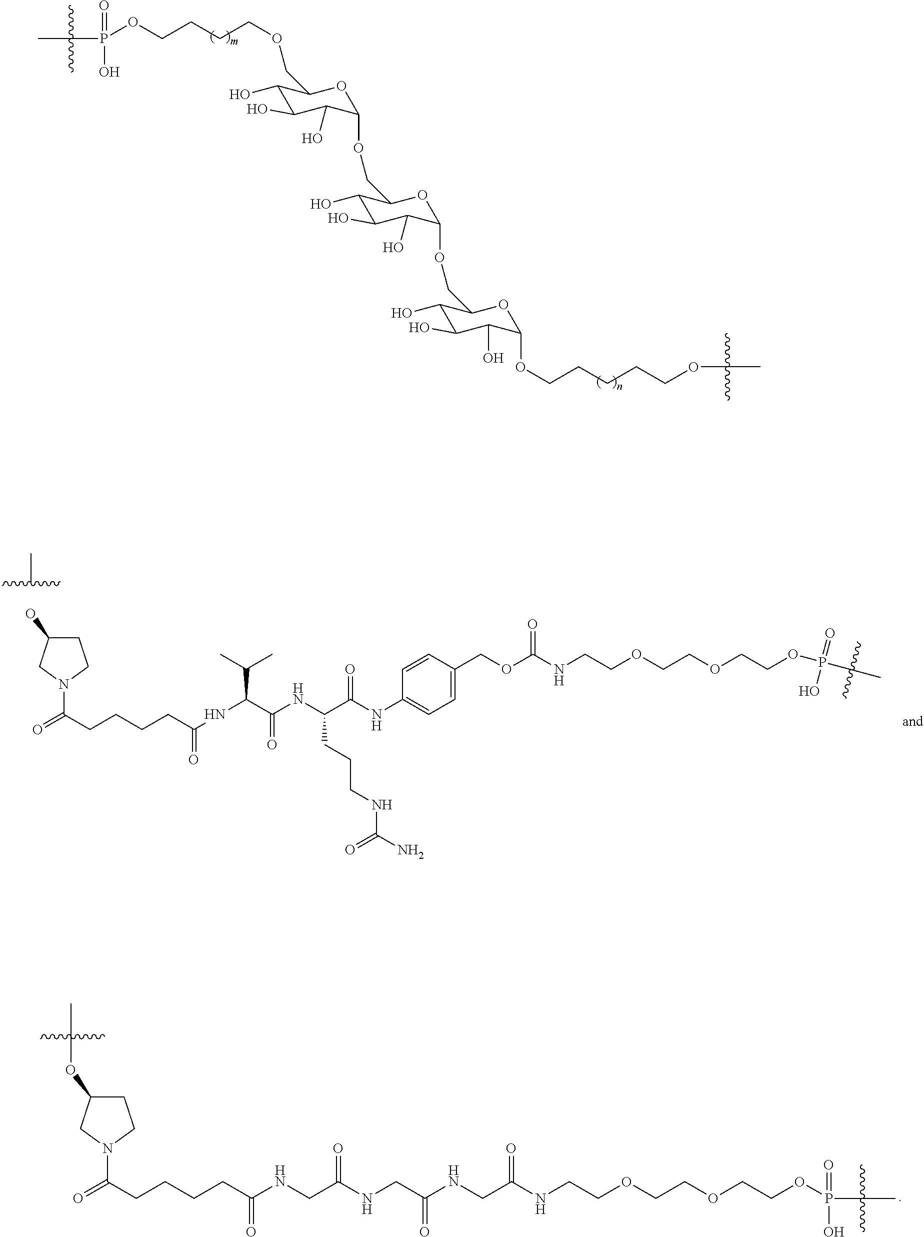

More discussion about the biocleavable linkers may be found in PCT application No. PCT/US18/14213, entitled “Endosomal Cleavable Linkers,” filed on Jan. 18, 2018, the content of which is incorporated herein by reference in its entirety.

### Carriers

In certain embodiments, the ligand is conjugated to the iRNA agent via a carrier that replaces one or more nucleotide(s).

The carrier can be a cyclic group or an acyclic group. In one embodiment, the cyclic group is selected from the group consisting of cyclohexyl, pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl, and decalin. In one embodiment, the acyclic group is a moiety based on a serinol backbone or a diethanolamine backbone.

In some embodiments, the carrier replaces one or more nucleotide(s) in the internal position(s) of the double-stranded iRNA agent.

In other embodiments, the carrier replaces the nucleotides at the terminal end of the sense strand or antisense strand. In one embodiment, the carrier replaces the terminal nucleotide on the 3′ end of the sense strand, thereby functioning as an end cap protecting the 3′ end of the sense strand. In one embodiment, the carrier is a cyclic group having an amine, for instance, the carrier may be cyclohexyl, pyrrolidinyl, pyrazolinyl, pyrazolidinyl, imidazolinyl, imidazolidinyl, piperidinyl, piperazinyl, [1,3]dioxolanyl, oxazolidinyl, isoxazolidinyl, morpholinyl, thiazolidinyl, isothiazolidinyl, quinoxalinyl, pyridazinonyl, tetrahydrofuranyl, or decalinyl.

A ribonucleotide subunit in which the ribose sugar of the subunit has been so replaced is referred to herein as a ribose replacement modification subunit (RRMS). The carrier can be a cyclic or acyclic moiety and include two “backbone attachment points” (e.g., hydroxyl groups) and a ligand (e.g., the carbohydrate-based ligand). The ligand can be directly attached to the carrier or indirectly attached to the carrier by an intervening linker/tether, as described above.

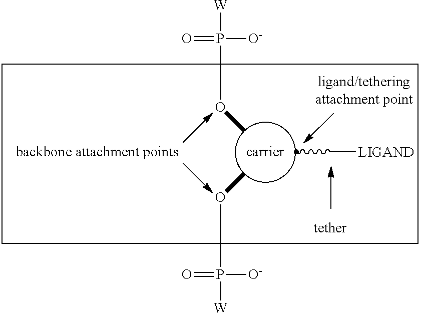

The ligand-conjugated monomer subunit may be the 5′ or 3′ terminal subunit of the iRNA molecule, i.e., one of the two “W” groups may be a hydroxyl group, and the other “W” group may be a chain of two or more unmodified or modified ribonucleotides. Alternatively, the ligand-conjugated monomer subunit may occupy an internal position, and both “W” groups may be one or more unmodified or modified ribonucleotides. More than one ligand-conjugated monomer subunit may be present in an iRNA agent.

### Sugar Replacement-Based Monomers, e.g., Ligand-Conjugated Monomers (Cyclic)

Cyclic sugar replacement-based monomers, e.g., sugar replacement-based ligand-conjugated monomers, are also referred to herein as RRMS monomer compounds. The carriers may have the general formula (LCM-2) provided below (In that structure preferred backbone attachment points can be chosen from R¹ or R²; R³ or R⁴; or R⁹ and R¹⁰ if Y is CR⁹R¹⁰ (two positions are chosen to give two backbone attachment points, e.g., R¹ and R⁴, or R⁴ and R⁹)). Preferred tethering attachment points include R⁷; R⁵ or R⁶ when X is CH₂. The carriers are described below as an entity, which can be incorporated into a strand. Thus, it is understood that the structures also encompass the situations wherein one (in the case of a terminal position) or two (in the case of an internal position) of the attachment points, e.g., R¹ or R²; R³ or R⁴; or R⁹ or R¹⁰ (when Y is CR⁹R¹⁰), is connected to the phosphate, or modified phosphate, e.g., sulfur containing, backbone. E.g., one of the above-named R groups can be —CH₂—, wherein one bond is connected to the carrier and one to a backbone atom, e.g., a linking oxygen or a central phosphorus atom.

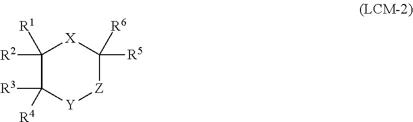

wherein:
X is N(CO)R⁷, NR⁷ or CH₂;
Y is NR⁸, O, S, CR⁹R¹⁰;
Z is CR¹¹R¹² or absent;
Each of R¹, R², R³, R⁴, R⁹, and R¹⁰ is, independently, H, OR^{a}, or (CH₂)ₙOR^{b}, provided that at least two of R¹, R², R³, R⁴, R⁹, and R¹⁰ are OR^{a} and/or (CH₂)ₙOR^{b};
Each of R⁵, R⁶, R¹¹, and R¹² is, independently, a ligand, H, C₁-C₆ alkyl optionally substituted with 1-3 R¹³, or C(O)NHR⁷; or R⁵ and R¹¹ together are C₃-C₈ cycloalkyl optionally substituted with R¹⁴;
R⁷ can be a ligand, e.g., R⁷ can be R^{d}, or R⁷ can be a ligand tethered indirectly to the carrier, e.g., through a tethering moiety, e.g., C₁-C₂₀ alkyl substituted with NR^{c}R^{d}; or C₁-C₂₀ alkyl substituted with NHC(O)R^{d};
R⁸ is H or C₁-C₆ alkyl;
R¹³ is hydroxy, C₁-C₄ alkoxy, or halo;
R¹⁴ is NR^{c}R⁷;
R¹⁵ is C₁-C₆ alkyl optionally substituted with cyano, or C₂-C₆ alkenyl;
R¹⁶ is C₁-C₁₀ alkyl;
R¹⁷ is a liquid or solid phase support reagent;
L is —C(O)(CH₂)_{q}C(O)—, or —C(O)(CH₂)_{q}S—;
R^{a} is a protecting group, e.g., CAr₃; (e.g., a dimethoxytrityl group) or Si(X^{5′})(X^{5″})(X^{5′″}) in which (X^{5′}),(X^{5″}), and (X^{5′″}) are as described elsewhere.
R^{b} is P(O)(O⁻)H, P(OR¹⁵)N(R¹⁶)₂ or L-R¹⁷;
R^{c} is H or C₁-C₆ alkyl;
R^{d} is H or a ligand;
Each Ar is, independently, C₆-C₁₀ aryl optionally substituted with C₁-C₄ alkoxy;
n is 1-4; and q is 0-4.

Exemplary carriers include those in which, e.g., X is N(CO)R⁷ or NR⁷, Y is CR⁹R¹⁰, and Z is absent; or X is N(CO)R⁷ or NR⁷, Y is CR⁹R¹⁰, and Z is CR¹¹R¹²; or X is N(CO)R⁷ or NR⁷, Y is NR⁸, and Z is CR¹¹R¹²; or X is N(CO)R⁷ or NR⁷, Y is O, and Z is CR¹¹R¹²; or X is CH₂; Y is CR⁹R¹⁰; Z is CR¹¹R¹², and R⁵ and R¹¹ together form C₆ cycloalkyl (H, z=2), or the indane ring system, e.g., X is CH₂; Y is CR⁹R¹⁰; Z is CR¹¹R¹², and R⁵ and R¹¹ together form C₅ cycloalkyl (H, z=1).

In certain embodiments, the carrier may be based on the pyrroline ring system or the 4-hydroxyproline ring system, e.g., X is N(CO)R⁷ or NR⁷, Y is CR⁹R¹⁰, and Z is absent (D).

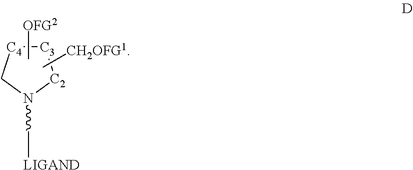

OFG¹ is preferably attached to a primary carbon, e.g., an exocyclic alkylene group, e.g., a methylene group, connected to one of the carbons in the five-membered ring (—CH₂OFG¹ in D). OFG² is preferably attached directly to one of the carbons in the five-membered ring (—OFG² in D). For the pyrroline-based carriers, —CH₂OFG¹ may be attached to C-2 and OFG² may be attached to C-3; or —CH₂OFG¹ may be attached to C-3 and OFG² may be attached to C-4. In certain embodiments, CH₂OFG¹ and OFG² may be geminally substituted to one of the above-referenced carbons. For the 3-hydroxyproline-based carriers, —CH₂OFG¹ may be attached to C-2 and OFG² may be attached to C-4. The pyrroline- and 4-hydroxyproline-based monomers may therefore contain linkages (e.g., carbon-carbon bonds) wherein bond rotation is restricted about that particular linkage, e.g. restriction resulting from the presence of a ring. Thus, CH₂OFG¹ and OFG² may be cis or trans with respect to one another in any of the pairings delineated above Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen. Preferred examples of carrier D include the following:

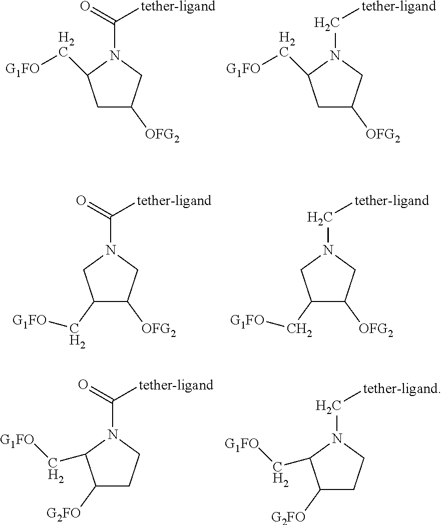

In certain embodiments, the carrier may be based on the piperidine ring system (E), e.g., X is N(CO)R⁷ or NR⁷, Y is CR⁹R¹⁰, and Z is CR¹¹R¹².

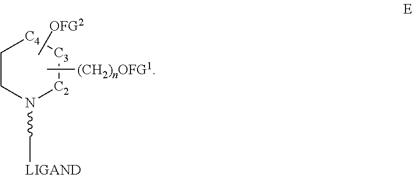

OFG¹ is preferably attached to a primary carbon, e.g., an exocyclic alkylene group, e.g., a methylene group (n=1) or ethylene group (n=2), connected to one of the carbons in the six-membered ring [—(CH₂)ₙOFG¹ in E]. OFG² is preferably attached directly to one of the carbons in the six-membered ring (—OFG² in E). —(CH₂)ₙOFG¹ and OFG² may be disposed in a geminal manner on the ring, i.e., both groups may be attached to the same carbon, e.g., at C-2, C-3, or C-4. Alternatively, —(CH₂)ₙOFG¹ and OFG² may be disposed in a vicinal manner on the ring, i.e., both groups may be attached to adjacent ring carbon atoms, e.g., —(CH₂)ₙOFG¹ may be attached to C-2 and OFG² may be attached to C-3; —(CH₂)ₙOFG¹ may be attached to C-3 and OFG² may be attached to C-2; —(CH₂)ₙOFG¹ may be attached to C-3 and OFG² may be attached to C-4; or —(CH₂)ₙOFG¹ may be attached to C-4 and OFG² may be attached to C-3. The piperidine-based monomers may therefore contain linkages (e.g., carbon-carbon bonds) wherein bond rotation is restricted about that particular linkage, e.g. restriction resulting from the presence of a ring. Thus, —(CH₂)ₙOFG¹ and OFG² may be cis or trans with respect to one another in any of the pairings delineated above. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen.

In certain embodiments, the carrier may be based on the piperazine ring system (F), e.g., X is N(CO)R⁷ or NR⁷, Y is NR⁸, and Z is CR¹¹R¹², or the morpholine ring system (G), e.g., X is N(CO)R⁷ or NR⁷, Y is O, and Z is CR¹¹R¹².

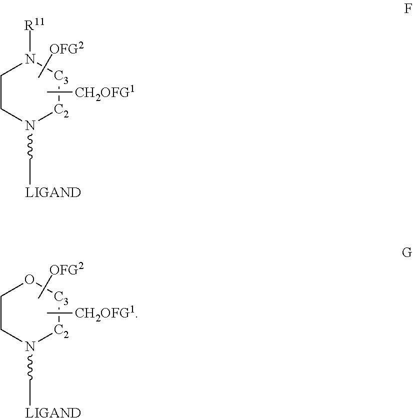

OFG¹ is preferably attached to a primary carbon, e.g., an exocyclic alkylene group, e.g., a methylene group, connected to one of the carbons in the six-membered ring (—CH₂OFG¹ in F or G). OFG² is preferably attached directly to one of the carbons in the six-membered rings (—OFG² in F or G). For both F and G, —CH₂OFG¹ may be attached to C-2 and OFG² may be attached to C-3; or vice versa. In certain embodiments, CH₂OFG¹ and OFG² may be geminally substituted to one of the above-referenced carbons. The piperazine- and morpholine-based monomers may therefore contain linkages (e.g., carbon-carbon bonds) wherein bond rotation is restricted about that particular linkage, e.g. restriction resulting from the presence of a ring. Thus, CH₂OFG¹ and OFG² may be cis or trans with respect to one another in any of the pairings delineated above. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). R′″ can be, e.g., C₁-C₆ alkyl, preferably CH₃. The tethering attachment point is preferably nitrogen in both F and G.

In certain embodiments, the carrier may be based on the decalin ring system, e.g., X is CH₂; Y is CR⁹R¹⁰; Z is CR¹¹R¹², and R⁵ and R¹¹ together form C₆ cycloalkyl (H, z=2), or the indane ring system, e.g., X is CH₂; Y is CR⁹R¹⁰; Z is CR¹¹R¹², and R⁵ and R¹¹ together form C₅ cycloalkyl (H, z=1).

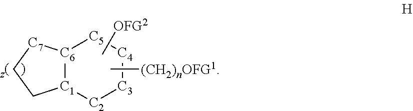

OFG¹ is preferably attached to a primary carbon, e.g., an exocyclic methylene group (n=1) or ethylene group (n=2) connected to one of C-2, C-3, C-4, or C-5 [—(CH₂)ₙOFG¹ in H]. OFG² is preferably attached directly to one of C-2, C-3, C-4, or C-5 (—OFG² in H). —(CH₂)ₙOFG^{I} and OFG² may be disposed in a geminal manner on the ring, i.e., both groups may be attached to the same carbon, e.g., at C-2, C-3, C-4, or C-5. Alternatively, —(CH₂)ₙOFG¹ and OFG² may be disposed in a vicinal manner on the ring, i.e., both groups may be attached to adjacent ring carbon atoms, e.g., —(CH₂)ₙOFG¹ may be attached to C-2 and OFG² may be attached to C-3; —(CH₂)ₙOFG¹ may be attached to C-3 and OFG² may be attached to C-2; —(CH₂)ₙOFG¹ may be attached to C-3 and OFG² may be attached to C-4; or —(CH₂)ₙOFG¹ may be attached to C-4 and OFG² may be attached to C-3; —(CH₂)ₙOFG¹ may be attached to C-4 and OFG² may be attached to C-5; or —(CH₂)ₙOFG¹ may be attached to C-5 and OFG² may be attached to C-4. The decalin or indane-based monomers may therefore contain linkages (e.g., carbon-carbon bonds) wherein bond rotation is restricted about that particular linkage, e.g. restriction resulting from the presence of a ring. Thus, —(CH₂)ₙOFG¹ and OFG² may be cis or trans with respect to one another in any of the pairings delineated above. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). In a preferred embodiment, the substituents at C-1 and C-6 are trans with respect to one another. The tethering attachment point is preferably C-6 or C-7.

Other carriers may include those based on 3-hydroxyproline (J).

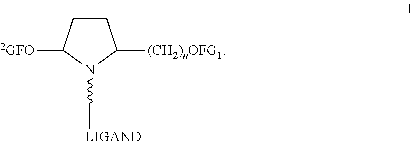

Thus, —(CH₂)ₙOFG¹ and OFG² may be cis or trans with respect to one another. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen.

Details about more representative cyclic, sugar replacement-based carriers can be found in U.S. Pat. Nos. 7,745,608 and 8,017,762, which are herein incorporated by reference in their entireties.

### Sugar Replacement-Based Monomers (Acyclic)

Acyclic sugar replacement-based monomers, e.g., sugar replacement-based ligand-conjugated monomers, are also referred to herein as ribose replacement monomer subunit (RRMS) monomer compounds. Preferred acyclic carriers can have formula LCM-3 or LCM-4:

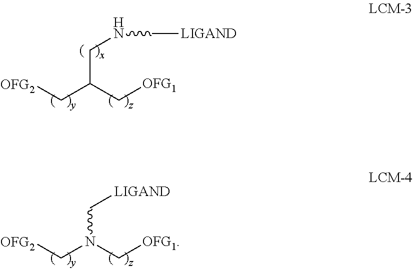

In some embodiments, each of x, y, and z can be, independently of one another, 0, 1, 2, or 3. In formula LCM-3, when y and z are different, then the tertiary carbon can have either the R or S configuration. In preferred embodiments, x is zero and y and z are each 1 in formula LCM-3 (e.g., based on serinol), and y and z are each 1 in formula LCM-3. Each of formula LCM-3 or LCM-4 below can optionally be substituted, e.g., with hydroxy, alkoxy, perhaloalkyl.

Details about more representative acyclic, sugar replacement-based carriers can be found in U.S. Pat. Nos. 7,745,608 and 8,017,762, which are herein incorporated by reference in their entireties.

In some embodiments, the double stranded iRNA agent comprises one or more ligands such as the carbohydrate-based ligands conjugated to the 5′ end of the sense strand or the 5′ end of the antisense strand.

In certain embodiments, the ligand such as the carbohydrate-based ligand is conjugated to the 5′-end of a strand via a carrier and/or linker. In one embodiment, the ligand such as the carbohydrate-based ligand is conjugated to the 5′-end of a strand via a carrier of a formula:

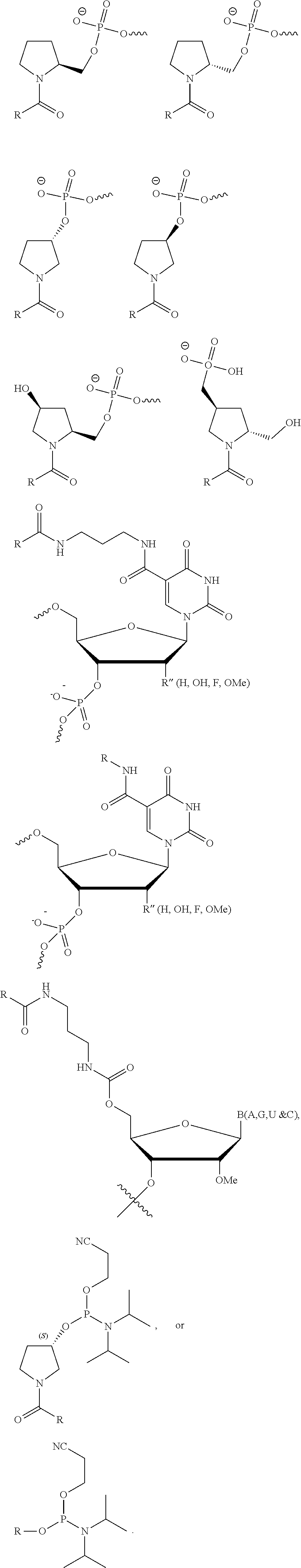

R is a ligand such as the carbohydrate-based ligand.

In some embodiments, the double stranded iRNA agent comprises one or more ligands such as the carbohydrate-based ligand conjugated to the 3′ end of the sense strand or the 3′ end of the antisense strand.

In certain embodiments, the ligand such as the carbohydrate-based ligand is conjugated to the 3′-end of a strand via a carrier and/or linker. In one embodiment, the ligand such as the carbohydrate-based ligand is conjugated to the 3′-end of a strand via a carrier of a formula:

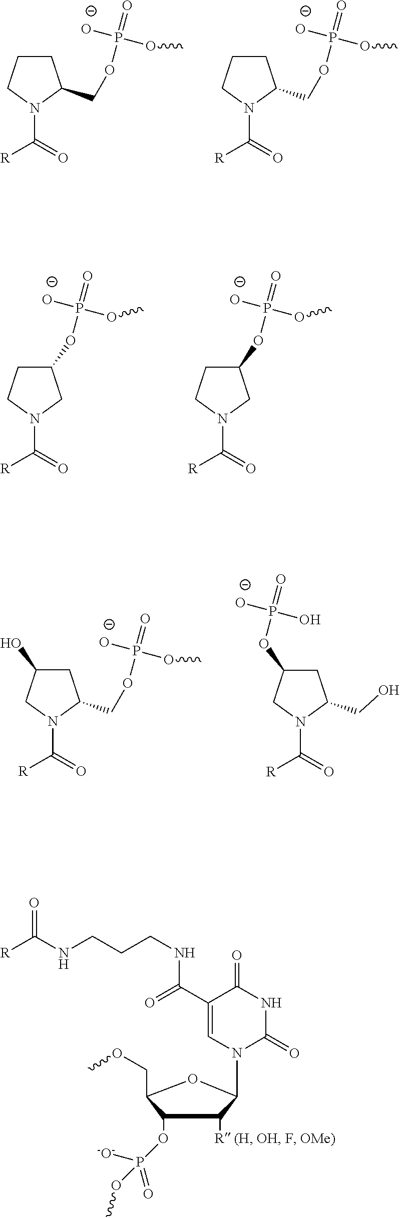

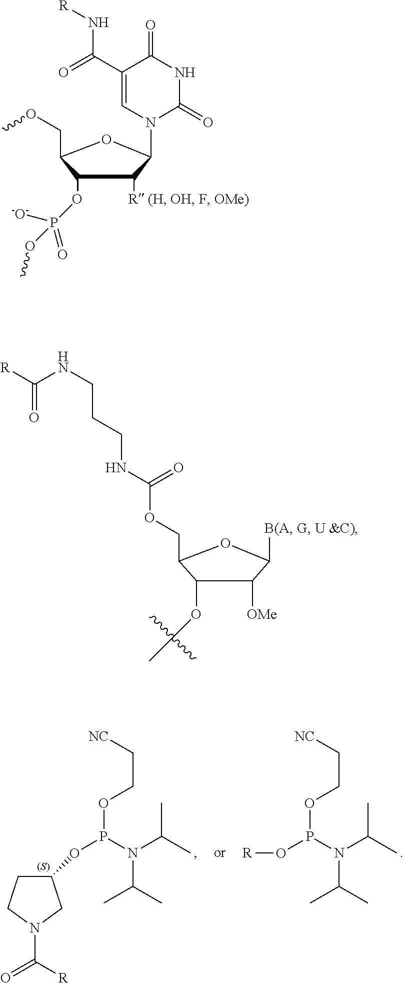

R is a ligand such as the carbohydrate-based ligand.

In some embodiments, the targeting ligand targets a liver tissue. In some embodiments, the targeting ligand is a carbohydrate-based ligand, such as an ASGPR ligand. In one embodiment, the targeting ligand is a GalNAc conjugate.

In some embodiments, the targeting ligand, such as a carbohydrate-based ligand (e.g., an ASGPR ligand), comprises one or more ligand moieties attached through a bivalent or trivalent branched linker.

In certain embodiments, the double-stranded iRNA agent comprises a targeting ligand having a structure shown below:

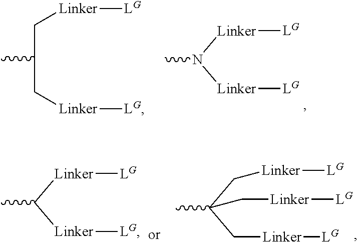

wherein:
L^{G} is independently for each occurrence a ligand, e.g., carbohydrate-based ligand, e.g. monosaccharide, disaccharide, trisaccharide, tetrasaccharide, polysaccharide; and
Z′, Z″, Z′″ and Z″″ are each independently for each occurrence O or S.

In certain embodiments, the double-stranded iRNA agent comprises a targeting ligand of Formula (II), (III), (IV) or (V):

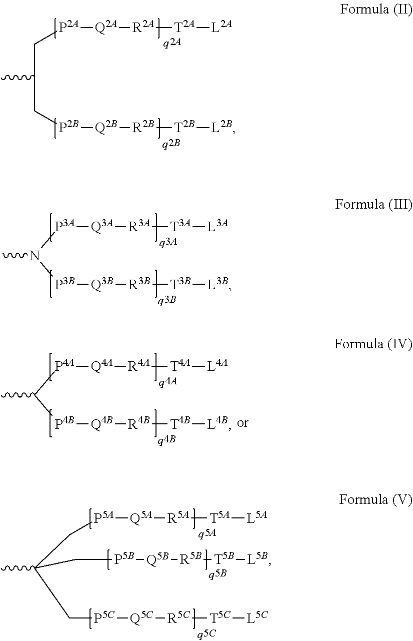

wherein:

q^{2A}, q^{2B}, q^{3A}, q^{3B}, q^{4A}, q^{4B}, q^{5A}, q^{5B}, and q^{5C} represent independently for each occurrence 0-20 and wherein the repeating unit can be the same or different;

Q and Q′ are independently for each occurrence is absent, —(P⁷-Q⁷-R⁷)ₚ-T⁷- or -T⁷-Q⁷-T^{7′}-B-T^{8′}-Q⁸-T⁸;

P^{2A}, P^{2B}, P^{3A}, P^{3B}, P^{4A}, P^{4B}, P^{5A}, P^{5B}, P^{5C}, P⁷, T^{2A}, T^{2B}, T^{3A}, T^{3B}, T^{4A}, T^{4B}, T^{4A}, T^{5B}, T^{5C}, T⁷, T^{7′}, T⁸ and T^{8′} are each independently for each occurrence absent, CO, NH, O, S, OC(O), NHC(O), CH₂, CH₂NH or CH₂O;

B is —CH₂—N(B^{L})—CH₂-;

B^{L} is -T^{B}-Q^{B}-T^{B′}-R^{x};

Q^{2A}, Q^{2B}, Q^{3A}, Q^{3B}, Q^{4A}, Q^{4B}, Q^{5A}, Q^{5B}, Q^{5C}, Q⁷, Q⁸ and Q^{B} are independently for each occurrence absent, alkylene, substituted alkylene and wherein one or more methylenes can be interrupted or terminated by one or more of O, S, S(O), SO₂, N(R^{N}), C(R′)═C(R′), C≡C or C(O);

T^{B} and T^{B′} are each independently for each occurrence absent, CO, NH, O, S, OC(O), OC(O)O, NHC(O), NHC(O)NH, NHC(O)O, CH₂, CH₂NH or CH₂O;

R^{x} is a lipophile (e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine); a vitamin (e.g., folate, vitamin A, vitamin E, biotin, pyridoxal); a peptide; a carbohydrate-based ligand, e.g. monosaccharide, disaccharide, trisaccharide, tetrasaccharide, oligosaccharide, polysaccharide; an endosomolytic component, a steroid (e.g., uvaol, hecigenin, diosgenin), a terpene (e.g., triterpene, e.g., sarsasapogenin, Friedelin, epifriedelanol derivatized lithocholic acid), or a cationic lipid;

R¹, R², R^{2A}, R^{2B}, R^{3A}, R^{3B}, R^{4A}, R^{4B}, R^{5A}, R^{5B}, R^{5C}, R⁷ are each independently for each occurrence absent, NH, O, S, CH₂, C(O)O, C(O)NH, NHCH(R^{a})C(O), —C(O)—CH(R^{a})—NH—, CO, CH═N—O,

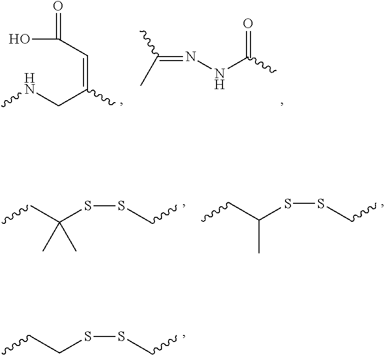

or heterocyclyl;

L¹, L^{2A}, L^{2B}, L^{3A}, L^{3B}, L^{4A}, L^{4B}, L^{5A}, L^{5B} and L^{5C} are each independently for each occurrence a carbohydrate, e.g., monosaccharide, disaccharide, trisaccharide, tetrasaccharide, oligosaccharide and polysaccharide;

R′ and R″ are each independently H, C₁-C₆ alkyl, OH, SH, or N(R^{N})₂;

R^{N} is independently for each occurrence H, methyl, ethyl, propyl, isopropyl, butyl or benzyl;

R^{a} is H or amino acid side chain;

Z′, Z″, Z′″ and Z″″ are each independently for each occurrence O or S;

p represent independently for each occurrence 0-20.

As discussed above, because the ligand can be conjugated to the iRNA agent via a linker or carrier, and because the linker or carrier can contain a branched linker, the iRNA agent can then contain multiple ligands via the same or different backbone attachment points to the carrier, or via the branched linker(s). For instance, the branchpoint of the branched linker may be a bivalent, trivalent, tetravalent, pentavalent, or hexavalent atom, or a group presenting such multiple valencies. In certain embodiments, the branchpoint is —N, —N(Q)-C, —O—C, —S—C, —SS—C, —C(O)N(Q)-C, —OC(O)N(Q)-C, —N(Q)C(O)—C, or —N(Q)C(O)O—C; wherein Q is independently for each occurrence H or optionally substituted alkyl. In other embodiment, the branchpoint is glycerol or glycerol derivative.

In certain embodiments, the double-stranded iRNA agent comprises a targeting ligand of structure:

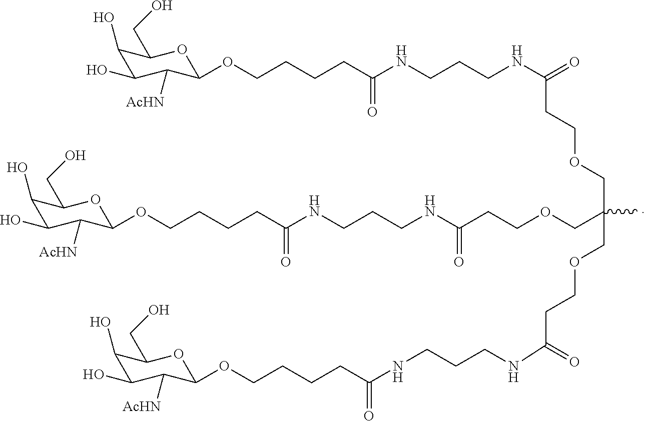 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In certain embodiments, the double-stranded iRNA agent comprises a targeting ligand of structure:

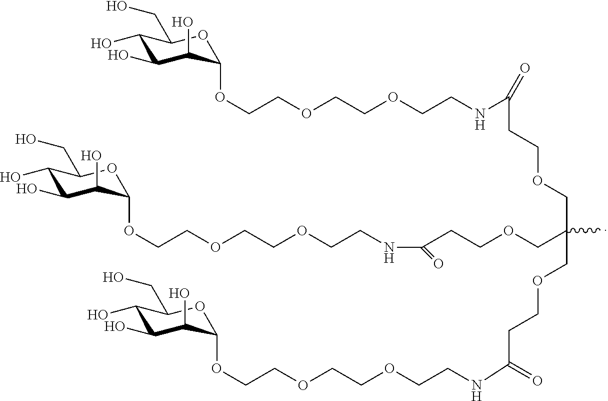

In certain embodiments, the double-stranded iRNA agent comprises a targeting ligand of structure:

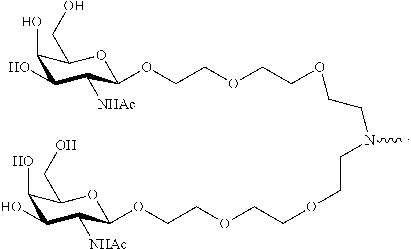

In certain embodiments, the double-stranded iRNA agent comprises a targeting ligand of structure:

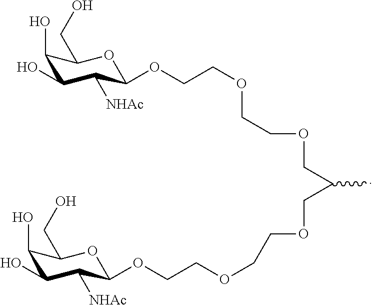

In certain embodiments, the double-stranded iRNA agent comprises a targeting ligand of structure:

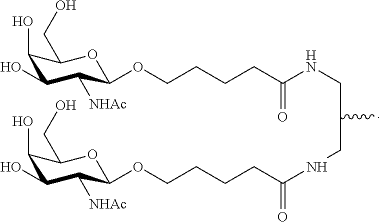

In certain embodiments, the double-stranded iRNA agent comprises a targeting ligand of structure:

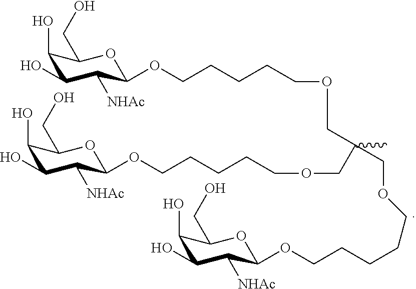

In certain embodiments, the double-stranded iRNA agent comprises a targeting ligand of structure:

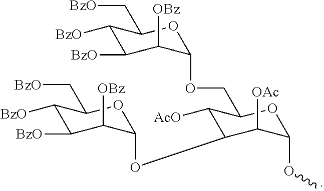

In certain embodiments, the double-stranded iRNA agent comprises a targeting ligand of structure:

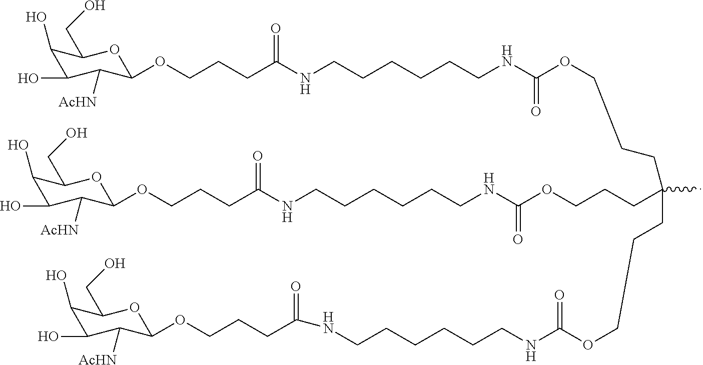 [SMILES: CC(=O)NC1C(OCCCC(=O)NCCCCCCNC(=O)OCCCC(C)(CCCOC(=O)NCCCCCCNC(=O)CCCOC2OC(CO)C(O)C(O)C2NC(C)=O)CCCOC(=O)NCCCCCCNC(=O)CCCOC2OC(CO)C(O)C(O)C2NC(C)=O)OC(CO)C(O)C1O]

In certain embodiments, the double-stranded iRNA agent comprises a targeting ligand of structure:

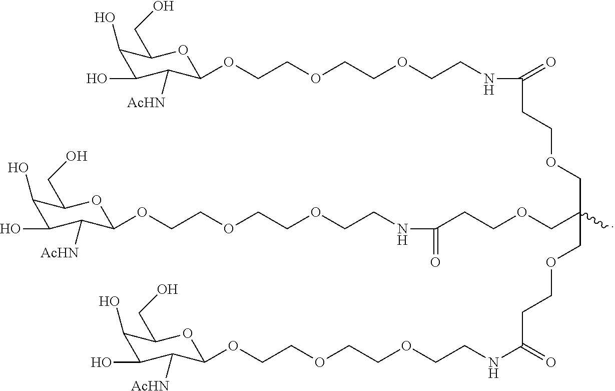 [SMILES: CC(=O)NC1C(OCCOCCOCCNC(=O)CCOCC(C)(COCCC(=O)NCCOCCOCCOC2OC(CO)C(O)C(O)C2NC(C)=O)COCCC(=O)NCCOCCOCCOC2OC(CO)C(O)C(O)C2NC(C)=O)OC(CO)C(O)C1O]

In certain embodiments, the double-stranded iRNA agent comprises a targeting ligand of structure:

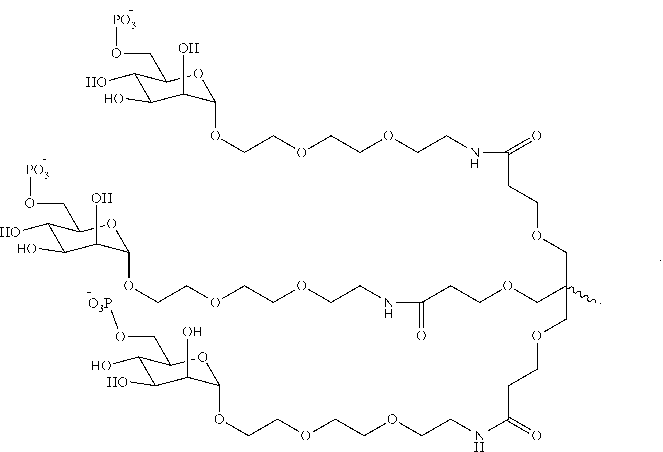

In certain embodiments, the double-stranded iRNA agent comprises a targeting ligand of structure:

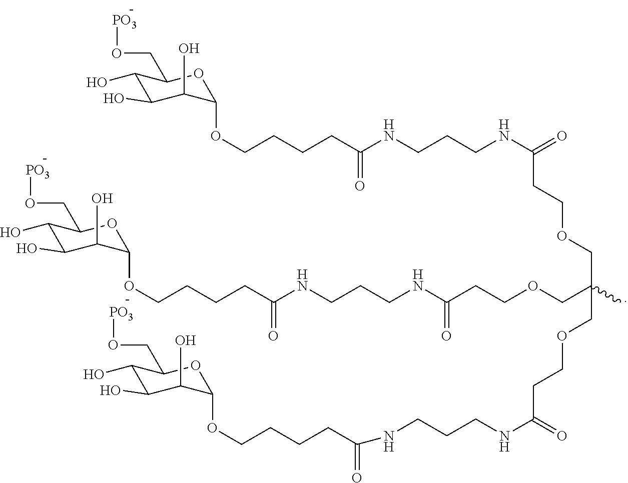

In certain embodiments, the double-stranded iRNA agent comprises a targeting monomer of structure:

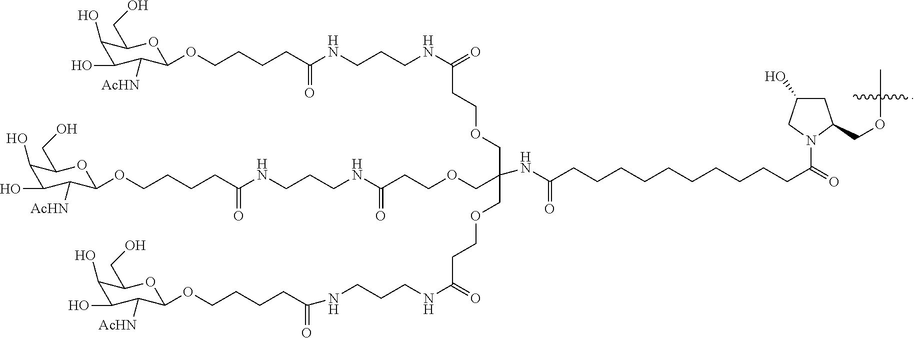 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

In certain embodiments, the double-stranded iRNA agent comprises a targeting ligand of structure:

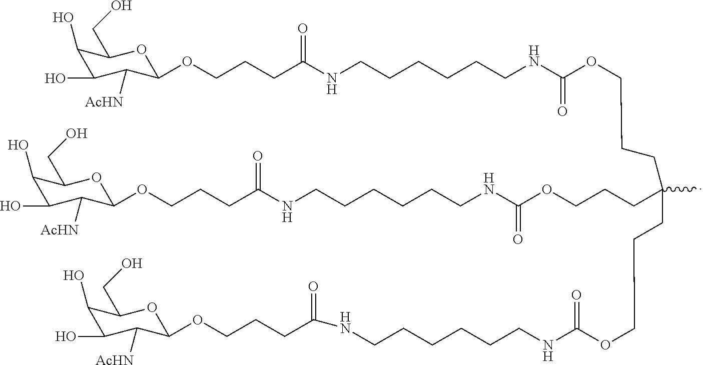 [SMILES: CC(=O)NC1C(OCCCC(=O)NCCCCCCNC(=O)OCCCC(C)(CCCOC(=O)NCCCCCCNC(=O)CCCOC2OC(CO)C(O)C(O)C2NC(C)=O)CCCOC(=O)NCCCCCCNC(=O)CCCOC2OC(CO)C(O)C(O)C2NC(C)=O)OC(CO)C(O)C1O]

In certain embodiments, the branched linker attaching the carbohydrate-based ligand to the double-stranded iRNA agent can be a branched aliphatic group comprising groups selected from the group consisting of alkyl, amide, disulfide, polyethylene glycol, ether, thioether, hydroxylamino groups, and combinations thereof.

In some embodiments, the bivalent or trivalent branched linker can have a lysine-based structure, such as

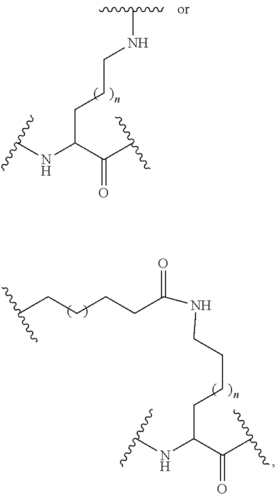

wherein n is independent from 1 to 20, for instance, from 1 to 10, from 1 to 5, from 1 to 3, or from 1 to 3. In one embodiment, the trivalent branched linker is

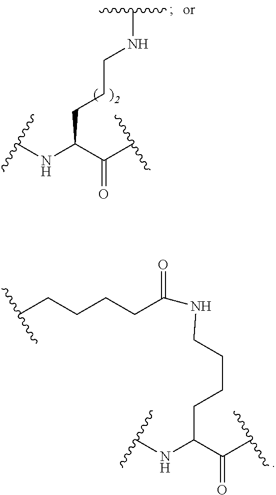

### Exemplary Ligand Monomers

In certain embodiments, the double-stranded iRNA agent comprises a targeting monomer of structure:

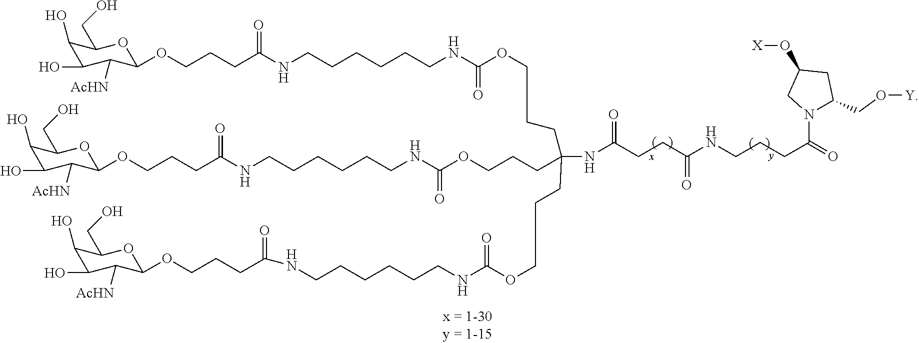

In certain embodiments, the double-stranded iRNA agent comprises a targeting monomer of structure:

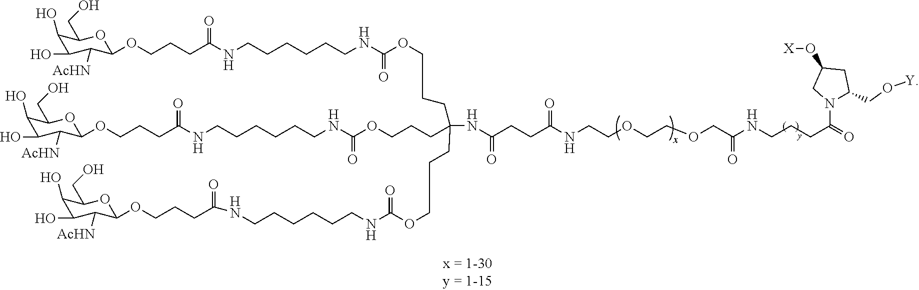

In certain embodiments, the double-stranded iRNA agent comprises a targeting monomer of structure:

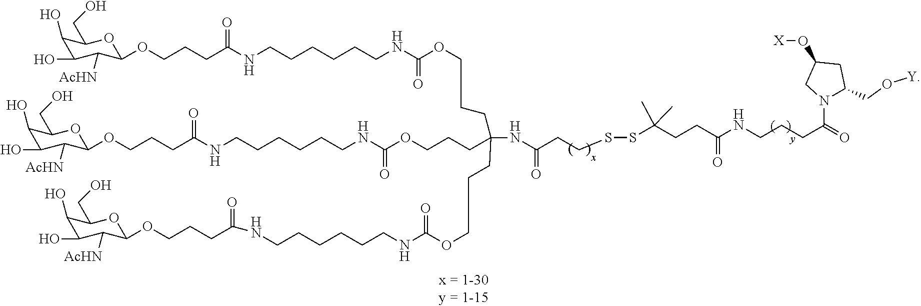

In certain embodiments, the double-stranded iRNA agent comprises a targeting monomer of structure:

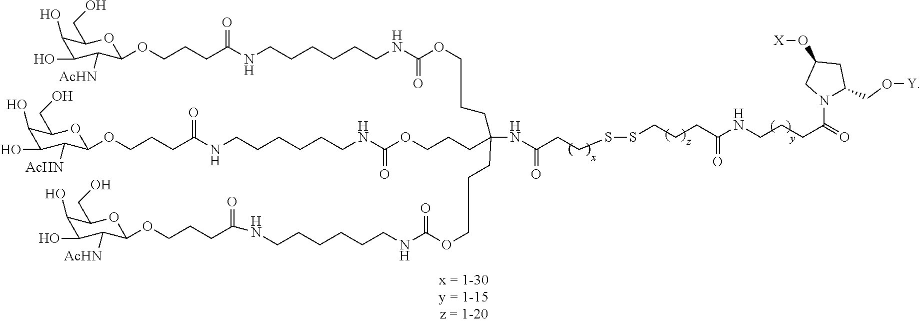

In certain embodiments, the double-stranded iRNA agent comprises a monomer of structure:

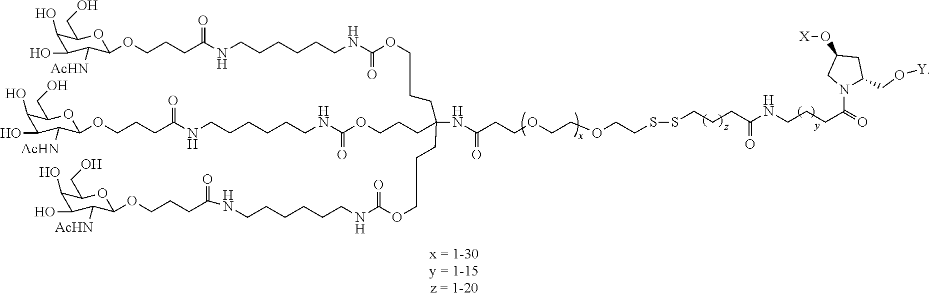

In certain embodiments, the double-stranded iRNA agent comprises a monomer of structure:

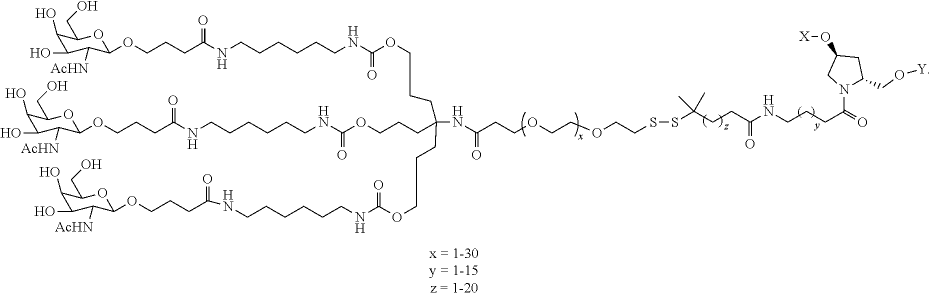

In certain embodiments, the double-stranded iRNA agent comprises a ligand of structure:

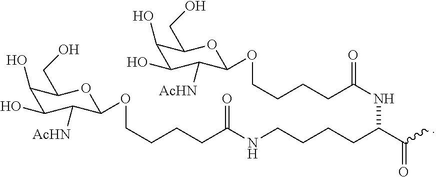

In certain embodiments, the double-stranded iRNA agent comprises a ligand of structure:

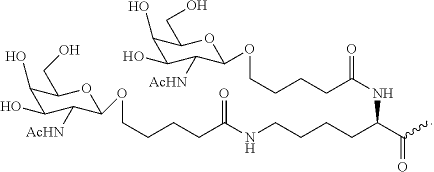

In certain embodiments, the double-stranded iRNA agent comprises a ligand of structure:

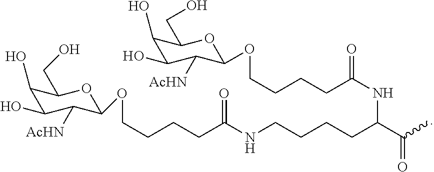

In certain embodiments, the double-stranded iRNA agent comprises a ligand of structure:

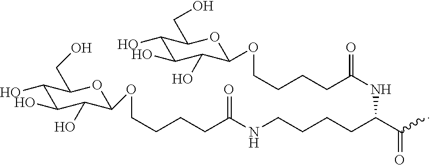

In certain embodiments, the double-stranded iRNA agent comprises a ligand of structure:

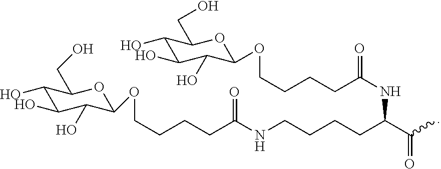

In certain embodiments, the double-stranded iRNA agent comprises a ligand of structure:

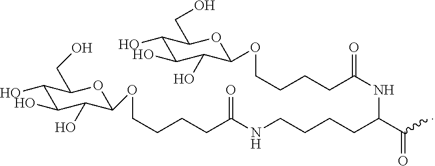

In certain embodiments, the double-stranded iRNA agent comprises a ligand of structure:

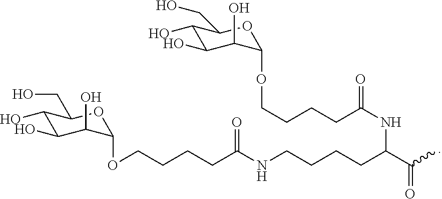

In certain embodiments, the double-stranded iRNA agent comprises a ligand of structure:

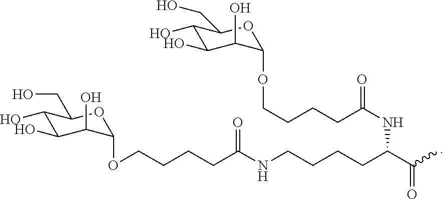

In certain embodiments, the double-stranded iRNA agent comprises a ligand of structure:

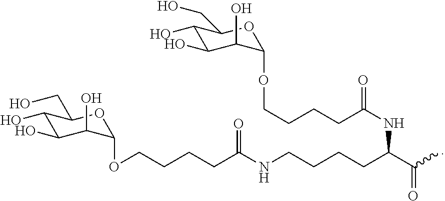

In certain embodiments, the double-stranded iRNA agent comprises a monomer of structure:

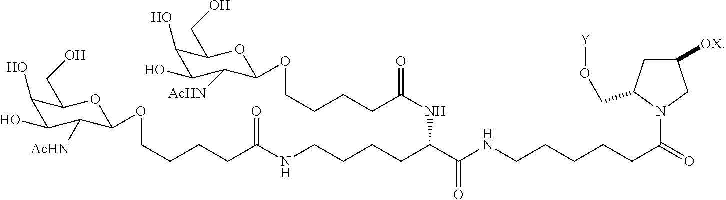

In certain embodiments, the double-stranded iRNA agent comprises a monomer of structure:

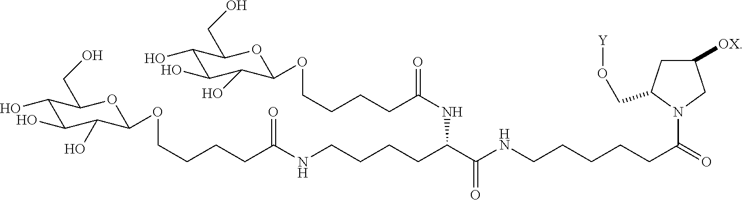

In certain embodiments, the double-stranded iRNA agent comprises a monomer of structure:

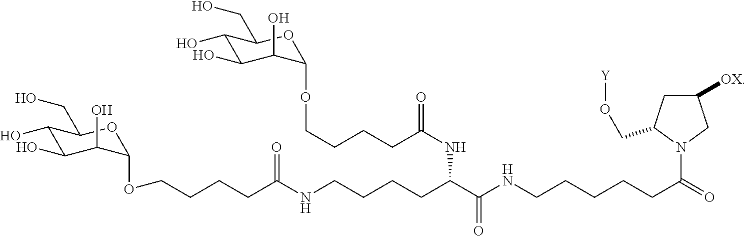

In certain embodiments, the double-stranded iRNA agent comprises a monomer of structure:

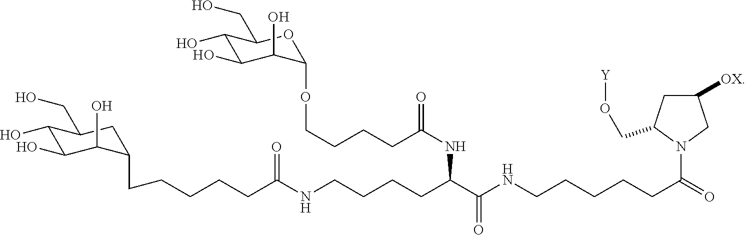

In certain embodiments, the double-stranded iRNA agent comprises a monomer of structure:

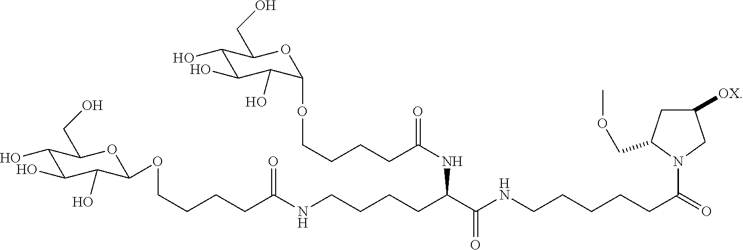

In certain embodiments, the double-stranded iRNA agent comprises a monomer of structure:

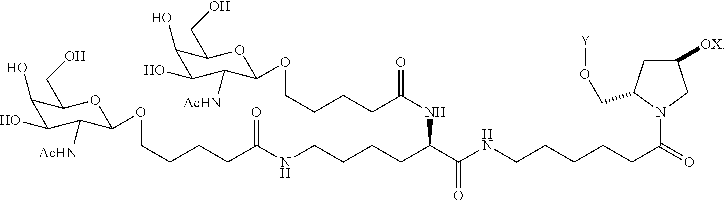

In some embodiments both L^{2A} and L^{2B} are different.

In some preferred embodiments both L^{3A} and L^{3B} are the same.

In some embodiments both L^{3A} and L^{3B} are different.

In some preferred embodiments both L^{4A} and L^{4B} are the same.

In some embodiments both L^{4A} and L^{4B} are different.

In some preferred embodiments all of L^{5A}, L^{5B} and L^{5C} are the same.

In some embodiments two of L^{5A}, L^{5B} and L^{5C} are the same

In some embodiments L^{5A} and L^{5B} are the same.

In some embodiments L^{5A} and L^{5C} are the same.

In some embodiments L^{5B} and L^{5C} are the same.

In certain embodiments, the double-stranded iRNA agent comprises a monomer of structure:

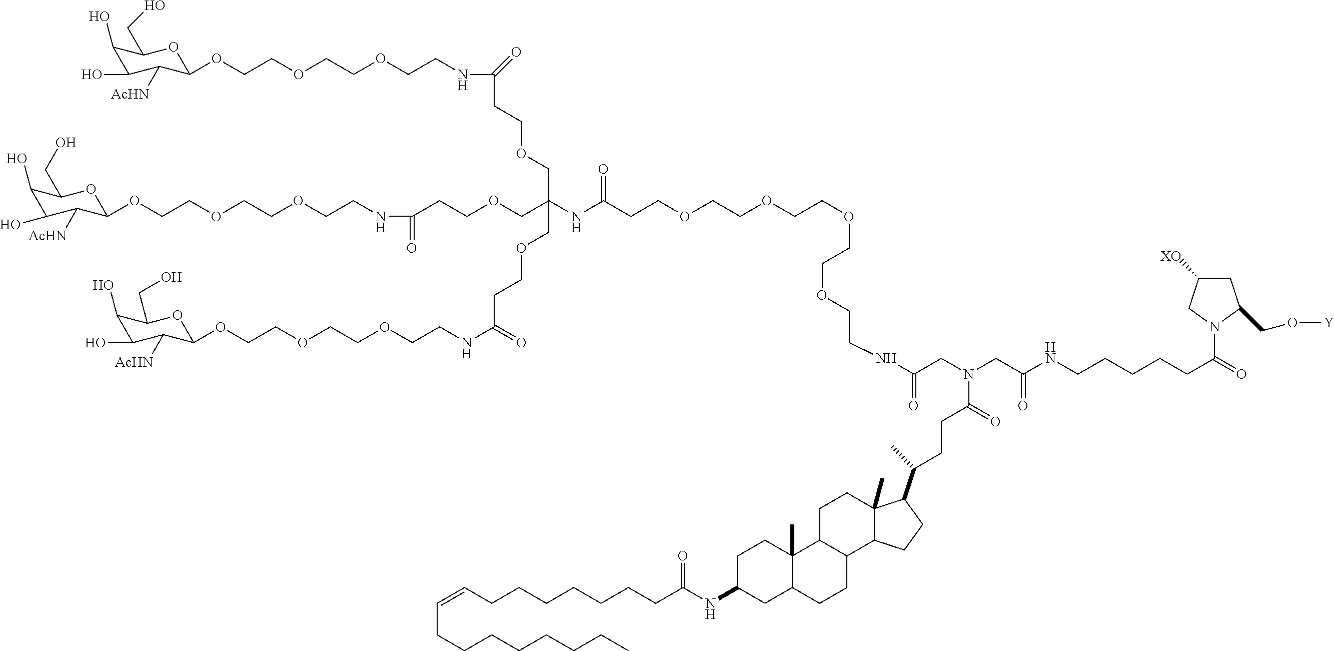

In certain embodiments, the double-stranded iRNA agent comprises a monomer of structure:

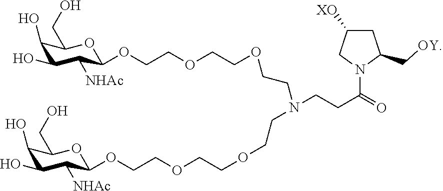

In certain embodiments, the double-stranded iRNA agent comprises a monomer of structure:

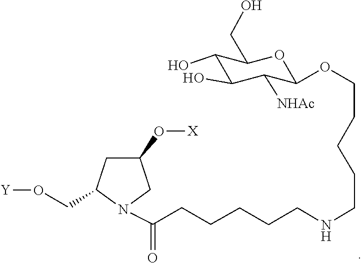

In certain embodiments, the double-stranded iRNA agent comprises a monomer of structure:

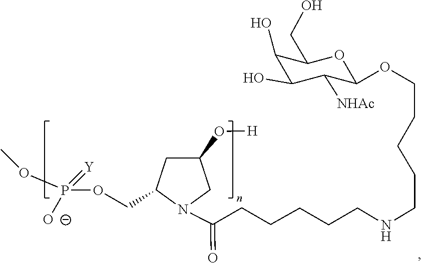

wherein Y is O or S, and n is 1-6.

In certain embodiments, the double-stranded iRNA agent comprises a monomer of structure:

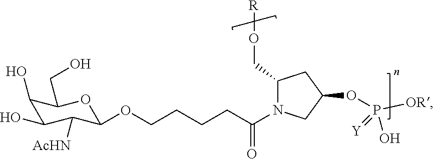

wherein Y is O or S, n is 1-6, R is hydrogen or nucleic acid, and R′ is nucleic acid.

In certain embodiments, the double-stranded iRNA agent comprises a monomer of structure:

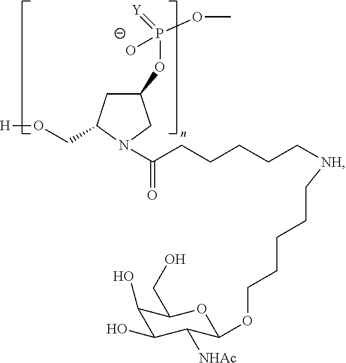

wherein Y is O or S, and n is 1-6.

In certain embodiments, the oligomeric compound described herein, including but not limited to double-stranded iRNA agent of the inventions, comprises a monomer of structure:

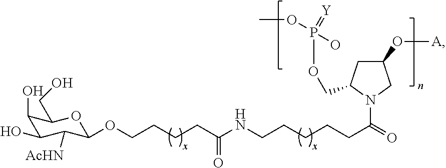

wherein Y is O or S, n is 2-6, x is 1-6, and A is H or a phosphate linkage.

In certain embodiments, the double-stranded iRNA agent comprises at least 1, 2, 3 or 4 monomer of structure:

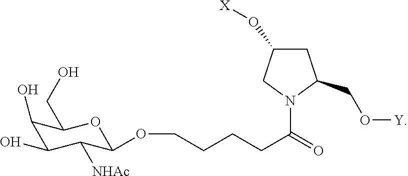

In certain embodiments, the double-stranded iRNA agent comprises a monomer of structure:

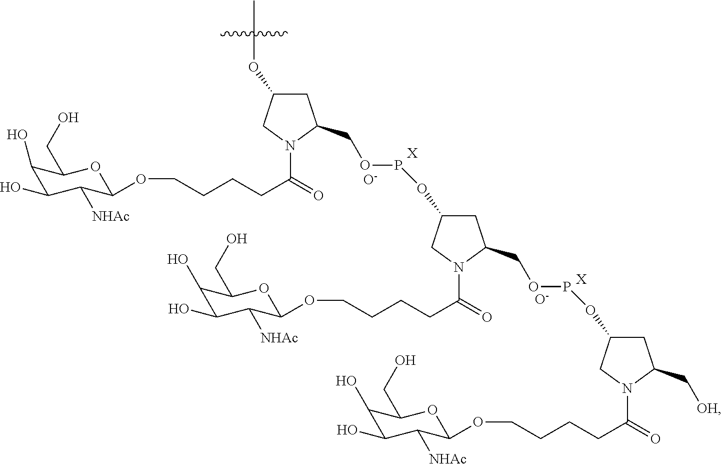

wherein X is O or S.

In certain embodiments, the oligomeric compound described herein, including but not limited to double-stranded iRNA agent of the inventions, comprises a monomer of structure:

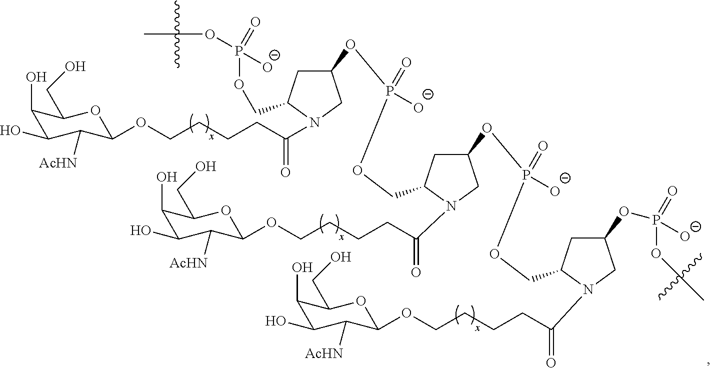

wherein x is 1-12.

In certain embodiments, the double-stranded iRNA agent comprises a monomer of structure:

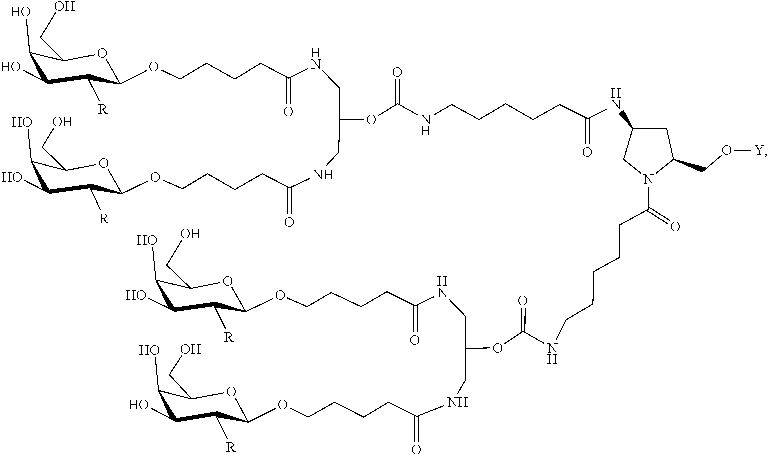

wherein R is OH or NHCOCH₃.

In certain embodiments, the double-stranded iRNA agent comprises a monomer of structure:

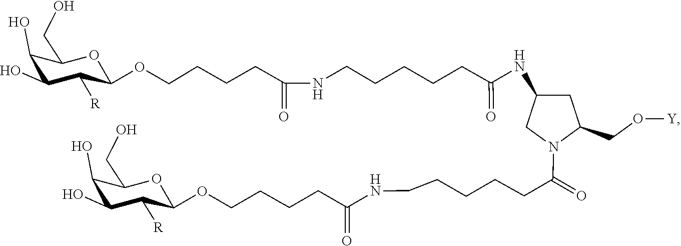

wherein R is OH or NHCOCH₃.

In certain embodiments, the double-stranded iRNA agent comprises a monomer of structure:

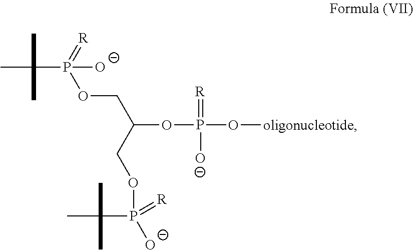

wherein R is O or S.

In certain embodiments, the double-stranded iRNA agent comprises a monomer of structure:

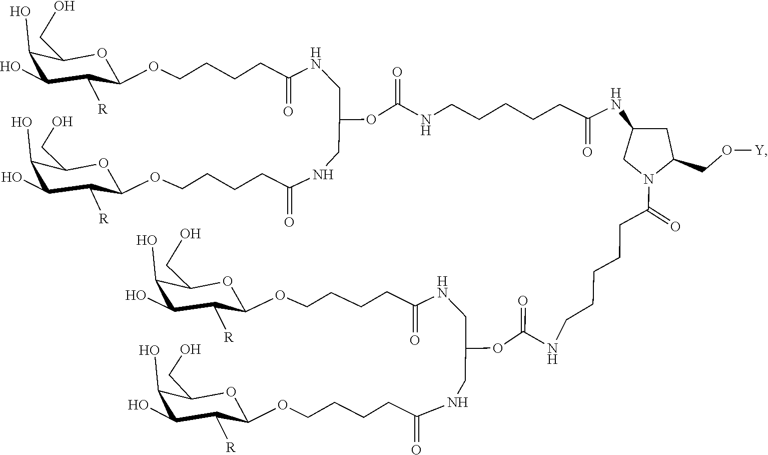

wherein R is OH or NHCOCH₃.

In certain embodiments, the double-stranded iRNA agent comprises a monomer of structure:

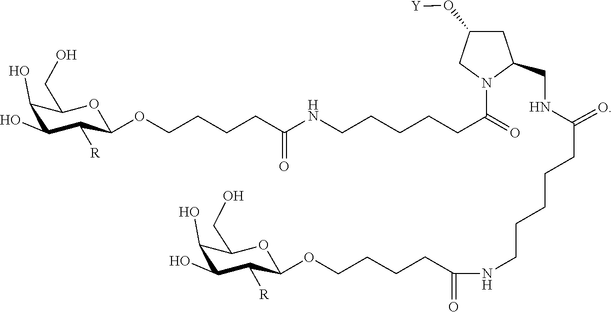

In certain embodiments, the double-stranded iRNA agent comprises a monomer of structure:

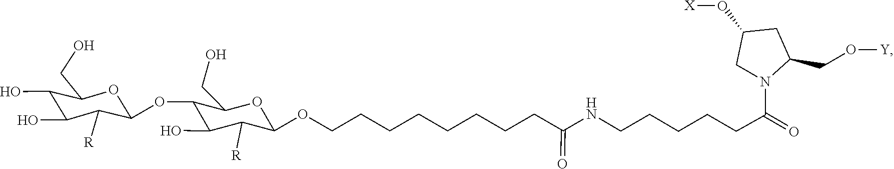

wherein R is OH or NHCOCH₃.

In certain embodiments, the double-stranded iRNA agent comprises a monomer of structure:

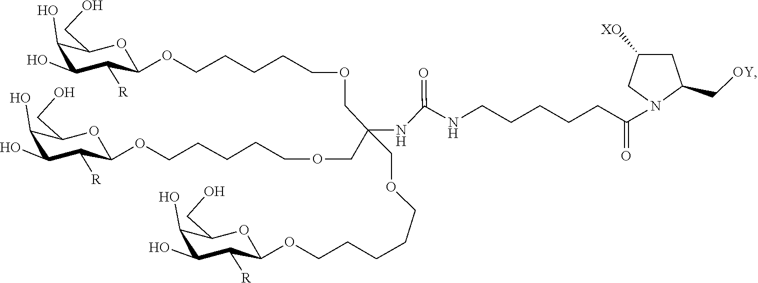

wherein R is OH or NHCOCH₃.

In certain embodiments, the double-stranded iRNA agent comprises a monomer of structure:

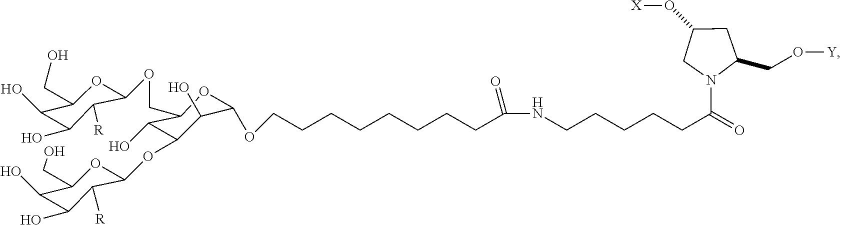

wherein R is OH or NHCOCH₃.

In certain embodiments, the double-stranded iRNA agent comprises a monomer of structure:

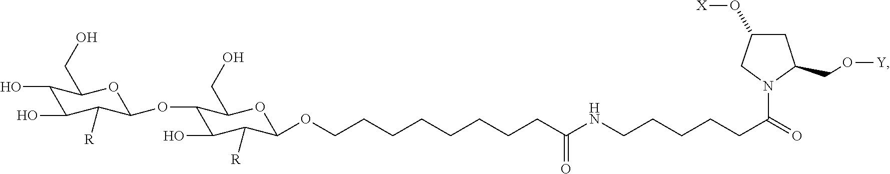

wherein R is OH or NHCOCH₃.

In certain embodiments, the double-stranded iRNA agent comprises a monomer of structure:

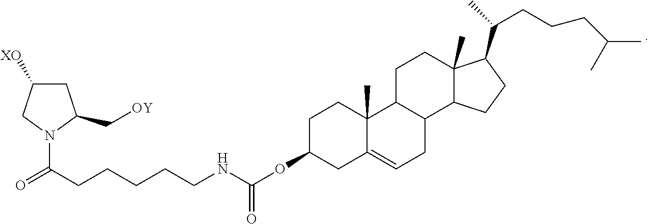

In the above described monomers, X and Y are each independently for each occurrence H, a protecting group, a phosphate group, a phosphodiester group, an activated phosphate group, an activated phosphite group, a phosphoramidite, a solid support, —P(Z′)(Z″)O-nucleoside, —P(Z′)(Z″)O-oligonucleotide, a lipid, a PEG, a steroid, a polymer, a nucleotide, a nucleoside, or an oligonucleotide; and Z′ and Z″ are each independently for each occurrence O or S.

In certain embodiments, the double-stranded iRNA agent is conjugated with a ligand of structure:

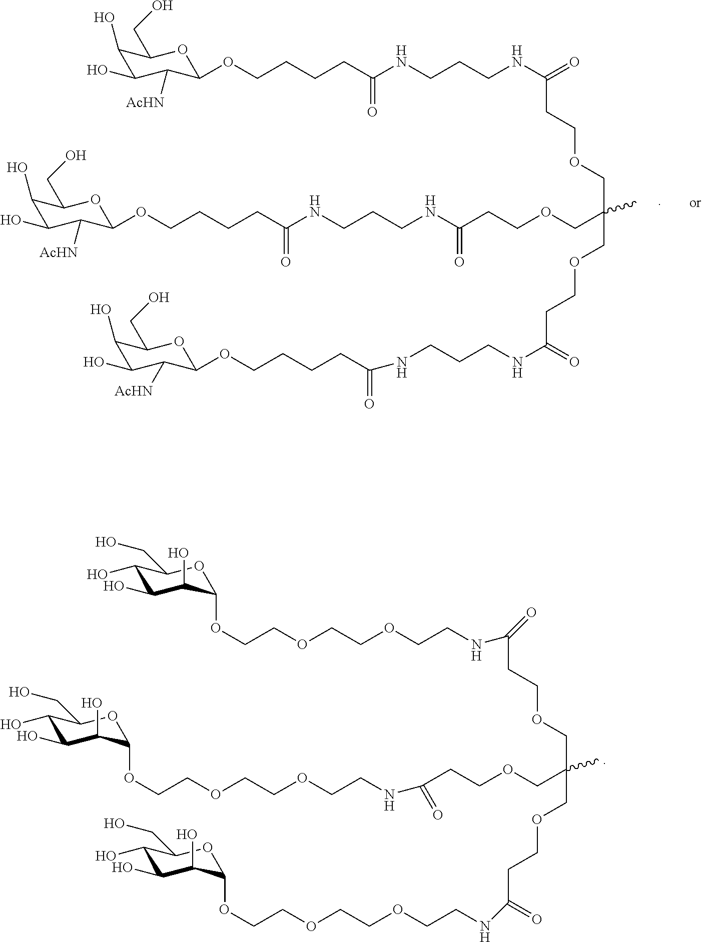

In certain embodiments, the double-stranded iRNA agent comprises a ligand of structure:

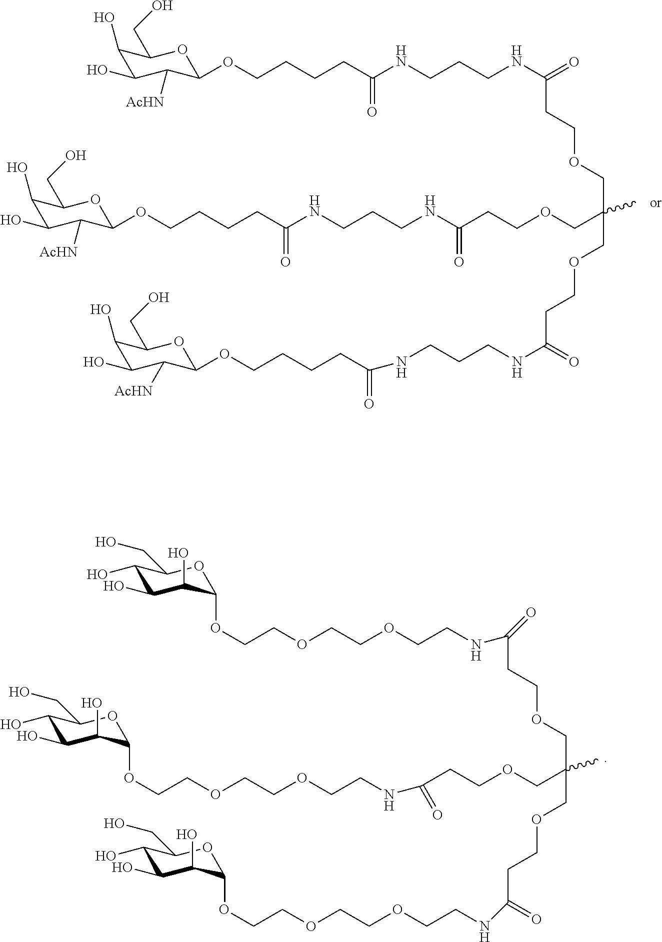

In certain embodiments, the double-stranded iRNA agent comprises a monomer of structure:

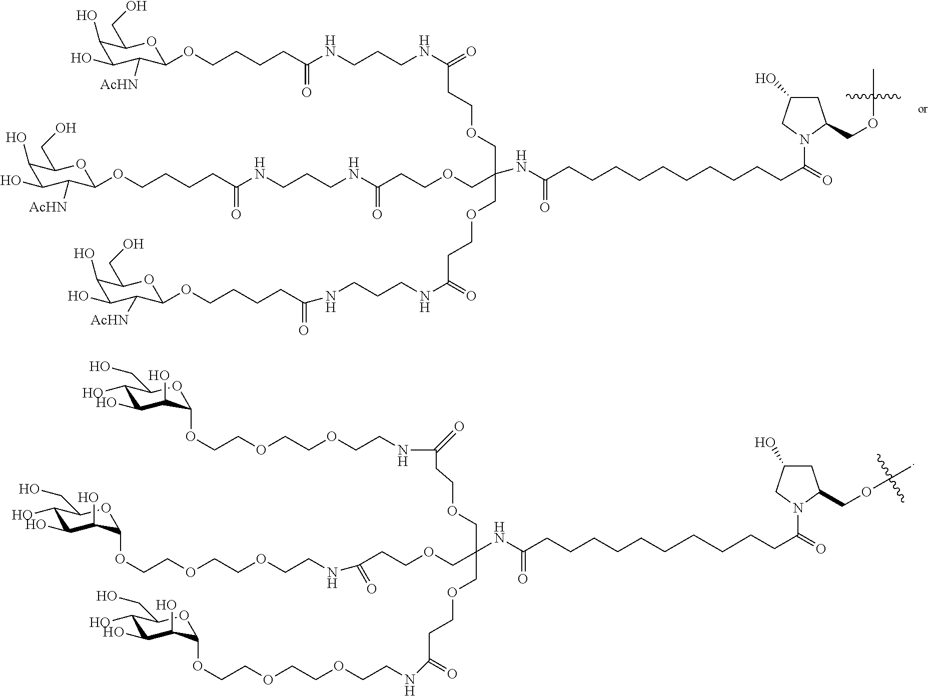

Synthesis of above described ligands and monomers is described, for example, in U.S. Pat. No. 8,106,022, content of which is incorporated herein by reference in its entirety.

In some embodiments, the double stranded iRNA agent comprises one or more ligand carbohydrate-based ligands conjugated to both ends of the sense strand.

In some embodiments, the double stranded iRNA agent comprises one or more ligand carbohydrate-based ligands conjugated to both ends of the antisense strand.

In some embodiments, the double stranded iRNA agent comprises one or more carbohydrate-based ligands conjugated to the 5′ end or 3′ end of the sense strand, and one or more carbohydrate-based ligands conjugated to the 5′ end or 3′ end of the antisense strand,

In some embodiments, the carbohydrate-based ligand is conjugated to the terminal end of a strand via one or more linkers (tethers) and/or a carrier.

In one embodiment, the carbohydrate-based ligand is conjugated to the terminal end of a strand via one or more linkers (tethers).

In one embodiment, the carbohydrate-based ligand is conjugated to the 5′ end of the sense strand or antisense strand via a cyclic carrier, optionally via one or more intervening linkers (tethers).

In some embodiments, the double stranded iRNA agent comprises one or more lipophilic moieties conjugated to one or more internal positions on at least one strand. Internal positions of a strand refers to the nucleotide on any position of the strand, except the terminal position from the 3′ end and 5′ end of the strand (e.g., excluding 2 positions: position 1 counting from the 3′ end and position 1 counting from the 5′ end).

In one embodiment, the double stranded iRNA agent comprises one or more lipophilic moieties conjugated to one or more internal positions on at least one strand, which include all positions except the terminal two positions from each end of the strand (e.g., excluding 4 positions: positions 1 and 2 counting from the 3′ end and positions 1 and 2 counting from the 5′ end). In one embodiment, the lipophilic moiety is conjugated to one or more internal positions on at least one strand, which include all positions except the terminal three positions from each end of the strand (e.g., excluding 6 positions: positions 1, 2, and 3 counting from the 3′ end and positions 1, 2, and 3 counting from the 5′ end).

In one embodiment, the double stranded iRNA agent comprises one or more lipophilic moieties conjugated to one or more internal positions on at least one strand, except the cleavage site region of the sense strand, for instance, the lipophilic moiety is not conjugated to positions 9-12 counting from the 5′-end of the sense strand, for example, the lipophilic moiety is not conjugated to positions 9-11 counting from the 5′-end of the sense strand. Alternatively, the internal positions exclude positions 11-13 counting from the 3′-end of the sense strand.

In one embodiment, the double stranded iRNA agent comprises one or more lipophilic moieties conjugated to one or more internal positions on at least one strand, which exclude the cleavage site region of the antisense strand. For instance, the internal positions exclude positions 12-14 counting from the 5′-end of the antisense strand.

In one embodiment, the double stranded iRNA agent comprises one or more lipophilic moieties conjugated to one or more internal positions on at least one strand, which exclude positions 11-13 on the sense strand, counting from the 3′-end, and positions 12-14 on the antisense strand, counting from the 5′-end.

In one embodiment, one or more lipophilic moieties are conjugated to one or more of the following internal positions: positions 4-8 and 13-18 on the sense strand, and positions 6-10 and 15-18 on the antisense strand, counting from the 5′end of each strand.

In one embodiment, one or more lipophilic moieties are conjugated to one or more of the following internal positions: positions 5, 6, 7, 15, and 17 on the sense strand, and positions 15 and 17 on the antisense strand, counting from the 5′end of each strand.

In some embodiments, the double stranded iRNA agent comprises one or more lipophilic moieties conjugated to a nucleobase, sugar moiety, or internucleosidic linkage of the double-stranded iRNA agent.

### Definitions

Unless specific definitions are provided, the nomenclature utilized in connection with, and the procedures and techniques of, analytical chemistry, synthetic organic chemistry, and medicinal and pharmaceutical chemistry described herein are those well-known and commonly used in the art. Standard techniques may be used for chemical synthesis, and chemical analysis. Certain such techniques and procedures may be found for example in “Carbohydrate Modifications in Antisense Research” Edited by Sangvi and Cook, American Chemical Society, Washington D.C., 1994; “Remington's Pharmaceutical Sciences,” Mack Publishing Co., Easton, Pa., 18th edition, 1990; and “Antisense Drug Technology, Principles, Strategies, and Applications” Edited by Stanley T. Crooke, CRC Press, Boca Raton, Fla.; and Sambrook et al., “Molecular Cloning, A laboratory Manual,” 2d Edition, Cold Spring Harbor Laboratory Press, 1989, which are hereby incorporated by reference for any purpose. Where permitted, all patents, applications, published applications and other publications and other data referred to throughout in the disclosure herein are incorporated by reference in their entirety.

Unless otherwise indicated, the following terms have the following meanings:

As used herein, the term “target nucleic acid” refers to any nucleic acid molecule the expression or activity of which is capable of being modulated by an siRNA compound. Target nucleic acids include, but are not limited to, RNA (including, but not limited to pre-mRNA and mRNA or portions thereof) transcribed from DNA encoding a target protein, and also cDNA derived from such RNA, and miRNA. For example, the target nucleic acid can be a cellular gene (or mRNA transcribed from the gene) whose expression is associated with a particular disorder or disease state. In some embodiments, a target nucleic acid can be a nucleic acid molecule from an infectious agent.

As used herein, the term “iRNA” refers to an agent that mediates the targeted cleavage of an RNA transcript. These agents associate with a cytoplasmic multi-protein complex known as RNAi-induced silencing complex (RISC). Agents that are effective in inducing RNA interference are also referred to as siRNA, RNAi agent, or iRNA agent, herein. Thus, these terms can be used interchangeably herein. As used herein, the term iRNA includes microRNAs and pre-microRNAs. Moreover, the “compound” or “compounds” of the invention as used herein, also refers to the iRNA agent, and can be used interchangeably with the iRNA agent.

The iRNA agent should include a region of sufficient homology to the target gene, and be of sufficient length in terms of nucleotides, such that the iRNA agent, or a fragment thereof, can mediate downregulation of the target gene. (For ease of exposition the term nucleotide or ribonucleotide is sometimes used herein in reference to one or more monomeric subunits of an iRNA agent. It will be understood herein that the usage of the term “ribonucleotide” or “nucleotide”, herein can, in the case of a modified RNA or nucleotide surrogate, also refer to a modified nucleotide, or surrogate replacement moiety at one or more positions.) Thus, the iRNA agent is or includes a region which is at least partially, and in some embodiments fully, complementary to the target RNA. It is not necessary that there be perfect complementarity between the iRNA agent and the target, but the correspondence must be sufficient to enable the iRNA agent, or a cleavage product thereof, to direct sequence specific silencing, e.g., by RNAi cleavage of the target RNA, e.g., mRNA. Complementarity, or degree of homology with the target strand, is most critical in the antisense strand. While perfect complementarity, particularly in the antisense strand, is often desired some embodiments can include, particularly in the antisense strand, one or more, or for example, 6, 5, 4, 3, 2, or fewer mismatches (with respect to the target RNA). The sense strand need only be sufficiently complementary with the antisense strand to maintain the over all double stranded character of the molecule.

iRNA agents include: molecules that are long enough to trigger the interferon response (which can be cleaved by Dicer (Bernstein et al. 2001. Nature, 409:363-366) and enter a RISC (RNAi-induced silencing complex)); and, molecules which are sufficiently short that they do not trigger the interferon response (which molecules can also be cleaved by Dicer and/or enter a RISC), e.g., molecules which are of a size which allows entry into a RISC, e.g., molecules which resemble Dicer-cleavage products. Molecules that are short enough that they do not trigger an interferon response are termed siRNA agents or shorter iRNA agents herein. “siRNA agent or shorter iRNA agent” as used herein, refers to an iRNA agent, e.g., a double stranded RNA agent or single strand agent, that is sufficiently short that it does not induce a deleterious interferon response in a human cell, e.g., it has a duplexed region of less than 60, 50, 40, or 30 nucleotide pairs. The siRNA agent, or a cleavage product thereof, can down regulate a target gene, e.g., by inducing RNAi with respect to a target RNA, wherein the target may comprise an endogenous or pathogen target RNA.

A “single strand iRNA agent” as used herein, is an iRNA agent which is made up of a single molecule. It may include a duplexed region, formed by intra-strand pairing, e.g., it may be, or include, a hairpin or pan-handle structure. Single strand iRNA agents may be antisense with regard to the target molecule. A single strand iRNA agent may be sufficiently long that it can enter the RISC and participate in RISC mediated cleavage of a target mRNA. A single strand iRNA agent is at least 14, and in other embodiments at least 15, 20, 25, 29, 35, 40, or 50 nucleotides in length. In certain embodiments, it is less than 200, 100, or 60 nucleotides in length.

A loop refers to a region of an iRNA strand that is unpaired with the opposing nucleotide in the duplex when a section of the iRNA strand forms base pairs with another strand or with another section of the same strand.

Hairpin iRNA agents will have a duplex region equal to or at least 17, 18, 19, 29, 21, 22, 23, 24, or 25 nucleotide pairs. The duplex region will may be equal to or less than 200, 100, or 50, in length. In certain embodiments, ranges for the duplex region are 15-30, 17 to 23, 19 to 23, and 19 to 21 nucleotides pairs in length. The hairpin may have a single strand overhang or terminal unpaired region, in some embodiments at the 3′, and in certain embodiments on the antisense side of the hairpin. In some embodiments, the overhangs are 2-3 nucleotides in length.

A “double stranded (ds) iRNA agent” as used herein, is an iRNA agent which includes more than one, and in some cases two, strands in which interchain hybridization can form a region of duplex structure.

As used herein, the terms “siRNA activity” and “RNAi activity” refer to gene silencing by an siRNA.

As used herein, “gene silencing” by a RNA interference molecule refers to a decrease in the mRNA level in a cell for a target gene by at least about 5%, at least about 10%, at least about 20%, at least about 30%, at least about 40%, at least about 50%, at least about 60%, at least about 70%, at least about 80%, at least about 90%, at least about 95%, at least about 99% up to and including 100%, and any integer in between of the mRNA level found in the cell without the presence of the miRNA or RNA interference molecule. In one preferred embodiment, the mRNA levels are decreased by at least about 70%, at least about 80%, at least about 90%, at least about 95%, at least about 99%, up to and including 100% and any integer in between 5% and 100%.”

As used herein the term “modulate gene expression” means that expression of the gene, or level of RNA molecule or equivalent RNA molecules encoding one or more proteins or protein subunits is up regulated or down regulated, such that expression, level, or activity is greater than or less than that observed in the absence of the modulator. For example, the term “modulate” can mean “inhibit,” but the use of the word “modulate” is not limited to this definition.

As used herein, gene expression modulation happens when the expression of the gene, or level of RNA molecule or equivalent RNA molecules encoding one or more proteins or protein subunits is at least 5%, 10%, 20%, 30%, 40%, 50%, 60%, 70%, 80%, 90%, 100%, 2-fold, 3-fold, 4-fold, 5-fold or more different from that observed in the absence of the siRNA, e.g., RNAi agent. The % and/or fold difference can be calculated relative to the control or the non-control, for example,

$$\% difference = \frac{\begin{matrix}[ expression with siRNA - \\ expression without siRNA ]\end{matrix}}{expression without siRNA} or$$

$$\% difference = \frac{\begin{matrix}[ expression with siRNA - \\ expression without siRNA ]\end{matrix}}{expression without siRNA}$$

As used herein, the term “inhibit”, “down-regulate”, or “reduce” in relation to gene expression, means that the expression of the gene, or level of RNA molecules or equivalent RNA molecules encoding one or more proteins or protein subunits, or activity of one or more proteins or protein subunits, is reduced below that observed in the absence of modulator. The gene expression is down-regulated when expression of the gene, or level of RNA molecules or equivalent RNA molecules encoding one or more proteins or protein subunits, or activity of one or more proteins or protein subunits, is reduced at least 10% lower relative to a corresponding non-modulated control, and preferably at least 10%, 20%, 30%, 40%, 50%, 60%, 70%, 80%, 90%, 95%, 98%, 99% or most preferably, 100% (i.e., no gene expression).

As used herein, the term “increase” or “up-regulate” in relation to gene expression means that the expression of the gene, or level of RNA molecules or equivalent RNA molecules encoding one or more proteins or protein subunits, or activity of one or more proteins or protein subunits, is increased above that observed in the absence of modulator. The gene expression is up-regulated when expression of the gene, or level of RNA molecules or equivalent RNA molecules encoding one or more proteins or protein subunits, or activity of one or more proteins or protein subunits, is increased at least 10% relative to a corresponding non-modulated control, and preferably at least 10%, 20%, 30%, 40%, 50%, 60%, 70%, 80%, 90%, 95%, 98%, 100%, 1.1-fold, 1.25-fold, 1.5-fold, 1.75-fold, 2-fold, 3-fold, 4-fold, 5-fold, 10-fold, 50-fold, 100-fold or more.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced” or “reduce” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The double-stranded iRNAs comprise two oligonucleotide strands that are sufficiently complementary to hybridize to form a duplex structure. Generally, the duplex structure is between 15 and 30, more generally between 18 and 25, yet more generally between 19 and 24, and most generally between 19 and 21 base pairs in length. In some embodiments, longer double-stranded iRNAs of between 25 and 30 base pairs in length are preferred. In some embodiments, shorter double-stranded iRNAs of between 10 and 15 base pairs in length are preferred. In another embodiment, the double-stranded iRNA is at least 21 nucleotides long.

In some embodiments, the double-stranded iRNA comprises a sense strand and an antisense strand, wherein the antisense RNA strand has a region of complementarity which is complementary to at least a part of a target sequence, and the duplex region is 14-30 nucleotides in length. Similarly, the region of complementarity to the target sequence is between 14 and 30, more generally between 18 and 25, yet more generally between 19 and 24, and most generally between 19 and 21 nucleotides in length.

The phrase “antisense strand” as used herein, refers to an oligomeric compound that is substantially or 100% complementary to a target sequence of interest. The phrase “antisense strand” includes the antisense region of both oligomeric compounds that are formed from two separate strands, as well as unimolecular oligomeric compounds that are capable of forming hairpin or dumbbell type structures. The terms “antisense strand” and “guide strand” are used interchangeably herein.

The phrase “sense strand” refers to an oligomeric compound that has the same nucleoside sequence, in whole or in part, as a target sequence such as a messenger RNA or a sequence of DNA. The terms “sense strand” and “passenger strand” are used interchangeably herein.

By “specifically hybridizable” and “complementary” is meant that a nucleic acid can form hydrogen bond(s) with another nucleic acid sequence by either traditional Watson-Crick or other non-traditional types. In reference to the nucleic molecules of the present invention, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al, 1987, CSH Symp. Quant. Biol. LII pp. 123-133; Frier et al., 1986, Proc. Nat. Acad. Sci. USA 83:9373-9377; Turner et al., 1987, I. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” or 100% complementarity means that all the contiguous residues of a nucleic acid sequence will hydrogen bond with the same number of contiguous residues in a second nucleic acid sequence. Less than perfect complementarity refers to the situation in which some, but not all, nucleoside units of two strands can hydrogen bond with each other. “Substantial complementarity” refers to polynucleotide strands exhibiting 90% or greater complementarity, excluding regions of the polynucleotide strands, such as overhangs, that are selected so as to be noncomplementary. Specific binding requires a sufficient degree of complementarity to avoid non-specific binding of the oligomeric compound to non-target sequences under conditions in which specific binding is desired, i.e., under physiological conditions in the case of in vivo assays or therapeutic treatment, or in the case of in vitro assays, under conditions in which the assays are performed. The non-target sequences typically differ by at least 5 nucleotides.

In some embodiments, the double-stranded region of a double-stranded iRNA agent is equal to or at least, 10, 11, 12, 13, 14, 15, 16, 17, 18, 19, 20, 21, 22, 23, 23, 24, 25, 26, 27, 28, 29, 30 or more nucleotide pairs in length.

In some embodiments, the antisense strand of a double-stranded iRNA agent is equal to or at least 14, 15, 16, 17, 18, 19, 20, 21, 22, 23, 23, 24, 25, 26, 27, 28, 29, or 30 nucleotides in length.

In some embodiments, the sense strand of a double-stranded iRNA agent is equal to or at least 10, 11, 12, 13, 14, 15, 16, 17, 18, 19, 20, 21, 22, 23, 23, 24, 25, 26, 27, 28, 29, or 30 nucleotides in length.

In one embodiment, the sense and antisense strands of the double-stranded iRNA agent are each 15 to 30 nucleotides in length.

In one embodiment, the sense and antisense strands of the double-stranded iRNA agent are each 19 to 25 nucleotides in length.

In one embodiment, the sense and antisense strands of the double-stranded iRNA agent are each 21 to 23 nucleotides in length.

In some embodiments, one strand has at least one stretch of 1-5 single-stranded nucleotides in the double-stranded region. By “stretch of single-stranded nucleotides in the double-stranded region” is meant that there is present at least one nucleotide base pair at both ends of the single-stranded stretch. In some embodiments, both strands have at least one stretch of 1-5 (e.g., 1, 2, 3, 4, or 5) single-stranded nucleotides in the double stranded region. When both strands have a stretch of 1-5 (e.g., 1, 2, 3, 4, or 5) single-stranded nucleotides in the double stranded region, such single-stranded nucleotides can be opposite to each other (e.g., a stretch of mismatches) or they can be located such that the second strand has no single-stranded nucleotides opposite to the single-stranded iRNAs of the first strand and vice versa (e.g., a single-stranded loop). In some embodiments, the single-stranded nucleotides are present within 8 nucleotides from either end, for example 8, 7, 6, 5, 4, 3, or 2 nucleotide from either the 5′ or 3′ end of the region of complementarity between the two strands.

In one embodiment, the double-stranded iRNA agent comprises a single-stranded overhang on at least one of the termini. In one embodiment, the single-stranded overhang is 1, 2, or 3 nucleotides in length.

In one embodiment, the sense strand of the iRNA agent is 21-nucleotides in length, and the antisense strand is 23-nucleotides in length, wherein the strands form a double-stranded region of 21 consecutive base pairs having a 2-nucleotide long single-stranded overhangs at the 3′-end.

In some embodiments, each strand of the double-stranded iRNA has a ZXY structure, such as is described in PCT Publication No. 2004080406, which is hereby incorporated by reference in its entirety.

In certain embodiment, the two strands of double-stranded oligomeric compound can be linked together. The two strands can be linked to each other at both ends, or at one end only. By linking at one end is meant that 5′-end of first strand is linked to the 3′-end of the second strand or 3′-end of first strand is linked to 5′-end of the second strand. When the two strands are linked to each other at both ends, 5′-end of first strand is linked to 3′-end of second strand and 3′-end of first strand is linked to 5′-end of second strand. The two strands can be linked together by an oligonucleotide linker including, but not limited to, (N)ₙ; wherein N is independently a modified or unmodified nucleotide and n is 3-23. In some embodiments, n is 3-10, e.g., 3, 4, 5, 6, 7, 8, 9, or 10. In some embodiments, the oligonucleotide linker is selected from the group consisting of GNRA, (G)₄, (U)₄, and (dT)₄, wherein N is a modified or unmodified nucleotide and R is a modified or unmodified purine nucleotide. Some of the nucleotides in the linker can be involved in base-pair interactions with other nucleotides in the linker. The two strands can also be linked together by a non-nucleosidic linker, e.g. a linker described herein. It will be appreciated by one of skill in the art that any oligonucleotide chemical modifications or variations describe herein can be used in the oligonucleotide linker.

Hairpin and dumbbell type oligomeric compounds will have a duplex region equal to or at least 14, 15, 15, 16, 17, 18, 19, 29, 21, 22, 23, 24, or 25 nucleotide pairs. The duplex region can be equal to or less than 200, 100, or 50, in length. In some embodiments, ranges for the duplex region are 15-30, 17 to 23, 19 to 23, and 19 to 21 nucleotides pairs in length.

The hairpin oligomeric compounds can have a single strand overhang or terminal unpaired region, in some embodiments at the 3′, and in some embodiments on the antisense side of the hairpin. In some embodiments, the overhangs are 1-4, more generally 2-3 nucleotides in length. The hairpin oligomeric compounds that can induce RNA interference are also referred to as “shRNA” herein.

In certain embodiments, two oligomeric strands specifically hybridize when there is a sufficient degree of complementarity to avoid non-specific binding of the antisense compound to non-target nucleic acid sequences under conditions in which specific binding is desired, i.e., under physiological conditions in the case of in vivo assays or therapeutic treatment, and under conditions in which assays are performed in the case of in vitro assays.

As used herein, “stringent hybridization conditions” or “stringent conditions” refers to conditions under which an antisense compound will hybridize to its target sequence, but to a minimal number of other sequences. Stringent conditions are sequence-dependent and will be different in different circumstances, and “stringent conditions” under which antisense compounds hybridize to a target sequence are determined by the nature and composition of the antisense compounds and the assays in which they are being investigated.

It is understood in the art that incorporation of nucleotide affinity modifications may allow for a greater number of mismatches compared to an unmodified compound. Similarly, certain oligonucleotide sequences may be more tolerant to mismatches than other oligonucleotide sequences. One of ordinary skill in the art is capable of determining an appropriate number of mismatches between oligonucleotides, or between an oligonucleotide and a target nucleic acid, such as by determining melting temperature (Tm). Tm or ΔTm can be calculated by techniques that are familiar to one of ordinary skill in the art. For example, techniques described in Freier et al. (Nucleic Acids Research, 1997, 25, 22: 4429-4443) allow one of ordinary skill in the art to evaluate nucleotide modifications for their ability to increase the melting temperature of an RNA:DNA duplex.

siRNA Design

In some embodiments, the double-strand iRNA agent discussed in all embodiments below, that has various siRNA designs, can further comprise a carbohydrate-based ligand (e.g., GalNAc₃). The double-stranded iRNA agent can further comprise a phosphate mimics, as described herein. The double-stranded iRNA agent can further comprise 2′-OMe modifications to more than fifteen, more than twenty, more than twenty-five, or more than thirty nucleotides.

In one embodiment, the iRNA agent of the invention is a double ended bluntmer of 19 nt in length, wherein the sense strand contains at least one motif of three 2′-F modifications on three consecutive nucleotides at positions 7,8,9 from the 5′end. The antisense strand contains at least one motif of three 2′-O-methyl modifications on three consecutive nucleotides at positions 11,12,13 from the 5′end.

In one embodiment, the iRNA agent of the invention is a double ended bluntmer of 20 nt in length, wherein the sense strand contains at least one motif of three 2′-F modifications on three consecutive nucleotides at positions 8,9,10 from the 5′end. The antisense strand contains at least one motif of three 2′-O-methyl modifications on three consecutive nucleotides at positions 11,12,13 from the 5′end.

In one embodiment, the iRNA agent of the invention is a double ended bluntmer of 21 nt in length, wherein the sense strand contains at least one motif of three 2′-F modifications on three consecutive nucleotides at positions 9,10,11 from the 5′end. The antisense strand contains at least one motif of three 2′-O-methyl modifications on three consecutive nucleotides at positions 11,12,13 from the 5′end.

In one embodiment, the iRNA agent of the invention comprises a 21 nucleotides (nt) sense strand and a 23 nucleotides (nt) antisense, wherein the sense strand contains at least one motif of three 2′-F modifications on three consecutive nucleotides at positions 9,10,11 from the 5′end; the antisense strand contains at least one motif of three 2′-O-methyl modifications on three consecutive nucleotides at positions 11,12,13 from the 5′end, wherein one end of the iRNA is blunt, while the other end is comprises a 2 nt overhang. Preferably, the 2 nt overhang is at the 3′-end of the antisense.

In one embodiment, the iRNA agent of the invention comprises a sense and antisense strands, wherein: the sense strand is 25-30 nucleotide residues in length, wherein starting from the 5′ terminal nucleotide (position 1) positions 1 to 23 of said first strand comprise at least 8 ribonucleotides; antisense strand is 36-66 nucleotide residues in length and, starting from the 3′ terminal nucleotide, comprises at least 8 ribonucleotides in the positions paired with positions 1-23 of sense strand to form a duplex; wherein at least the 3′ terminal nucleotide of antisense strand is unpaired with sense strand, and up to 6 consecutive 3′ terminal nucleotides are unpaired with sense strand, thereby forming a 3′ single stranded overhang of 1-6 nucleotides; wherein the 5′ terminus of antisense strand comprises from 10-30 consecutive nucleotides which are unpaired with sense strand, thereby forming a 10-30 nucleotide single stranded 5′ overhang; wherein at least the sense strand 5′ terminal and 3′ terminal nucleotides are base paired with nucleotides of antisense strand when sense and antisense strands are aligned for maximum complementarity, thereby forming a substantially duplexed region between sense and antisense strands; and antisense strand is sufficiently complementary to a target RNA along at least 19 ribonucleotides of antisense strand length to reduce target gene expression when said double stranded nucleic acid is introduced into a mammalian cell; and wherein the sense strand contains at least one motif of three 2′-F modifications on three consecutive nucleotides, where at least one of the motifs occurs at or near the cleavage site. The antisense strand contains at least one motif of three 2′-O-methyl modifications on three consecutive nucleotides at or near the cleavage site.

In one embodiment, the iRNA agent of the invention comprises a sense and antisense strands, wherein said iRNA agent comprises a first strand having a length which is at least 25 and at most 29 nucleotides and a second strand having a length which is at most 30 nucleotides with at least one motif of three 2′-O-methyl modifications on three consecutive nucleotides at position 11,12,13 from the 5′ end; wherein said 3′ end of said first strand and said 5′ end of said second strand form a blunt end and said second strand is 1-4 nucleotides longer at its 3′ end than the first strand, wherein the duplex region which is at least 25 nucleotides in length, and said second strand is sufficiently complementary to a target mRNA along at least 19 nt of said second strand length to reduce target gene expression when said iRNA agent is introduced into a mammalian cell, and wherein dicer cleavage of said iRNA preferentially results in an siRNA comprising said 3′ end of said second strand, thereby reducing expression of the target gene in the mammal.

In one embodiment, the sense strand of the iRNA agent contains at least one motif of three identical modifications on three consecutive nucleotides, where one of the motifs occurs at the cleavage site in the sense strand. For instance, the sense strand can contain at least one motif of three 2′-F modifications on three consecutive nucleotides within 7-15 positions from the 5′end.

In one embodiment, the antisense strand of the iRNA agent can also contain at least one motif of three identical modifications on three consecutive nucleotides, where one of the motifs occurs at or near the cleavage site in the antisense strand. For instance, the antisense strand can contain at least one motif of three 2′-O-methyl modifications on three consecutive nucleotides within 9-15 positions from the 5′end.

For iRNA agent having a duplex region of 17-23 nt in length, the cleavage site of the antisense strand is typically around the 10, 11 and 12 positions from the 5′-end. Thus the motifs of three identical modifications may occur at the 9, 10, 11 positions; 10, 11, 12 positions; 11, 12, 13 positions; 12, 13, 14 positions; or 13, 14, 15 positions of the antisense strand, the count starting from the 1^{st} nucleotide from the 5′-end of the antisense strand, or, the count starting from the 1^{st} paired nucleotide within the duplex region from the 5′-end of the antisense strand. The cleavage site in the antisense strand may also change according to the length of the duplex region of the iRNA from the 5′-end.

In some embodiments, the iRNA agent comprises a sense strand and antisense strand each having 14 to 30 nucleotides, wherein the sense strand contains at least two motifs of three identical modifications on three consecutive nucleotides, where at least one of the motifs occurs at or near the cleavage site within the strand and at least one of the motifs occurs at another portion of the strand that is separated from the motif at the cleavage site by at least one nucleotide. In one embodiment, the antisense strand also contains at least one motif of three identical modifications on three consecutive nucleotides, where at least one of the motifs occurs at or near the cleavage site within the strand. The modification in the motif occurring at or near the cleavage site in the sense strand is different than the modification in the motif occurring at or near the cleavage site in the antisense strand.

In some embodiments, the iRNA agent comprises a sense strand and antisense strand each having 14 to 30 nucleotides, wherein the sense strand contains at least one motif of three 2′-F modifications on three consecutive nucleotides, where at least one of the motifs occurs at or near the cleavage site in the strand. In one embodiment, the antisense strand also contains at least one motif of three 2′-O-methyl modifications on three consecutive nucleotides at or near the cleavage site.

In some embodiments, the iRNA agent comprises a sense strand and antisense strand each having 14 to 30 nucleotides, wherein the sense strand contains at least one motif of three 2′-F modifications on three consecutive nucleotides at positions 9,10,11 from the 5′end, and wherein the antisense strand contains at least one motif of three 2′-O-methyl modifications on three consecutive nucleotides at positions 11,12,13 from the 5′end.

In one embodiment, the iRNA agent of the invention comprises mismatch(es) with the target, within the duplex, or combinations thereof. The mistmatch can occur in the overhang region or the duplex region. The base pair can be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In one embodiment, the iRNA agent of the invention comprises at least one of the first 1, 2, 3, 4, or 5 base pairs within the duplex regions from the 5′-end of the antisense strand can be chosen independently from the group of: A:U, G:U, I:C, and mismatched pairs, e.g., non-canonical or other than canonical pairings or pairings which include a universal base, to promote the dissociation of the antisense strand at the 5′-end of the duplex.

In one embodiment, the nucleotide at the 1 position within the duplex region from the 5′-end in the antisense strand is selected from the group consisting of A, dA, dU, U, and dT. Alternatively, at least one of the first 1, 2 or 3 base pair within the duplex region from the 5′-end of the antisense strand is an AU base pair. For example, the first base pair within the duplex region from the 5′-end of the antisense strand is an AU base pair.

In some embodiments, the double-stranded iRNA (dsRNA) agent comprises a sense strand and an antisense strand, each strand having 14 to 40 nucleotides. The dsRNA agent is represented by formula (I):

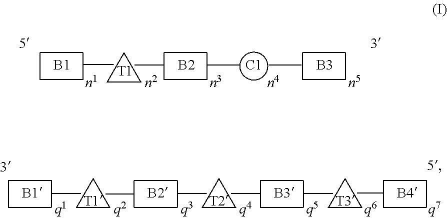

In formula (I), B1, B2, B3, B1′, B2′, B3′, and B4′ each are independently a nucleotide containing a modification selected from the group consisting of 2′-O-alkyl, 2′-substituted alkoxy, 2′-substituted alkyl, 2′-halo, ENA, and BNA/LNA. In one embodiment, B1, B2, B3, B1′, B2′, B3′, and B4′ each contain 2′-OMe modifications. In one embodiment, B1, B2, B3, B1′, B2′, B3′, and B4′ each contain 2′-OMe or 2′-F modifications. In one embodiment, at least one of B1, B2, B3, B1′, B2′, B3′, and B4′ contain 2′-O-N-methylacetamido (2′-O-NMA) modification.

C1 is a thermally destabilizing nucleotide placed at a site opposite to the seed region of the antisense strand (i.e., at positions 2-8 of the 5′-end of the antisense strand). For example, C1 is at a position of the sense strand that pairs with a nucleotide at positions 2-8 of the 5′-end of the antisense strand. In one example, C1 is at position 15 from the 5′-end of the sense strand. C1 nucleotide bears the thermally destabilizing modification which can include abasic modification; mismatch with the opposing nucleotide in the duplex; and sugar modification such as 2′-deoxy modification or acyclic nucleotide e.g., unlocked nucleic acids (UNA) or glycerol nucleic acid (GNA). In one embodiment, C1 has thermally destabilizing modification selected from the group consisting of: i) mismatch with the opposing nucleotide in the antisense strand; ii) abasic modification selected from the group consisting of:

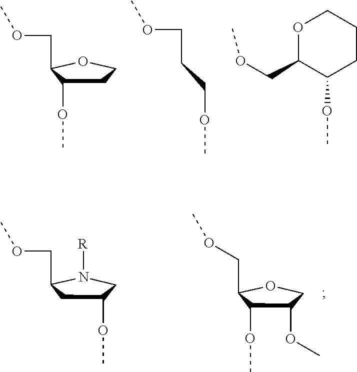

and iii) sugar modification selected from the group consisting of:

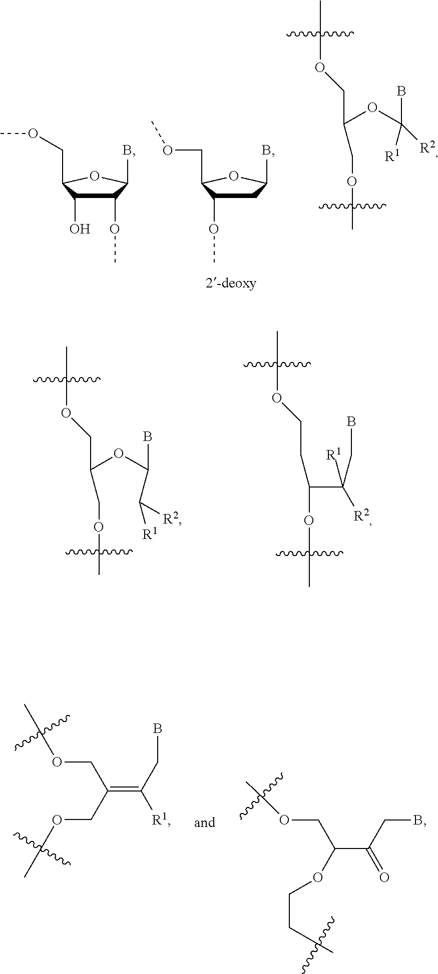

wherein B is a modified or unmodified nucleobase, R¹ and R² independently are H, halogen, OR₃, or alkyl; and R₃ is H, alkyl, cycloalkyl, aryl, aralkyl, heteroaryl or sugar. In one embodiment, the thermally destabilizing modification in C1 is a mismatch selected from the group consisting of G:G, G:A, G:U, G:T, A:A, A:C, C:C, C:U, C:T, U:U, T:T, and U:T; and optionally, at least one nucleobase in the mismatch pair is a 2′-deoxy nucleobase. In one example, the thermally destabilizing modification in C1 is GNA or

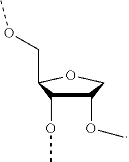

T1, T1′, T2′, and T3′ each independently represent a nucleotide comprising a modification providing the nucleotide a steric bulk that is less or equal to the steric bulk of a 2′-OMe modification. A steric bulk refers to the sum of steric effects of a modification. Methods for determining steric effects of a modification of a nucleotide are known to one skilled in the art. The modification can be at the 2′ position of a ribose sugar of the nucleotide, or a modification to a non-ribose nucleotide, acyclic nucleotide, or the backbone of the nucleotide that is similar or equivalent to the 2′ position of the ribose sugar, and provides the nucleotide a steric bulk that is less than or equal to the steric bulk of a 2′-OMe modification. For example, T1, T1′, T2′, and T3′ are each independently selected from DNA, RNA, LNA, 2′-F, and 2′-F-5′-methyl. In one embodiment, T1 is DNA. In one embodiment, T1′ is DNA, RNA or LNA. In one embodiment, T2′ is DNA or RNA. In one embodiment, T3′ is DNA or RNA.

n¹, n³, and q¹ are independently 4 to 15 nucleotides in length.

n⁵, q³, and q⁷ are independently 1-6 nucleotide(s) in length.

n⁴, q², and q⁶ are independently 1-3 nucleotide(s) in length; alternatively, n⁴ is 0. q⁵ is independently 0-10 nucleotide(s) in length.

n² and q⁴ are independently 0-3 nucleotide(s) in length.

Alternatively, n⁴ is 0-3 nucleotide(s) in length.

In one embodiment, n⁴ can be 0. In one example, n⁴ is 0, and q² and q⁶ are 1. In another example, n⁴ is 0, and q² and q⁶ are 1, with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, n⁴, q², and q⁶ are each 1.

In one embodiment, n², n⁴, q², q⁴, and q⁶ are each 1.

In one embodiment, C1 is at position 14-17 of the 5′-end of the sense strand, when the sense strand is 19-22 nucleotides in length, and n⁴ is 1. In one embodiment, C1 is at position 15 of the 5′-end of the sense strand

In one embodiment, T3′ starts at position 2 from the 5′ end of the antisense strand. In one example, T3′ is at position 2 from the 5′ end of the antisense strand and q⁶ is equal to 1.

In one embodiment, T1′ starts at position 14 from the 5′ end of the antisense strand. In one example, T1′ is at position 14 from the 5′ end of the antisense strand and q² is equal to 1.

In an exemplary embodiment, T3′ starts from position 2 from the 5′ end of the antisense strand and T1′ starts from position 14 from the 5′ end of the antisense strand. In one example, T3′ starts from position 2 from the 5′ end of the antisense strand and q⁶ is equal to 1 and T1′ starts from position 14 from the 5′ end of the antisense strand and q² is equal to 1.

In one embodiment, T1′ and T3′ are separated by 11 nucleotides in length (i.e. not counting the T1′ and T3′ nucleotides).

In one embodiment, T1′ is at position 14 from the 5′ end of the antisense strand. In one example, T1′ is at position 14 from the 5′ end of the antisense strand and q² is equal to 1, and the modification at the 2′ position or positions in a non-ribose, acyclic or backbone that provide less steric bulk than a 2′-OMe ribose.

In one embodiment, T3′ is at position 2 from the 5′ end of the antisense strand. In one example, T3′ is at position 2 from the 5′ end of the antisense strand and q⁶ is equal to 1, and the modification at the 2′ position or positions in a non-ribose, acyclic or backbone that provide less than or equal to steric bulk than a 2′-OMe ribose.

In one embodiment, T1 is at the cleavage site of the sense strand. In one example, T1 is at position 11 from the 5′ end of the sense strand, when the sense strand is 19-22 nucleotides in length, and n² is 1. In an exemplary embodiment, T1 is at the cleavage site of the sense strand at position 11 from the 5′ end of the sense strand, when the sense strand is 19-22 nucleotides in length, and n² is 1,

In one embodiment, T2′ starts at position 6 from the 5′ end of the antisense strand. In one example, T2′ is at positions 6-10 from the 5′ end of the antisense strand, and q⁴ is 1.

In an exemplary embodiment, T1 is at the cleavage site of the sense strand, for instance, at position 11 from the 5′ end of the sense strand, when the sense strand is 19-22 nucleotides in length, and n² is 1; T1′ is at position 14 from the 5′ end of the antisense strand, and q² is equal to 1, and the modification to T1′ is at the 2′ position of a ribose sugar or at positions in a non-ribose, acyclic or backbone that provide less steric bulk than a 2′-OMe ribose; T2′ is at positions 6-10 from the 5′ end of the antisense strand, and q⁴ is 1; and T3′ is at position 2 from the 5′ end of the antisense strand, and q⁶ is equal to 1, and the modification to T3′ is at the 2′ position or at positions in a non-ribose, acyclic or backbone that provide less than or equal to steric bulk than a 2′-OMe ribose.

In one embodiment, T2′ starts at position 8 from the 5′ end of the antisense strand. In one example, T2′ starts at position 8 from the 5′ end of the antisense strand, and q⁴ is 2.

In one embodiment, T2′ starts at position 9 from the 5′ end of the antisense strand. In one example, T2′ is at position 9 from the 5′ end of the antisense strand, and q⁴ is 1.

In one embodiment, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, n⁴ is 0, B3 is 2′-OMe, n^{s} is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 6, T1 is 2′F, n² is 3, B2 is 2′-OMe, n³ is 7, n⁴ is 0, B3 is 2′OMe, n⁵ is 3, B1′ is 2′-OMe or 2′-F, q¹ is 7, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1.

In one embodiment, B1 is 2′-OMe or 2′-F, n¹ is 6, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 7, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 5, T2′ is 2′-F, q⁴ is 1, B3′ is 2′-OMe or 2′-F, q⁵ is 5, T3′ is 2′-F, q⁶ is 1, B4′ is 2′-OMe, and q⁷ is 1; optionally with at least 2 additional TT at the 3′-end of the antisense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 5, T2′ is 2′-F, q⁴ is 1, B3′ is 2′-OMe or 2′-F, q⁵ is 5, T3′ is 2′-F, q⁶ is 1, B4′ is 2′-OMe, and q⁷ is 1; optionally with at least 2 additional TT at the 3′-end of the antisense strand; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within positions 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

The dsRNA agent can comprise a phosphorus-containing group, such as a phosphate or a phosphate mimic, at the 5′-end of the sense strand or antisense strand. The 5′-end phosphorus-containing group can be 5′-end phosphate (5′-P), 5′-end phosphorothioate (5′-PS), 5′-end phosphorodithioate (5′-PS₂), 5′-end vinylphosphonate (5′-VP), 5′-end methylphosphonate (MePhos), or 5′-deoxy-5′-C-malonyl

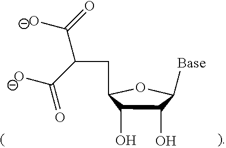

When the 5′-end phosphorus-containing group is 5′-end vinylphosphonate (5′-VP), the 5′-VP can be either 5′-E-VP isomer (i.e., trans-vinylphosphate,

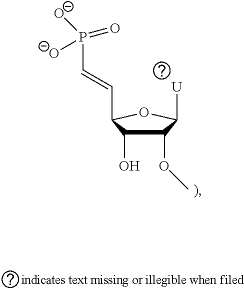

5′-Z-VP isomer (i.e., cis-vinylphosphate,

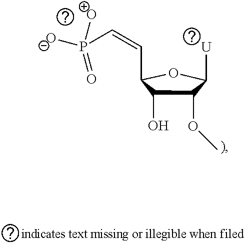

or mixtures thereof.

In one embodiment, the dsRNA agent comprises a phosphorus-containing group at the 5′-end of the sense strand. In one embodiment, the dsRNA agent comprises a phosphorus-containing group at the 5′-end of the antisense strand.

In one embodiment, the dsRNA agent comprises a 5′-P. In one embodiment, the dsRNA agent comprises a 5′-P in the antisense strand.

In one embodiment, the dsRNA agent comprises a 5′-PS. In one embodiment, the dsRNA agent comprises a 5′-PS in the antisense strand.

In one embodiment, the dsRNA agent comprises a 5′-VP. In one embodiment, the dsRNA agent comprises a 5′-VP in the antisense strand. In one embodiment, the dsRNA agent comprises a 5′-E-VP in the antisense strand. In one embodiment, the dsRNA agent comprises a 5′-Z-VP in the antisense strand.

In one embodiment, the dsRNA agent comprises a 5′-PS₂. In one embodiment, the dsRNA agent comprises a 5′-PS₂ in the antisense strand.

In one embodiment, the dsRNA agent comprises a 5′-PS₂. In one embodiment, the dsRNA agent comprises a 5′-deoxy-5′-C-malonyl in the antisense strand.

In some embodiments, the phosphate mimics are represented by Formula PM-I:

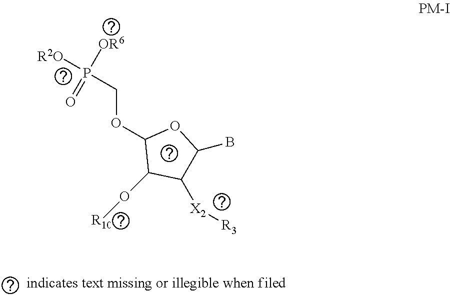

wherein:
R^{c} and R^{d} is each independently selected from CH₃, CH₂CH₃, CH₂CH₂CN, CH₂OCOC(CH₃)₃, CH₂OCH₂CH₂Si(CH₃)₃, or a protecting group;
B is a natural nucleobase, a modified nucleobase, a universal base or absent;
R₁₀ is a phosphoramidite; and
X₂ is OH, F, OCH₃, or OCH₂CH₂OCH₃ and Rₛ is absent; or X₂ is O and
R₈ is a glutathione sensitive moiety.

In certain embodiments, B is a natural nucleobase.

In certain embodiments, R^{c} and R^{d} is each independently selected from CH₃ and

In certain embodiments, X₂ is F or OCH₃ and R₈ is absent.

In certain embodiments, X₂ is O and R₈ is a glutathione sensitive moiety.

In certain embodiments, R^{c} and R^{d} are CH₃, R₈ is absent, and X₂ is F or OCH₃.

In certain embodiments, R^{c} and R^{d} are CH₂CH₃, R₈ is absent, and X₂ is F or OCH₃.

In certain embodiments, the phosphate mimics are represented by Formula PM-II:

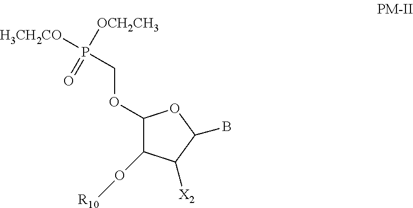

wherein:
B is a natural nucleobase, a modified nucleobase, a universal base or absent;
R₁₀ is a phosphoramidite; and
X₂ is OH, F, OCH₃, or OCH₂CH₂OCH₃.

In certain embodiments, B is a natural nucleobase.

In certain embodiments, X₂ is F or OCH₃.

In certain embodiments, the phosphate mimics are represented by Formula PM-III:

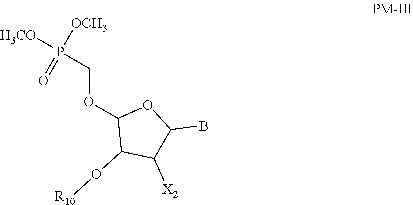

wherein:
B is a natural nucleobase, a modified nucleobase, a universal base or absent;
R₁₀ is a phosphoramidite; and
X₂ is OH, F, OCH₃, or OCH₂CH₂OCH₃.

In certain embodiments, B is a natural nucleobase.

In certain embodiments, X₂ is F or OCH₃.

In some embodiments, the phosphate mimics are represented by Formula PM-IV:

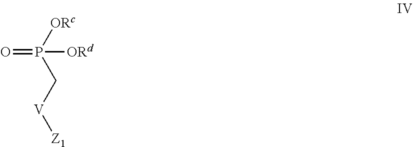

wherein:
R^{c} and R^{d} is each independently selected from CH₃, CH₂CH₃, CH₂CH₂CN, CH₂OCOC(CH₃)₃, CH₂OCH₂CH₂Si(CH₃)₃, or a protecting group;
V is O;
Z₁ is a nucleoside comprising a phosphoramidite and a sugar moiety; and
V is bound to the 4′-carbon of the sugar moiety.

Typically, the sugar moiety is a furanose and V is bound to the 4′-carbon of the furanose.

In certain embodiments, R^{c} and R^{d} are CH₃. In certain embodiments, R^{c} and R^{d} are CH₂CH₃.

Additional exemplary phosphate mimics suitable for the double-stranded iRNA agent herein may be found in WO 2018/045317; U.S. Pat. Nos. 8,927,513; and 11,119,136; which are incorporated herein by reference in their entirety.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The dsRNA agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The dsRNA agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The dsRNA agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The dsRNA agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The dsRNA agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The dsRNA agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The dsRNA agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The dsRNA agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The dsRNA agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The dsRNA agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The dsRNA agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The dsRNA agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The dsRNA agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The dsRNA agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, 4′ is 2′-F, and q⁷ is 1. The dsRNA agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The dsRNA agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The dsRNA agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The dsRNA agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The dsRNA agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The dsRNA agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-deoxy-5′-C-malonyl.

In one embodiment, 100%, 95%, 90%, 85%, 80%, 75%, 70%, 65%, 60%, 55%, 50%, 45%, 40%, 35% or 30% of the dsRNA agent of the invention is modified. For example, when 50% of the dsRNA agent is modified, 50% of all nucleotides present in the dsRNA agent contain a modification as described herein.

In one embodiment, each of the sense and antisense strands of the dsRNA agent is independently modified with acyclic nucleotides, LNA, HNA, CeNA, 2′-methoxyethyl, 2′-O-methyl, 2′-O-allyl, 2′-C-allyl, 2′-deoxy, 2′-fluoro, 2′-O-N-methylacetamido (2′-O-NMA), a 2′-O-dimethylaminoethoxyethyl (2′-O-DMAEOE), 2′-O-aminopropyl (2′-O-AP), or 2′-ara-F.

In one embodiment, each of the sense and antisense strands of the dsRNA agent contains at least two different modifications.

In one embodiment, the dsRNA agent of Formula (I) further comprises 3′ and/or 5′ overhang(s) of 1-10 nucleotides in length. In one example, dsRNA agent of formula (I) comprises a 3′ overhang at the 3′-end of the antisense strand and a blunt end at the 5′-end of the antisense strand. In another example, the dsRNA agent has a 5′ overhang at the 5′-end of the sense strand.

In one embodiment, the dsRNA agent of the invention does not contain any 2′-F modification.

In one embodiment, the sense strand and/or antisense strand of the dsRNA agent comprises one or more blocks of phosphorothioate or methylphosphonate internucleotide linkages. In one example, the sense strand comprises one block of two phosphorothioate or methylphosphonate internucleotide linkages. In one example, the antisense strand comprises two blocks of two phosphorothioate or methylphosphonate internucleotide linkages. For example, the two blocks of phosphorothioate or methylphosphonate internucleotide linkages are separated by 16-18 phosphate internucleotide linkages.

In one embodiment, each of the sense and antisense strands of the dsRNA agent has 15-30 nucleotides. In one example, the sense strand has 19-22 nucleotides, and the antisense strand has 19-25 nucleotides. In another example, the sense strand has 21 nucleotides, and the antisense strand has 23 nucleotides.

In one embodiment, the nucleotide at position 1 of the 5′-end of the antisense strand in the duplex is selected from the group consisting of A, dA, dU, U, and dT. In one embodiment, at least one of the first, second, and third base pair from the 5′-end of the antisense strand is an AU base pair.

In one embodiment, the antisense strand of the dsRNA agent of the invention is 100% complementary to a target RNA to hybridize thereto and inhibits its expression through RNA interference. In another embodiment, the antisense strand of the dsRNA agent of the invention is at least 95%, at least 90%, at least 85%, at least 80%, at least 75%, at least 70%, at least 65%, at least 60%, at least 55%, or at least 50% complementary to a target RNA.

In one aspect, the invention relates to a dsRNA agent as defined herein capable of inhibiting the expression of a target gene. The dsRNA agent comprises a sense strand and an antisense strand, each strand having 14 to 40 nucleotides. The sense strand contains at least one thermally destabilizing nucleotide, wherein at least one of said thermally destabilizing nucleotide occurs at or near the site that is opposite to the seed region of the antisense strand (i.e. at position 2-8 of the 5′-end of the antisense strand). Each of the embodiments and aspects described in this specification relating to the dsRNA represented by formula (I) can also apply to the dsRNA containing the thermally destabilizing nucleotide.

The thermally destabilizing nucleotide can occur, for example, between positions 14-17 of the 5′-end of the sense strand when the sense strand is 21 nucleotides in length. The antisense strand contains at least two modified nucleic acids that are smaller than a sterically demanding 2′-OMe modification. Preferably, the two modified nucleic acids that are smaller than a sterically demanding 2′-OMe are separated by 11 nucleotides in length. For example, the two modified nucleic acids are at positions 2 and 14 of the 5′ end of the antisense strand.

In one embodiment, the dsRNA agent further comprises at least one ASGPR ligand. For example, the ASGPR ligand is one or more GalNAc derivatives attached through a bivalent or trivalent branched linker, such as:

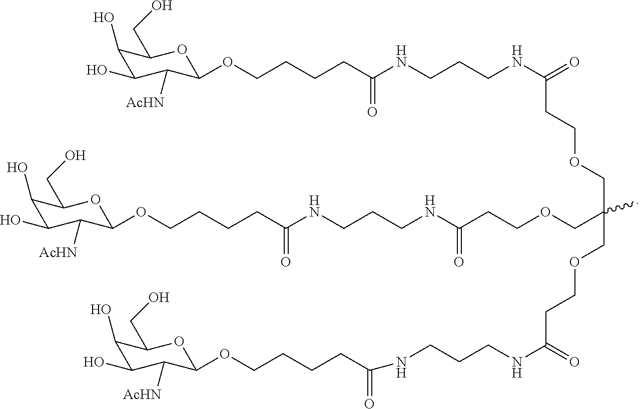 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O ; "."]

In one example, the ASGPR ligand is attached to the 3′ end or the 5′-end of the sense strand.

For example, the dsRNA agent as defined herein can comprise i) a phosphorus-containing group at the 5′-end of the sense strand or antisense strand; ii) with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand); and iii) a ligand, such as a ASGPR ligand (e.g., one or more GalNAc derivatives attached directly, or through a bivalent or trivalent branched linker) at the 5′-end or 3′-end of the sense strand or antisense strand. For instance, the ligand may be at the 3′-end or the 5′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-P and a targeting ligand. In one embodiment, the 5′-P is at the 5′-end of the antisense strand, and the targeting ligand is at the 3′-end or the 5′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS and a targeting ligand. In one embodiment, the 5′-PS is at the 5′-end of the antisense strand, and the targeting ligand is at the 3′-end or the 5′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-VP (e.g., a 5′-E-VP, 5′-Z-VP, or combination thereof), and a targeting ligand. In one embodiment, the 5′-VP is at the 5′-end of the antisense strand, and the targeting ligand is at the 3′-end or the 5′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS₂ and a targeting ligand. In one embodiment, the 5′-PS₂ is at the 5′-end of the antisense strand, and the targeting ligand is at the 3′-end or the 5′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end or the 5′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-P and a targeting ligand. In one embodiment, the 5′-P is at the 5′-end of the antisense strand, and the targeting ligand is at the 3′-end or the 5′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-PS and a targeting ligand. In one embodiment, the 5′-PS is at the 5′-end of the antisense strand, and the targeting ligand is at the 3′-end or the 5′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-VP (e.g., a 5′-E-VP, 5′-Z-VP, or combination thereof) and a targeting ligand. In one embodiment, the 5′-VP is at the 5′-end of the antisense strand, and the targeting ligand is at the 3′-end or the 5′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-PS₂ and a targeting ligand. In one embodiment, the 5′-PS₂ is at the 5′-end of the antisense strand, and the targeting ligand is at the 3′-end or the 5′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The dsRNA agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end or the 5′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-P and a targeting ligand. In one embodiment, the 5′-P is at the 5′-end of the antisense strand, and the targeting ligand is at the 3′-end or the 5′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS and a targeting ligand. In one embodiment, the 5′-PS is at the 5′-end of the antisense strand, and the targeting ligand is at the 3′-end or the 5′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-VP (e.g., a 5′-E-VP, 5′-Z-VP, or combination thereof) and a targeting ligand. In one embodiment, the 5′-VP is at the 5′-end of the antisense strand, and the targeting ligand is at the 3′-end or the 5′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS₂ and a targeting ligand. In one embodiment, the 5′-PS₂ is at the 5′-end of the antisense strand, and the targeting ligand is at the 3′-end or the 5′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end or the 5′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-P and a targeting ligand. In one embodiment, the 5′-P is at the 5′-end of the antisense strand, and the targeting ligand is at the 3′-end or the 5′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS and a targeting ligand. In one embodiment, the 5′-PS is at the 5′-end of the antisense strand, and the targeting ligand is at the 3′-end or the 5′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-VP (e.g., a 5′-E-VP, 5′-Z-VP, or combination thereof) and a targeting ligand. In one embodiment, the 5′-VP is at the 5′-end of the antisense strand, and the targeting ligand is at the 3′-end or the 5′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-PS₂ and a targeting ligand. In one embodiment, the 5′-PS₂ is at the 5′-end of the antisense strand, and the targeting ligand is at the 3′-end or the 5′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The dsRNA agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end or the 5′-end of the sense strand.

In a particular embodiment, the dsRNA agents of the present invention comprise:

(a) a sense strand having:
(i) a length of 21 nucleotides;
(ii) an ASGPR ligand attached to the 3′-end or the 5′-end, wherein said ASGPR ligand comprises one, two, or three GalNAc derivatives attached through a bivalent or trivalent branched linker; and
(iii) 2′-F modifications at positions 1, 3, 5, 7, 9 to 11, 13, 17, 19, and 21, and 2′-OMe modifications at positions 2, 4, 6, 8, 12, 14 to 16, 18, and 20 (counting from the 5′ end);
and
   (b) an antisense strand having:
(i) a length of 23 nucleotides;
(ii) 2′-OMe modifications at positions 1, 3, 5, 9, 11 to 13, 15, 17, 19, 21, and 23, and 2′F modifications at positions 2, 4, 6 to 8, 10, 14, 16, 18, 20, and 22 (counting from the 5′ end); and
(iii) phosphorothioate internucleotide linkages between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, the dsRNA agents of the present invention comprise:

(a) a sense strand having:
(i) a length of 21 nucleotides;
(ii) an ASGPR ligand attached to the 3′-end or the 5′-end, wherein said ASGPR ligand comprises one, two, or three GalNAc derivatives attached through a bivalent or trivalent branched linker;
(iii) 2′-F modifications at positions 1, 3, 5, 7, 9 to 11, 13, 15, 17, 19, and 21, and 2′-OMe modifications at positions 2, 4, 6, 8, 12, 14, 16, 18, and 20 (counting from the 5′ end); and
(iv) phosphorothioate internucleotide linkages between nucleotide positions 1 and 2, and between nucleotide positions 2 and 3 (counting from the 5′ end);
and
   (b) an antisense strand having:
(i) a length of 23 nucleotides;
(ii) 2′-OMe modifications at positions 1, 3, 5, 7, 9, 11 to 13, 15, 17, 19, and 21 to 23, and 2′F modifications at positions 2, 4, 6, 8, 10, 14, 16, 18, and 20 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
   wherein the dsRNA agents have a two nucleotide overhang at the 3′-end of the antisense strand, and a blunt end at the 5′-end of the antisense strand.

In another particular embodiment, the dsRNA agents of the present invention comprise:

(a) a sense strand having:
(i) a length of 21 nucleotides;
(ii) an ASGPR ligand attached to the 3′-end or the 5′-end, wherein said ASGPR ligand comprises one, two, or three GalNAc derivatives attached through a bivalent or trivalent branched linker;
(iii) 2′-OMe modifications at positions 1 to 6, 8, 10, and 12 to 21, 2′-F modifications at positions 7, and 9, and a desoxy-nucleotide (e.g. dT) at position 11 (counting from the 5′ end); and
(iv) phosphorothioate internucleotide linkages between nucleotide positions 1 and 2, and between nucleotide positions 2 and 3 (counting from the 5′ end);
and
   (b) an antisense strand having:
(i) a length of 23 nucleotides;
(ii) 2′-OMe modifications at positions 1, 3, 7, 9, 11, 13, 15, 17, and 19 to 23, and 2′-F modifications at positions 2, 4 to 6, 8, 10, 12, 14, 16, and 18 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
   wherein the dsRNA agents have a two nucleotide overhang at the 3′-end of the antisense strand, and a blunt end at the 5′-end of the antisense strand.

In another particular embodiment, the dsRNA agents of the present invention comprise:

(a) a sense strand having:
(i) a length of 21 nucleotides;
(ii) an ASGPR ligand attached to the 3′-end or the 5′-end, wherein said ASGPR ligand comprises one, two, or three GalNAc derivatives attached through a bivalent or trivalent branched linker;
(iii) 2′-OMe modifications at positions 1 to 6, 8, 10, 12, 14, and 16 to 21, and 2′-F modifications at positions 7, 9, 11, 13, and 15; and
(iv) phosphorothioate internucleotide linkages between nucleotide positions 1 and 2, and between nucleotide positions 2 and 3 (counting from the 5′ end);
and
   (b) an antisense strand having:
(i) a length of 23 nucleotides;
(ii) 2′-OMe modifications at positions 1, 5, 7, 9, 11, 13, 15, 17, 19, and 21 to 23, and 2′-F modifications at positions 2 to 4, 6, 8, 10, 12, 14, 16, 18, and 20 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
   wherein the dsRNA agents have a two nucleotide overhang at the 3′-end of the antisense strand, and a blunt end at the 5′-end of the antisense strand.

In another particular embodiment, the dsRNA agents of the present invention comprise:

(a) a sense strand having:
(i) a length of 21 nucleotides;
(ii) an ASGPR ligand attached to the 3′-end or the 5′-end, wherein said ASGPR ligand comprises one, two, or three GalNAc derivatives attached through a bivalent or trivalent branched linker;
(iii) 2′-OMe modifications at positions 1 to 9, and 12 to 21, and 2′-F modifications at positions 10, and 11; and
(iv) phosphorothioate internucleotide linkages between nucleotide positions 1 and 2, and between nucleotide positions 2 and 3 (counting from the 5′ end);
and
   (b) an antisense strand having:
(i) a length of 23 nucleotides;
(ii) 2′-OMe modifications at positions 1, 3, 5, 7, 9, 11 to 13, 15, 17, 19, and 21 to 23, and 2′-F modifications at positions 2, 4, 6, 8, 10, 14, 16, 18, and 20 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
   wherein the dsRNA agents have a two nucleotide overhang at the 3′-end of the antisense strand, and a blunt end at the 5′-end of the antisense strand.

In another particular embodiment, the dsRNA agents of the present invention comprise:

(a) a sense strand having:
(i) a length of 21 nucleotides;
(ii) an ASGPR ligand attached to the 3′-end or the 5′-end, wherein said ASGPR ligand comprises one, two, or three GalNAc derivatives attached through a bivalent or trivalent branched linker;
(iii) 2′-F modifications at positions 1, 3, 5, 7, 9 to 11, and 13, and 2′-OMe modifications at positions 2, 4, 6, 8, 12, and 14 to 21; and
(iv) phosphorothioate internucleotide linkages between nucleotide positions 1 and 2, and between nucleotide positions 2 and 3 (counting from the 5′ end);
and
   (b) an antisense strand having:
(i) a length of 23 nucleotides;
(ii) 2′-OMe modifications at positions 1, 3, 5 to 7, 9, 11 to 13, 15, 17 to 19, and 21 to 23, and 2′-F modifications at positions 2, 4, 8, 10, 14, 16, and 20 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
   wherein the dsRNA agents have a two nucleotide overhang at the 3′-end of the antisense strand, and a blunt end at the 5′-end of the antisense strand.

In another particular embodiment, the dsRNA agents of the present invention comprise:

(a) a sense strand having:
(i) a length of 21 nucleotides;
(ii) an ASGPR ligand attached to the 3′-end or the 5′-end, wherein said ASGPR ligand comprises one, two, or three GalNAc derivatives attached through a bivalent or trivalent branched linker;
(iii) 2′-OMe modifications at positions 1, 2, 4, 6, 8, 12, 14, 15, 17, and 19 to 21, and 2′-F modifications at positions 3, 5, 7, 9 to 11, 13, 16, and 18; and
(iv) phosphorothioate internucleotide linkages between nucleotide positions 1 and 2, and between nucleotide positions 2 and 3 (counting from the 5′ end);
and
   (b) an antisense strand having:
(i) a length of 25 nucleotides;
(ii) 2′-OMe modifications at positions 1, 4, 6, 7, 9, 11 to 13, 15, 17, and 19 to 23, 2′-F modifications at positions 2, 3, 5, 8, 10, 14, 16, and 18, and desoxy-nucleotides (e.g. dT) at positions 24 and 25 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
   wherein the dsRNA agents have a four nucleotide overhang at the 3′-end of the antisense strand, and a blunt end at the 5′-end of the antisense strand.

In another particular embodiment, the dsRNA agents of the present invention comprise:

(a) a sense strand having:
(i) a length of 21 nucleotides;
(ii) an ASGPR ligand attached to the 3′-end or the 5′-end, wherein said ASGPR ligand comprises one, two, or three GalNAc derivatives attached through a bivalent or trivalent branched linker;
(iii) 2′-OMe modifications at positions 1 to 6, 8, and 12 to 21, and 2′-F modifications at positions 7, and 9 to 11; and
(iv) phosphorothioate internucleotide linkages between nucleotide positions 1 and 2, and between nucleotide positions 2 and 3 (counting from the 5′ end);
and
   (b) an antisense strand having:
(i) a length of 23 nucleotides;
(ii) 2′-OMe modifications at positions 1, 3 to 5, 7, 8, 10 to 13, 15, and 17 to 23, and 2′-F modifications at positions 2, 6, 9, 14, and 16 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
   wherein the dsRNA agents have a two nucleotide overhang at the 3′-end of the antisense strand, and a blunt end at the 5′-end of the antisense strand.

In another particular embodiment, the dsRNA agents of the present invention comprise:

(a) a sense strand having:
(i) a length of 21 nucleotides;
(ii) an ASGPR ligand attached to the 3′-end or the 5′-end, wherein said ASGPR ligand comprises one, two, or three GalNAc derivatives attached through a bivalent or trivalent branched linker;
(iii) 2′-OMe modifications at positions 1 to 6, 8, and 12 to 21, and 2′-F modifications at positions 7, and 9 to 11; and
(iv) phosphorothioate internucleotide linkages between nucleotide positions 1 and 2, and between nucleotide positions 2 and 3 (counting from the 5′ end);
and
   (b) an antisense strand having:
(i) a length of 23 nucleotides;
(ii) 2′-OMe modifications at positions 1, 3 to 5, 7, 10 to 13, 15, and 17 to 23, and 2′-F modifications at positions 2, 6, 8, 9, 14, and 16 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
   wherein the dsRNA agents have a two nucleotide overhang at the 3′-end of the antisense strand, and a blunt end at the 5′-end of the antisense strand.

In another particular embodiment, the dsRNA agents of the present invention comprise:

(a) a sense strand having:
(i) a length of 19 nucleotides;
(ii) an ASGPR ligand attached to the 3′-end or the 5′-end, wherein said ASGPR ligand comprises one, two, or three GalNAc derivatives attached through a bivalent or trivalent branched linker;
(iii) 2′-OMe modifications at positions 1 to 4, 6, and 10 to 19, and 2′-F modifications at positions 5, and 7 to 9; and
(iv) phosphorothioate internucleotide linkages between nucleotide positions 1 and 2, and between nucleotide positions 2 and 3 (counting from the 5′ end);
and
   (b) an antisense strand having:
(i) a length of 21 nucleotides;
(ii) 2′-OMe modifications at positions 1, 3 to 5, 7, 10 to 13, 15, and 17 to 21, and 2′-F modifications at positions 2, 6, 8, 9, 14, and 16 (counting from the 5′ end); and
(iii) phosphorothioate internucleotide linkages between nucleotide positions 1 and 2, between nucleotide positions 2 and 3, between nucleotide positions 19 and 20, and between nucleotide positions 20 and 21 (counting from the 5′ end);
   wherein the dsRNA agents have a two nucleotide overhang at the 3′-end of the antisense strand, and a blunt end at the 5′-end of the antisense strand.

In one embodiment, the dsRNA agents of the present invention comprise:

(a) a sense strand having:
(i) a length of 18-23 nucleotides;
(ii) an ASGPR ligand attached to the 3′-end or the 5′-end, wherein said ASGPR ligand comprises one, two, or three GalNAc derivatives attached through a bivalent or trivalent branched linker;
(iii) three consecutive 2′-F modifications at positions 7-15; and
   (b) an antisense strand having:
(i) a length of 18-23 nucleotides;
(ii) at least 2′-F modifications anywhere on the strand; and
(iii) at least two phosphorothioate internucleotide linkages at the first five nucleotides (counting from the 5′ end);
   wherein the dsRNA agents have optionally one or more lipophilic moieties conjugated to one or more positions on at least one strand; and
   wherein the dsRNA agents either have two nucleotides overhang at the 3′-end of the antisense strand, and a blunt end at the 5′-end of the antisense strand; or blunt end both ends of the duplex.

In one embodiment, the dsRNA agents of the present invention comprise:

(a) a sense strand having:
(i) a length of 18-23 nucleotides;
(ii) less than four 2′-F modifications;
(iii) an ASGPR ligand attached to the 3′-end or the 5′-end, wherein said ASGPR ligand comprises one, two, or three GalNAc derivatives attached through a bivalent or trivalent branched linker;
   (b) an antisense strand having:
(i) a length of 18-23 nucleotides;
(ii) at less than twelve 2′-F modification; and
(iii) at least two phosphorothioate internucleotide linkages at the first five nucleotides (counting from the 5′ end);
   wherein the dsRNA agents have optionally one or more lipophilic moieties conjugated to one or more positions on at least one strand; and
   wherein the dsRNA agents either have two nucleotides overhang at the 3′-end of the antisense strand, and a blunt end at the 5′-end of the antisense strand; or blunt end both ends of the duplex.

In one embodiment, the dsRNA agents of the present invention comprise:

(a) a sense strand having:
(i) a length of 19-35 nucleotides;
(ii) less than four 2′-F modifications;
(iii) an ASGPR ligand attached to the 3′-end or the 5′-end, wherein said ASGPR ligand comprises one, two, or three GalNAc derivatives attached through a bivalent or trivalent branched linker;
   (b) an antisense strand having:
(i) a length of 19-35 nucleotides;
(ii) at less than twelve 2′-F modification; and
(iii) at least two phosphorothioate internucleotide linkages at the first five nucleotides (counting from the 5′ end);
   wherein the duplex region is between 19 to 25 base pairs (preferably 19, 20, 21 or 22); and
   wherein the dsRNA agents have optionally one or more lipophilic moieties conjugated to one or more positions on at least one strand; and
   wherein the dsRNA agents either have two nucleotides overhang at the 3′-end of the antisense strand, and a blunt end at the 5′-end of the antisense strand; or blunt end both ends of the duplex.

In one embodiment, the dsRNA agents of the present invention comprise a sense strand and antisense strands having a length of 15-30 nucleotides; at least two phosphorothioate internucleotide linkages at the first five nucleotides on the antisense strand (counting from the 5′ end); wherein the duplex region is between 19 to 25 base pairs (preferably 19, 20, 21 or 22); wherein the dsRNA agents have an ASGPR ligand, comprising one, two, or three GalNAc derivatives attached through a bivalent or trivalent branched linker, attached on at least one strand; and wherein the dsRNA agents have less than 20%, less than 15% and less than 10% non-natural nucleotide.

Examples of non-natural nucleotide includes acyclic nucleotides, LNA, HNA, CeNA, 2′-methoxyethyl, 2′-O-allyl, 2′-C-allyl, 2′-deoxy, 2′-fluoro, 2′-O-N-methylacetamido (2′-O-NMA), a 2′-O-dimethylaminoethoxyethyl (2′-O-DMAEOE), 2′-O-aminopropyl (2′-O-AP), or 2′-ara-F, and others.

In one embodiment, the dsRNA agents of the present invention comprise a sense strand and antisense strands having a length of 15-30 nucleotides; at least two phosphorothioate internucleotide linkages at the first five nucleotides on the antisense strand (counting from the 5′ end); wherein the duplex region is between 19 to 25 base pairs (preferably 19, 20, 21 or 22); wherein the dsRNA agents have an ASGPR ligand, comprising three GalNAc derivatives attached through a trivalent branched linker, attached on at least one strand; and wherein the dsRNA agents have greater than 80%, greater than 85% and greater than 90% natural nucleotide, such as 2′-OH, 2′-deoxy and 2′-OMe are natural nucleotides.

In one embodiment, the dsRNA agents of the present invention comprise a sense strand and antisense strands having a length of 15-30 nucleotides; at least two phosphorothioate internucleotide linkages at the first five nucleotides on the antisense strand (counting from the 5′ end); wherein the duplex region is between 19 to 25 base pairs (preferably 19, 20, 21 or 22); wherein the dsRNA agents have an ASGPR ligand, comprising three GalNAc derivatives attached through a trivalent branched linker, attached on at least one strand; and wherein the dsRNA agents have 100% natural nucleotide, such as 2′-OH, 2′-deoxy and 2′-OMe are natural nucleotides.

In one embodiment, the dsRNA agents of the present invention a sense strand and an antisense strand, each strand having 14 to 30 nucleotides, wherein the sense strand sequence is represented by formula (I):

5′nₚ-Nₐ-(XXX)ᵢ-N_{b}-YYY-N_{b}-(ZZZ)ⱼ-Nₐ-n_{q}3′ (I)

wherein:

i and j are each independently 0 or 1;

p and q are each independently 0-6;

each Nₐ independently represents an oligonucleotide sequence comprising 0-25 modified nucleotides, each sequence comprising at least two differently modified nucleotides;

each N_{b} independently represents an oligonucleotide sequence comprising 1, 2, 3, 4, 5, or 6 modified nucleotides;

each nₚ and n_{q} independently represent an overhang nucleotide;

wherein N_{b} and Y do not have the same modification;

wherein XXX, YYY and ZZZ each independently represent one motif of three identical modifications on three consecutive nucleotides;

wherein the dsRNA agents have an ASGPR ligand, comprising one, two, or three GalNAc derivatives attached through a bivalent or trivalent branched linker, attached on at least one strand; and

wherein the antisense strand of the dsRNA comprises two blocks of one, two pr three phosphorothioate internucleotide linkages separated by 1, 2, 3, 4, 5, 6, 7, 8, 9, 10, 11, 12, 13, 14, 15, 16, 17, or 18 phosphate internucleotide linkages.

Various publications described multimeric siRNA and can all be used with the iRNA of the invention. Such publications include WO2007/091269, U.S. Pat. No. 7,858,769, WO2010/141511, WO2007/117686, WO2009/014887 and WO2011/031520, which are hereby incorporated by reference in their entirety.

In some embodiments, 100%, 95%, 90%, 85%, 80%, 75%, 70%, 65%, 60%, 55%, 50%, 45%, 40%, 35% or 30% of the iRNA agent of the invention is modified.

In some embodiments, each of the sense and antisense strands of the iRNA agent is independently modified with acyclic nucleotides, LNA, HNA, CeNA, 2′-methoxyethyl, 2′-O-methyl, 2′-O-allyl, 2′-C-allyl, 2′-deoxy, 2′-fluoro, 2′-O-N-methylacetamido (2′-O-NMA), a 2′-O-dimethylaminoethoxyethyl (2′-O-DMAEOE), 2′-O-aminopropyl (2′-O-AP), or 2′-ara-F.

In some embodiments, each of the sense and antisense strands of the iRNA agent contains at least two different modifications.

In some embodiments, the double-stranded iRNA agent of the invention of the invention does not contain any 2′-F modification.

In some embodiments, the double-stranded iRNA agent of the invention contains one, two, three, four, five, six, seven, eight, nine, ten, eleven or twelve 2′-F modification(s). In one example, double-stranded iRNA agent of the invention contains nine or ten 2′-F modifications.

The iRNA agent of the invention may further comprise at least one phosphorothioate or methylphosphonate internucleotide linkage. The phosphorothioate or methylphosphonate internucleotide linkage modification may occur on any nucleotide of the sense strand or antisense strand or both in any position of the strand. For instance, the internucleotide linkage modification may occur on every nucleotide on the sense strand or antisense strand; each internucleotide linkage modification may occur in an alternating pattern on the sense strand or antisense strand; or the sense strand or antisense strand may contain both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand.

In one embodiment, the iRNA comprises the phosphorothioate or methylphosphonate internucleotide linkage modification in the overhang region. For example, the overhang region may contain two nucleotides having a phosphorothioate or methylphosphonate internucleotide linkage between the two nucleotides. Internucleotide linkage modifications also may be made to link the overhang nucleotides with the terminal paired nucleotides within duplex region. For example, at least 2, 3, 4, or all the overhang nucleotides may be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. Preferably, these terminal three nucleotides may be at the 3′-end of the antisense strand.

In some embodiments, the sense strand and/or antisense strand of the iRNA agent comprises one or more blocks of phosphorothioate or methylphosphonate internucleotide linkages. In one example, the sense strand comprises one block of two phosphorothioate or methylphosphonate internucleotide linkages. In one example, the antisense strand comprises two blocks of two phosphorothioate or methylphosphonate internucleotide linkages. For example, the two blocks of phosphorothioate or methylphosphonate internucleotide linkages are separated by 16-18 phosphate internucleotide linkages.

In some embodiments, the antisense strand of the iRNA agent of the invention is 100% complementary to a target RNA to hybridize thereto and inhibits its expression through RNA interference. In another embodiment, the antisense strand of the iRNA agent of the invention is at least 95%, at least 90%, at least 85%, at least 80%, at least 75%, at least 70%, at least 65%, at least 60%, at least 55%, or at least 50% complementary to a target RNA.

In one aspect, the invention relates to a double-stranded iRNA agent capable of inhibiting the expression of a target gene. The iRNA agent comprises a sense strand and an antisense strand, each strand having 14 to 40 nucleotides. The sense strand contains at least one thermally destabilizing nucleotide, wherein at at least one said thermally destabilizing nucleotide occurs at or near the site that is opposite to the seed region of the antisense strand (i.e.at position 2-8 of the 5′-end of the antisense strand), For example, the thermally destabilizing nucleotide occurs between positions 14-17 of the 5′-end of the sense strand when the sense strand is 21 nucleotides in length. The antisense strand contains at least two modified nucleic acids that are smaller than a sterically demanding 2′-OMe modification. Preferably, the two modified nucleic acids that is smaller than a sterically demanding 2′-OMe are separated by 11 nucleotides in length. For example, the two modified nucleic acids are at positions 2 and 14 of the 5′end of the antisense strand.

In some embodiments, the compound of the invention disclosed herein is a miRNA mimic. In one design, miRNA mimics are double stranded molecules (e.g., with a duplex region of between about 16 and about 31 nucleotides in length) and contain one or more sequences that have identity with the mature strand of a given miRNA. Double-stranded miRNA mimics have designs similar to as described above for double-stranded iRNAs. In some embodiments, a miRNA mimic comprises a duplex region of between 16 and 31 nucleotides and one or more of the following chemical modification patterns: the sense strand contains 2′-O-methyl modifications of nucleotides 1 and 2 (counting from the 5′ end of the sense oligonucleotide), and all of the Cs and Us; the antisense strand modifications can comprise 2′ F modification of all of the Cs and Us, phosphorylation of the 5′ end of the oligonucleotide, and stabilized internucleotide linkages associated with a 2 nucleotide 3′ overhang.

In some embodiments, the compound of the invention disclosed herein is an antimir. In some embodiments, compound of the invention comprises at least two antimirs covalently linked to each other via a nucleotide-based or non-nucleotide-based linker, for example a linker described in the disclosure, or non-covalently linked to each other. The terms “antimir” “microRNA inhibitor” or “miR inhibitor” are synonymous and refer to oligonucleotides or modified oligonucleotides that interfere with the activity of specific miRNAs. Inhibitors can adopt a variety of configurations including single stranded, double stranded (RNA/RNA or RNA/DNA duplexes), and hairpin designs, in general, microRNA inhibitors comprise one or more sequences or portions of sequences that are complementary or partially complementary with the mature strand (or strands) of the miRNA to be targeted, in addition, the miRNA inhibitor can also comprise additional sequences located 5′ and 3′ to the sequence that is the reverse complement of the mature miRNA. The additional sequences can be the reverse complements of the sequences that are adjacent to the mature miRNA in the pri-miRNA from which the mature miRNA is derived, or the additional sequences can be arbitrary sequences (having a mixture of A, G, C, U, or dT). In some embodiments, one or both of the additional sequences are arbitrary sequences capable of forming hairpins. Thus, in some embodiments, the sequence that is the reverse complement of the miRNA is flanked on the 5′ side and on the 3′ side by hairpin structures. MicroRNA inhibitors, when double stranded, can include mismatches between nucleotides on opposite strands. Furthermore, microRNA inhibitors can be linked to conjugate moieties in order to facilitate uptake of the inhibitor into a cell.

MicroRNA inhibitors, including hairpin miRNA inhibitors, are described in detail in Vermeulen et al., “Double-Stranded Regions Are Essential Design Components Of Potent Inhibitors of RISC Function,” RNA 13: 723-730 (2007) and in WO2007/095387 and WO 2008/036825 each of which is incorporated herein by reference in its entirety. A person of ordinary skill in the art can select a sequence from the database for a desired miRNA and design an inhibitor useful for the methods disclosed herein.

In some embodiments, the compound of the invention disclosed herein is an antagomir. In some embodiments, the compound of the invention comprises at least two antagomirs covalently linked to each other via a nucleotide-based or non-nucleotide-based linker, for example a linker described in the disclosure, or non-covalently linked to each other. Antagomirs are RNA-like oligonucleotides that harbor various modifications for RNAse protection and pharmacologic properties, such as enhanced tissue and cellular uptake. They differ from normal RNA by, for example, complete 2′-O-methylation of sugar, phosphorothioate intersugar linkage and, for example, a cholesterol-moiety at 3′-end. In a preferred embodiment, antagomir comprises a 2′-O-methyl modification at all nucleotides, a cholesterol moiety at 3′-end, two phosphorothioate intersugar linkages at the first two positions at the 5′-end and four phosphorothioate linkages at the 3′-end of the molecule. Antagomirs can be used to efficiently silence endogenous miRNAs by forming duplexes comprising the antagomir and endogenous miRNA, thereby preventing miRNA-induced gene silencing. An example of antagomir-mediated miRNA silencing is the silencing of miR-122, described in Krutzfeldt et al, Nature, 2005, 438: 685-689, which is expressly incorporated by reference herein in its entirety.

Recent studies have found that dsRNA can also activate gene expression, a mechanism that has been termed “small RNA-induced gene activation” or RNAa (activating RNA). See for example Li, L. C. et al. Proc Natl Acad Sci USA. (2006), 103(46):17337-42 and Li L. C. (2008). “Small RNA-Mediated Gene Activation”. RNA and the Regulation of Gene Expression: A Hidden Layer of Complexity. Caister Academic Press. ISBN 978-1-904455-25-7. It has been shown that dsRNAs targeting gene promoters induce potent transcriptional activation of associated genes. Endogenous miRNA that cause RNAa has also been found in humans. Check E. Nature (2007). 448 (7156): 855-858.

Another surprising observation is that gene activation by RNAa is long-lasting. Induction of gene expression has been seen to last for over ten days. The prolonged effect of RNAa could be attributed to epigenetic changes at dsRNA target sites. In some embodiments, the RNA activator can increase the expression of a gene. In some embodiments, increased gene expression inhibits viability, growth development, and/or reproduction.

Accordingly, in some embodiments, the compound of the invention disclosed herein is activating RNA. In some embodiments, the compound of the invention comprises at least two activating RNAs scovalently linked to each other via a nucleotide-based or non-nucleotide-based linker, for example a linker described in the disclosure, or non-covalently linked to each other.

Accordingly, in some embodiments, the compound of the invention disclosed herein is a triplex forming oligonuclotide (TFO). In some embodiments, the compound of the invention comprises at least two TFOs covalently linked to each other via a nucleotide-based or non-nucleotide-based linker, for example a linker described in the disclosure, or non-covalently linked to each other. Recent studies have shown that triplex forming oligonucleotides can be designed which can recognize and bind to polypurine/polypyrimidine regions in double-stranded helical DNA in a sequence-specific manner. These recognition rules are outline by Maher III, L. J., et al., Science (1989) vol. 245, pp 725-730; Moser, H. E., et al., Science (1987) vol. 238, pp 645-630; Beal, P. A., et al., Science (1992) vol. 251, pp 1360-1363; Conney, M., et al., Science (1988) vol. 241, pp 456-459 and Hogan, M. E., et al., EP Publication 375408. Modification of the oligonucleotides, such as the introduction of intercalators and intersugar linkage substitutions, and optimization of binding conditions (pH and cation concentration) have aided in overcoming inherent obstacles to TFO activity such as charge repulsion and instability, and it was recently shown that synthetic oligonucleotides can be targeted to specific sequences (for a recent review see Seidman and Glazer, J Clin Invest 2003; 112:487-94). In general, the triplex-forming oligonucleotide has the sequence correspondence:

oligo
3′-A G G T
duplex
5′-A G C T
duplex
3′-T C G A

However, it has been shown that the A-AT and G-GC triplets have the greatest triple helical stability (Reither and Jeltsch, BMC Biochem, 2002, September 12, Epub). The same authors have demonstrated that TFOs designed according to the A-AT and G-GC rule do not form non-specific triplexes, indicating that the triplex formation is indeed sequence specific.

Thus for any given sequence a triplex forming sequence can be devised. Triplex-forming oligonucleotides preferably are at least 15, more preferably 25, still more preferably 30 or more nucleotides in length, up to 50 or 100 nucleotides.

Formation of the triple helical structure with the target DNA induces steric and functional changes, blocking transcription initiation and elongation, allowing the introduction of desired sequence changes in the endogenous DNA and resulting in the specific down-regulation of gene expression. Examples of such suppression of gene expression in cells treated with TFOs include knockout of episomal supFG1 and endogenous HPRT genes in mammalian cells (Vasquez et al., Nucl Acids Res. 1999; 27: 1176-81, and Puri, et al, J Biol Chem, 2001; 276:28991-98), and the sequence- and target specific downregulation of expression of the Ets2 transcription factor, important in prostate cancer etiology (Carbone, et al, Nucl Acid Res. 2003; 31:833-43), and the pro-inflammatory ICAM-I gene (Besch et al, J Biol Chem, 2002; 277:32473-79). In addition, Vuyisich and Beal have recently shown that sequence specific TFOs can bind to dsRNA, inhibiting activity of dsRNA-dependent enzymes such as RNA-dependent kinases (Vuyisich and Beal, Nuc. Acids Res 2000; 28:2369-74).

Additionally, TFOs designed according to the abovementioned principles can induce directed mutagenesis capable of effecting DNA repair, thus providing both down-regulation and up-regulation of expression of endogenous genes (Seidman and Glazer, J Clin Invest 2003; 112:487-94). Detailed description of the design, synthesis and administration of effective TFOs can be found in U.S. Pat. App. Nos. 2003 017068 and 2003 0096980 to Froehler et al, and 2002 0128218 and 2002 0123476 to Emanuele et al, and U.S. Pat. No. 5,721,138 to Lawn, contents of which are herein incorporated in their entireties.

### Nucleic Acid Modifications

In some embodiments, the double-stranded iRNA agent of the invention comprises at least one nucleic acid modification described herein. For example, at least one modification selected from the group consisting of modified internucleoside linkage, modified nucleobase, modified sugar, and any combinations thereof. Without limitations, such a modification can be present anywhere in the double-stranded iRNA agent of the invention. For example, the modification can be present in one of the RNA molecules.

### Nucleic Acid Modifications (Nucleobases)

The naturally occurring base portion of a nucleoside is typically a heterocyclic base. The two most common classes of such heterocyclic bases are the purines and the pyrimidines. For those nucleosides that include a pentofuranosyl sugar, a phosphate group can be linked to the 2′, 3′ or 5′ hydroxyl moiety of the sugar. In forming oligonucleotides, those phosphate groups covalently link adjacent nucleosides to one another to form a linear polymeric compound. Within oligonucleotides, the phosphate groups are commonly referred to as forming the internucleoside backbone of the oligonucleotide. The naturally occurring linkage or backbone of RNA and of DNA is a 3′ to 5′ phosphodiester linkage.

In addition to “unmodified” or “natural” nucleobases such as the purine nucleobases adenine (A) and guanine (G), and the pyrimidine nucleobases thymine (T), cytosine (C) and uracil (U), many modified nucleobases or nucleobase mimetics known to those skilled in the art are amenable with the compounds described herein. The unmodified or natural nucleobases can be modified or replaced to provide iRNAs having improved properties. For example, nuclease resistant oligonucleotides can be prepared with these bases or with synthetic and natural nucleobases (e.g., inosine, xanthine, hypoxanthine, nubularine, isoguanisine, or tubercidine) and any one of the oligomer modifications described herein. Alternatively, substituted or modified analogs of any of the above bases and “universal bases” can be employed. When a natural base is replaced by a non-natural and/or universal base, the nucleotide is said to comprise a modified nucleobase and/or a nucleobase modification herein. Modified nucleobase and/or nucleobase modifications also include natural, non-natural and universal bases, which comprise conjugated moieties, e.g. a ligand described herein. Preferred conjugate moieties for conjugation with nucleobases include cationic amino groups which can be conjugated to the nucleobase via an appropriate alkyl, alkenyl or a linker with an amide linkage.

An oligomeric compound described herein can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Exemplary modified nucleobases include, but are not limited to, other synthetic and natural nucleobases such as inosine, xanthine, hypoxanthine, nubularine, isoguanisine, tubercidine, 2-(halo)adenine, 2-(alkyl)adenine, 2-(propyl)adenine, 2-(amino)adenine, 2-(aminoalkyll)adenine, 2-(aminopropyl)adenine, 2-(methylthio)-N⁶-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶, N⁶-(dimethyl)adenine, 2-(alkyl)guanine, 2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N⁴-(acetyl)cytosine, 3-(3-amino-3-carboxypropyl)uracil, 2-(thio)uracil, 5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouracil, 4-(thio)pseudouracil, 2,4-(dithio)psuedouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, O⁶-substituted purines, substituted 1,2,4-triazoles, pyrrolo-pyrimidin-2-on-3-yl, 6-phenyl-pyrrolo-pyrimidin-2-on-3-yl, para-substituted-6-phenyl-pyrrolo-pyrimidin-2-on-3-yl, ortho-substituted-6-phenyl-pyrrolo-pyrimidin-2-on-3-yl, bis-ortho-substituted-6-phenyl-pyrrolo-pyrimidin-2-on-3-yl, para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho-(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof. Alternatively, substituted or modified analogs of any of the above bases and “universal bases” can be employed.

As used herein, a universal nucleobase is any nucleobase that can base pair with all of the four naturally occurring nucleobases without substantially affecting the melting behavior, recognition by intracellular enzymes or activity of the iRNA duplex. Some exemplary universal nucleobases include, but are not limited to, 2,4-difluorotoluene, nitropyrrolyl, nitroindolyl, 8-aza-7-deazaadenine, 4-fluoro-6-methylbenzimidazle, 4-methylbenzimidazle, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylinolyl, 4,6-dimethylindolyl, phenyl, napthalenyl, anthracenyl, phenanthracenyl, pyrenyl, stilbenyl, tetracenyl, pentacenyl, and structural derivatives thereof (see for example, Loakes, 2001, Nucleic Acids Research, 29, 2437-2447).

Further nucleobases include those disclosed in U.S. Pat. No. 3,687,808; those disclosed in International Application No. PCT/US09/038425, filed Mar. 26, 2009; those disclosed in the Concise Encyclopedia Of Polymer Science And Engineering, pages 858-859, Kroschwitz, J. I., ed. John Wiley & Sons, 1990; those disclosed by English et al., Angewandte Chemie, International Edition, 1991, 30, 613; those disclosed in Modified Nucleosides in Biochemistry, Biotechnology and Medicine, Herdewijin, P. Ed. Wiley-VCH, 2008; and those disclosed by Sanghvi, Y. S., Chapter 15, dsRNA Research and Applications, pages 289-302, Crooke, S. T. and Lebleu, B., Eds., CRC Press, 1993. Contents of all of the above are herein incorporated by reference.

In certain embodiments, a modified nucleobase is a nucleobase that is fairly similar in structure to the parent nucleobase, such as for example a 7-deaza purine, a 5-methyl cytosine, or a G-clamp. In certain embodiments, nucleobase mimetic include more complicated structures, such as for example a tricyclic phenoxazine nucleobase mimetic. Methods for preparation of the above noted modified nucleobases are well known to those skilled in the art.

### Nucleic Acid Modifications (Sugar)

Double-stranded iRNA agent of the inventions provided herein can comprise one or more (e.g., 1, 2, 3, 4, 5, 6, 7, 8, 9, 10, 11, 12, 13, 14, 15 or more) monomer, including a nucleoside or nucleotide, having a modified sugar moiety. For example, the furanosyl sugar ring of a nucleoside can be modified in a number of ways including, but not limited to, addition of a substituent group, bridging of two non-geminal ring atoms to form a locked nucleic acid or bicyclic nucleic acid. In certain embodiments, oligomeric compounds comprise one or more (e.g., 1, 2, 3, 4, 5, 6, 7, 8, 9, 10, 11, 12, 13, 14, 15 or more) monomers that are LNA.

In some embodiments of a locked nucleic acid, the 2′ position of furnaosyl is connected to the 4′ position by a linker selected independently from —[C(R1)(R2)]ₙ-, -[C(R1)(R2)]ₙ-O—, —[C(R1)(R2)]ₙ-N(R1)-, —[C(R1)(R2)]ₙ-N(R1)-O—, [C(R1R2)]ₙ-O—N(R1)-, —C(R1)=C(R2)-O—, —C(R1)=N—, —C(R1)=N—O—, —C(═NR1)-, C(═NR1)-O—, —C(═O)—, —C(═O)O—, —C(═S)—, —C(═S)O—, —C(═S)S—, —O—, —Si(R1)₂-, —S(═O)ₓ— and —N(R1)-;

wherein:

x is 0, 1, or 2;

n is 1, 2, 3, or 4;

each R1 and R2 is, independently, H, a protecting group, hydroxyl, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, heterocycle radical, substituted heterocycle radical, heteroaryl, substituted heteroaryl, C5-C7 alicyclic radical, substituted C5-C7 alicyclic radical, halogen, OJ1, NJ1J2, SJ1, N3, COOJ1, acyl (C(═O)—H), substituted acyl, CN, sulfonyl (S(═O)2-J1), or sulfoxyl (S(═O)-J1); and

each J1 and J2 is, independently, H, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, acyl (C(═O)—H), substituted acyl, a heterocycle radical, a substituted heterocycle radical, C1-C12 aminoalkyl, substituted C1-C12 aminoalkyl or a protecting group.

In some embodiments, each of the linkers of the LNA compounds is, independently, —[C(R1)(R2)]n-, [C(R1)(R2)]n-O—, C(R1R2)-N(R1)-O— or —C(R1R2)-O-N(R1)-. In another embodiment, each of said linkers is, independently, 4′-CH₂-2′, 4′-(CH₂)₂-2′, 4′-(CH₂)₃-2′, 4′-CH₂—O-2′, 4′-(CH₂)₂-O-2′, 4′-CH₂—O-N(R1)-2′ and 4′-CH₂—N(R1)-O-2′- wherein each R1 is, independently, H, a protecting group or C1-C12 alkyl.

Certain LNA's have been prepared and disclosed in the patent literature as well as in scientific literature (Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; WO 94/14226; WO 2005/021570; Singh et al., J. Org. Chem., 1998, 63, 10035-10039; Examples of issued US patents and published applications that disclose LNA s include, for example, U.S. Pat. Nos. 7,053,207; 6,268,490; 6,770,748; 6,794,499; 7,034,133; and 6,525,191; and U.S. Pre-Grant Publication Nos. 2004-0171570; 2004-0219565; 2004-0014959; 2003-0207841; 2004-0143114; and 20030082807.

Also provided herein are LNAs in which the 2′-hydroxyl group of the ribosyl sugar ring is linked to the 4′ carbon atom of the sugar ring thereby forming a methyleneoxy (4′-CH₂—O-2′) linkage to form the bicyclic sugar moiety (reviewed in Elayadi et al., Curr. Opinion Invens. Drugs, 2001, 2, 558-561; Braasch et al., Chem. Biol., 2001, 8 1-7; and Orum et al., Curr. Opinion Mol. Ther., 2001, 3, 239-243; see also U.S. Pat. Nos. 6,268,490 and 6,670,461). The linkage can be a methylene (—CH₂—) group bridging the 2′ oxygen atom and the 4′ carbon atom, for which the term methyleneoxy (4′-CH₂—O-2′) LNA is used for the bicyclic moiety; in the case of an ethylene group in this position, the term ethyleneoxy (4′-CH₂CH₂—O-2′) LNA is used (Singh et al., Chem. Commun., 1998, 4, 455-456: Morita et al., Bioorganic Medicinal Chemistry, 2003, 11, 2211-2226). Methyleneoxy (4′-CH₂—O-2′) LNA and other bicyclic sugar analogs display very high duplex thermal stabilities with complementary DNA and RNA (Tm=+3 to +100 C.), stability towards 3′-exonucleolytic degradation and good solubility properties. Potent and nontoxic antisense oligonucleotides comprising BNAs have been described (Wahlestedt et al., Proc. Natl. Acad. Sci. U.S.A., 2000, 97, 5633-5638).

An isomer of methyleneoxy (4′-CH₂—O-2′) LNA that has also been discussed is alpha-L-methyleneoxy (4′-CH₂—O-2′) LNA which has been shown to have superior stability against a 3′-exonuclease. The alpha-L-methyleneoxy (4′-CH₂—O-2′) LNA's were incorporated into antisense gapmers and chimeras that showed potent antisense activity (Frieden et al., Nucleic Acids Research, 2003, 21, 6365-6372).

The synthesis and preparation of the methyleneoxy (4′-CH₂—O-2′) LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and preparation thereof are also described in WO 98/39352 and WO 99/14226.

Analogs of methyleneoxy (4′-CH₂—O-2′) LNA, phosphorothioate-methyleneoxy (4′-CH₂—O-2′) LNA and 2′-thio-LNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (Wengel et al., WO 99/14226). Furthermore, synthesis of 2′-amino-LNA, a novel conformationally restricted high-affinity oligonucleotide analog has been described in the art (Singh et al., J. Org. Chem., 1998, 63, 10035-10039). In addition, 2′-Amino- and 2′-methylamino-LNA's have been prepared and the thermal stability of their duplexes with complementary RNA and DNA strands has been previously reported.

Modified sugar moieties are well known and can be used to alter, typically increase, the affinity of the antisense compound for its target and/or increase nuclease resistance. A representative list of preferred modified sugars includes but is not limited to bicyclic modified sugars, including methyleneoxy (4′-CH₂—O-2′) LNA and ethyleneoxy (4′-(CH₂)2-O-2′ bridge) ENA; substituted sugars, especially 2′-substituted sugars having a 2′-F, 2′-OCH₃ or a 2′-O(CH₂)2-OCH₃ substituent group; and 4′-thio modified sugars. Sugars can also be replaced with sugar mimetic groups among others. Methods for the preparations of modified sugars are well known to those skilled in the art. Some representative patents and publications that teach the preparation of such modified sugars include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; 6,531,584; and 6,600,032; and WO 2005/121371.

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)CH₂CH₂OR, n=1-50; “locked” nucleic acids (LNA) in which the furanose portion of the nucleoside includes a bridge connecting two carbon atoms on the furanose ring, thereby forming a bicyclic ring system; O-AMINE or O—(CH₂)ₙAMINE (n=1-10, AMINE=NH₂; alkylamino, dialkylamino, heterocyclyl, arylamino, diaryl amino, heteroaryl amino, diheteroaryl amino, ethylene diamine or polyamino); and O—CH₂CH₂(NCH₂CH₂NMe₂)₂.

“Deoxy” modifications include hydrogen (i.e. deoxyribose sugars, which are of particular relevance to the single-strand overhangs); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino); —NHC(O)R (R=alkyl, cycloalkyl, aryl, aralkyl, heteroaryl or sugar); cyano; mercapto; alkyl-thio-alkyl; thioalkoxy; thioalkyl; alkyl; cycloalkyl; aryl; alkenyl and alkynyl, which can be optionally substituted with e.g., an amino functionality.

Other suitable 2′-modifications, e.g., modified MOE, are described in U.S. Patent Application Publication No. 20130130378, contents of which are herein incorporated by reference.

A modification at the 2′ position can be present in the arabinose configuration The term “arabinose configuration” refers to the placement of a substituent on the C2′ of ribose in the same configuration as the 2′-OH is in the arabinose.

The sugar can comprise two different modifications at the same carbon in the sugar, e.g., gem modification. The sugar group can also contain one or more carbons that possess the opposite stereochemical configuration than that of the corresponding carbon in ribose. Thus, an oligomeric compound can include one or more monomers containing e.g., arabinose, as the sugar. The monomer can have an alpha linkage at the 1′ position on the sugar, e.g., alpha-nucleosides. The monomer can also have the opposite configuration at the 4′-position, e.g., C5′ and H4′ or substituents replacing them are interchanged with each other. When the C5′ and H4′ or substituents replacing them are interchanged with each other, the sugar is said to be modified at the 4′ position.

Double-stranded iRNA agent of the inventions disclosed herein can also include abasic sugars, i.e., a sugar which lack a nucleobase at C-1′ or has other chemical groups in place of a nucleobase at C1′. See for example U.S. Pat. No. 5,998,203, content of which is herein incorporated in its entirety. These abasic sugars can also be further containing modifications at one or more of the constituent sugar atoms. Double-stranded iRNA agent of the inventions can also contain one or more sugars that are the L isomer, e.g. L-nucleosides. Modification to the sugar group can also include replacement of the 4′-O with a sulfur, optionally substituted nitrogen or CH₂ group. In some embodiments, linkage between C1′ and nucleobase is in a configuration.

Sugar modifications can also include acyclic nucleotides, wherein a C-C bonds between ribose carbons (e.g., C1′-C2′, C2′-C3′, C3′-C4′, C4′-O4′, C1′-O4′) is absent and/or at least one of ribose carbons or oxygen (e.g., C1′, C2′, C3′, C4′ or O4′) are independently or in combination absent from the nucleotide. In some embodiments, acyclic nucleotide is

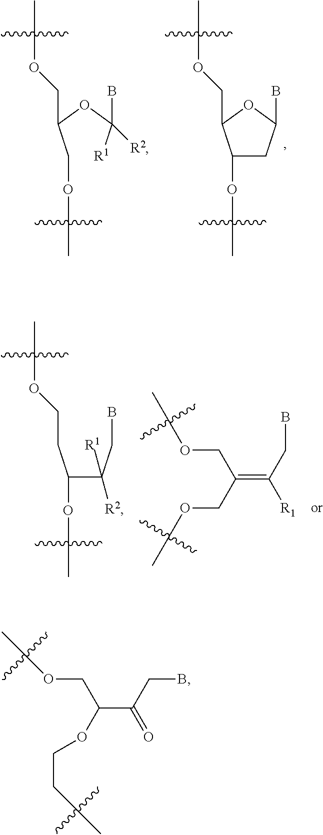

wherein B is a modified or unmodified nucleobase, R₁ and R₂ independently are H, halogen, OR₃, or alkyl; and R₃ is H, alkyl, cycloalkyl, aryl, aralkyl, heteroaryl or sugar).

In some embodiments, sugar modifications are selected from the group consisting of 2′-H, 2′-O-Me (2′-O-methyl), 2′-O-MOE (2′-O-methoxyethyl), 2′-F, 2′-O—[2-(methylamino)-2-oxoethyl] (2′-O-NMA), 2′-S-methyl, 2′-O—CH₂-(4′-C) (LNA), 2′-O—CH₂CH₂-(4′-C) (ENA), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE) and gem 2′-OMe/2′F with 2′-O-Me in the arabinose configuration.

It is to be understood that when a particular nucleotide is linked through its 2′-position to the next nucleotide, the sugar modifications described herein can be placed at the 3′-position of the sugar for that particular nucleotide, e.g., the nucleotide that is linked through its 2′-position. A modification at the 3′ position can be present in the xylose configuration The term “xylose configuration” refers to the placement of a substituent on the C3′ of ribose in the same configuration as the 3′-OH is in the xylose sugar.

The hydrogen attached to C4′ and/or C1′ can be replaced by a straight- or branched-optionally substituted alkyl, optionally substituted alkenyl, optionally substituted alkynyl, wherein backbone of the alkyl, alkenyl and alkynyl can contain one or more of O, S, S(O), SO₂, N(R′), C(O), N(R′)C(O)O, OC(O)N(R′), CH(Z′), phosphorous containing linkage, optionally substituted aryl, optionally substituted heteroaryl, optionally substituted heterocyclic or optionally substituted cycloalkyl, where R′ is hydrogen, acyl or optionally substituted aliphatic, Z′ is selected from the group consisting of OR₁₁, COR₁₁, CO₂R₁₁,

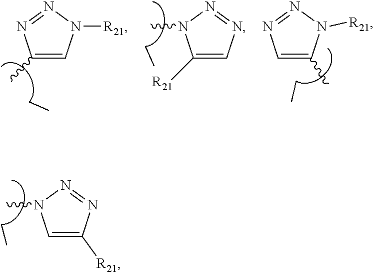

NR₂₁R₃₁, CONR₂₁R₃₁, CON(H)NR₂₁R₃₁, ONR₂₁R₃₁, CON(H)N═CR₄₁R₅₁, N(R₂₁)C(═NR₃₁)NR₂₁R₃₁, N(R₂₁)C(O)NR₂₁R₃₁, N(R₂₁)C(S)NR₂₁R₃₁, OC(O)NR₂₁R₃₁, SC(O)NR₂₁R₃₁, N(R₂₁)C(S)OR₁₁, N(R₂₁)C(O)OR₁₁, N(R₂₁)C(O)SR₁₁, N(R₂₁)N═CR₄₁R₅₁, ON═CR₄₁R₅₁, SO₂R₁₁, SOR₁₁, SR₁₁, and substituted or unsubstituted heterocyclic; R₂₁ and R₃₁ for each occurrence are independently hydrogen, acyl, unsubstituted or substituted aliphatic, aryl, heteroaryl, heterocyclic, OR₁₁, COR₁₁, CO₂R₁₁, or NR₁₁R₁₁′; or R₂₁ and R₃₁, taken together with the atoms to which they are attached, form a heterocyclic ring; R₄₁ and R₅₁ for each occurrence are independently hydrogen, acyl, unsubstituted or substituted aliphatic, aryl, heteroaryl, heterocyclic, OR₁₁, COR₁₁, or CO₂R₁₁, or NR₁₁R₁₁′; and R₁₁ and R₁₁′ are independently hydrogen, aliphatic, substituted aliphatic, aryl, heteroaryl, or heterocyclic. In some embodiments, the hydrogen attached to the C4′ of the 5′ terminal nucleotide is replaced.

In some embodiments, C4′ and C5′ together form an optionally substituted heterocyclic, preferably comprising at least one —PX(Y)—, wherein X is H, OH, OM, SH, optionally substituted alkyl, optionally substituted alkoxy, optionally substituted alkylthio, optionally substituted alkylamino or optionally substituted dialkylamino, where M is independently for each occurrence an alki metal or transition metal with an overall charge of +1; and Y is O, S, or NR′, where R′ is hydrogen, optionally substituted aliphatic. Preferably this modification is at the 5 terminal of the iRNA.

In certain embodiments, LNA's include bicyclic nucleoside having the formula:

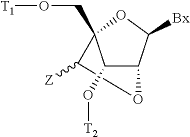

wherein:
Bx is a heterocyclic base moiety;
T₁ is H or a hydroxyl protecting group;
T₂ is H, a hydroxyl protecting group or a reactive phosphorus group;
Z is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, or substituted amide.

In some embodiments, each of the substituted groups, is, independently, mono or poly substituted with optionally protected substituent groups independently selected from halogen, oxo, hydroxyl, OJ1, NJ1J2, SJ1, N3, OC(═X)J1, OC(═X)NJ1J2, NJ3C(═X)NJ1J2 and CN, wherein each J1, J2 and J3 is, independently, H or C₁-C₆ alkyl, and X is 0, S or NJ1.

In certain such embodiments, each of the substituted groups, is, independently, mono or poly substituted with substituent groups independently selected from halogen, oxo, hydroxyl, OJ1, NJ1J2, SJ1, N3, OC(═X)J1, and NJ3C(═X)NJ1J2, wherein each J1, J2 and J3 is, independently, H, C₁-C₆ alkyl, or substituted C₁-C₆ alkyl and X is 0 or NJ1.

In certain embodiments, the Z group is C₁-C₆ alkyl substituted with one or more Xx, wherein each Xx is independently OJ1, NJ1J2, SJ1, N3, OC(═X)J1, OC(═X)NJ1J2, NJ3C(═X)NJ1J2 or CN; wherein each J1, J2 and J3 is, independently, H or C₁-C₆ alkyl, and X is 0, S or NJ1. In another embodiment, the Z group is C₁-C₆ alkyl substituted with one or more Xx, wherein each Xx is independently halo (e.g., fluoro), hydroxyl, alkoxy (e.g., CH₃O—), substituted alkoxy or azido.

In certain embodiments, the Z group is CH₂Xx, wherein Xx is OJ1, NJ1J2, SJ1, N3, OC(═X)J1, OC(═X)NJ1J2, NJ3C(═X)NJ1J2 or CN; wherein each J1, J2 and J3 is, independently, H or C₁-C₆ alkyl, and X is 0, S or NJ1. In another embodiment, the Z group is —CH₂Xx, wherein Xx is halo (e.g., fluoro), hydroxyl, alkoxy (e.g., CH₃O—) or azido.

In certain such embodiments, the Z group is in the (R)-configuration:

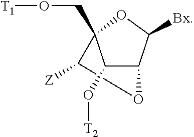

In certain such embodiments, the Z group is in the (S)-configuration:

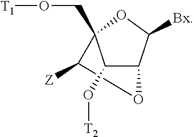

In certain embodiments, each T₁ and T₂ is a hydroxyl protecting group. A preferred list of hydroxyl protecting groups includes benzyl, benzoyl, 2,6-dichlorobenzyl, t-butyldimethylsilyl, t-butyldiphenylsilyl, mesylate, tosylate, dimethoxytrityl (DMT), 9-phenylxanthine-9-yl (Pixyl) and 9-(p-methoxyphenyl)xanthine-9-yl (MOX). In certain embodiments, T₁ is a hydroxyl protecting group selected from acetyl, benzyl, t-butyldimethylsilyl, t-butyldiphenylsilyl and dimethoxytrityl wherein a more preferred hydroxyl protecting group is T₁ is 4,4′-dimethoxytrityl.

In certain embodiments, T₂ is a reactive phosphorus group wherein preferred reactive phosphorus groups include diisopropylcyanoethoxy phosphoramidite and H-phosphonate. In certain embodiments T₁ is 4,4′-dimethoxytrityl and T₂ is diisopropylcyanoethoxy phosphoramidite.

In certain embodiments, the compounds of the invention comprise at least one monomer of the formula:

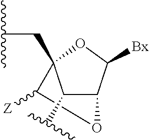

or of the formula:

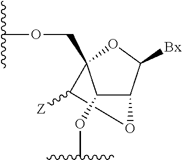

or of the formula:

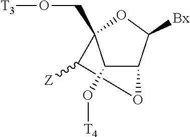

wherein
Bx is a heterocyclic base moiety;
T₃ is H, a hydroxyl protecting group, a linked conjugate group or an internucleoside linking group attached to a nucleoside, a nucleotide, an oligonucleoside, an oligonucleotide, a monomeric subunit or an oligomeric compound;
T₄ is H, a hydroxyl protecting group, a linked conjugate group or an internucleoside linking group attached to a nucleoside, a nucleotide, an oligonucleoside, an oligonucleotide, a monomeric subunit or an oligomeric compound;
wherein at least one of T₃ and T₄ is an internucleoside linking group attached to a nucleoside, a nucleotide, an oligonucleoside, an oligonucleotide, a monomeric subunit or an oligomeric compound; and
Z is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, or substituted amide.

In some embodiments, each of the substituted groups, is, independently, mono or poly substituted with optionally protected substituent groups independently selected from halogen, oxo, hydroxyl, OJ1, NJ1J2, SJ1, N3, OC(═X)J1, OC(═X)NJ1J2, NJ3C(═X)NJ1J2 and CN, wherein each J1, J2 and J3 is, independently, H or C1-C6 alkyl, and X is 0, S or NJ1.

In some embodiments, each of the substituted groups, is, independently, mono or poly substituted with substituent groups independently selected from halogen, oxo, hydroxyl, OJ1, NJ1J2, SJ1, N3, OC(═X)J1, and NJ3C(═X)NJ1J2, wherein each J1, J2 and J3 is, independently, H or C₁-C₆ alkyl, and X is O or NJ1.

In certain such embodiments, at least one Z is C₁-C₆ alkyl or substituted C₁-C₆ alkyl. In certain embodiments, each Z is, independently, C₁-C₆ alkyl or substituted C₁-C₆ alkyl. In certain embodiments, at least one Z is C₁-C₆ alkyl. In certain embodiments, each Z is, independently, C₁-C₆ alkyl. In certain embodiments, at least one Z is methyl. In certain embodiments, each Z is methyl. In certain embodiments, at least one Z is ethyl. In certain embodiments, each Z is ethyl. In certain embodiments, at least one Z is substituted C₁-C₆ alkyl. In certain embodiments, each Z is, independently, substituted C₁-C₆ alkyl. In certain embodiments, at least one Z is substituted methyl. In certain embodiments, each Z is substituted methyl. In certain embodiments, at least one Z is substituted ethyl. In certain embodiments, each Z is substituted ethyl.

In certain embodiments, at least one substituent group is C₁-C₆ alkoxy (e.g., at least one Z is C₁-C₆ alkyl substituted with one or more C₁-C₆ alkoxy). In another embodiment, each substituent group is, independently, C₁-C₆ alkoxy (e.g., each Z is, independently, C₁-C₆ alkyl substituted with one or more C₁-C₆ alkoxy).

In certain embodiments, at least one C₁-C₆ alkoxy substituent group is CH₃O— (e.g., at least one Z is CH₃OCH₂—). In another embodiment, each C₁-C₆ alkoxy substituent group is CH₃O— (e.g., each Z is CH₃OCH₂—).

In certain embodiments, at least one substituent group is halogen (e.g., at least one Z is C₁-C₆ alkyl substituted with one or more halogen). In certain embodiments, each substituent group is, independently, halogen (e.g., each Z is, independently, C₁-C₆ alkyl substituted with one or more halogen). In certain embodiments, at least one halogen substituent group is fluoro (e.g., at least one Z is CH₂FCH₂—, CHF₂CH₂— or CF₃CH₂—). In certain embodiments, each halo substituent group is fluoro (e.g., each Z is, independently, CH₂FCH₂—, CHF₂CH₂— or CF₃CH₂—).

In certain embodiments, at least one substituent group is hydroxyl (e.g., at least one Z is C1-C6 alkyl substituted with one or more hydroxyl). In certain embodiments, each substituent group is, independently, hydroxyl (e.g., each Z is, independently, C1-C₆ alkyl substituted with one or more hydroxyl). In certain embodiments, at least one Z is HOCH₂—. In another embodiment, each Z is HOCH₂—.

In certain embodiments, at least one Z is CH₃—, CH₃CH₂—, CH₂OCH₃—, CH₂F or HOCH₂—. In certain embodiments, each Z is, independently, CH₃—, CH₃CH₂—, CH₂OCH₃—, CH₂F or HOCH₂—.

In certain embodiments, at least one Z group is C₁-C₆ alkyl substituted with one or more Xx, wherein each Xx is, independently, OJ1, NJ1J2, SJ1, N3, OC(═X)J1, OC(═X)NJ1J2, NJ3C(═X)NJ1J2 or CN; wherein each J1, J2 and J3 is, independently, H or C₁-C₆ alkyl, and X is 0, S or NJ1. In another embodiment, at least one Z group is C₁-C₆ alkyl substituted with one or more Xx, wherein each Xx is, independently, halo (e.g., fluoro), hydroxyl, alkoxy (e.g., CH₃O—) or azido.

In certain embodiments, each Z group is, independently, C₁-C₆ alkyl substituted with one or more Xx, wherein each Xx is independently OJ1, NJ1J2, SJ1, N3, OC(═X)J1, OC(═X)NJ1J2, NJ3C(═X)NJ1J2 or CN; wherein each J1, J2 and J3 is, independently, H or C₁-C₆ alkyl, and X is 0, S or NJ1. In another embodiment, each Z group is, independently, C1-C₆ alkyl substituted with one or more Xx, wherein each Xx is independently halo (e.g., fluoro), hydroxyl, alkoxy (e.g., CH₃O—) or azido.

In certain embodiments, at least one Z group is CH₂Xx, wherein Xx is OJ1, NJ1J2, SJ1, N3, OC(═X)J1, OC(═X)NJ1J2, NJ3C(═X)NJ1J2 or CN; wherein each J1, J2 and J3 is, independently, H or C₁-C₆ alkyl, and X is 0, S or NJ1 In certain embodiments, at least one Z group is CH₂Xx, wherein Xx is halo (e.g., fluoro), hydroxyl, alkoxy (e.g., CH₃O—) or azido.

In certain embodiments, each Z group is, independently, CH₂Xx, wherein each Xx is, independently, OJ1, NJ1J2, SJ1, N3, OC(═X)J1, OC(═X)NJ1J2, NJ3C(═X)NJ1J2 or CN; wherein each J1, J2 and J3 is, independently, H or C₁-C₆ alkyl, and X is 0, S or NJ1. In another embodiment, each Z group is, independently, CH₂Xx, wherein each Xx is, independently, halo (e.g., fluoro), hydroxyl, alkoxy (e.g., CH₃O—) or azido.

In certain embodiments, at least one Z is CH₃-. In another embodiment, each Z is, CH₃—.

In certain embodiments, the Z group of at least one monomer is in the (R) configuration represented by the formula:

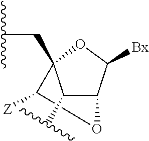

or the formula:

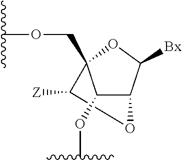

or the formula:

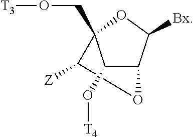

IN certain embodiments, the Z group of each monomer of the formula is in the (R)-configuration.

In certain embodiments, the Z group of at least one monomer is in the (S)-configuration represented by the formula:

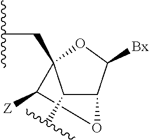

or the formula:

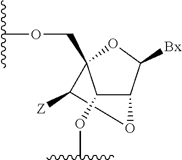

or the formula:

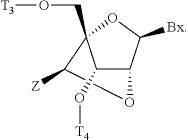

In certain embodiments, the Z group of each monomer of the formula is in the (S)-configuration.

In certain embodiments, T₃ is H or a hydroxyl protecting group. In certain embodiments, T₄ is H or a hydroxyl protecting group. In a further embodiment T₃ is an internucleoside linking group attached to a nucleoside, a nucleotide or a monomeric subunit. In certain embodiments, T₄ is an internucleoside linking group attached to a nucleoside, a nucleotide or a monomeric subunit. In certain embodiments, T₃ is an internucleoside linking group attached to an oligonucleoside or an oligonucleotide. In certain embodiments, T₄ is an internucleoside linking group attached to an oligonucleoside or an oligonucleotide. In certain embodiments, T₃ is an internucleoside linking group attached to an oligomeric compound. In certain embodiments, T₄ is an internucleoside linking group attached to an oligomeric compound. In certain embodiments, at least one of T₃ and T₄ comprises an internucleoside linking group selected from phosphodiester or phosphorothioate.

In certain embodiments, double-stranded iRNA agent of the invention comprise at least one region of at least two contiguous monomers of the formula:

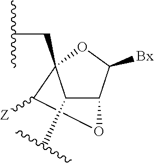

or of the formula:

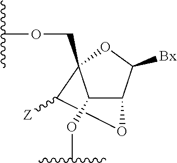

or of the formula:

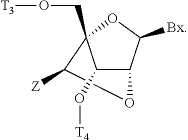

In certain such embodiments, LNAs include, but are not limited to, (A) α-L-Methyleneoxy (4′-CH₂—O-2′) LNA, (B) β-D-Methyleneoxy (4′-CH₂—O-2′) LNA, (C) Ethyleneoxy (4′-(CH₂)₂-O-2′) LNA, (D) Aminooxy (4′-CH₂—O—N(R)-2′) LNA and (E) Oxyamino (4′-CH₂—N(R)-O-2′) LNA, as depicted below:

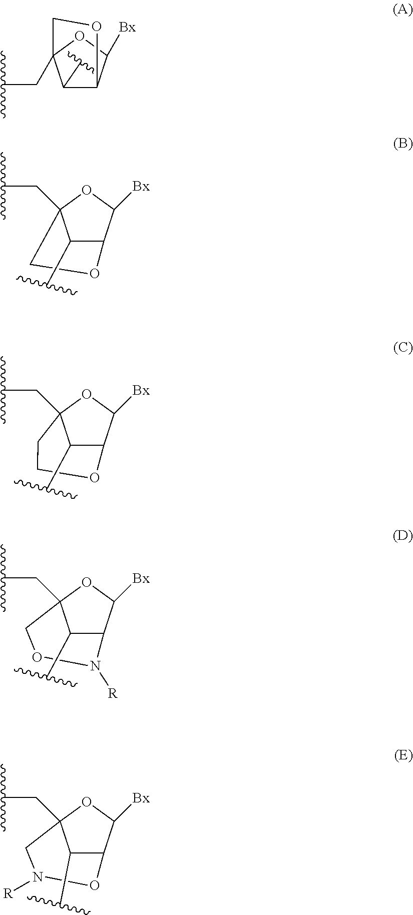

In certain embodiments, the double-stranded iRNA agent of the invention comprises at least two regions of at least two contiguous monomers of the above formula. In certain embodiments, the double-stranded iRNA agent of the invention comprises a gapped motif In certain embodiments, the double-stranded iRNA agent of the invention comprises at least one region of from about 8 to about 14 contiguous P-D-2′-deoxyribofuranosyl nucleosides. In certain embodiments, the Double-stranded iRNA agent of the invention comprises at least one region of from about 9 to about 12 contiguous β-D-2′-deoxyribofuranosyl nucleosides.

In certain embodiments, the double-stranded iRNA agent of the invention comprises at least one (e.g., 1, 2, 3, 4, 5, 6, 7, 8, 9, 10, 11, 12, 13, 14, 15 or more) comprises at least one (S)-cEt monomer of the formula:

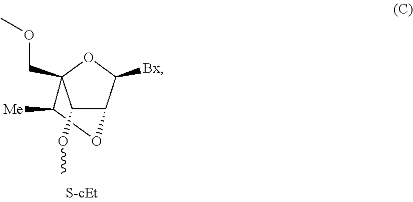

wherein Bx is heterocyclic base moiety.

In certain embodiments, monomers include sugar mimetics. In certain such embodiments, a mimetic is used in place of the sugar or sugar-internucleoside linkage combination, and the nucleobase is maintained for hybridization to a selected target. Representative examples of a sugar mimetics include, but are not limited to, cyclohexenyl or morpholino. Representative examples of a mimetic for a sugar-internucleoside linkage combination include, but are not limited to, peptide nucleic acids (PNA) and morpholino groups linked by uncharged achiral linkages. In some instances a mimetic is used in place of the nucleobase. Representative nucleobase mimetics are well known in the art and include, but are not limited to, tricyclic phenoxazine analogs and universal bases (Berger et al., Nuc Acid Res. 2000, 28:2911-14, incorporated herein by reference). Methods of synthesis of sugar, nucleoside and nucleobase mimetics are well known to those skilled in the art.

### Nucleic Acid Modifications (Intersugar Linkage)

Described herein are linking groups that link monomers (including, but not limited to, modified and unmodified nucleosides and nucleotides) together, thereby forming an oligomeric compound, e.g., an oligonucleotide. Such linking groups are also referred to as intersugar linkage. The two main classes of linking groups are defined by the presence or absence of a phosphorus atom. Representative phosphorus containing linkages include, but are not limited to, phosphodiesters (P═O), phosphotriesters, methylphosphonates, phosphoramidate, and phosphorothioates (P═S). Representative non-phosphorus containing linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Modified linkages, compared to natural phosphodiester linkages, can be used to alter, typically increase, nuclease resistance of the oligonucleotides. In certain embodiments, linkages having a chiral atom can be prepared as racemic mixtures, as separate enantomers. Representative chiral linkages include, but are not limited to, alkylphosphonates and phosphorothioates. Methods of preparation of phosphorous-containing and non-phosphorous-containing linkages are well known to those skilled in the art.

The phosphate group in the linking group can be modified by replacing one of the oxygens with a different substituent. One result of this modification can be increased resistance of the oligonucleotide to nucleolytic breakdown. Examples of modified phosphate groups include phosphorothioate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the linkage can be replaced by any of the following: S, Se, BR₃ (R is hydrogen, alkyl, aryl), C (i.e. an alkyl group, an aryl group, etc. . . . ), H, NR₂ (R is hydrogen, optionally substituted alkyl, aryl), or OR (R is optionally substituted alkyl or aryl). The phosphorous atom in an unmodified phosphate group is achiral. However, replacement of one of the non-bridging oxygens with one of the above atoms or groups of atoms renders the phosphorous atom chiral; in other words a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligonucleotides diastereomers. Thus, while not wishing to be bound by theory, modifications to both non-bridging oxygens, which eliminate the chiral center, e.g. phosphorodithioate formation, can be desirable in that they cannot produce diastereomer mixtures. Thus, the non-bridging oxygens can be independently any one of O, S, Se, B, C, H, N, or OR (R is alkyl or aryl).

The phosphate linker can also be modified by replacement of bridging oxygen, (i.e. oxygen that links the phosphate to the sugar of the monomer), with nitrogen (bridged phosphoroamidates), sulfur (bridged phosphorothioates) and carbon (bridged methylenephosphonates). The replacement can occur at the either one of the linking oxygens or at both linking oxygens. When the bridging oxygen is the 3′-oxygen of a nucleoside, replacement with carbon is preferred. When the bridging oxygen is the 5′-oxygen of a nucleoside, replacement with nitrogen is preferred.

Modified phosphate linkages where at least one of the oxygen linked to the phosphate has been replaced or the phosphate group has been replaced by a non-phosphorous group, are also referred to as “non-phosphodiester intersugar linkage” or “non-phosphodiester linker.”

In certain embodiments, the phosphate group can be replaced by non-phosphorus containing connectors, e.g. dephospho linkers. Dephospho linkers are also referred to as non-phosphodiester linkers herein. While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include, but are not limited to, amides (for example amide-3 (3′-CH₂—C(═O)—N(H)-5′) and amide-4 (3′-CH₂—N(H)-C(═O)-5′)), hydroxylamino, siloxane (dialkylsiloxxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methykenecarbonylamino, methylenemethylimino (MMI, 3′-CH₂—N(CH₃)—O-5′), methylenehydrazo, methylenedimethylhydrazo, methyleneoxymethylimino, ethers (C3′-O-C5′), thioethers (C3′-S-C5′), thioacetamido (C3′-N(H)-C(═O)—CH₂—S-C5′, C3′-O—P(O)—O—SS-C5′, C3′-CH₂—NH—NH-C5′, 3′-NHP(O)(OCH₃)—O-5′ and 3′-NHP(O)(OCH₃)—O-5′ and nonionic linkages containing mixed N, O, S and CH₂ component parts. See for example, Carbohydrate Modifications in Antisense Research; Y. S. Sanghvi and P. D. Cook Eds. ACS Symposium Series 580; Chapters 3 and 4, (pp. 40-65). Preferred embodiments include methylenemethylimino (MMI),methylenecarbonylamino, amides, carbamate and ethylene oxide linker.

One skilled in the art is well aware that in certain instances replacement of a non-bridging oxygen can lead to enhanced cleavage of the intersugar linkage by the neighboring 2′-OH, thus in many instances, a modification of a non-bridging oxygen can necessitate modification of 2′-OH, e.g., a modification that does not participate in cleavage of the neighboring intersugar linkage, e.g., arabinose sugar, 2′-O-alkyl, 2′-F, LNA and ENA.

Preferred non-phosphodiester intersugar linkages include phosphorothioates, phosphorothioates with an at least 1%, 5%, 10%, 20%, 30%, 40%, 50%, 60%, 70%, 80%, 90% 95% or more enantiomeric excess of Sp isomer, phosphorothioates with an at least 1%, 5%, 10%, 20%, 30%, 40%, 50%, 60%, 70%, 80%, 90% 95% or more enantiomeric excess of Rp isomer, phosphorodithioates, phsophotriesters, aminoalkylphosphotrioesters, alkyl-phosphonaters (e.g., methyl-phosphonate), selenophosphates, phosphoramidates (e.g., N-alkylphosphoramidate), and boranophosphonates.

In some embodiments, the double-stranded iRNA agent of the invention comprises at least one (e.g., 1, 2, 3, 4, 5, 6, 7, 8, 9, 10, 11, 12, 13, 14, 15 or more and upto including all) modified or nonphosphodiester linkages. In some embodiments, the double-stranded iRNA agent of the invention comprises at least one (e.g., 1, 2, 3, 4, 5, 6, 7, 8, 9, 10, 11, 12, 13, 14, 15 or more and upto including all) phosphorothioate linkages.

The double-stranded iRNA agent of the inventions can also be constructed wherein the phosphate linker and the sugar are replaced by nuclease resistant nucleoside or nucleotide surrogates. While not wishing to be bound by theory, it is believed that the absence of a repetitively charged backbone diminishes binding to proteins that recognize polyanions (e.g. nucleases). Again, while not wishing to be bound by theory, it can be desirable in some embodiment, to introduce alterations in which the bases are tethered by a neutral surrogate backbone. Examples include the morpholino, cyclobutyl, pyrrolidine, peptide nucleic acid (PNA), aminoethylglycyl PNA (aegPNA) and backnone-extended pyrrolidine PNA (bepPNA) nucleoside surrogates. A preferred surrogate is a PNA surrogate.

The double-stranded iRNA agent of the inventions described herein can contain one or more asymmetric centers and thus give rise to enantiomers, diastereomers, and other stereoisomeric configurations that may be defined, in terms of absolute stereochemistry, as (R) or (S), such as for sugar anomers, or as (D) or (L) such as for amino acids et al. Included in the double-stranded iRNA agent of the inventions provided herein are all such possible isomers, as well as their racemic and optically pure forms.

### Nucleic Acid Modifications (Terminal Modifications)

In some embodiments, the double-stranded iRNA agent further comprises a phosphate or phosphate mimic at the 5′-end of the antisense strand. In one embodiment, the phosphate mimic is a 5′-vinyl phosphonate (VP).

In some embodiments, the 5′-end of the antisense strand of the double-stranded iRNA agent does not contain a 5′-vinyl phosphonate (VP).

Ends of the iRNA agent of the invention can be modified. Such modifications can be at one end or both ends. For example, the 3′ and/or 5′ ends of an iRNA can be conjugated to other functional molecular entities such as labeling moieties, e.g., fluorophores (e.g., pyrene, TAMRA, fluorescein, Cy3 or Cy5 dyes) or protecting groups (based e.g., on sulfur, silicon, boron or ester). The functional molecular entities can be attached to the sugar through a phosphate group and/or a linker. The terminal atom of the linker can connect to or replace the linking atom of the phosphate group or the C-3′ or C-5′ O, N, S or C group of the sugar. Alternatively, the linker can connect to or replace the terminal atom of a nucleotide surrogate (e.g., PNAs).

When a linker/phosphate-functional molecular entity-linker/phosphate array is interposed between two strands of a double stranded oligomeric compound, this array can substitute for a hairpin loop in a hairpin-type oligomeric compound.

Terminal modifications useful for modulating activity include modification of the 5′ end of iRNAs with phosphate or phosphate analogs. In certain embodiments, the 5′end of an iRNA is phosphorylated or includes a phosphoryl analog. Exemplary 5′-phosphate modifications include those which are compatible with RISC mediated gene silencing. Modifications at the 5′-terminal end can also be useful in stimulating or inhibiting the immune system of a subject. In some embodiments, the 5′-end of the oligomeric compound comprises the modification

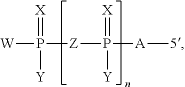

wherein W, X and Y are each independently selected from the group consisting of O, OR (R is hydrogen, alkyl, aryl), S, Se, BR₃ (R is hydrogen, alkyl, aryl), BH₃⁻, C (i.e. an alkyl group, an aryl group, etc. . . . ), H, NR₂ (R is hydrogen, alkyl, aryl), or OR (R is hydrogen, alkyl or aryl); A and Z are each independently for each occurrence absent, O, S, CH₂, NR (R is hydrogen, alkyl, aryl), or optionally substituted alkylene, wherein backbone of the alkylene can comprise one or more of O, S, SS and NR (R is hydrogen, alkyl, aryl) internally and/or at the end; and n is 0-2. In some embodiments, n is 1 or 2. It is understood that A is replacing the oxygen linked to 5′ carbon of sugar. When n is 0, W and Y together with the P to which they are attached can form an optionally substituted 5-8 membered heterocyclic, wherein W an Y are each independently O, S, NR′ or alkylene. Preferably the heterocyclic is substituted with an aryl or heteroaryl. In some embodiments, one or both hydrogen on C5′ of the 5′-terminal nucleotides are replaced with a halogen, e.g., F.

Exemplary 5′-modifications include, but are not limited to, 5′-monophosphate ((HO)₂(O)P-O-5′); 5′-diphosphate ((HO)₂(O)P—O—P(HO)(O)-O-5′); 5′-triphosphate ((HO)₂(O)P-O—(HO)(O)P—O—P(HO)(O)-O-5′); 5′-monothiophosphate (phosphorothioate; (HO)2(S)P-O-5′); 5′-monodithiophosphate (phosphorodithioate; (HO)(HS)(S)P-O-5′), 5′-phosphorothiolate ((HO)2(O)P-S-5′); 5′-alpha-thiotriphosphate; 5′-beta-thiotriphosphate; 5′-gamma-thiotriphosphate; 5′-phosphoramidates ((HO)₂(O)P-NH—5′, (HO)(NH₂)(O)P-O-5′). Other 5′-modification include 5′-alkylphosphonates (R(OH)(O)P-O-5′, R═alkyl, e.g., methyl, ethyl, isopropyl, propyl, etc. . . . ), 5′-alkyletherphosphonates (R(OH)(O)P-O-5′, R═alkylether, e.g., methoxymethyl (CH₂OMe), ethoxymethyl, etc. . . . ). Other exemplary 5′-modifications include where Z is optionally substituted alkyl at least once, e.g., ((HO)₂(X)P—O[—(CH₂)ₐ-O—P(X)(OH)-O]_{b}-5′, ((HO)₂(X)P—O[—(CH₂)ₐ—P(X)(OH)—O]_{b}-5′, ((HO)₂(X)P—[—(CH₂)ₐ—O—P(X)(OH)—O]_{b}-5′; dialkyl terminal phosphates and phosphate mimics: HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)-O]_{b}-5′, wherein a and b are each independently 1-10. Other embodiments, include replacement of oxygen and/or sulfur with BH₃, BH₃⁻ and/or Se.

Terminal modifications can also be useful for monitoring distribution, and in such cases the preferred groups to be added include fluorophores, e.g., fluorescein or an Alexa dye, e.g., Alexa 488. Terminal modifications can also be useful for enhancing uptake, useful modifications for this include targeting ligands. Terminal modifications can also be useful for cross-linking an oligonucleotide to another moiety; modifications useful for this include mitomycin C, psoralen, and derivatives thereof.

### Thermally Destabilizing Modifications

The compounds of the invention, such as iRNAs or dsRNA agents, can be optimized for RNA interference by increasing the propensity of the iRNA duplex to disassociate or melt (decreasing the free energy of duplex association) by introducing a thermally destabilizing modification in the sense strand at a site opposite to the seed region of the antisense strand (i.e., at positions 2-8 of the 5′-end of the antisense strand). This modification can increase the propensity of the duplex to disassociate or melt in the seed region of the antisense strand.

The thermally destabilizing modifications can include abasic modification; mismatch with the opposing nucleotide in the opposing strand; and sugar modification such as 2′-deoxy modification or acyclic nucleotide, e.g., unlocked nucleic acids (UNA) or glycerol nucleic acid (GNA).

Exemplified Abasic Modifications are:

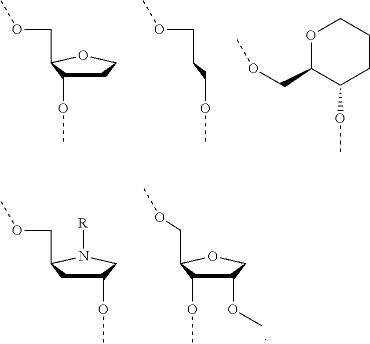

Exemplified Sugar Modifications are:

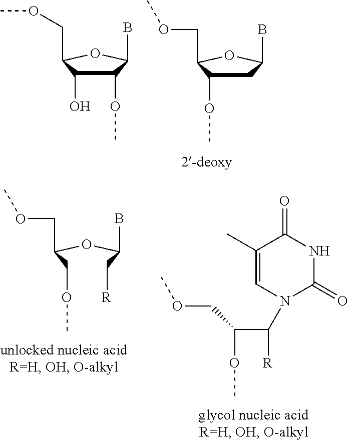

The term “acyclic nucleotide” refers to any nucleotide having an acyclic ribose sugar, for example, where any of bonds between the ribose carbons (e.g., C1′-C2′, C2′-C3′, C3′-C4′, C4′-O4′, or C1′-O4′) is absent and/or at least one of ribose carbons or oxygen (e.g., C1′, C2′, C3′, C4′ or O4′) are independently or in combination absent from the nucleotide. In some embodiments, acyclic nucleotide is

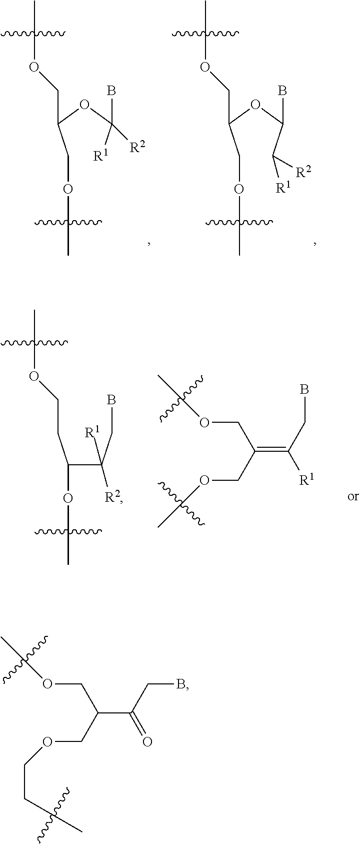

wherein B is a modified or unmodified nucleobase, R¹ and R² independently are H, halogen, OR₃, or alkyl; and R₃ is H, alkyl, cycloalkyl, aryl, aralkyl, heteroaryl or sugar). The term “UNA” refers to unlocked acyclic nucleic acid, wherein any of the bonds of the sugar has been removed, forming an unlocked “sugar” residue. In one example, UNA also encompasses monomers with bonds between C1′-C4′ being removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar is removed (see Mikhailov et. al., Tetrahedron Letters, 26 (17): 2059 (1985); and Fluiter et al., Mol. Biosyst., 10: 1039 (2009), which are hereby incorporated by reference in their entirety). The acyclic derivative provides greater backbone flexibility without affecting the Watson-Crick pairings. The acyclic nucleotide can be linked via 2′-5′ or 3′-5′ linkage.

The term ‘GNA’ refers to glycol nucleic acid which is a polymer similar to DNA or RNA but differing in the composition of its “backbone” in that is composed of repeating glycerol units linked by phosphodiester bonds:

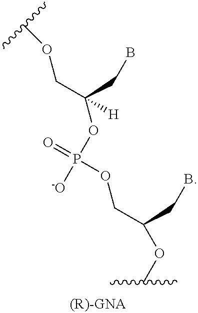

The thermally destabilizing modification can be mismatches (i.e., noncomplementary base pairs) between the thermally destabilizing nucleotide and the opposing nucleotide in the opposite strand within the dsRNA duplex. Exemplary mismatch basepairs include G:G, G:A, G:U, G:T, A:A, A:C, C:C, C:U, C:T, U:U, T:T, U:T, or a combination thereof. Other mismatch base pairings known in the art are also amenable to the present invention. A mismatch can occur between nucleotides that are either naturally occurring nucleotides or modified nucleotides, i.e., the mismatch base pairing can occur between the nucleobases from respective nucleotides independent of the modifications on the ribose sugars of the nucleotides. In certain embodiments, the compounds of the invention, such as siRNA or iRNA agent, contains at least one nucleobase in the mismatch pairing that is a 2′-deoxy nucleobase; e.g., the 2′-deoxy nucleobase is in the sense strand.

More examples of abasic nucleotide, acyclic nucleotide modifications (including UNA and GNA), and mismatch modifications have been described in detail in WO 2011/133876, which is herein incorporated by reference in its entirety.

The thermally destabilizing modifications may also include universal base with reduced or abolished capability to form hydrogen bonds with the opposing bases, and phosphate modifications.

Nucleobase modifications with impaired or completely abolished capability to form hydrogen bonds with bases in the opposite strand have been evaluated for destabilization of the central region of the dsRNA duplex as described in WO 2010/0011895, which is herein incorporated by reference in its entirety. Exemplary nucleobase modifications are:

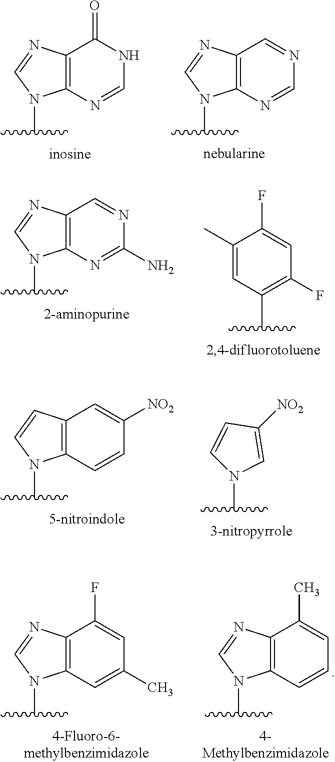

Exemplary phosphate modifications known to decrease the thermal stability of dsRNA duplexes compared to natural phosphodiester linkages are:

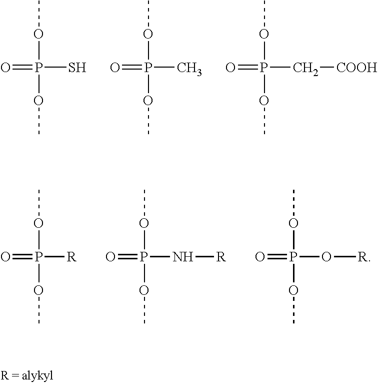

In some embodiments, compounds of the invention can comprise 2′-5′ linkages (with 2′-H, 2′-OH and 2′-OMe and with P═O or P═S). For example, the 2′-5′ linkages modifications can be used to promote nuclease resistance or to inhibit binding of the sense to the antisense strand, or can be used at the 5′ end of the sense strand to avoid sense strand activation by RISC.

In another embodiment, compounds of the invention can comprise L sugars (e.g., L ribose, L-arabinose with 2′-H, 2′-OH and 2′-OMe). For example, these L sugar modifications can be used to promote nuclease resistance or to inhibit binding of the sense to the antisense strand, or can be used at the 5′ end of the sense strand to avoid sense strand activation by RISC.

In one embodiment the iRNA agent of the invention is conjugated to a ligand via a carrier, wherein the carrier can be cyclic group or acyclic group; preferably, the cyclic group is selected from cyclohexyl, pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl and and decalin; preferably, the acyclic group is selected from serinol backbone or diethanolamine backbone.

In some embodiments, at least one strand of the iRNA agent of the invention disclosed herein is 5′ phosphorylated or includes a phosphoryl analog at the 5′ prime terminus. 5′-phosphate modifications include those which are compatible with RISC mediated gene silencing. Suitable modifications include: 5′-monophosphate ((HO)₂(O)P-O-5′); 5′-diphosphate ((HO)₂(O)P—O—P(HO)(O)-O-5′); 5′-triphosphate ((HO)₂(O)P-O—(HO)(O)P—O—P(HO)(O)-O-5′); 5′-guanosine cap (7-methylated or non-methylated) (7m-G-O-5′-(HO)(O)P-O—(HO)(O)P—O—P(HO)(O)-O-5′); 5′-adenosine cap (Appp), and any modified or unmodified nucleotide cap structure (N—O-5′-(HO)(O)P-O—(HO)(O)P—O—P(HO)(O)-O-5′); 5′-monothiophosphate (phosphorothioate; (HO)₂(S)P-O-5′); 5′-monodithiophosphate (phosphorodithioate; (HO)(HS)(S)P-O-5′), 5′-phosphorothiolate ((HO)₂(O)P-S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)₂(O)P-NH—5′, (HO)(NH2)(O)P-O-5′), 5′-alkylphosphonates (R=alkyl=methyl, ethyl, isopropyl, propyl, etc., e.g. RP(OH)(O)-O-5′-, 5′-alkenylphosphonates (i.e. vinyl, substituted vinyl), (OH)₂(O)P-5′-CH₂—), 5′-alkyletherphosphonates (R=alkylether=methoxymethyl (MeOCH2-), ethoxymethyl, etc., e.g. RP(OH)(O)-O-5′-).

### Target Genes

Without limitations, target genes for the double-stranded iRNAs include, but are not limited to genes promoting unwanted cell proliferation, growth factor gene, growth factor receptor gene, genes expressing kinases, an adaptor protein gene, a gene encoding a G protein super family molecule, a gene encoding a transcription factor, a gene which mediates angiogenesis, a viral gene, a gene required for viral replication, a cellular gene which mediates viral function, a gene of a bacterial pathogen, a gene of an amoebic pathogen, a gene of a parasitic pathogen, a gene of a fungal pathogen, a gene which mediates an unwanted immune response, a gene which mediates the processing of pain, a gene which mediates a neurological disease, an allene gene found in cells characterized by loss of heterozygosity, or one allege gene of a polymorphic gene.

Specific exemplary target genes for the double-stranded iRNAs include, but are not limited to, PCSK-9, ApoC3, AT3, AGT, ALAS1, TMPR, HAO1, AGT, C5, CCR-5, PDGF beta gene; Erb-B gene, Src gene; CRK gene; GRB2 gene; RAS gene; MEKK gene; JNK gene; RAF gene; Erk½ gene; PCNA(p21) gene; MYB gene; c-MYC gene; JUN gene; FOS gene; BCL-2 gene; Cyclin D gene; VEGF gene; EGFR gene; Cyclin A gene; Cyclin E gene; WNT-1 gene; beta-catenin gene; c-MET gene; PKC gene; NFKB gene; STAT3 gene; survivin gene; Her2/Neu gene; topoisomerase I gene; topoisomerase II alpha gene; p73 gene; p21(WAF1/CIP1) gene, p27(KIP1) gene; PPM1D gene; caveolin I gene; MIB I gene; MTAI gene; M68 gene; tumor suppressor genes; p53 gene; DN-p63 gene; pRb tumor suppressor gene; APC1 tumor suppressor gene; BRCA1 tumor suppressor gene; PTEN tumor suppressor gene; MLL fusion genes, e.g., MLL-AF9, BCR/ABL fusion gene; TEL/AML1 fusion gene; EWS/FLIl fusion gene; TLS/FUS1 fusion gene; PAX3/FKHR fusion gene; AML1/ETO fusion gene; alpha v-integrin gene; Flt-1 receptor gene; tubulin gene; Human Papilloma Virus gene, a gene required for Human Papilloma Virus replication, Human Immunodeficiency Virus gene, a gene required for Human Immunodeficiency Virus replication, Hepatitis A Virus gene, a gene required for Hepatitis A Virus replication, Hepatitis B Virus gene, a gene required for Hepatitis B Virus replication, Hepatitis C Virus gene, a gene required for Hepatitis C Virus replication, Hepatitis D Virus gene, a gene required for Hepatitis D Virus replication, Hepatitis E Virus gene, a gene required for Hepatitis E Virus replication, Hepatitis F Virus gene, a gene required for Hepatitis F Virus replication, Hepatitis G Virus gene, a gene required for Hepatitis G Virus replication, Hepatitis H Virus gene, a gene required for Hepatitis H Virus replication, Respiratory Syncytial Virus gene, a gene that is required for Respiratory Syncytial Virus replication, Herpes Simplex Virus gene, a gene that is required for Herpes Simplex Virus replication, herpes Cytomegalovirus gene, a gene that is required for herpes Cytomegalovirus replication, herpes Epstein Barr Virus gene, a gene that is required for herpes Epstein Barr Virus replication, Kaposi's Sarcoma-associated Herpes Virus gene, a gene that is required for Kaposi's Sarcoma-associated Herpes Virus replication, JC Virus gene, human gene that is required for JC Virus replication, myxovirus gene, a gene that is required for myxovirus gene replication, rhinovirus gene, a gene that is required for rhinovirus replication, coronavirus gene, a gene that is required for coronavirus replication, West Nile Virus gene, a gene that is required for West Nile Virus replication, St. Louis Encephalitis gene, a gene that is required for St. Louis Encephalitis replication, Tick-borne encephalitis virus gene, a gene that is required for Tick-borne encephalitis virus replication, Murray Valley encephalitis virus gene, a gene that is required for Murray Valley encephalitis virus replication, dengue virus gene, a gene that is required for dengue virus gene replication, Simian Virus 40 gene, a gene that is required for Simian Virus 40 replication, Human T Cell Lymphotropic Virus gene, a gene that is required for Human T Cell Lymphotropic Virus replication, Moloney-Murine Leukemia Virus gene, a gene that is required for Moloney-Murine Leukemia Virus replication, encephalomyocarditis virus gene, a gene that is required for encephalomyocarditis virus replication, measles virus gene, a gene that is required for measles virus replication, Vericella zoster virus gene, a gene that is required for Vericella zoster virus replication, adenovirus gene, a gene that is required for adenovirus replication, yellow fever virus gene, a gene that is required for yellow fever virus replication, poliovirus gene, a gene that is required for poliovirus replication, poxvirus gene, a gene that is required for poxvirus replication, plasmodium gene, a gene that is required for plasmodium gene replication, Mycobacterium ulcerans gene, a gene that is required for Mycobacterium ulcerans replication, Mycobacterium tuberculosis gene, a gene that is required for Mycobacterium tuberculosis replication, Mycobacterium leprae gene, a gene that is required for Mycobacterium leprae replication, Staphylococcus aureus gene, a gene that is required for Staphylococcus aureus replication, Streptococcus pneumoniae gene, a gene that is required for Streptococcus pneumoniae replication, Streptococcus pyogenes gene, a gene that is required for Streptococcus pyogenes replication, Chlamydia pneumoniae gene, a gene that is required for Chlamydia pneumoniae replication, Mycoplasma pneumoniae gene, a gene that is required for Mycoplasma pneumoniae replication, an integrin gene, a selectin gene, complement system gene, chemokine gene, chemokine receptor gene, GCSF gene, Gro1 gene, Gro2 gene, Gro3 gene, PF4 gene, MIG gene, Pro-Platelet Basic Protein gene, MIP-1I gene, MIP-1J gene, RANTES gene, MCP-1 gene, MCP-2 gene, MCP-3 gene, CMBKR1 gene, CMBKR2 gene, CMBKR3 gene, CMBKR5v, AIF-1 gene, I-309 gene, a gene to a component of an ion channel, a gene to a neurotransmitter receptor, a gene to a neurotransmitter ligand, amyloid-family gene, presenilin gene, HD gene, DRPLA gene, SCA1 gene, SCA2 gene, MJD1 gene, CACNL1A4 gene, SCA7 gene, SCA8 gene, allele gene found in loss of heterozygosity (LOH) cells, one allele gene of a polymorphic gene and combinations thereof.

The loss of heterozygosity (LOH) can result in hemizygosity for sequence, e.g., genes, in the area of LOH. This can result in a significant genetic difference between normal and disease-state cells, e.g., cancer cells, and provides a useful difference between normal and disease-state cells, e.g., cancer cells. This difference can arise because a gene or other sequence is heterozygous in duploid cells but is hemizygous in cells having LOH. The regions of LOH will often include a gene, the loss of which promotes unwanted proliferation, e.g., a tumor suppressor gene, and other sequences including, e.g., other genes, in some cases a gene which is essential for normal function, e.g., growth. Methods of the invention rely, in part, on the specific modulation of one allele of an essential gene with a composition of the invention.

In certain embodiments, the invention provides a double-stranded iRNA agent of the invention that modulates a micro-RNA.

Evaluation of Candidate iRNAs

One can evaluate a candidate iRNA agent, e.g., a modified RNA, for a selected property by exposing the agent or modified molecule and a control molecule to the appropriate conditions and evaluating for the presence of the selected property. For example, resistance to a degradent can be evaluated as follows. A candidate modified RNA (and a control molecule, usually the unmodified form) can be exposed to degradative conditions, e.g., exposed to a milieu, which includes a degradative agent, e.g., a nuclease. E.g., one can use a biological sample, e.g., one that is similar to a milieu, which might be encountered, in therapeutic use, e.g., blood or a cellular fraction, e.g., a cell-free homogenate or disrupted cells. The candidate and control could then be evaluated for resistance to degradation by any of a number of approaches. For example, the candidate and control could be labeled prior to exposure, with, e.g., a radioactive or enzymatic label, or a fluorescent label, such as Cy3 or Cy5. Control and modified RNA's can be incubated with the degradative agent, and optionally a control, e.g., an inactivated, e.g., heat inactivated, degradative agent. A physical parameter, e.g., size, of the modified and control molecules are then determined. They can be determined by a physical method, e.g., by polyacrylamide gel electrophoresis or a sizing column, to assess whether the molecule has maintained its original length, or assessed functionally. Alternatively, Northern blot analysis can be used to assay the length of an unlabeled modified molecule.

A functional assay can also be used to evaluate the candidate agent. A functional assay can be applied initially or after an earlier non-functional assay, (e.g., assay for resistance to degradation) to determine if the modification alters the ability of the molecule to silence gene expression. For example, a cell, e.g., a mammalian cell, such as a mouse or human cell, can be co-transfected with a plasmid expressing a fluorescent protein, e.g., GFP, and a candidate RNA agent homologous to the transcript encoding the fluorescent protein (see, e.g., WO 00/44914). For example, a modified dsiRNA homologous to the GFP mRNA can be assayed for the ability to inhibit GFP expression by monitoring for a decrease in cell fluorescence, as compared to a control cell, in which the transfection did not include the candidate dsiRNA, e.g., controls with no agent added and/or controls with a non-modified RNA added. Efficacy of the candidate agent on gene expression can be assessed by comparing cell fluorescence in the presence of the modified and unmodified dssiRNA compounds.

In an alternative functional assay, a candidate dssiRNA compound homologous to an endogenous mouse gene, for example, a maternally expressed gene, such as c-mos, can be injected into an immature mouse oocyte to assess the ability of the agent to inhibit gene expression in vivo (see, e.g., WO 01/36646). A phenotype of the oocyte, e.g., the ability to maintain arrest in metaphase II, can be monitored as an indicator that the agent is inhibiting expression. For example, cleavage of c-mos mRNA by a dssiRNA compound would cause the oocyte to exit metaphase arrest and initiate parthenogenetic development (Colledge et al. Nature 370: 65-68, 1994; Hashimoto et al. Nature, 370:68-71, 1994). The effect of the modified agent on target RNA levels can be verified by Northern blot to assay for a decrease in the level of target mRNA, or by Western blot to assay for a decrease in the level of target protein, as compared to a negative control. Controls can include cells in which with no agent is added and/or cells in which a non-modified RNA is added.

### Physiological Effects

The siRNA compounds described herein can be designed such that determining therapeutic toxicity is made easier by the complementarity of the siRNA with both a human and a non-human animal sequence. By these methods, an siRNA can consist of a sequence that is fully complementary to a nucleic acid sequence from a human and a nucleic acid sequence from at least one non-human animal, e.g., a non-human mammal, such as a rodent, ruminant or primate. For example, the non-human mammal can be a mouse, rat, dog, pig, goat, sheep, cow, monkey, Pan paniscus, Pan troglodytes, Macaca mulatto, or Cynomolgus monkey. The sequence of the siRNA compound could be complementary to sequences within homologous genes, e.g., oncogenes or tumor suppressor genes, of the non-human mammal and the human. By determining the toxicity of the siRNA compound in the non-human mammal, one can extrapolate the toxicity of the siRNA compound in a human. For a more strenuous toxicity test, the siRNA can be complementary to a human and more than one, e.g., two or three or more, non-human animals.

The methods described herein can be used to correlate any physiological effect of an siRNA compound on a human, e.g., any unwanted effect, such as a toxic effect, or any positive, or desired effect.

Increasing Cellular Uptake of siRNAs

Described herein are various siRNA compositions that contain covalently attached conjugates that increase cellular uptake and/or intracellular targeting of the siRNAs.

Additionally provided are methods of the invention that include administering an siRNA compound and a drug that affects the uptake of the siRNA into the cell. The drug can be administered before, after, or at the same time that the siRNA compound is administered. The drug can be covalently or non-covalently linked to the siRNA compound. The drug can be, for example, a lipopolysaccharide, an activator of p38 MAP kinase, or an activator of NF-κB. The drug can have a transient effect on the cell. The drug can increase the uptake of the siRNA compound into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin. The drug can also increase the uptake of the siRNA compound into a given cell or tissue by activating an inflammatory response, for example. Exemplary drugs that would have such an effect include tumor necrosis factor alpha (TNFalpha), interleukin-1 beta, a CpG motif, gamma interferon or more generally an agent that activates a toll-like receptor.

siRNA Production

An siRNA can be produced, e.g., in bulk, by a variety of methods. Exemplary methods include: organic synthesis and RNA cleavage, e.g., in vitro cleavage.

Organic Synthesis. An siRNA can be made by separately synthesizing a single stranded RNA molecule, or each respective strand of a double-stranded RNA molecule, after which the component strands can then be annealed.

A large bioreactor, e.g., the OligoPilot II from Pharmacia Biotec AB (Uppsala Sweden), can be used to produce a large amount of a particular RNA strand for a given siRNA. The OligoPilotII reactor can efficiently couple a nucleotide using only a 1.5 molar excess of a phosphoramidite nucleotide. To make an RNA strand, ribonucleotides amidites are used. Standard cycles of monomer addition can be used to synthesize the 21 to 23 nucleotide strand for the siRNA. Typically, the two complementary strands are produced separately and then annealed, e.g., after release from the solid support and deprotection.

Organic synthesis can be used to produce a discrete siRNA species. The complementary of the species to a particular target gene can be precisely specified. For example, the species may be complementary to a region that includes a polymorphism, e.g., a single nucleotide polymorphism. Further the location of the polymorphism can be precisely defined. In some embodiments, the polymorphism is located in an internal region, e.g., at least 4, 5, 7, or 9 nucleotides from one or both of the termini.

dsiRNA Cleavage. siRNAs can also be made by cleaving a larger siRNA. The cleavage can be mediated in vitro or in vivo. For example, to produce iRNAs by cleavage in vitro, the following method can be used:

In vitro transcription. dsiRNA is produced by transcribing a nucleic acid (DNA) segment in both directions. For example, the HiScribe™ RNAi transcription kit (New England Biolabs) provides a vector and a method for producing a dsiRNA for a nucleic acid segment that is cloned into the vector at a position flanked on either side by a T7 promoter. Separate templates are generated for T7 transcription of the two complementary strands for the dsiRNA. The templates are transcribed in vitro by addition of T7 RNA polymerase and dsiRNA is produced. Similar methods using PCR and/or other RNA polymerases (e.g., T3 or SP6 polymerase) can also be dotoxins that may contaminate preparations of the recombinant enzymes.

In Vitro Cleavage. In one embodiment, RNA generated by this method is carefully purified to remove endsiRNA is cleaved in vitro into siRNAs, for example, using a Dicer or comparable RNAse III-based activity. For example, the dsiRNA can be incubated in an in vitro extract from Drosophila or using purified components, e.g., a purified RNAse or RISC complex (RNA-induced silencing complex). See, e.g., Ketting et al. Genes Dev 2001 Oct. 15; 15(20):2654-9. and Hammond Science 2001 Aug. 10; 293(5532):1146-50.

dsiRNA cleavage generally produces a plurality of siRNA species, each being a particular 21 to 23 nt fragment of a source dsiRNA molecule. For example, siRNAs that include sequences complementary to overlapping regions and adjacent regions of a source dsiRNA molecule may be present.

Regardless of the method of synthesis, the siRNA preparation can be prepared in a solution (e.g., an aqueous and/or organic solution) that is appropriate for formulation. For example, the siRNA preparation can be precipitated and redissolved in pure double-distilled water, and lyophilized. The dried siRNA can then be resuspended in a solution appropriate for the intended formulation process.

### Formulation

Exemplary formulations which can be used for administering the double-stranded iRNA agent are discussed below.

### Penetration Enhancer

The formulation includes an siRNA compound, e.g., a double-stranded iRNA compound, or ssiRNA compound (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) admixed with a penetration enhancer.

In some embodiments, the penetration enhancer is selected from the group consisting of a fatty acid or pharmaceutically acceptable salt thereof, a fatty acid derivative or pharmaceutically acceptable salt thereof, a bile acid or pharmaceutically acceptable salt thereof, a chelating agent, a surfactant, a non-chelating non-surfactant agent, and a chitosan or derivative thereof.

In one embodiment, the penetration enhancer is a fatty acid or pharmaceutically acceptable salt thereof, or ester thereof. Suitable fatty acids and their derivatives include C₈-C₂₀ saturated or unsaturated, linear, branched or cyclic compounds. For instance, the fatty acid can be arachidonic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine; or a C₁₋₁₀ alkyl ester, monoglyceride, or diglyceride thereof, or a pharmaceutically acceptable salt thereof (such as a sodium salt).

In another embodiment, the penetration enhancer is a bile acid or pharmaceutical acceptable salt thereof. The bile salt can be cholic acid, dehydrocholic acid, deoxycholic acid, glucholic acid, glycholic acid, glycodeoxycholic acid, taurocholic acid, taurodeoxycholic acid, chenodeoxycholic acid, ursodeoxycholic acid, sodium tauro-24,25-dihydro-fusidate, sodium glycodihydrofusidate, polyoxyethylene-9-lauryl ether, or a pharmaceutically acceptable salt thereof (such as a sodium salt).

In another embodiment, the penetration enhancer is a chelating agent. The chelating agent can be EDTA, EGTA, citric acid, a salicyclate, a N-acyl derivative of collagen, laureth-9, an N-amino acyl derivative of a beta-diketone or a mixture thereof.

In another embodiment, the penetration enhancer is a surfactant, e.g., an ionic or nonionic surfactant. The surfactant can be sodium lauryl sulfate, polyoxyethylene-9-lauryl ether, polyoxyethylene-20-cetyl ether, a perfluorchemical emulsion, or mixture thereof. Other surfactance described, infra, are also suitable penetration enhancers.

In another embodiment, the penetration enhancer can be a non-chelating non-surfactant selected from a group consisting of unsaturated cyclic ureas, 1-alkyl-alkones, 1-alkenylazacyclo-alakanones, steroidal anti-inflammatory agents and mixtures thereof. In yet another embodiment the penetration enhancer can be a glycol, a pyrrol, an azone, or a terpenes.

In one embodiment, the penetration enhancer is chitosan or trimethyl chitosan chloride. In one embodiment, the penetration enhancer is cyclodextrin inclusion complex or saponins.

In one embodiment, the penetration enhancer is caprylic acid (C8), capric acid (C10), lauric acid (C12), oleic acid (C18), or pharmaceutically acceptable salt thereof, such as a sodium salt of the aforementioned fatty acid.

In one embodiment, the penetration enhancer includes one or more compounds and or mixtures, selected from the group consisting of sodium caprate (C10), either alone or in conjunction with sodium caprylate (C12); transcellular N-[8-2-hydroxybenzoyl) aminol caprylate (an acetylated amino acid); C12, sodium caprylate, as an adjunct to C10; UDCA, also used as an adjunct to C10; sodium laurate; bile salts, fatty acids mixture (C10, C12, UDCA); POE; lecithin; C20 (sodium-2-ocyldodecanoate); PEG 3350; Gantrex AN-169; 5% Gantrex AN-169 and 5% carbopol 974P; 5% Gantrex AN-169; 1% Eudragit; cumulase, labrasol; alkyl saccharide; lipids; EDTA; Gantrez with bioadhesives; sodium phosphate tribasic and UDC.

### Liposomes.

For ease of exposition the formulations, compositions and methods in this section are discussed largely with regard to unmodified siRNA compounds. It may be understood, however, that these formulations, compositions and methods can be practiced with other siRNA compounds, e.g., modified siRNAs, and such practice is within the invention. An siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) preparation can be formulated for delivery in a membranous molecular assembly, e.g., a liposome or a micelle. As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the siRNA composition. The lipophilic material isolates the aqueous interior from an aqueous exterior, which typically does not include the siRNA composition, although in some examples, it may. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the siRNA are delivered into the cell where the siRNA can specifically bind to a target RNA and can mediate RNAi. In some cases the liposomes are also specifically targeted, e.g., to direct the siRNA to particular cell types.

A liposome containing a siRNA can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The siRNA preparation is then added to the micelles that include the lipid component. The cationic groups on the lipid interact with the siRNA and condense around the siRNA to form a liposome. After condensation, the detergent is removed, e.g., by dialysis, to yield a liposomal preparation of siRNA.

If necessary a carrier compound that assists in condensation can be added during the condensation reaction, e.g., by controlled addition. For example, the carrier compound can be a polymer other than a nucleic acid (e.g., spermine or spermidine). pH can also be adjusted to favor condensation.

Further description of methods for producing stable polynucleotide delivery vehicles, which incorporate a polynucleotide/cationic lipid complex as structural components of the delivery vehicle, are described in, e.g., WO 96/37194. Liposome formation can also include one or more aspects of exemplary methods described in Felgner, P. L. et al., Proc. Natl. Acad. Sci., USA 8:7413-7417, 1987; U.S. Pat. Nos. 4,897,355; 5,171,678; Bangham, et al. M. Mol. Biol. 23:238, 1965; Olson, et al. Biochim. Biophys. Acta 557:9, 1979; Szoka, et al. Proc. Natl. Acad. Sci. 75: 4194, 1978; Mayhew, et al. Biochim. Biophys. Acta 775:169, 1984; Kim, et al. Biochim. Biophys. Acta 728:339, 1983; and Fukunaga, et al. Endocrinol. 115:757, 1984, which are incorporated by reference in their entirety. Commonly used techniques for preparing lipid aggregates of appropriate size for use as delivery vehicles include sonication and freeze-thaw plus extrusion (see, e.g., Mayer, et al. Biochim. Biophys. Acta 858:161, 1986, which is incorporated by reference in its entirety). Microfluidization can be used when consistently small (50 to 200 nm) and relatively uniform aggregates are desired (Mayhew, et al. Biochim. Biophys. Acta 775:169, 1984, which is incorporated by reference in its entirety). These methods are readily adapted to packaging siRNA preparations into liposomes.

Liposomes that are pH-sensitive or negatively-charged entrap nucleic acid molecules rather than complex with them. Since both the nucleic acid molecules and the lipid are similarly charged, repulsion rather than complex formation occurs. Nevertheless, some nucleic acid molecules are entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 19, (1992) 269-274, which is incorporated by reference in its entirety).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Examples of other methods to introduce liposomes into cells in vitro and include U.S. Pat. Nos. 5,283,185; 5,171,678; WO 94/00569; WO 93/24640; WO 91/16024; Felgner, J. Biol. Chem. 269:2550, 1994; Nabel, Proc. Natl. Acad. Sci. 90:11307, 1993; Nabel, Human Gene Ther. 3:649, 1992; Gershon, Biochem. 32:7143, 1993; and Strauss EMBO J. 11:417, 1992.

In one embodiment, cationic liposomes are used. Cationic liposomes possess the advantage of being able to fuse to the cell membrane. Non-cationic liposomes, although not able to fuse as efficiently with the plasma membrane, are taken up by macrophages in vivo and can be used to deliver siRNAs to macrophages.

Further advantages of liposomes include: liposomes obtained from natural phospholipids are biocompatible and biodegradable; liposomes can incorporate a wide range of water and lipid soluble drugs; liposomes can protect encapsulated siRNAs in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of siRNA (see, e.g., Felgner, P. L. et al., Proc. Natl. Acad. Sci., USA 8:7413-7417, 1987 and U.S. Pat. No. 4,897,355 for a description of DOTMA and its use with DNA, which are incorporated by reference in their entirety).

A DOTMA analogue, 1,2-bis(oleoyloxy)-3-(trimethylammonia)propane (DOTAP) can be used in combination with a phospholipid to form DNA-complexing vehicles.

Lipofectin™ Bethesda Research Laboratories, Gaithersburg, Md.) is an effective agent for the delivery of highly anionic nucleic acids into living tissue culture cells that comprise positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonia)propane (“DOTAP”) (Boehringer Mannheim, Indianapolis, Ind.) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (Transfectam™, Promega, Madison, Wis.) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., Biochim. Biophys. Res. Commun. 179:280, 1991). Lipopolylysine, made by conjugating polylysine to DOPE, has been reported to be effective for transfection in the presence of serum (Zhou, X. et al., Biochim. Biophys. Acta 1065:8, 1991, which is incorporated by reference in its entirety). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, Calif.) and Lipofectamine (DOSPA) (Life Technology, Inc., Gaithersburg, Md.). Other cationic lipids suitable for the delivery of oligonucleotides are described in WO 98/39359 and WO 96/37194.

Liposomal formulations are particularly suited for topical administration, liposomes present several advantages over other formulations. Such advantages include reduced side effects related to high systemic absorption of the administered drug, increased accumulation of the administered drug at the desired target, and the ability to administer siRNA, into the skin. In some implementations, liposomes are used for delivering siRNA to epidermal cells and also to enhance the penetration of siRNA into dermal tissues, e.g., into skin. For example, the liposomes can be applied topically. Topical delivery of drugs formulated as liposomes to the skin has been documented (see, e.g., Weiner et al., Journal of Drug Targeting, 1992, vol. 2, 405-410 and du Plessis et al., Antiviral Research, 18, 1992, 259-265; Mannino, R. J. and Fould-Fogerite, S., Biotechniques 6:682-690, 1988; Itani, T. et al. Gene 56:267-276. 1987; Nicolau, C. et al. Meth. Enz. 149:157-176, 1987; Straubinger, R. M. and Papahadjopoulos, D. Meth. Enz. 101:512-527, 1983; Wang, C. Y. and Huang, L., Proc. Natl. Acad. Sci. USA 84:7851-7855, 1987, which are incorporated by reference in their entirety).

Non-ionic liposomal systems have also been examined to determine their utility in the delivery of drugs to the skin, in particular systems comprising non-ionic surfactant and cholesterol. Non-ionic liposomal formulations comprising Novasome I (glyceryl dilaurate/cholesterol/polyoxyethylene-10-stearyl ether) and Novasome II (glyceryl distearate/cholesterol/polyoxyethylene-10-stearyl ether) were used to deliver a drug into the dermis of mouse skin. Such formulations with siRNA are useful for treating a dermatological disorder.

Liposomes that include siRNA can be made highly deformable. Such deformability can enable the liposomes to penetrate through pore that are smaller than the average radius of the liposome. For example, transfersomes are a type of deformable liposomes. Transfersomes can be made by adding surface edge activators, usually surfactants, to a standard liposomal composition. Transfersomes that include siRNA can be delivered, for example, subcutaneously by infection in order to deliver siRNA to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transfersomes can be self-optimizing (adaptive to the shape of pores, e.g., in the skin), self-repairing, and can frequently reach their targets without fragmenting, and often self-loading.

Other formulations amenable to the present invention are described in U.S. provisional application Ser. No. 61/018,616, filed Jan. 2, 2008; 61/018,611, filed Jan. 2, 2008; 61/039,748, filed Mar. 26, 2008; 61/047,087, filed Apr. 22, 2008 and 61/051,528, filed May 8, 2008. PCT application no PCT/US2007/080331, filed Oct. 3, 2007 also describes formulations that are amenable to the present invention.

### Surfactants.

For ease of exposition the formulations, compositions and methods in this section are discussed largely with regard to unmodified siRNA compounds. It may be understood, however, that these formulations, compositions and methods can be practiced with other siRNA compounds, e.g., modified siRNA compounds, and such practice is within the scope of the invention. Surfactants find wide application in formulations such as emulsions (including microemulsions) and liposomes (see above). siRNA (or a precursor, e.g., a larger dsiRNA which can be processed into a siRNA, or a DNA which encodes a siRNA or precursor) compositions can include a surfactant. In one embodiment, the siRNA is formulated as an emulsion that includes a surfactant. The most common way of classifying and ranking the properties of the many different types of surfactants, both natural and synthetic, is by the use of the hydrophile/lipophile balance (HLB). The nature of the hydrophilic group provides the most useful means for categorizing the different surfactants used in formulations (Rieger, in “Pharmaceutical Dosage Forms,” Marcel Dekker, Inc., New York, N.Y., 1988, p. 285).

If the surfactant molecule is not ionized, it is classified as a nonionic surfactant. Nonionic surfactants find wide application in pharmaceutical products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in “Pharmaceutical Dosage Forms,” Marcel Dekker, Inc., New York, N.Y., 1988, p. 285).

### Micelles and Other Membranous Formulations.

For ease of exposition the micelles and other formulations, compositions and methods in this section are discussed largely with regard to unmodified siRNA compounds. It may be understood, however, that these micelles and other formulations, compositions and methods can be practiced with other siRNA compounds, e.g., modified siRNA compounds, and such practice is within the invention. The siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof)) composition can be provided as a micellar formulation. “Micelles” are defined herein as a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

A mixed micellar formulation suitable for delivery through transdermal membranes may be prepared by mixing an aqueous solution of the siRNA composition, an alkali metal C₈ to C₂₂ alkyl sulphate, and a micelle forming compounds. Exemplary micelle forming compounds include lecithin, hyaluronic acid, pharmaceutically acceptable salts of hyaluronic acid, glycolic acid, lactic acid, chamomile extract, cucumber extract, oleic acid, linoleic acid, linolenic acid, monoolein, monooleates, monolaurates, borage oil, evening of primrose oil, menthol, trihydroxy oxo cholanyl glycine and pharmaceutically acceptable salts thereof, glycerin, polyglycerin, lysine, polylysine, triolein, polyoxyethylene ethers and analogues thereof, polidocanol alkyl ethers and analogues thereof, chenodeoxycholate, deoxycholate, and mixtures thereof. The micelle forming compounds may be added at the same time or after addition of the alkali metal alkyl sulphate. Mixed micelles will form with substantially any kind of mixing of the ingredients but vigorous mixing in order to provide smaller size micelles.

In one method a first micellar composition is prepared which contains the siRNA composition and at least the alkali metal alkyl sulphate. The first micellar composition is then mixed with at least three micelle forming compounds to form a mixed micellar composition. In another method, the micellar composition is prepared by mixing the siRNA composition, the alkali metal alkyl sulphate and at least one of the micelle forming compounds, followed by addition of the remaining micelle forming compounds, with vigorous mixing.

Phenol and/or m-cresol may be added to the mixed micellar composition to stabilize the formulation and protect against bacterial growth. Alternatively, phenol and/or m-cresol may be added with the micelle forming ingredients. An isotonic agent such as glycerin may also be added after formation of the mixed micellar composition.

For delivery of the micellar formulation as a spray, the formulation can be put into an aerosol dispenser and the dispenser is charged with a propellant. The propellant, which is under pressure, is in liquid form in the dispenser. The ratios of the ingredients are adjusted so that the aqueous and propellant phases become one, i.e., there is one phase. If there are two phases, it is necessary to shake the dispenser prior to dispensing a portion of the contents, e.g., through a metered valve. The dispensed dose of pharmaceutical agent is propelled from the metered valve in a fine spray.

Propellants may include hydrogen-containing chlorofluorocarbons, hydrogen-containing fluorocarbons, dimethyl ether and diethyl ether. In certain embodiments, HFA 134a (1,1,1,2 tetrafluoroethane) may be used.

The specific concentrations of the essential ingredients can be determined by relatively straightforward experimentation. For absorption through the oral cavities, it is often desirable to increase, e.g., at least double or triple, the dosage for through injection or administration through the gastrointestinal tract.

### Particles.

For ease of exposition the particles, formulations, compositions and methods in this section are discussed largely with regard to modified siRNA compounds. It may be understood, however, that these particles, formulations, compositions and methods can be practiced with other siRNA compounds, e.g., unmodified siRNA compounds, and such practice is within the invention. In another embodiment, an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) preparations may be incorporated into a particle, e.g., a microparticle. Microparticles can be produced by spray-drying, but may also be produced by other methods including lyophilization, evaporation, fluid bed drying, vacuum drying, or a combination of these techniques.

The formulation is suitable for pharmaceutical use, which can be formulated together with one or more pharmaceutically acceptable carriers (additives), excipient and/or diluents, in a form suitable for oral delivery.

In one embodiment, oral delivery can be used to deliver an siRNA compound composition to a cell or a region of the gastro-intestinal tract, e.g., small intestine, colon (e.g., to treat a colon cancer), and so forth. The oral delivery form can be tablets, capsules or gel capsules. In one embodiment, the dsRNA compound of the formulation modulates expression of a cellular adhesion protein, modulates a rate of cellular proliferation, or has biological activity against eukaryotic pathogens or retroviruses. In another embodiment, the formulation includes an enteric material that substantially prevents dissolution of the tablets, capsules or gel capsules in a mammalian stomach. In some embodiments the enteric material is a coating. The coating can be acetate phthalate, propylene glycol, sorbitan monoleate, cellulose acetate trimellitate, hydroxy propyl methylcellulose phthalate or cellulose acetate phthalate.

In another embodiment, the oral dosage form of the formulation includes a penetration enhancer. The penetration enhancer can be a bile salt or a fatty acid. The bile salt can be ursodeoxycholic acid, chenodeoxycholic acid, and salts thereof. The fatty acid can be capric acid, lauric acid, and salts thereof.

In another embodiment, the oral dosage form of the formulation includes an excipient. In one example the excipient is polyethyleneglycol. In another example the excipient is precirol.

In another embodiment, the oral dosage form of the formulation includes a plasticizer. The plasticizer can be diethyl phthalate, triacetin dibutyl sebacate, dibutyl phthalate or triethyl citrate.

In one aspect, the invention features a formulation including an double-stranded iRNA compound and a delivery vehicle. In one embodiment, the dsRNA compound is (a) is 19-25 nucleotides long, for example, 21-23 nucleotides, (b) is complementary to an endogenous target RNA, and, optionally, (c) includes at least one 3′ overhang 1-5 nucleotides long.

In one embodiment, the delivery vehicle can deliver an siRNA compound, e.g., a double-stranded iRNA compound, or ssiRNA compound, to a cell by a topical route of administration. The delivery vehicle can be microscopic vesicles. In one example the microscopic vesicles are liposomes. In some embodiments the liposomes are cationic liposomes. In another example the microscopic vesicles are micelles. In one aspect, the invention features a formulation including an siRNA compound, e.g., a double-stranded iRNA compound, or ssiRNA compound, in an injectable dosage form. In one embodiment, the injectable dosage form of the formulation includes sterile aqueous solutions or dispersions and sterile powders. In some embodiments the sterile solution can include a diluent such as water; saline solution; fixed oils, polyethylene glycols, glycerin, or propylene glycol.

In one aspect, the invention features a formulation including an siRNA compound, e.g., a double-stranded iRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof) in oral dosage form. In one embodiment, the oral dosage form is selected from the group consisting of tablets, capsules and gel capsules. In another embodiment, the formulation includes an enteric material that substantially prevents dissolution of the tablets, capsules or gel capsules in a mammalian stomach. In some embodiments the enteric material is a coating. The coating can be acetate phthalate, propylene glycol, sorbitan monoleate, cellulose acetate trimellitate, hydroxy propyl methyl cellulose phthalate or cellulose acetate phthalate. In one embodiment, the oral dosage form of the formulation includes a penetration enhancer, e.g., a penetration enhancer described herein.

In another embodiment, the oral dosage form of the formulation includes an excipient. In one example the excipient is polyethyleneglycol. In another example the excipient is precirol.

In another embodiment, the oral dosage form of the formulation includes a plasticizer. The plasticizer can be diethyl phthalate, triacetin dibutyl sebacate, dibutyl phthalate or triethyl citrate.

In one aspect, the invention features a formulation including an siRNA compound, e.g., a double-stranded iRNA compound, or ssiRNA compound, in a rectal dosage form. In one embodiment, the rectal dosage form is an enema. In another embodiment, the rectal dosage form is a suppository.

In one aspect, the invention features a formulation including an siRNA compound, e.g., a double-stranded iRNA compound, or ssiRNA compound, in a vaginal dosage form. In one embodiment, the vaginal dosage form is a suppository. In another embodiment, the vaginal dosage form is a foam, cream, or gel.

In one aspect, the invention features a formulation including an siRNA compound, e.g., a double-stranded iRNA compound, or ssiRNA compound, in a pulmonary or nasal dosage form. In one embodiment, the siRNA compound is incorporated into a particle, e.g., a macroparticle, e.g., a microsphere. The particle can be produced by spray drying, lyophilization, evaporation, fluid bed drying, vacuum drying, or a combination thereof. The microsphere can be formulated as a suspension, a powder, or an implantable solid.

In some embodiments, the formulation is adapted for delivery as a capsule, soft elastic gelatin capsule, hard gelatin capsule, caplet, aerosol, spray, solution, suspension, or an emulsion.

In some embodiments, the formulation is a solid formulation, in a solid form, such as a tablet, capsule, caplet, pill, beads, powders or granules, sachets, troches, SEC (soft elastic capsule or “caplet”), or hard glatin capsule.

In some embodiments, the formulation is in a form of suspension, solution in water or non-aqueous media, an emulsion, aerosol, or spray.

In some embodiments, the formulation is a capsule, tablet, compression coated tablet, bilayer tablet, trilayer tablet, sachet, liquid-filled capsule or capsule comprising both liquid and solid components. In some embodiments, bioadhesive carrier particles are utilized. Suitable carrier particles include but are not limited to poly-amino acids, polyimines, polyacrylates, polyalkylacrylates, polyoxethanes, polyalkylcyanoacrylates, cationized gelatins, albumins, starches, acrylates, polyethylene glycol, DEAE-derivatized polyimines, pollulans, celluloses, chitosan, poly-L-lysine, polyhistidine, polyornithine, polyspermines, protamine, polyvinylpyridine, polythiodiethylamino-methylene P (TDAE), polyaminostyrene, poly (methylcyanoacrylate), poly (ethylcyanoacrylate), poly (butylcyanoacrylate), poly (isobutylcyanoacrylate), poly (isohexylcyanoacrylate), DEAE-methacrylate, DEAE-ethyhexylacrylate, DEAE-acrylamide, DEAE-albumin, DEAE-dextran, polymethylacrylate, polyhexylacrylate, poly (D, L-lactic acid), poly (DL-lactic-coglycolic acid) (PLGA) or polyethylene glycol (PEG). In some embodiments, the carrier particles are cationic. In some embodiments, the carrier particles comprise a complex of poly-L-lysine and alginate, a complex of protamine and alginate, lysine, dilysine, trilysine, calcium, albumin, glucosamine, arginine, galactosamine, nicotinamide, creatine, lysine-ethyl ester or arginine ethyl-ester. Preferably, the delayed release coating or matrix is acetate phthalate, propylene glycol, sorbitan monoleate, cellulose acetate phthalate (CAP), cellulose acetate trimellitate, hydroxypropyl methyl cellulose phthalate (HPMCP), methacrylates, chitosan, guar gum, polyethylene glycol (PEG), hydroxypropylmethylcellulose (HPMC), hydroxypropylethylcellulose, ethylcellulose or hydroxypropylmethylcellulose acetate succinate (HPMC-AS).

In some embodiments, the formulations comprise an enteric outer coating which resists degradation in the stomach and dissolves in the intestinal lumen. In one embodiment, the formulation comprises an enteric material effective in protecting the nucleic acid from pH extremes of the stomach, or in releasing the nucleic acid over time to optimize the delivery thereof to a particular mucosal site. Enteric materials for acid-resistant tablets, capsules and caplets are known in the art and typically include acetate phthalate, propylene glycol, sorbitan monoleate, cellulose acetate phthalate (CAP), cellulose acetate trimellitate, hydroxypropyl methyl cellulose phthalate (HPMCP), methacrylates, chitosan, guar gum, pectin, locust bean gum and polyethylene glycol (PEG).

Enteric materials may be incorporated within the dosage form or may be a coating substantially covering the entire surface of tablets, capsules or caplets. Enteric materials may also be accompanied by plasticizers that impart flexible resiliency to the material for resisting fracturing, for example during tablet curing or aging. Plasticizers are known in the art and typically include diethyl phthalate (DEP), triacetin, dibutyl sebacate (DBS), dibutyl phthalate (DBP) and triethyl citrate (TEC).

The formulation may also comprise a pharmaceutically acceptable organic or inorganic carrier substances suitable for oral administration which do not deleteriously react with nucleic acids. Suitable pharmaceutically acceptable carriers include, but are not limited to, water, salt solutions, alcohols, polyethylene glycols, gelatin, mannitol, lactose and other sugars and sugar derivatives, amylose, magnesium stearate, talc, silicic acid, viscous paraffin, colloidal silicon dioxide, hydroxymethylcellulose, polyvinylpyrrolidone and the like. The formulations can be sterilized and, if desired, mixed with auxiliary agents, e.g., lubricants, preservatives, flavorants, stabilizers, wetting agents, emulsifiers, salts for influencing osmotic pressure, buffers, bulking agents, colorings flavorings and/or aromatic substances and the like which do not deleteriously interact with the nucleic acid (s) of the formulation.

The formulation may also comprise an excipient. Typical pharmaceutical excipients include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, EXPLOTAB); and wetting agents (e.g., sodium lauryl sulphate, etc.). It is preferable that excipients with significant peroxide impurities are avoided.

In some embodiments, the formulation is administered via mucosal delivery. Formulation for mucosal administration can include powders or granules, beads, suspensions or solutions in water or non-aqueous media, capsules, sachets, troches, tablets or SECs. Thickeners, flavoring agents, colorants, emulsifiers, dispersing aids, carrier substances or binders may be desirably added to such formulations. A tablet may be made by compression or molding, optionally with one or more accessory ingredients.

Formulations for mucosal administration may include sterile and non-sterile aqueous solutions or suspensions, non-aqueous solutions in common solvents such as alcohols, or solutions or suspensions in liquid or solid oil bases. The solutions may also contain buffers, diluents and other suitable additives. Suspensions may contain substances that increase the viscosity of the suspension including, for example, sodium carboxymethylcellulose, sorbitol and/or dextran. The suspension may also contain stabilizers. Treatment Methods and Routes of Delivery

Another aspect of the invention relates to a method of reducing the expression of a target gene in a subject, comprising administering to the subject the double-stranded iRNA agent and a penetration enhancer.

For ease of exposition the formulations, compositions and methods in this section are discussed largely with regard to modified siRNA compounds. It may be understood, however, that these formulations, compositions and methods can be practiced with other siRNA compounds, e.g., unmodified siRNA compounds, and such practice is within the invention. A composition that includes a iRNA can be delivered to a subject by a variety of routes. Exemplary routes include: intravenous, topical, rectal, anal, vaginal, nasal, pulmonary, ocular.

The iRNA molecules of the invention can be incorporated into pharmaceutical compositions suitable for administration. Such compositions typically include one or more species of iRNA and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The formulation may be administered in a number of ways depending upon whether local or systemic treatment is desired and upon the area to be treated. Administration may be topical (including ophthalmic, vaginal, rectal, intranasal, transdermal), oral or parenteral.

In one embodiment, the formulation is for oral delivery of the double-stranded iRNA agent. Compositions for oral administration include powders or granules, suspensions or solutions in water, syrups, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the nucleic acid compositions can be combined with emulsifying and suspending agents. If desired, certain sweetening and/or flavoring agents can be added.

In one embodiment, the administration of the siRNA compound, e.g., a double-stranded siRNA compound, or ssiRNA compound, composition is parenteral, e.g, rectal, oral, or vaginal.

Administration can be provided by the subject or by another person, e.g., a health care provider. The medication can be provided in measured doses or in a dispenser which delivers a metered dose. Selected modes of delivery are discussed in more detail below.

The term “therapeutically effective amount” is the amount present in the composition that is needed to provide the desired level of drug in the subject to be treated to give the anticipated physiological response.

The term “physiologically effective amount” is that amount delivered to a subject to give the desired palliative or curative effect.

The term “pharmaceutically acceptable carrier” means that the carrier can be taken into the lungs with no significant adverse toxicological effects on the lungs.

The types of pharmaceutical excipients that are useful as carrier include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Bulking agents that are particularly valuable include compatible carbohydrates, polypeptides, amino acids or combinations thereof. Suitable carbohydrates include monosaccharides such as galactose, D-mannose, sorbose, and the like; disaccharides, such as lactose, trehalose, and the like; cyclodextrins, such as 2-hydroxypropyl-.beta.-cyclodextrin; and polysaccharides, such as raffinose, maltodextrins, dextrans, and the like; alditols, such as mannitol, xylitol, and the like. A group of carbohydrates may include lactose, threhalose, raffinose maltodextrins, and mannitol. Suitable polypeptides include aspartame. Amino acids include alanine and glycine, with glycine being used in some embodiments.

Additives, which are minor components of the composition of this invention, may be included for conformational stability during spray drying and for improving dispersibility of the powder. These additives include hydrophobic amino acids such as tryptophan, tyrosine, leucine, phenylalanine, and the like.

Suitable pH adjusters or buffers include organic salts prepared from organic acids and bases, such as sodium citrate, sodium ascorbate, and the like; sodium citrate may be used in some embodiments.

Administration of a micellar iRNA formulation may be achieved through metered dose spray devices with propellants such as tetrafluoroethane, heptafluoroethane, dimethylfluoropropane, tetrafluoropropane, butane, isobutane, dimethyl ether and other non-CFC and CFC propellants.

Oral. Any of the siRNA compounds described herein can be administered orally, e.g., in the form of tablets, capsules, gel capsules, lozenges, troches or liquid syrups. Further, the composition can be applied topically to a surface of the oral cavity.

For ease of exposition the formulations, compositions and methods in this section are discussed largely with regard to modified siRNA compounds. It may be understood, however, that these formulations, compositions and methods can be practiced with other siRNA compounds, e.g., unmodified siRNA compounds, and such practice is within the invention. Both the oral and nasal membranes offer advantages over other routes of administration. For example, drugs administered through these membranes have a rapid onset of action, provide therapeutic plasma levels, avoid first pass effect of hepatic metabolism, and avoid exposure of the drug to the hostile gastrointestinal (GI) environment. Additional advantages include easy access to the membrane sites so that the drug can be applied, localized and removed easily.

In oral delivery, compositions can be targeted to a surface of the oral cavity, e.g., to sublingual mucosa which includes the membrane of ventral surface of the tongue and the floor of the mouth or the buccal mucosa which constitutes the lining of the cheek. The sublingual mucosa is relatively permeable thus giving rapid absorption and acceptable bioavailability of many drugs. Further, the sublingual mucosa is convenient, acceptable and easily accessible.

The ability of molecules to permeate through the oral mucosa appears to be related to molecular size, lipid solubility and peptide protein ionization. Small molecules, less than 1000 daltons appear to cross mucosa rapidly. As molecular size increases, the permeability decreases rapidly. Lipid soluble compounds are more permeable than non-lipid soluble molecules. Maximum absorption occurs when molecules are un-ionized or neutral in electrical charges. Therefore charged molecules present the biggest challenges to absorption through the oral mucosae.

A pharmaceutical composition of the iRNA may also be administered to the buccal cavity of a human being by spraying into the cavity, without inhalation, from a metered dose spray dispenser, a mixed micellar pharmaceutical formulation as described above and a propellant. In one embodiment, the dispenser is first shaken prior to spraying the pharmaceutical formulation and propellant into the buccal cavity. For example, the medication can be sprayed into the buccal cavity or applied directly, e.g., in a liquid, solid, or gel form to a surface in the buccal cavity. This administration is particularly desirable for the treatment of inflammations of the buccal cavity, e.g., the gums or tongue, e.g., in one embodiment, the buccal administration is by spraying into the cavity, e.g., without inhalation, from a dispenser, e.g., a metered dose spray dispenser that dispenses the pharmaceutical composition and a propellant.

A pharmaceutical composition of the iRNA may also be administered to a subject via oral tubing or oral gavage, using a cannular, tube, or gavage needle. Dosage

In one aspect, the invention features a method of orally administering the formulation containing the dsRNA agent and the penetration enhancer to a subject (e.g., a human subject). In another aspect, the invention relates to the dsRNA agent as defined herein for use in inhibiting expression of a target gene in a subject. The method or the medical use includes administering a unit dose of the dsRNA agent.

The dosage of the oral composition is dependent on severity and responsiveness of the disease state to be treated, and the course of treatment lasting from several days to several months, or until a cure is effected or a diminution of the disease state is achieved. Optimal dosing schedules can be calculated from measurements of drug accumulation in the body of the patient. Optimum dosages may vary depending on the relative potency of individual oligonucleotides. Generally it can be estimated based on EC₅₀s or EC₇₀s found to be effective in vivo animal models. In general, dosage is from 0.01 μg to 1 g per kg of body weight, and may be given once or more daily, weekly, monthly, for example. The repetition rates for dosing can be estimated based on measured residence times and concentrations of the drug in bodily fluids or tissues.

In one embodiment, the unit dose is less than 50 mg per kg of bodyweight, or less than 40, 30, 25, 20, 15, 10, 5, 3, 2, 1, 0.5, 0.1, 0.05, 0.01, 0.005, 0.001, 0.0005, 0.0001, 0.00005 or 0.00001 mg per kg of bodyweight, and less than 200 nmole of dsRNA agent (e.g., about 4.4×10¹⁶ copies) per kg of bodyweight, or less than 1500, 750, 300, 150, 75, 15, 7.5, 1.5, 0.75, 0.15, 0.075, 0.015, 0.0075, 0.0015, 0.00075, 0.00015 nmole of dsRNA agent per kg of bodyweight.

The defined amount can be an amount effective to treat or prevent a disease or disorder, e.g., a disease or disorder associated with the target RNA. The unit dose, for example, can be administered by injection (e.g., intravenous, subcutaneous or intramuscular), an oral dose, an inhaled dose, or a topical application. In some embodiments dosages may be less than 50, 40, 30, 25, 20, 15, 10, 5, 3, 2, 1, or 0.1 mg/kg of body weight.

In some embodiments, the unit dose of the double-stranded iRNA agent is orally administered at no more than about 50 mg per kg body weight, for instance, no more than about 40 mg per kg body weight, no more than about 30 mg per kg body weight, no more than about 25 mg per kg body weight, no more than about 20 mg per kg body weight, no more than about 15 mg per kg body weight, no more than about 10 mg per kg body weight, no more than about 5 mg per kg body weight, no more than about 3 mg per kg body weight, no more than about 2 mg per kg body weight, no more than about 1 mg per kg body weight, no more than about 0.5 mg per kg body weight, or no more than about 0.1 mg per kg body weight. In some embodiments, the unit dose of the double-stranded iRNA agent is orally administered at about 1 to about 30 mg per kg body weight, for instance, about 3 to about 25 mg per kg body weight. In one embodiment, the dosage is calculated according to the oral bioavailability of the individual oligomer, to obtain a dosage that will allow maintenance of an effective concentration of the oligomer in the target tissue.

In some embodiments, the concentration of the penetration enhancer in the formulation is no more than about 200 mM, for instance, no more than about 150 mM, no more than about 100 mM, no more than about 80 mM, no more than about 60 mM, no more than about 50 mM, no more than about 45 mM, no more than about 40 mM, no more than about 35 mM, or no more than about 30 mM.

In some embodiments, the unit dose is administered less frequently than once a day, e.g., less than every 2, 4, 8 or 30 days. In another embodiment, the unit dose is not administered with a frequency (e.g., not a regular frequency). For example, the unit dose may be administered a single time.

In one embodiment, the effective dose is administered with other traditional therapeutic modalities. In one embodiment, the subject has a viral infection and the modality is an antiviral agent other than a dsRNA agent, e.g., other than a siRNA agent. In another embodiment, the subject has atherosclerosis and the effective dose of a dsRNA agent, e.g., a siRNA agent, is administered in combination with, e.g., after surgical intervention, e.g., angioplasty.

In one embodiment, a subject is administered an initial dose and one or more maintenance doses of the formulation containing the dsRNA agent. The maintenance dose or doses can be the same or lower than the initial dose, e.g., one-half less of the initial dose. A maintenance regimen can include treating the subject with a dose or doses ranging from 0.01 μg to 50 mg/kg of body weight per day, e.g., 40, 30, 25, 20, 15, 10, 5, 3, 2, 1, 0.1, 0.01, 0.001, or 0.00001 mg per kg of bodyweight per day.

The maintenance doses are, for example, administered no more than once every 2, 3, 4, 5, 6, 7, 8, 9, 10, 11, 12, 13, 14, 15, 20, or 30 days. Further, the treatment regimen may last for a period of time which will vary depending upon the nature of the particular disease, its severity and the overall condition of the patient. In certain embodiments the dosage may be delivered no more than twice or once per day, e.g., no more than once per 12, 24, 36, 48, or more hours, e.g., no more than once for every 2 days, every 3 days, every 4 days, every 5 days, every 6 days, every 7, every 8 days, every 9 days, every 10 days, every 11 days, every 12 days, every 13 days, or every 14 days.

Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable.

### Kits

In certain other aspects, the invention provides kits that include a suitable container containing a pharmaceutical formulation of an siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof). In certain embodiments the individual components of the pharmaceutical formulation may be provided in one container. Alternatively, it may be desirable to provide the components of the pharmaceutical formulation separately in two or more containers, e.g., one container for an siRNA compound preparation, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

The invention is further illustrated by the following examples, which should not be construed as further limiting. The contents of all references, pending patent applications and published patents, cited throughout this application are hereby expressly incorporated by reference.

### EXAMPLES

The invention now being generally described, it will be more readily understood by reference to the following examples which are included merely for purposes of illustration of certain aspects and embodiments of the present invention, and are not intended to limit the invention.

### Example 1. RNA Synthesis and Duplex Annealing

1. Oligonucleotide Synthesis:

All oligonucleotides were synthesized on an AKTAoligopilot synthesizer or an ABI 394 synthesizer. Commercially available controlled pore glass solid support (dT-CPG, 500 \acute{Å}, Prime Synthesis) and RNA phosphoramidites with standard protecting groups, 5′-O-dimethoxytrityl N6-benzoyl-2′-t-butyldimethylsilyl-adenosine-3′-O-N,N′-diisopropyl-2-cyanoethylphosphoramidite, 5′-O-dimethoxytrityl-N4-acetyl-2′-t-butyldimethylsilyl-cytidine-3′-O-N,N′-diisopropyl-2-cyanoethylphosphoramidite, 5′-O-dimethoxytrityl-N2-isobutyryl-2′-t-butyldimethylsilyl-guanosine-3′-O-N,N′-diisopropyl-2-cyanoethylphosphoramidite, and 5′-O-dimethoxytrityl-2′-t-butyldimethylsilyl-uridine-3′-O-N,N′-diisopropyl-2-cyanoethylphosphoramidite (Pierce Nucleic Acids Technologies) were used for the oligonucleotide synthesis unless otherwise specified. The 2′-F phosphoramidites, 5′-O-dimethoxytrityl-N4-acetyl-2′-fluoro-cytidine-3′-O-N,N′-diisopropyl-2-cyanoethyl-phosphoramidite and 5′-O-dimethoxytrityl-2′-fluoro-uridine-3′-O-N,N′-diisopropyl-2-cyanoethyl-phosphoramidite were purchased from (Promega). All phosphoramidites were used at a concentration of 0.2M in acetonitrile (CH₃CN) except for guanosine which was used at 0.2M concentration in 10% THF/ANC (v/v). Coupling/recycling time of 16 minutes was used. The activator was 5-ethyl thiotetrazole (0.75M, American International Chemicals), for the PO-oxidation Iodine/Water/Pyridine was used and the PS-oxidation PADS (2%) in 2,6-lutidine/ACN (1:1 v/v) was used.

Ligand conjugated strands were synthesized using solid support containing the corresponding ligand. For example, the introduction of carbohydrate moiety/ligand (for e.g., GalNAc) at the 3′-end of a sequence was achieved by starting the synthesis with the corresponding carbohydrate solid support. Similarly a cholesterol moiety at the 3′-end was introduced by starting the synthesis on the cholesterol support. In general, the ligand moiety was tethered to trans-4-hydroxyprolinol via a tether of choice as described in the previous examples to obtain a hydroxyprolinol-ligand moiety. The hydroxyprolinol-ligand moiety was then coupled to a solid support via a succinate linker or was converted to phosphoramidite via standard phosphitylation conditions to obtain the desired carbohydrate conjugate building blocks. Fluorophore labeled siRNAs were synthesized from the corresponding phosphoramidite or solid support, purchased from Biosearch Technologies. The oleyl lithocholic (GalNAc)₃ polymer support made in house at a loading of 38.6 μmol/gram. The Mannose (Man)₃ polymer support was also made in house at a loading of 42.0 μmol/gram.

Conjugation of the ligand of choice at desired position, for example at the 5′-end of the sequence, was achieved by coupling of the corresponding phosphoramidite to the growing chain under standard phosphoramidite coupling conditions unless otherwise specified. An extended 15 minutes coupling of 0.1M solution of phosphoramidite in anhydrous CH₃CN in the presence of 5-(ethylthio)-1H-tetrazole activator to a solid bound oligonucleotide. Oxidation of the internucleotide phosphite to the phosphate was carried out using standard iodine-water as reported (1) or by treatment with tert-butyl hydroperoxide/acetonitrile/water (10: 87: 3) with 10 minutes oxidation wait time conjugated oligonucleotide. Phosphorothioate was introduced by the oxidation of phosphite to phosphorothioate by using a sulfur transfer reagent such as DDTT (purchased from AM Chemicals), PADS and or Beaucage reagent The cholesterol phosphoramidite was synthesized in house, and used at a concentration of 0.1 M in dichloromethane. Coupling time for the cholesterol phosphoramidite was 16 minutes.

2. Deprotection-I (Nucleobase Deprotection)

After completion of synthesis, the support was transferred to a 100 ml glass bottle (VWR). The oligonucleotide was cleaved from the support with simultaneous deprotection of base and phosphate groups with 80 mL of a mixture of ethanolic ammonia [ammonia: ethanol (3:1)] for 6.5h at 55° C. The bottle was cooled briefly on ice and then the ethanolic ammonia mixture was filtered into a new 250 ml bottle. The CPG was washed with 2×40 mL portions of ethanol/water (1:1 v/v). The volume of the mixture was then reduced to ˜ 30 ml by roto-vap. The mixture was then frozen on dry ice and dried under vacuum on a speed vac.

3. Deprotection-H (Removal of 2′ TBDMS Group)

The dried residue was resuspended in 26 ml of triethylamine, triethylamine trihydrofluoride (TEA.3H1F) or pyridine-HF and DMSO (3:4:6) and heated at 60° C. for 90 minutes to remove the tert-butyldimethylsilyl (TBDMS) groups at the 2′ position. The reaction was then quenched with 50 ml of 20 mM sodium acetate and pH adjusted to 6.5, and stored in freezer until purification.

4. Analysis

The oligonucleotides were analyzed by high-performance liquid chromatography (HPLC) prior to purification and selection of buffer and column depends on nature of the sequence and or conjugated ligand.

5. HPLC Purification

The ligand conjugated oligonucleotides were purified reverse phase preparative HPLC. The unconjugated oligonucleotides were purified by anion-exchange HPLC on a TSK gel column packed in house. The buffers were 20 mM sodium phosphate (pH 8.5) in 10% CH₃CN (buffer A) and 20 mM sodium phosphate (pH 8.5) in 10% CH₃CN, 1M NaBr (buffer B). Fractions containing full-length oligonucleotides were pooled, desalted, and lyophilized. Approximately 0.15 OD of desalted oligonucleotides were diluted in water to 150 μl and then pipetted in special vials for CGE and LC/MS analysis. Compounds were finally analyzed by LC-ESMS and CGE.

6. siRNA Preparation

For the preparation of siRNA, equimolar amounts of sense and antisense strand were heated in 1×PBS at 95° C. for 5 minutes and slowly cooled to room temperature. Integrity of the duplex was confirmed by HPLC analysis.

### Example 2A. In Vivo Evaluation of Oral Delivery of the Formulation Containing GalNAc-siRNA Conjugates in Mice

### Experimental Design

An exemplary siRNA targeting F12 ELF with GalNAc conjugate and a 5′-vinyl phosphonate (VP) modification shown in the table below was used in the oral formulation for the in vivo mouse study. Oral formulation containing the same siRNA targeting F12, without the GalNAc conjugation, was used as the control group.

siRNA's for oral delivery of siRNA conjugates

| Duplex | Oligo | | | | Molecular | MW |
|---|---|---|---|---|---|---|
| Id | Id | Strand | Target | OligoSeq | Weight | Found |
| AD- | A- | sense | F12 | gsasaacuCfaAfUfAfaag | 8756.561 | 8752.058 |
| 291897 | 147454 | | | ugcuuuaL96 | | |
| | A- | antis | F12 | VPuAfaagCfacuuuauUf | 7653.905 | 7650.14 |
| | 447593 | | | gAfguuucsusg | | |
| AD- | A- | sense | F12 | gsasaacuCfaAfUfAfaag | 6999.731 | 6996.093 |
| 392979 | 163316 | | | ugcuususa | | |
| | A- | antis | F12 | VPuAfaagCfacuuuauUf | 7653.905 | 7650.14 |
| | 447593 | | | gAfguuucsusg | | |

L96: TriGalNAc;
VP: vinyl phosphonate

The formulation also contains sodium caprate (C10) (food additive status) as an intestinal permeation enhancer. The formulation was prepared as follows. A solution of sodium caprate (150 mM) was prepared by dissolving 1.45 g in 50 mL of water and kept at ambient temperature. siRNA duplexes were lyophilized to a powder and dissolved in 150 mM sodium caprate solution to generate formulations having different concentrations used in this example. These formulations were kept refrigerated until in vivo experiments.

### In Vivo Evaluation

In vivo studies were performed in mice (C57BL-6J black mouse, 3 animals/group) with or without fasting, and dosed either via oral gavage or subcutaneously. siRNA formulations were given to mice via oral gavage using oral gavage needles (10 μL/g).

For these experiments three doses were administrated either at days 0, 2, and 8 or at days 0, 7, and 14. F12 protein levels from plasma were determined at days 0, 5, 8, 14, 21, 28, 35 and 42 after bleeding.

Formulation with and without Sodium Caprate (CO)

Fasted animals (5 hours prior to dosing) were orally administered a dose of a formulation containing a GalNAc-siRNA of 25 mg/kg (with 37.5 mM C10) at days 0, 2, and 5. A comparison was made to fasted animals (5 hours prior to dosing) that were orally administered a dose of the same siRNA of 25 mg/kg (without C10) at days 0, 2, and 5. A comparison was also made employing the same siRNA administered subcutaneously at a single dose of 0.75 mg/kg.

The results are shown in FIG. 1. FIG. 1 illustrates the effect of the formulation of the siRNA with sodium caprate on the oral delivery of GalNAc-conjugated siRNA. As shown in FIG. 1, the formulation containing the penetration enhancer C10 (with 37.5 mM C10) had a significantly better activity than the formulation without C10, illustrating that C10 is beneficial for oral delivery of GalNAc-siRNA in mice.

### Dose Response

Fasted animals (5 hours prior to dosing) were orally administered a dose of a formulation containing a GalNAc-siRNA of 3 mg/kg (with 45.4 mM C10 in solution), 10 mg/kg (with 150 mM C10), and 25 mg/kg (with 37.5 mM C10), respectively, at days 0, 2, and 5. A comparison was made employing the same siRNA administered subcutaneously at a single dose of 0.75 mg/kg.

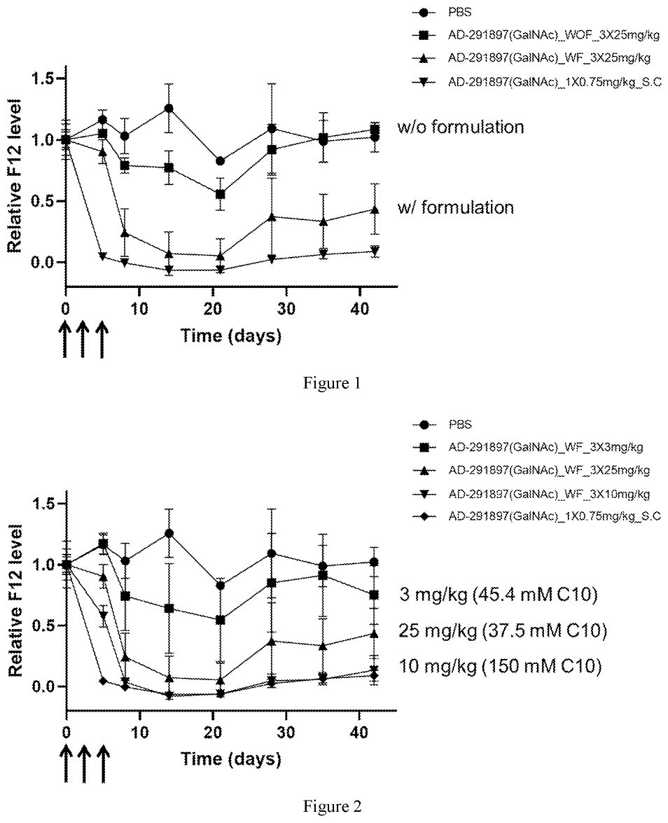

The results are shown in FIG. 2. FIG. 2 indicates the dose response and the effect of the concentration of sodium caprate in the formulation prior to dosing. As shown in FIG. 2, robust activity was observed for oral administration of the formulation containing GalNAc-siRNA and C10. The max KD for this oral formulation was observed for a siRNA dose at 10 mg/kg (with 150 mM C10), which was comparable to the result of a single subcutaneous dose of the same siRNA at 0.75 mg/kg. The better activity obtained from the siRNA dosage at 10 mg/kg (with 150 mM C10), as compared against the activity obtained from siRNA dosage at 25 mg/kg (with 37.5 mM C10), may be resulted from a higher concentration of the penetration enhancer C10 in the formulation (150 mM v. 37.5 mM.

Fasting v. Non-Fasting

Fasted animals (5 hours prior to dosing) were orally administered a dose of a formulation containing a GalNAc-siRNA of 25 mg/kg (with 37.5 mM C10) at days 0, 2, and 5. A comparison was made to unfasted animals that were orally administered a dose of the same siRNA of 25 mg/kg (with 37.5 mM C10) at days 0, 2, and 5. A comparison was also made employing the same siRNA administered subcutaneously at a single dose of 0.75 mg/kg.

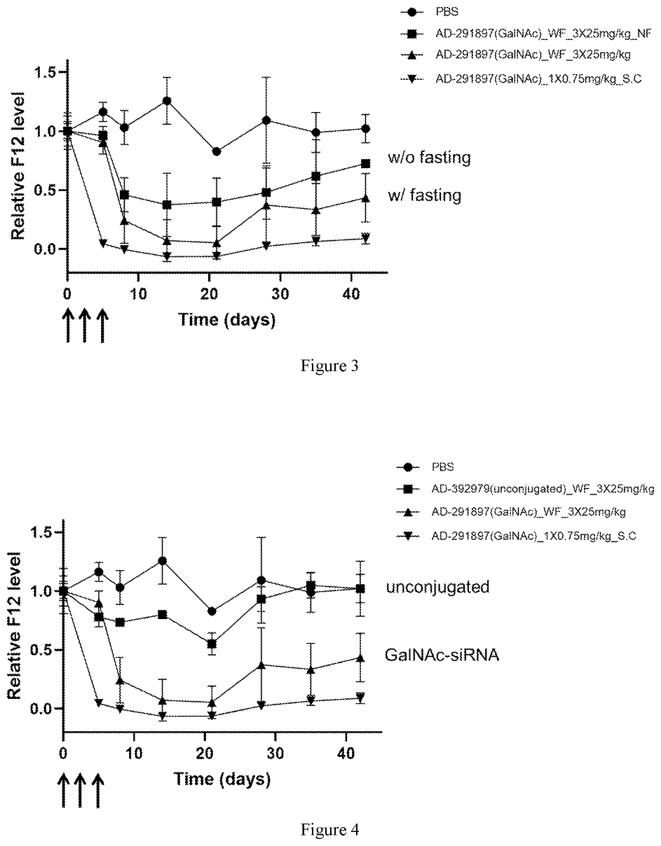

The results are shown in FIG. 3. FIG. 3 illustrates the effect of fasting on the oral delivery of the GalNAc-conjugated siRNA formulated in sodium caprate. As shown in FIG. 3, fasting conditions improved the activity by oral delivery.

GalNAc Conjugation v. Unconjugated

Fasted animals (5 hours prior to dosing) were orally administered a dose of a formulation containing a GalNAc-conjugated siRNA of 25 mg/kg (with 37.5 mM C10) at days 0, 2, and 5. A comparison was made to fasted animals (5 hours prior to dosing) that were orally administered a dose of a formulation containing an unconjugated siRNA of 25 mg/kg (with 37.5 mM C10) at days 0, 2, and 5. A comparison was also made employing the same siRNA administered subcutaneously at a single dose of 0.75 mg/kg.

The results are shown in FIG. 4. FIG. 4 compares the oral delivery of siRNA with and without conjugation of GalNAc. As shown in FIG. 4, the formulation containing the GalNAc-conjugated siRNA had a significantly better activity than the formulation containing the unconjugated siRNA, illustrating that GalNAc conjugation is beneficial for oral delivery of siRNA in mice.

### Dosing Time Intervals for Multi-Dosage

The first dosing paradigm: Fasted animals (5 hours prior to dosing) were orally administered a dose of a formulation containing a GalNAc-siRNA of 25 mg/kg (with 37.5 mM C10) at days 0, 2, and 5. The second dosing paradigm: fasted animals (5 hours prior to dosing) were orally administered a dose of a formulation containing the same siRNA of 25 mg/kg (with 37.5 mM C10) at days 0, 8, and 14. A comparison was made employing the same siRNA administered subcutaneously at a single dose of 0.75 mg/kg.

The results are shown in FIG. 5. FIG. 5 illustrates the effect of dosing paradigm for oral delivery of GalNAc-conjugated siRNA. As shown in FIG. 5, both dosing paradigms of the formulation appeared to result in good, durable activities.

### Example 2B. In Vivo Evaluation of Oral Delivery of the Formulation Containing GalNAc-siRNA Conjugates in Mice

### Experimental Design

An exemplary siRNA targeting F12 with GalNAc conjugate and a 5′-vinyl phosphonate (VP) modification shown in the table below was used in the oral formulation for the in vivo mouse study. Oral formulations containing the same siRNA targeting F12, without the GalNAc conjugation or without the 5′-VP modification, respectively, were used as the control groups.

siRNA's for oral delivery of siRNA conjugates

| Duplex | Oligo | | | | Molecular | MW |
|---|---|---|---|---|---|---|
| Id | Id | Strand | Target | OligoSeq (5′-3′) | Weight | Found |
| AD- | A- | sense | F12 | gsasaacuCfaAfUfAfaagu | 8756.561 | 8752.058 |
| 291897 | 147454 | | | gcuuuaL96 | | |
| | A- | antis | F12 | VPuAfaagCfacuuuauUfg | 7653.905 | 7650.14 |
| | 447593 | | | Afguuucsusg | | |
| AD- | A- | sense | F12 | gsasaacuCfaAfUfAfaagu | 6999.731 | 6996.093 |
| 392979 | 163316 | | | gcuususa | | |
| | A- | antis | F12 | VPuAfaagCfacuuuauUfg | 7653.905 | 7650.14 |
| | 447593 | | | Afguuucsusg | | |
| AD- | A- | sense | F12 | gsasaacuCfaAfUfAfaagu | 8756.561 | 8752.058 |
| 74210 | 147454 | | | gcuuuaL96 | | |
| | A- | antis | F12 | usAfsaagCfacuuuauUfgA | 7610.037 | 7606.115 |
| | 148543 | | | fguuucsusg | | |

L96: TriGalNAc;
VP: vinyl phosphonate;
Uhd: 2′-O-hexadexyl-uridine-3′-phosphate

The formulation also contains sodium caprate (C10) (food additive status) as an intestinal permeation enhancer. The formulation was prepared as follows. A solution of sodium caprate (150 mM) was prepared by dissolving 1.45 g in 50 mL of water and kept at ambient temperature. siRNA duplexes were lyophilized from water to a powder and dissolved in 150 mM sodium caprate solution to generate formulations having different concentrations used in this example. These formulations were kept refrigerated until in vivo experiments.

### In Vivo Evaluation

In vivo studies were performed in mice (C57BL-6J black wild type mouse or ASGR KO mouse, 3 or 4 animals/group) with or without fasting, and dosed either via oral gavage or subcutaneously. siRNA formulations were given to mice via oral gavage using oral gavage needles (10 μL/g).

For these experiments, either single dose or three doses were administrated. A single-dose regimen included dosing via oral gavage at day 0. A three-dose regimen included dosing via oral gavage for every 4 hours on day 0; dosing at days 0, 2, and 5; or dosing at days 0, 1, and 2. Day 0 was defined as the first day of the study. F12 protein levels from plasma were determined up to day 42, for example, F12 levels in plasma were determined at days 0 (pre-dose), 5, 8, 14, 21, 28, 35 and 42 after bleeding.

Comparison of Pharmacodynamics (PD) with and without GalNAc Conjugation

Fasted mice (5 hours prior to dosing) were orally administered a dose of a formulation containing a GalNAc-siRNA of 10 mg/kg (with 150 mM C10) at days 0, 2, and 5. A comparison was made to fasted mice (5 hours prior to dosing) that were orally administered a dose of the same siRNA without GalNAc conjugation of 10 mg/kg (with 150 mM C10) at days 0, 2, and 5. Comparisons were also made employing the same GalNAc-siRNA administered subcutaneously at a single dose of 0.75 mg/kg, and PBS administered orally.

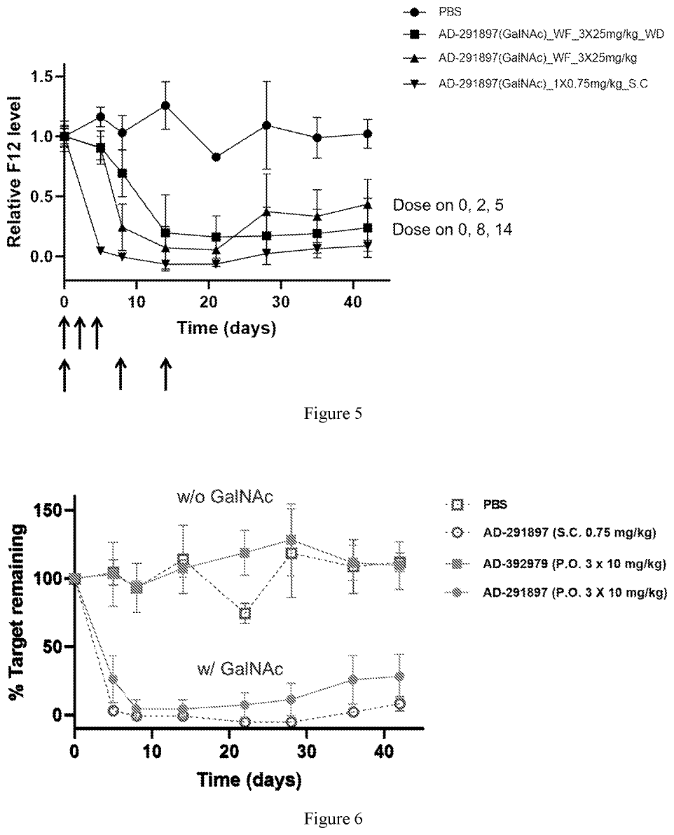

FIG. 6 compares the oral delivery of siRNA with and without conjugation of GalNAc, demonstrating that GalNAc conjugation significantly improved the activity after oral gavage. Three doses of the siRNA without GalNAc conjugation, via oral gavage at 10 mg/kg, did not cause activity, whereas three doses of a formulation containing GalNAc-siRNA, via oral gavage at 10 mg/kg, resulted in a PD profile similar to that resulted from a single dose of a formulation containing the same GalNAc-siRNA, via subcutaneous injection at 0.75 mg/kg. These results suggest that GalNAc conjugation plays a significant role in oral delivery of liver-targeting siRNA, similar to the role it plays in systemic administration.

Wild Type (WT) Vs. ASGR KO Mice

Fasted wild-type mice (5 hours prior to dosing) or fed wild-type mice were orally administered a dose of a formulation containing a GalNAc-siRNA of 30 mg/kg (with 150 mM C10) at day 0. A comparison was made to fasted ASGR-knockout mice (5 hours prior to dosing) or fed ASGR-knockout mice that were orally administered a dose of the same GalNAc-siRNA of 30 mg/kg (with 150 mM C10) at day 0.

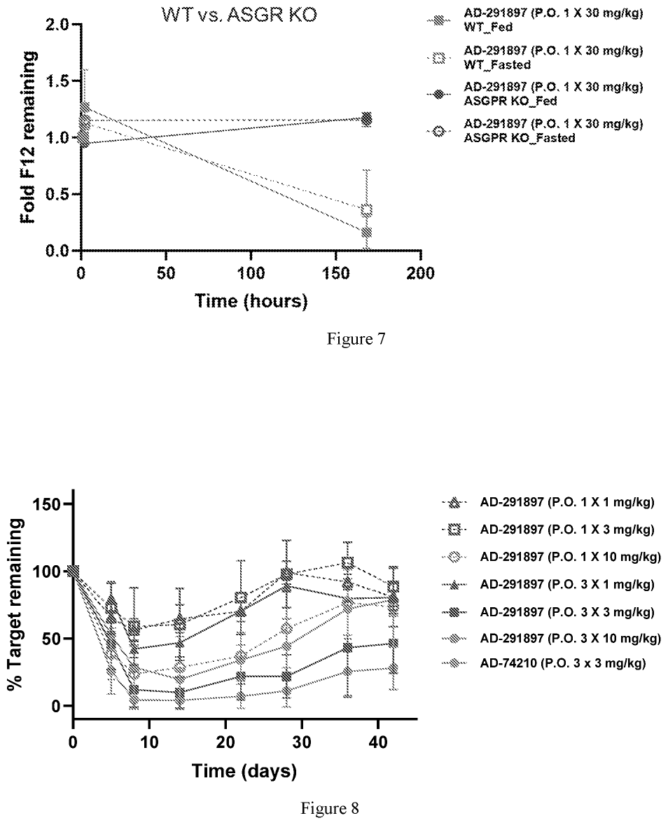

FIG. 7 demonstrates the ASGR-mediated uptake of GalNAc-siRNA in the liver of the mice. Significant knock down (KD) in plasma F12 level was observed on day 7 after oral administration of a GalNAc-siRNA (with 150 mM C10) to WT mice, whereas plasma F12 level after oral administration of a GalNAc-siRNA (with 150 mM C10) to ASGR KO mice was not changed. These results suggest that deficiency in ASGR expression in the liver may have reduced localization of GalNAc-siRNA in hepatocyte and resulted in loss of activity. For either mouse strain, the PD profiles after oral administration of a GalNAc-siRNA (with 150 mM C10) to fasted mice were comparable to the results of those in fed mice at the single dose level of 30 mg/kg.

### Dose Response

Fasted mice (5 hours prior to dosing) were orally administered under a three-dose regimen, including dosing a formulation containing a GalNAc-siRNA of 1 mg/kg, 3 mg/kg, and 10 mg/kg (with 150 mM C10 in solution), respectively, at days 0, 2, and 5; or a single-dose regimen, including dosing a formulation containing a GalNAc-siRNA of 1 mg/kg, 3 mg/kg, and 10 mg/kg (with 150 mM C10), respectively, on day 0.

FIG. 8 demonstrates the dose response of oral delivery of a formulation containing GalNAc-siRNA conjugates and the effect of the dosing regimen. As shown in the figure, the activity increased as the dosage increased from 3 mg/kg to 10 mg/kg at a single-dose regimen. When three doses were administered via oral gavage, gradual increase in activity was observed as the dosage increased from 1 mg/kg to 10 mg/kg. For a three-dose regimen on days 0, 2, and 5, approximately 95% of KD and 88% of KD were observed at nadir for each dosage at 10 mg/kg (with 150 mM C10) and at 3 mg/kg (with 150 mM C10), respectively.

### Impact of Vinylphosphonate (VP) Modification

A comparison was also made to fasted mice (5 hours prior to dosing) that were orally administered a dose of the same siRNA without VP modification of 3 mg/kg (with 150 mM C10) at days 0, 2, and 5.

FIG. 8 illustrates that VP modification at the 5′-end of the antisense strand of the GalNAc-siRNA did not significantly change the PD profile of the mice orally administered formulation containing the GalNAc-siRNA (with 150 mM C10), indicating that VP modification may have a minimal effect on the activity of the liver-targeting GalNAc-siRNA in oral delivery.

### Dosing Regimen

The first dosing paradigm: fasted mice (5 hours prior to dosing) were orally administered a dose of a formulation containing a GalNAc-siRNA of 3 mg/kg (with 150 mM C10) at days 0, 2, and 5. The second dosing paradigm: fasted mice (5 hours prior to dosing) were orally administered a dose of a formulation containing the same siRNA of 3 mg/kg (with 150 mM C10) at days 0, 1, and 2. The third dosing paradigm: fed mice were orally administered a dose of a formulation containing the same siRNA of 3 mg/kg (with 150 mM C10) for every 4 hours, three times, on day 0.

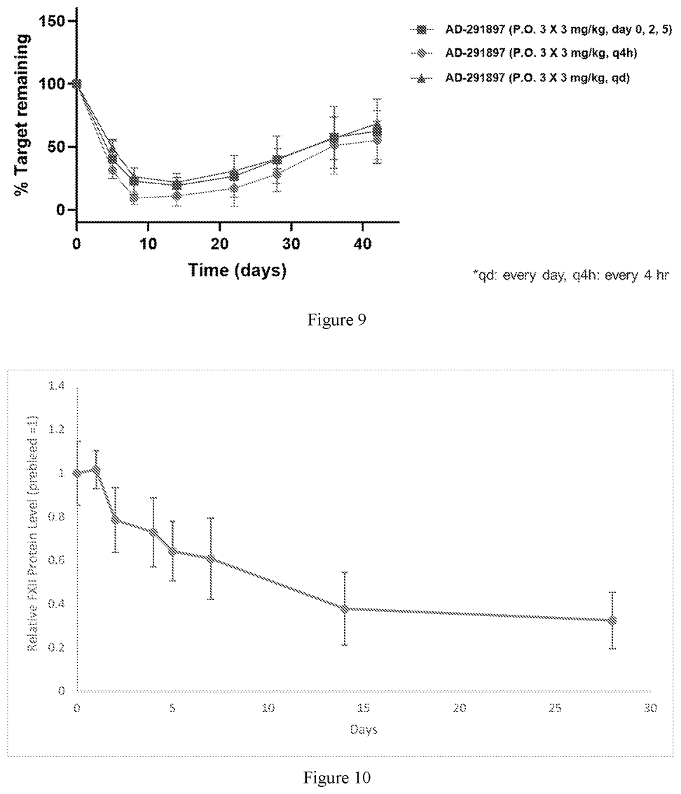

FIG. 9 illustrates the effect of the dosing paradigm for the oral delivery of GalNAc-conjugated siRNA. As shown in the figure, all three dosing paradigms of the formulation appeared to result in good, durable activities at 9 mg/kg dose level with 78-91% of KD level at nadir.

### Example 3A. In Vivo Evaluation of Oral Delivery of the Formulation Containing GalNAc-siRNA Conjugates in Non-Human Primates

The exemplary siRNA used in this example was the same as AD-291897 Duplex shown in the table in Example 2A. The experimental design was the same as in Example 2A, except that the siRNA targeting F12 was used in the oral formulation for the in vivo NHP (non-human primates) study.

The procedures for formulation preparation in this example was the same as the procedures for formulation preparation described in Example 2A, using sodium caprate (C10, 150 mM) as an intestinal permeation enhancer.

NHP was orally administered a formulation containing a GalNAc-siRNA with 150 mM C10. The study results are shown in FIG. 10. FIG. 10 shows the relative F12 levels following oral delivery to a NHP of a formulation containing a GalNAc-siRNA (with 150 mM C10 in solution.

### Example 3B. In Vivo Evaluation of Oral Delivery of the Formulation Containing GalNAc-siRNA Conjugates in Non-Human Primates

### Experimental Design

Exemplary siRNAs targeting F12 or transthyretin (TTR) with GalNAc conjugate and a 5′-vinyl phosphonate (VP) modification shown in the table below were used in the oral formulation for the in vivo non-human primate (NHP) study.

siRNA's for oral delivery of siRNA conjugates

| Duplex | Oligo | | | | Molecular | MW |
|---|---|---|---|---|---|---|
| Id | Id | Strand | Target | OligoSeq (5′-3′) | Weight | Found |
| AD- | A- | sense | F12 | gsasaacuCfaAfUfAfaagugc | 8756.561 | 8752.058 |
| 291897 | 147454 | | | uuuaL96 | | |
| | A- | antis | F12 | VPuAfaagCfacuuuauUfgA | 7653.905 | 7650.14 |
| | 447593 | | | fguuucsusg | | |
| AD- | A- | sense | TTR | usgsggauUfuCfAfUfguaac | 8788.558 | 8784.048 |
| 157687 | 131354 | | | caagaL96 | | |
| | A- | antis | TTR | VPuCfuugGfuuAfcaugAfa | 7600.875 | 7596.146 |
| | 265470 | | | Afucccasusc | | |
| AD- | A- | sense | TTR | usgsggauUfuCfAfUfguaac | 8788.558 | 8784.048 |
| 87404 | 131354 | | | caagaL96 | | |
| | A- | antis | TTR | usCfsuugGf(Tgn)uAfcaug | 7497.970 | 7494.115 |
| | 173307 | | | AfaAfucccasusc | | |

L96: TriGalNAc;
VP: vinyl phosphonate;
Tgn: thymidine-glycol nucleic acid (GNA) S-isomer

The procedures for formulation preparation in this example were the same as the procedures for formulation preparation described in Example 2A, using sodium caprate (C10, 150 mM) as an intestinal permeation enhancer.

### In Vivo Evaluation

In vivo studies were performed in NHP (Cynomolgus monkey, Macaca fascicularis, 4 animals/group for pilot study and 3 animals/group for the other studies) and dosed either via oral gavage or subcutaneously. siRNA formulations were given to NHP via oral gavage using a syringe with an attached gavage tube. Each dose was followed by a tap water flush of 5 mL. For oral delivery experiments, three doses were administrated at days 1, 3, and 6. Day 1 was defined as the first day of the study.

PD analysis: F12 or TTR protein levels from plasma were determined at days 0 (pre-dose), 4, 8, 15, 22, 29, 36, and 43 after bleeding.

PK analysis: Blood collection was conducted at pre-dose, 15 minutes, 30 minutes, 1 hour, 2 hours, 4 hours, 8 hours, 12 hours, 24 hours, and 48 hours. Plasma samples were reverse transcribed to cDNA and then quantified by qPCR on a ViiA™ 7 Real-Time PCR System. Liver biopsy was collected from 1 animal/group/time point according to the following table, for a total of up to four non-terminal biopsies per animal. Liver lysates were extracted using Clarity OTX™ SPE 96-well plate cartridges and quantified by LC-MS.

| Animal | Time points (postdose relative to day 1 dosing) | | | | | | | | |
|---|---|---|---|---|---|---|---|---|---|
| No. | Day 1: 2 h | Day 1: 6 h | Day 2 | Day 7 | Day 8 | Day 15 | Day 22 | Day 29 | Day 43 |
| 1 | X | — | — | X | — | — | X | — | — |
| 2 | — | X | — | — | X | — | — | X | — |
| 3 | — | — | X | — | — | X | — | — | X |

X: sample to be collected;
—: not applicable;
h: hours

Because GalNAc-siRNA was targeted to liver during first pass after oral dosing, it may be possible that plasma concentrations may not reflect the absorption. Therefore, PD results and liver concentrations were used as measures of absorption as potential surrogate in NHP.

Bioavaiblility (% F) from PD was calculated by the formula below:

Bioavailability (% F)=(KD_{max, oral}×Dose_{subcutaneous})/(KD_{max, subcutaneous}×Doseₒᵣₐₗ)×100.

Bioavaiblility (% F) from liver concentration was calculated by the formula below:

Bioavailability (% F)=(AUCₒᵣₐₗ×Dose_{subcutaneous})/(AUC_{subcutaneous}×Doseₒᵣₐₗ)×100.

NHP PD Study with F12 Sequence

NHP (n=4) were orally administered a dose of a formulation containing a GalNAc-siRNA (AD-291897) of 10 mg/kg (with 150 mM C10 in solution) at days 1, 3, and 6.

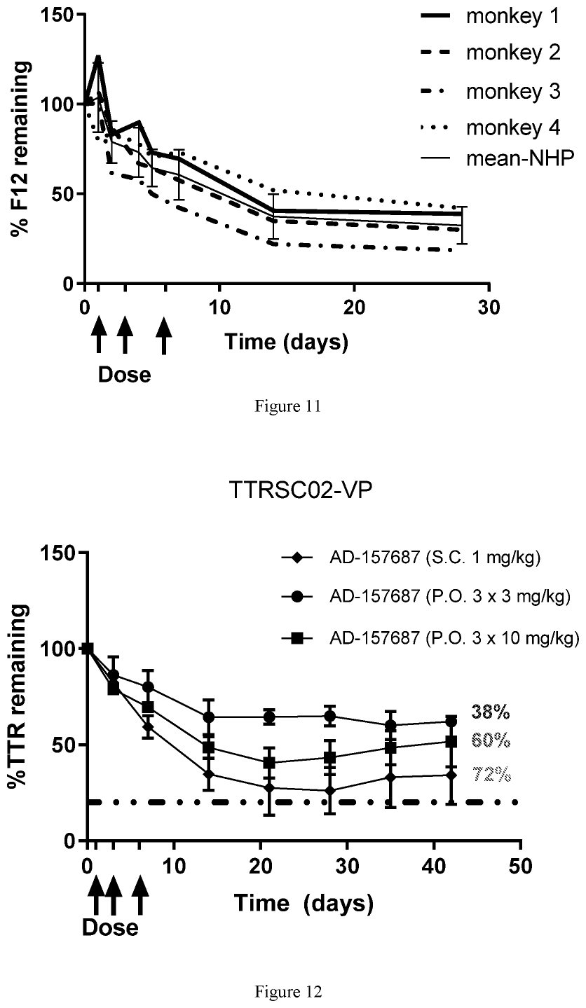

FIG. 11 demonstrates a robust and durable reduction in circulating F12 with approximately 68% of mean value and 81% of max level of KD in NHP following the oral gavage of a formulation containing a GalNAc-siRNA, suggesting significant activity in NHP.

NHP PD Study with TTR Sequence of AD-157687

NHP (n=3) were orally administered a dose of a formulation containing a GalNAc-siRNA (AD-157687) of 3 mg/kg and 10 mg/kg (with 150 mM C10 in solution), respectively, at days 1, 3, and 6. A comparison was made employing the same GalNAc-siRNA administered subcutaneously at a single dose of 1 mg/kg.

FIG. 12 demonstrates a robust and durable reduction in circulating TTR with approximately 38% and 60% of KD in NHP following oral gavage of a formulation containing a GalNAc-siRNA of 3 mg/kg and 10 mg/kg, respectively. The results also show the dose response in oral dosing containing GalNAc-siRNA. The variability following the oral dosing was consistent with that observed following the subcutaneous dosing.

Apparent % F obtained from the PD results were 5.7% and 2.7%, respectively, in 3 mg/kg and 10 mg/kg oral dosing at days 1, 3, and 6. Apparent % F obtained from the liver concentrations were 2.0% and 1.4%, respectively, in 3 mg/kg and 10 mg/kg oral dosing at days 1, 3, and 6.

NHP PD Study with TTR Sequence of AD-87404

NHP (n=3) were orally administered a dose of a formulation containing a GalNAc-siRNA (AD-87404) of 3 mg/kg, 10 mg/kg, and 30 mg/kg (with 150 mM C10 in solution), respectively, at days 1, 3, and 6. A comparison was made employing the same GalNAc-siRNA administered subcutaneously at a single dose of 3 mg/kg.

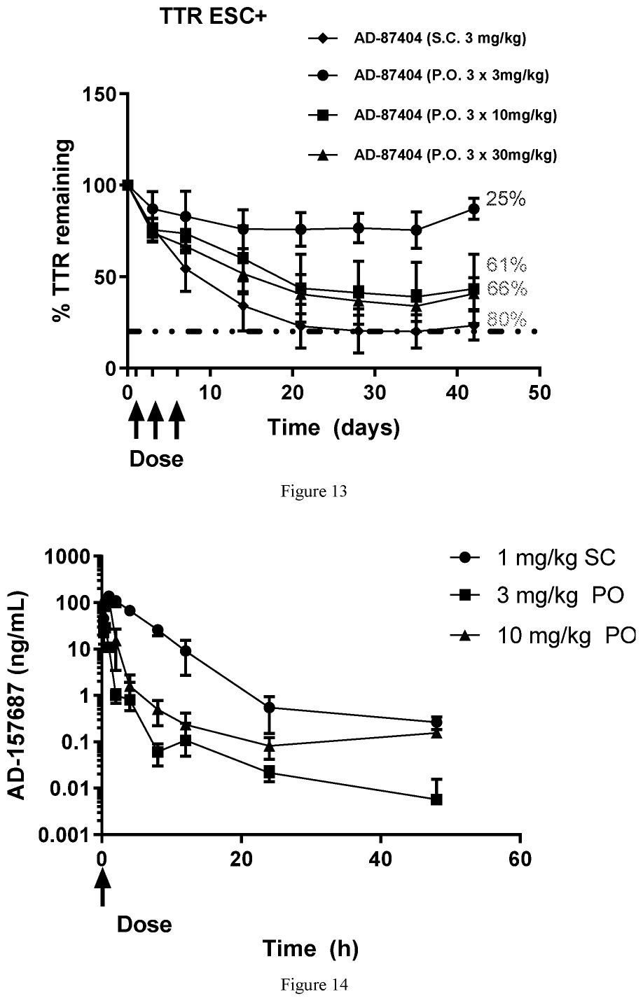

FIG. 13 demonstrates a robust and durable reduction in circulating TTR with approximately 25%, 61%, and 66% of KD in NHP following oral gavage of a formulation containing a GalNAc-siRNA of 3 mg/kg, 10 mg/kg, and 30 mg/kg, respectively. Saturation was observed in the group of oral dosing at 30 mg/kg on days 1, 3, and 6, which may be due to ASGR saturation or narrow absorption window of the C10 formulation.

Apparent % F obtained from the PD results were 10.0%, 7.7%, and 2.8%, respectively, in 3 mg/kg, 10 mg/kg, and 30 mg/kg oral dosing at days 1, 3, and 6. Apparent % F obtained from the liver concentrations were 1.5%, 1.9%, and 1.9%, respectively, in 3 mg/kg, 10 mg/kg, and 30 mg/kg oral dosing at days 1, 3, and 6.

NHP PK Study with TTR Sequence AD-157687 and AD-87404

NHP (n=3) were orally administered a dose of a formulation containing a GalNAc-siRNA (AD-157687) of 3 mg/kg and 10 mg/kg (with 150 mM C10 in solution), respectively, at days 1, 3, and 6. A comparison was made employing the same GalNAc-siRNA administered subcutaneously at a single dose of 1 mg/kg.

NHP (n=3) were orally administered a dose of a formulation containing a GalNAc-siRNA (AD-87404) of 3 mg/kg, 10 mg/kg, and 30 mg/kg (with 150 mM C10 in solution), respectively, at days 1, 3, and 6. A comparison was made employing the same GalNAc-siRNA administered subcutaneously at a single dose of 3 mg/kg.

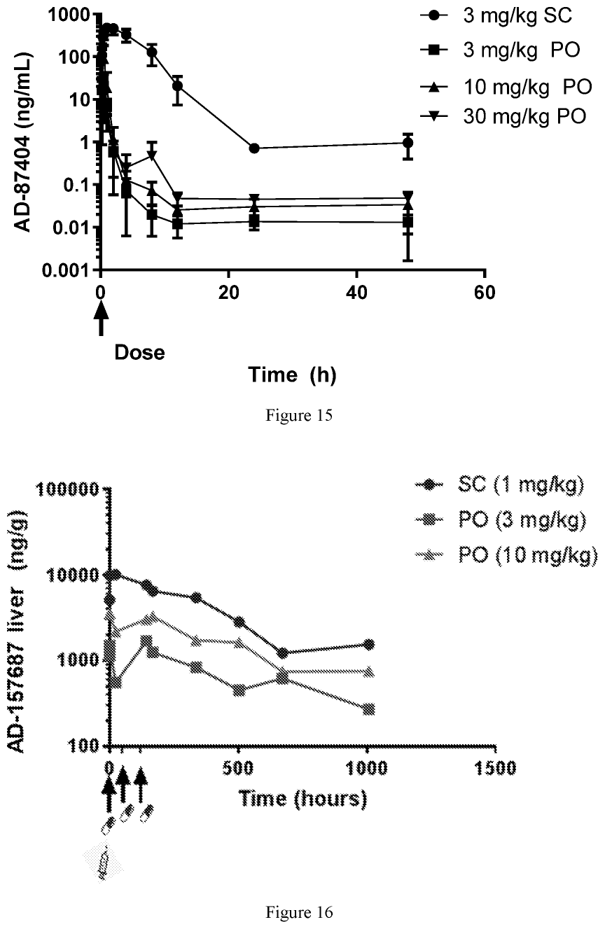

FIG. 14 and FIG. 15 indicate the plasma PK results of the orally administered GalNAc-siRNA in NHP. Most of the GalNAc-siRNA is likely absorbed in the liver during first pass due to the binding between GalNAc ligand of the siRNA and ASGR of the hepatocyte, followed by endocytosis of the GalNAc-siRNA. Once the GalNAc-siRNA was present in the plasma, a rapid decrease in plasma concentration was observed.

Table 1 lists the results of plasma PK parameters, such as Tₘₐₓ, Cₘₐₓ, and AUCₗₐₛₜ, of the orally delivered GalNAc-siRNA in NHP. As shown in Table 1, after a single oral gavage administration of GalNAc-siRNA (AD-157687) at 3 to 10 mg/kg (with 150 mM C10 in solution), plasma exposure Cₘₐₓ increased approximately in a dose-proportional manner over the dose range evaluated, with a slightly greater than dose-proportional increase in AUC. After a single oral gavage administration of GalNAc-siRNA (AD-87404) between 3 and 30 mg/kg (with 150 mM C10 in solution), plasma exposure (Cₘₐₓ and AUCₗₐₛₜ) increased approximately in dose-proportional manner over the dose range evaluated. Similar plasma Cₘₐₓ in AD-157687 and AD-87404 were observed. Both AD-157687 and AD-87404 reached maximum plasma exposure within 1 hour with Tₘₐₓ at 0.25-0.5 hour, suggesting rapid absorption into the systemic circulation following oral administration.

TABLE 1
Plasma PK parameters of the orally delivered GalNAc-siRNA in NHP
Plasma PK

| | Dose | | | AD-157687 | AD-87404 |
|---|---|---|---|---|---|
| | Level | AD-157687 | AD-87404 | SC | SC |
| End point | (mg/kg) | PO | PO | (1 mg/kg) | (3 mg/kg) |
| Tₘₐₓ (h) | 3 | 0.25-0.5 | 0.25-0.5 | 1 | 1-2 |
| | 10 | 1 | 0.25-0.5 | | |
| | 30 | N/A | 0.25-0.5 | | |
| Cₘₐₓ | 3 | 31.2 ± 4.24 | 21.5 ± 16.6 | 138 ± 10.3 | 508 ± 114 |
| (ng/mL) | 10 | 120 ± 36.6 | 112 ± 89.4 | | |
| | 30 | N/A | 310 ± 64.7 | | |
| AUCₗₐₛₜ | 3 | 30.1 ± 3.88 | 19.0 ± 13.1 | 709 ± 151 | 290 ± 348 |
| (h*ng/mL) | 10 | 174 ± 58.3 | 76.0 ± 70.7 | | |
| | 30 | N/A | 166 ± 21.5 | | |

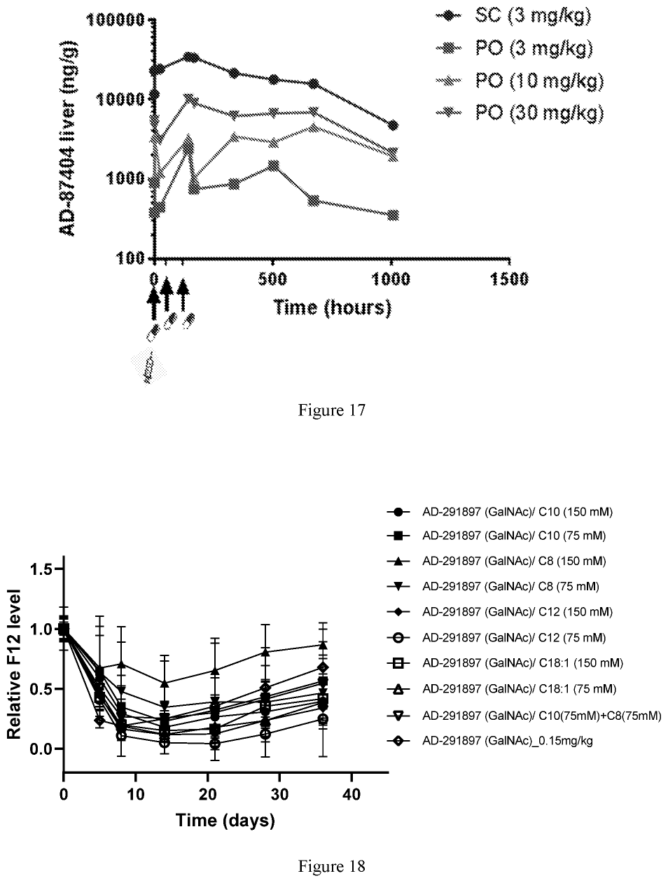

FIG. 16 and FIG. 17 indicate liver PK results of the orally administered GalNAc-siRNA in NHP. Slightly less than dose proportional increase in exposure was observed between 3 mg/kg and 10 mg/kg following oral administration of AD-157687, and between 10 mg/kg and 30 mg/kg following oral administration of AD-87404, respectively.

Table 2 lists the results of liver PK parameters, such as t_{1/2}, Tₘₐₓ, Cₘₐₓ, and AUCₗₐₛₜ, of the orally delivered GalNAc-siRNA in NHP. As shown in Table 2, similar liver Tₘₐₓ results were observed for various dose levels and for oral and subcutaneous administration of AD-87404. Comparing the results in Table 1 and Table 2, the liver AUCₗₐₛₜ following oral administration was substantially higher than the plasma AUCₗₐₛₜ, because both GalNAc-siRNAs (AD-157687 and AD-87404) targeted to liver during the first pass. A higher plasma exposure and lower liver exposure were observed for AD-157687 than for AD-87404, particularly at 10 mg/kg.

TABLE 2
Liver PK parameters such as t_{1/2}, Tₘₐₓ,
were indicated Cₘₐₓ, and AUCₗₐₛₜ in Table 2.
Liver PK

| | Dose | | | AD-157687 | AD-87404 |
|---|---|---|---|---|---|
| | Level | AD-157687 | AD-87404 | SC | SC |
| End point | (mg/kg) | PO | PO | (1 mg/kg) | (3 mg/kg) |
| t_{1/2} (h) | 3 | 416 | 267 | 328 | 319 |
| | 10 | 382 | 913 | | |
| | 30 | N/A | 285 | | |
| Tₘₐₓ (h) | 3 | 144 | 144 | 24 | 144 |
| | 10 | 6 | 168 | | |
| | 30 | N/A | 144 | | |
| Cₘₐₓ | 3 | 1.69 | 2.4 | 10.1 | 34.2 |
| (ng/mL) | 10 | 3.55 | 7.02 | | |
| | 30 | N/A | 10 | | |
| AUCₗₐₛₜ | 3 | 719 | 869 | 3940 | 18800 |
| (h*ng/mL) | 10 | 1610 | 3570 | | |
| | 30 | N/A | 6170 | | |

### Example 4A. PD Study in Mice for Different Penetration Enhancers

The exemplary siRNA used in this example was the same as AD-291897 Duplex shown in the table in Example 2A. The experimental design was the same as in Example 2A.

In this example, various intestinal permeation enhancer were used, including the sodium salt of caprylic acid (C8), capric acid (C10), lauric acid (C12), and oleic acid (C18:1), according to the protocols shown in the table below. The procedures for formulation preparation, formulations with and without the penetration enhancer, in vivo evaluation, fasting, and dose response are the same as those described in Example 2A, but according to the protocols shown in the table below. F12 protein levels from plasma were determined at days 0, 5, 8, 14, and 21 after bleeding.

| | Test/Control | | | Dose | Route and | Blood |
|---|---|---|---|---|---|---|
| Group | Article | N | Target | (mg/kg) | Regimen | collection |
| 1 | AD-291897 | 4 | F12 | 3 | Oral | Day |
| | (GalNAc)/ | | | | (fasting) | 0, 5, |
| | C10 (150 mM) | | | | on Days | 8, 14, |
| 2 | AD-291897 | 4 | | 3 | 0, 2, 5 | and 21 |
| | (GalNAc)/ | | | | | |
| | C10 (75 mM) | | | | | |
| 3 | AD-291897 | 4 | | 3 | | |
| | (GalNAc)/ | | | | | |
| | C8 (150 mM) | | | | | |
| 4 | AD-291897 | 4 | | 3 | | |
| | (GalNAc)/ | | | | | |
| | C8 (75 mM) | | | | | |
| 5 | AD-291897 | 4 | | 3 | | |
| | (GalNAc)/ | | | | | |
| | C12 (150 mM) | | | | | |
| 6 | AD-291897 | 4 | | 3 | | |
| | (GalNAc)/ | | | | | |
| | C12 (75 mM) | | | | | |
| 7 | AD-291897 | 4 | | 3 | | |
| | (GalNAc)/ | | | | | |
| | C18:1 (150 mM) | | | | | |
| 8 | AD-291897 | 4 | | 3 | | |
| | (GalNAc)/ | | | | | |
| | C18:1 (75 mM) | | | | | |
| 9 | AD-291897 | 4 | | 3 | | |
| | (GalNAc)/ | | | | | |
| | C10 (75 mM) + | | | | | |
| | C8 (75 mM) | | | | | |
| 10 | AD-291897 | 4 | | 0.15 | S.C. on | |
| | (GalNAc) | | | | Day 0 | |

The results are shown in FIGS. 18-22. FIG. 18 summarizes the results in one graph showing the relative F12 levels following oral delivery to fasting mice of a dose of a formulation containing a GalNAc-siRNA of 3 mg/kg at days 0, 2, and 5, when the penetration enhancer was C10 at 150 mM or 75 mM, C8 at 150 mM or 75 mM, C12 at 150 mM or 75 mM, C18:1 at 150 mM or 75 mM, or a combination of 75 mM C10 and 75 mM C8. A comparison was made employing the same siRNA administered subcutaneously at a single dose of 0.15 mg/kg.

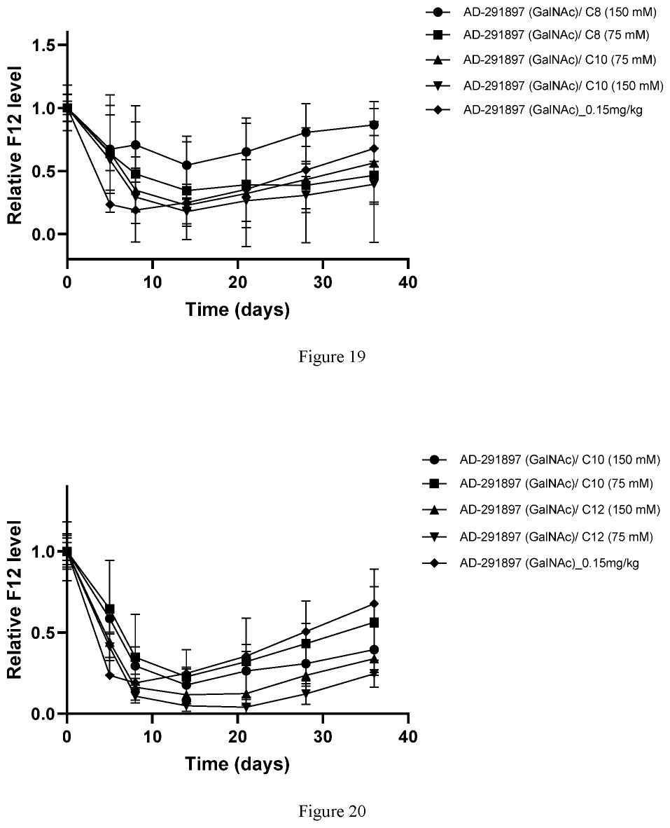

FIG. 19 illustrates the relative F12 levels following oral delivery to fasting mice of a dose of a formulation containing a GalNAc-siRNA of 3 mg/kg (with C10 at 150 mM or 75 mM) at days 0, 2, and 5, as compared against the relative F12 levels following oral delivery to fasting mice of a dose of the same siRNA of 3 mg/kg (with C8 at 150 mM or 75 mM) at days 0, 2, and 5. A comparison was also made employing the same siRNA administered subcutaneously at a single dose of 0.15 mg/kg.

FIG. 20 illustrates the relative F12 levels following oral delivery to fasting mice of a dose of a formulation containing a GalNAc-siRNA of 3 mg/kg (with C10 at 150 mM or 75 mM) at days 0, 2, and 5, as compared against the relative F12 levels following oral delivery to fasting mice of a dose of the same siRNA of 3 mg/kg (with C12 at 150 mM or 75 mM) at days 0, 2, and 5. A comparison was also made employing the same siRNA administered subcutaneously at a single dose of 0.15 mg/kg.

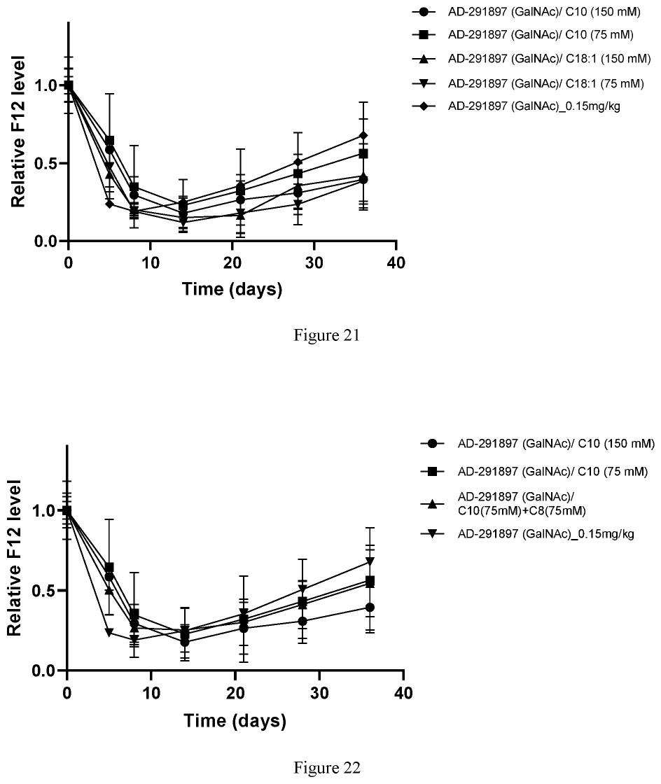

FIG. 21 is a graph showing the relative F12 levels following oral delivery to fasting mice of a dose of a formulation containing a GalNAc-siRNA of 3 mg/kg (with C10 at 150 mM or 75 mM) at days 0, 2, and 5, as compared against the relative F12 levels following oral delivery to fasting mice of a dose of the same siRNA of 3 mg/kg (with C18:1 at 150 mM or 75 mM) at days 0, 2, and 5. A comparison was also made employing the same siRNA administered subcutaneously at a single dose of 0.15 mg/kg.

FIG. 22 is a graph showing the relative F12 levels following oral delivery to fasting mice of a dose of a formulation containing a GalNAc-siRNA of 3 mg/kg (with C10 at 150 mM or 75 mM) at days 0, 2, and 5, as compared against the relative F12 levels following oral delivery to fasting mice of a dose of the same siRNA of 3 mg/kg (with 75 mM C10 in combination with 75 mM C8) at days 0, 2, and 5. A comparison was also made employing the same siRNA administered subcutaneously at a single dose of 0.15 mg/kg.

### Example 4B. PD Study in Mice for Different Permeation Enhancers

The exemplary siRNA used in this example was the same as AD-291897 Duplex shown in the table in Example 2A. The experimental design was the same as in Example 2A.

In this example, various intestinal permeation enhancers were used, including the salcaprozate sodium (SNAC), ethylenediaminetetraacetic acid (EDTA), C8, C10, C12, and C18:1, according to the protocols shown in the table below. The procedures for formulation preparation, in vivo evaluation, and fasting procedure are the same as those described in Example 2A, but according to the protocols shown in the table below. F12 protein levels from plasma were determined at days 0 (pre-dose), 5, 8, 14, 21, and 28 after bleeding.

| | Test/Control | | | Dose | Route and | Blood |
|---|---|---|---|---|---|---|
| Group | Article | N | Target | (mg/kg) | Regimen | collection |
| 1 | AD-291897 | 4 | F12 | 3 | Oral | Days |
| | (GalNAc)/ | | | | (fasting) | 0, 5, 8, |
| | C10 (75 mM) | | | | on Days | 14, 21, |
| 2 | AD-291897 | | | | 0, 2, 5 | and 28 |
| | (GalNAc)/ | | | | | |
| | SNAC (75 mM) | | | | | |
| 3 | AD-291897 | | | | | |
| | (GalNAc)/ | | | | | |
| | EDTA (75 mM) | | | | | |
| 4 | AD-291897 | | | | | |
| | (GalNAc)/ | | | | | |
| | C18:1 (75 mM) | | | | | |
| 5 | AD-291897 | | | | | |
| | (GalNAc)/ | | | | | |
| | C12 (75 mM) | | | | | |
| 6 | AD-291897 | | | | | |
| | (GalNAc)/ | | | | | |
| | C8 (75 mM) | | | | | |
| 7 | AD-291897 | | | 0.15 | S.C. on | |
| | (GalNAc) | | | | Day 0 | |

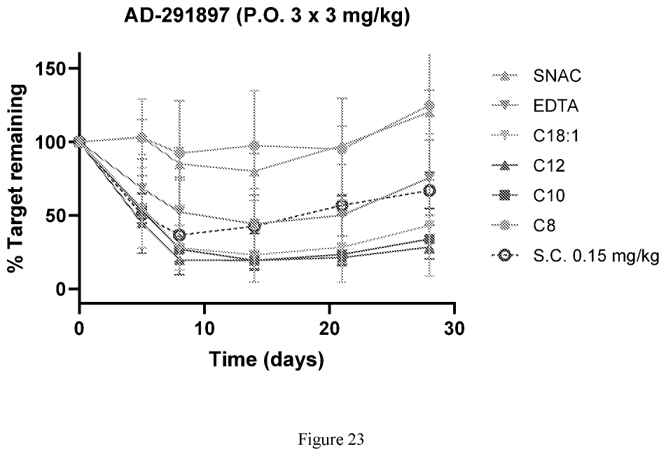

FIG. 23 illustrates the relative F12 levels in plasma following oral delivery to fasting mice of a dose of a formulation containing a GalNAc-siRNA of 3 mg/kg on days 0, 2, and 5, when different permeation enhancers of C10, SNAC, EDTA, C18:1, C12, and C8 were included at a concentration of 75 mM in the oral formulations of GalNAc-siRNA, respectively. A comparison was made employing the same siRNA administered subcutaneously at a single dose of 0.15 mg/kg. As shown in the figure, maximum KD was achieved through oral administration of a formulation containing GalNAc-siRNA with 75 mM of C10, C12, or C18:1; for the formulation containing any of these three penetration enhancers, the level of KD was greater and more durable than that obtained from a subcutaneous administration of the same GalNAc-siRNA. Oral administration of a formulation containing GalNAc-siRNA with 75 mM EDTA resulted in a PD profile similar to that obtained from a subcutaneous dosing of the same GalNAc-siRNA. Oral administration of a formulation containing GalNAc-siRNA with 75 mM of SNAC or C8 did not appear to have significant impact on the plasma F12 level at this dosing regimen.

### REFERENCES

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

1. A method for reducing or inhibiting the expression of a target gene in a subject, comprising:
orally administering to the subject in need thereof a formulation comprising:
a) a double-stranded iRNA agent comprising:
an antisense strand which is complementary to the target gene;
a sense strand which is complementary to said antisense strand;
2′-OMe modifications to more than fifteen, more than twenty, more than twenty-five, or more than thirty nucleotides; and
a carbohydrate-based ligand conjugated to at least one of the strands, optionally via a linker or carrier, and
b) a penetration enhancer.

2. The method of claim 1, wherein the double-stranded iRNA agent is administered at no more than about 50 mg per kg body weight, and

3. The method of claim 2, wherein the double-stranded iRNA agent is administered at about 1 to about 30 mg per kg body weight.

4. The method of claim 3, wherein the double-stranded iRNA agent is administered at about 3 to about 25 mg per kg body weight.

5. The method of claim 1, wherein the concentration of the penetration enhancer in the formulation is no more than about 200 mM

6. The method of claim 5, wherein the concentration of the penetration enhancer in the formulation is no more than about 150 mM.

7. The method of claim 1, wherein the formulation is administered in a single dosage.

8. The method of claim 1, wherein the formulation is administered in multiple dosages.

9. The method of claim 1, wherein the sense and antisense strands are each 15 to 30 nucleotides in length.

10. The method of claim 9, wherein the sense and antisense strands are each 21 to 23 nucleotides in length.

11. The method of claim 9, wherein the double-stranded iRNA agent comprises a single-stranded overhang of 1, 2 or 3 nucleotides in length on at least one of the termini.

12. The method of claim 11, wherein the sense strand is 21-nucleotide in length, and the antisense strand is 23-nucleotide in length, wherein the strands form a double-stranded region of 21 consecutive base pairs having a 2-nucleotide long single-stranded overhangs at the 3′-end.

13. The method of claim 1, wherein the double-stranded iRNA agent comprises a phosphate mimic at the 5′ end of a strand, selected from the group consisting of 5′-phosphorodithioate (5′-PS₂), 5′-vinylphosphonate (5′-VP), 5′-methylphosphonate (5′-MePhos), and 5′-deoxy-5′-C-malonyl.

14. The method of claim 13, wherein the phosphate mimic is 5′-vinylphosphonate (5′-VP).

15. The method of claim 13, wherein the phosphate mimic is at the 5′ end of the antisense strand.

16. The method of claim 1, wherein the double-stranded iRNA agent comprises at least two blocks of two consecutive phosphorothioate or methylphosphonate internucleotide linkage modifications.

17. The method of claim 16, wherein the antisense strand comprises at least two consecutive phosphorothioate internucleotide linkage modifications within positions 18-23 of the antisense strand, counting from the 5′-end of the antisense strand; and the sense strand comprises at least two consecutive phosphorothioate internucleotide linkage modifications within position 1-5 of the sense strand, counting from the 5′-end of the sense strand.

18. The method of claim 1, wherein the double-stranded iRNA agent comprises less than 12, less than 11, less than 10, less than 9, less than 8, less than 7, less than 6, less than 5, less than 4, or less than 3 of 2′-F modifications.

19. The method of claim 1, wherein the sense strand comprises 2′-F modifications at positions 7 and 9-11, counting from the 5′-end of the sense strand.

20. The method of claim 1, wherein the antisense strand comprises 2′-F modifications at positions 2, 6, 14, and 16, counting from the 5′-end of the antisense strand; or at positions 2, 6, 8-9, 14, and 16, counting from the 5′-end of the antisense strand.

21. The method of claim 1, wherein the carbohydrate-based ligand is conjugated to the double-stranded iRNA agent via a carrier that replaces one or more nucleotide(s) in the terminal position(s), wherein the carrier is a cyclic group selected from the group consisting of cyclohexyl, pyrrolidinyl, pyrazolinyl, pyrazolidinyl, imidazolinyl, imidazolidinyl, piperidinyl, piperazinyl, [1,3]dioxolanyl, oxazolidinyl, isoxazolidinyl, morpholinyl, thiazolidinyl, isothiazolidinyl, quinoxalinyl, pyridazinonyl, tetrahydrofuranyl, and decalinyl; or is an acyclic moiety based on a serinol backbone or a diethanolamine backbone.

22. The method of claim 1, wherein the carbohydrate-based ligand is an ASGPR ligand attached to the 3′ end or the 5′ end of the sense strand.

23. The method of claim 22, wherein the ASGPR ligand is one or more GalNAc derivatives attached through a bivalent or trivalent branched linker.

24. The method of claim 23, wherein the ASGPR ligand is:

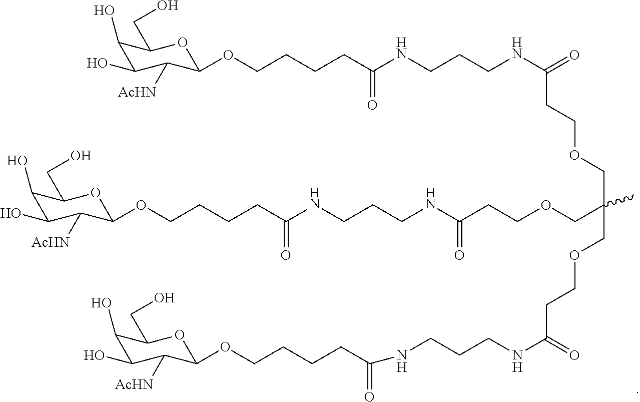 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

25. The method of claim 1, wherein the penetration enhancer is selected from the group consisting of a fatty acid or pharmaceutically acceptable salt thereof, a bile acid or pharmaceutically acceptable salt thereof, a chelating agent, a surfactant, a non-chelating non-surfactant agent, and a chitosan or derivative thereof.

26. The method of claim 25, wherein the penetration enhancer is a fatty acid or pharmaceutically acceptable salt thereof, selected from the group consisting of arachidonic acid, oleic acid, lauric acid, capric acid, caprylic acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, a C₁₋₁₀ alkyl ester, monoglyceride, diglyceride, and a pharmaceutically acceptable salt thereof.

27. The method of claim 25, wherein the penetration enhancer is sodium salt of caprylic acid (C8), capric acid (C10), lauric acid (C12), or oleic acid (C18); an ethylenediaminetetraacetic acid; or salcaprozate sodium.

28. The method of claim 25, wherein the penetration enhancer is chitosan or trimethyl chitosan chloride.

29. The method of claim 1, wherein the formulation is adapted for delivery as a capsule, soft elastic gelatin capsule, hard gelatin capsule, caplet, aerosol, spray, solution, suspension, or an emulsion.

30.-53. (canceled)


SEQUENCE LISTING (42 sequences)

SEQ ID NO: 1 - protein, 29 aa, Artificial Sequence
  Description of Artificial Sequence Synthetic peptide
AALEALAEALEALAEALEALAEAAAAGGC

SEQ ID NO: 2 - protein, 30 aa, Artificial Sequence
  Description of Artificial Sequence Synthetic polypeptide
AALAEALAEALAEALAEALAEALAAAAGGC

SEQ ID NO: 3 - protein, 15 aa, Artificial Sequence
  Description of Artificial Sequence Synthetic peptide
ALEALAEALEALAEA

SEQ ID NO: 4 - protein, 22 aa, Artificial Sequence
  Description of Artificial Sequence Synthetic peptide
GLFEAIEGFIENGWEGMIWDYG

SEQ ID NO: 5 - protein, 23 aa, Artificial Sequence
  Description of Artificial Sequence Synthetic peptide
GLFGAIAGFIENGWEGMIDGWYG

SEQ ID NO: 6 - protein, 48 aa, Artificial Sequence
  Description of Artificial Sequence Synthetic polypeptide
GLFEAIEGFIENGWEGMIDGWYGCGLFEAIEGFIENGWEGMIDGWYGC

SEQ ID NO: 7 - protein, 44 aa, Artificial Sequence
  Description of Artificial Sequence Synthetic polypeptide
GLFEAIEGFIENGWEGMIDGGCGLFEAIEGFIENGWEGMIDGGC

SEQ ID NO: 8 - protein, 35 aa, Artificial Sequence
  Description of Artificial Sequence Synthetic polypeptide
GLFGALAEALAEALAEHLAEALAEALEALAAGGSC

SEQ ID NO: 9 - protein, 34 aa, Artificial Sequence
  Description of Artificial Sequence Synthetic polypeptide
GLFEAIEGFIENGWEGLAEALAEALEALAAGGSC

SEQ ID NO: 10 - protein, 35 aa, Artificial Sequence
  Description of Artificial Sequence Synthetic polypeptide
  MOD_RES (17)..(17): Norleucine
GLFEAIEGFIENGWEGXIDGKGLFEAIEGFIENGW

SEQ ID NO: 11 - protein, 19 aa, Artificial Sequence
  Description of Artificial Sequence Synthetic peptide
LFEALLELLESLWELLLEA

SEQ ID NO: 12 - protein, 20 aa, Artificial Sequence
  Description of Artificial Sequence Synthetic peptide
GLFKALLKLLKSLWKLLLKA

SEQ ID NO: 13 - protein, 20 aa, Artificial Sequence
  Description of Artificial Sequence Synthetic peptide
GLFRALLRLLRSLWRLLLRA

SEQ ID NO: 14 - protein, 30 aa, Artificial Sequence
  Description of Artificial Sequence Synthetic polypeptide
WEAKLAKALAKALAKHLAKALAKALKACEA

SEQ ID NO: 15 - protein, 22 aa, Artificial Sequence
  Description of Artificial Sequence Synthetic peptide
GLFFEAIAEFIEGGWEGLIEGC

SEQ ID NO: 16 - protein, 26 aa, Artificial Sequence
  Description of Artificial Sequence Synthetic peptide
GIGAVLKVLTTGLPALISWIKRKRQQ

SEQ ID NO: 17 - protein, 8 aa, Artificial Sequence
  Description of Artificial Sequence Synthetic peptide
HHHHHWYG

SEQ ID NO: 18 - protein, 10 aa, Artificial Sequence
  Description of Artificial Sequence Synthetic peptide
CHKKKKKKHC

SEQ ID NO: 19 - protein, 16 aa, Drosophila sp.
RQIKIWFQNRRMKWKK

SEQ ID NO: 20 - protein, 14 aa, Human immunodeficiency virus
GRKKRRQRRRPPQC

SEQ ID NO: 21 - protein, 27 aa, Unknown
  Description of Unknown Signal sequence based peptide
GALFLGWLGAAGSTMGAWSQPKKKRKV

SEQ ID NO: 22 - protein, 18 aa, Mus sp.
LLIILRRRIRKQAHAHSK

SEQ ID NO: 23 - protein, 26 aa, Artificial Sequence
  Description of Artificial Sequence Synthetic peptide
GWTLNSAGYLLKINLKALAALAKKIL

SEQ ID NO: 24 - protein, 18 aa, Artificial Sequence
  Description of Artificial Sequence Synthetic peptide
KLALKLALKALKAALKLA

SEQ ID NO: 25 - protein, 9 aa, Artificial Sequence
  Description of Artificial Sequence Synthetic peptide
RRRRRRRRR

SEQ ID NO: 26 - protein, 10 aa, Artificial Sequence
  Description of Artificial Sequence Synthetic peptide
KFFKFFKFFK

SEQ ID NO: 27 - protein, 37 aa, Unknown
  Description of Unknown LL37 sequence
LLGDFFRKSKEKIGKEFKRIVQRIKDFLRNLVPRTES

SEQ ID NO: 28 - protein, 31 aa, Unknown
  Description of Unknown Cecropin P1 sequence
SWLSKTAKKLENSAKKRISEGIAIAIQGGPR

SEQ ID NO: 29 - protein, 30 aa, Unknown
  Description of Unknown Alpha-defensin sequence
ACYCRIPACIAGERRYGTCIYQGRLWAFCC

SEQ ID NO: 30 - protein, 36 aa, Unknown
  Description of Unknown Beta-defensin sequence
DHYNCVSSGGQCLYSACPIFTKIQGTCYRGKAKCCK

SEQ ID NO: 31 - protein, 42 aa, Unknown
  Description of Unknown PR-39 sequence
RRRPRPPYLPRPRPPPFFPPRLPPRIPPGFPPRFPPRFPGKR

SEQ ID NO: 32 - protein, 13 aa, Unknown
  Description of Unknown Indolicidin sequence
ILPWKWPWWPWRR

SEQ ID NO: 33 - protein, 16 aa, Unknown
  Description of Unknown RFGF peptide sequence
AAVALLPAVLLALLAP

SEQ ID NO: 34 - protein, 11 aa, Unknown
  Description of Unknown RFGF peptide analogue sequence
AALLPVLLAAP

SEQ ID NO: 35 - protein, 12 aa, Artificial Sequence
  Description of Artificial Sequence Synthetic peptide
RKCRIVVIRVCR

SEQ ID NO: 36 - RNA, 21 nt, Artificial Sequence
  Description of Artificial Sequence Synthetic oligonucleotide
gaaacucaauaaagugcuuua

SEQ ID NO: 37 - RNA, 23 nt, Artificial Sequence
  Description of Artificial Sequence Synthetic oligonucleotide
uaaagcacuuuauugaguuucug

SEQ ID NO: 38 - RNA, 21 nt, Artificial Sequence
  Description of Artificial Sequence Synthetic oligonucleotide
gaaacucaauaaagugcuuua

SEQ ID NO: 39 - RNA, 23 nt, Artificial Sequence
  Description of Artificial Sequence Synthetic oligonucleotide
uaaagcacuuuauugaguuucug

SEQ ID NO: 40 - RNA, 21 nt, Artificial Sequence
  Description of Artificial Sequence Synthetic oligonucleotide
ugggauuucauguaaccaaga

SEQ ID NO: 41 - RNA, 23 nt, Artificial Sequence
  Description of Artificial Sequence Synthetic oligonucleotide
ucuugguuacaugaaaucccauc

SEQ ID NO: 42 - DNA, 23 bp, Artificial Sequence
  Description of Artificial Sequence Synthetic oligonucleotide
  Description of Combined DNA/RNA Molecule Synthetic oligonucleotide
ucuuggtuacaugaaaucccauc